SEQUENCE LISTING (335 sequences)

SEQ ID NO: 1 - DNA, 62 bp, Bos taurus
ctctcttatttattcaacaaacatgggtgagcatctctacatgaagcactgaggaagagggg

SEQ ID NO: 2 - DNA, 841 bp, Bos taurus
cgcagtgttttgcggttctggtggcttctgcctgttcctcgggagccctcatccagagaaggctccggagcgggagattggacgcggccggttccgttccctagctgtccagccgcgcagggcggccagcgcggcccgggcgcacgcggaggcgccgggacctgcccgcctccaggctcccaggaccgtggccccgcgcgcgcccgcccgccgcctcccgctagctctgcgcccggggacccccgcccccgggaccagcccccggtgaggccccctactcaccgcggggcccgccaggaacaggagcgcccgtagctcccgccggaggtcggccagccgcagacagcgcggcgcgcggcgctccgggctgagcgcccgggctcccggcccgggtgtaagctcttcggacgcctccatgtaccgggcgccggccacggggagcgcgcaggaagccgaggggaggcccggccgggccagccgcgctccagtccccgccccgggccgcccgccgcccaccttggacgcgcccccgccgcccggcccaggtcgcccgcttagctggccaaggcctgccaactccgggactccgaatacacagttgaaggaagctccggaaagaagcagaaagtggaaagcgcggcgggggctgggtcctccggccctcgccggctccacacctcccagggctgggggtctgaaagccgcgtggaagcgctggcgggctggccactgagagcccgcgcccgcagccagaaacctccggctcccggagcgcctgctctgcacgcaggctgggagaccgcacaggacttgactgtgcggacgctcttcttcccgcccc

SEQ ID NO: 3 - DNA, 86 bp, Bos taurus
aagagaagggcatggcctcccagctcctagcacgttccaccaagcacttgcagtcatggtcactgccccaagtgtggagccttggc

SEQ ID NO: 4 - DNA, 312 bp, Bos taurus
agccatgctccctggggacacaaggctgtcccctgagcaccccgacccgccccaccagggattgattgttgcttctcagatgacacagcgtgcgaccagagctggttccagttgccagcagccacttgaggtgacggcagggcccctcctgcctgaaaccttcatgcttaagtttctcagctcgtgttcttctctgggcagacttgcccactcagggcccttgctgctgtgaagatcaaactaattaattacctacgaaactacgcggaagagtcttcccagcccgctggcgccatttgcctcttaagacaa

SEQ ID NO: 5 - DNA, 103 bp, Bos taurus
tccacccccacccgctgccctttaaatgcttgcctgagaaaaatcaaagctacctcaatcatttactatttattccagcaacatctgaaacaaaggcccctgc

SEQ ID NO: 6 - DNA, 64 bp, Bos taurus
aaaataaaaaatggggagatttgagagaatggcattgaaacatgtataatatcatgtatgaaac

SEQ ID NO: 7 - DNA, 63 bp, Bos taurus
accagccccagctggccacacttccctcagcagtccatttaccaaccatgtaatgtatccttc

SEQ ID NO: 8 - DNA, 309 bp, Bos taurus
aatctgttgtcaggtggagataagccgaggtagcaaagggcagtgtgctctcagcccctcccacgaacttctgcatccaccgtatggcagtgaccacaaaggctgggcctcctcatgtgggtcattgggactaaagacactagttccgtccctgccagcagggctaagtctagacggatctgatcaaaagggatggggggaagtgcatggacagagtggctgaggggtggccttaggcccctcttctaggtagcatgggagcagaataggaaactgtggcctctataggcgaggtcacggagggcttct

SEQ ID NO: 9 - DNA, 138 bp, Bos taurus
atcactgggtgacagtcgggacatcgcaggcagtctcgggtagaagcagaagctttttcaaactctgtgtgccccagggatctccttcagcccacctttgtgggctgtaaattgtagggcccatgggagagcaggaag

SEQ ID NO: 10 - DNA, 694 bp, Bos taurus
ttccctggaagccacttccctcccctgcctggcccagggtccctgggtgccctcacctgggcccagcccgctggtcccactcagaacacccttgggtccaggtcattgccagaaacaggttgggaggtgcctccaccccagcgagcaggccagcaggttgggcctgaggtgaccgagcccagacagccccatgagtgtccctgtcctgcccccaccatcgcgccaaggtcccacggcacgccacaccactggcctggccccgttaggaggcgtctctgctggaaccaacaccctatccccatggccgcggaccgcaccccttggacctgacctcctgcagtgcctggggacggagaggacagcagtgtgcgtcctgcccccaagggtccagggtcctcctcctgctgacctgttaggtctctggggcaaccctgggagggaggagaccgcgaggtgcccggcctcaggacaggtgggccacagcctccagacctgggtctgacgtctgtcagggagcgcagcgggtccaccccctgcaaaccaccccctgctcccccactgccgcccccgctgtctccccgtctgccctcccccagcccactcctccatcaccagctcctggagggctctcgggtttccaggcctgccccaagggctctgggagctctgcatgccctgggccccagccaagcac

SEQ ID NO: 11 - DNA, 184 bp, Bos taurus
agaagccgcactgcctgcacctgcccagctcactggagccccactggcacctctgaggacagccctgaccctgggctgtaggggctccactccgcaggcagactgaagccaggcgtccctgaggccgacacagcgtgggtgtgggtcagcatgggctgggggtatgtatactcgtgtgtgtgcc

SEQ ID NO: 12 - DNA, 1089 bp, Bos taurus
cgcggaagcatcaccgctgggcgctgcaccaaagggcagccaatcacgcaccgccttcgcccagattcctccgccggaggccgcgccgcagccgcaggtggcccggcgagcgcgtccacccagcgccgccaatcgcgacccccgccgcccgtcccgtgatgccccgcggctgacctgcgacctcgcggctcgcctttccctgaggccagctcttctccccgccccccctcacacccccccggcgagcttgttagggctctagcggccgcgaggcgtgctgccgggagcagctcctccctgagcgaccgcgggcctcctggcaggcgccttcccttaggctctcctcgcggcggggcccggcgggcgggtagccgcgaccgcgtccgggaggcactacccgtcggtgagtgggccgggcgcgaactccgcggggcggggaagcgggcgggcgccggcggcggcgcggggcggggcggcgggaggagcgggaagggggtggggcgtcggggcctcgcttcctccctcagcgccattttgtggcagcgagacccgcaaataaaggggagcgccggggttgcggcgcggaggaggagcgcgggggccggcggcggcggcgcggtcgggcggcctgggcggcgagaggacagcgcggggtcggcctatgtgactgggcgggcgccggcgcggcctccgtctggaaaggtaggtgcgggccgccgccggccgcagcggaggcgaggacctccgagtccccaggcggctgaggtgcggccgcccggccccggaaggggccagctgagcgcaggaggcggactcgggattcccgggccccccggcctcccgttagccccccgccatctgcccgctcacagggacgacacttccgccgtctcgaggccattttacctcggttctcagggagccgggggcgaggtgggcgctcccgcgggaccggcaacgccgggaggccgtcccctcggagcgcgcgtcccgtcccgtcagcgcctcggcgtcgcgggctcccccacctccctcggcaggatcgtcaggagtggcggcgccgatgagccgaggggaggaaagccgcgt

SEQ ID NO: 13 - DNA, 162 bp, Bos taurus
gtcacgagggtgccaggcctcaccatccttcgtctctggccgccctgctgtaaggacaccaagaagcccgtggagaggcctgagcgatggggacgaaggccacctgcccttccagcccctcgcccaaccaaggcggcgtggccaggccaaggcagagccaac

SEQ ID NO: 14 - DNA, 498 bp, Bos taurus
ctgagacagaagggccagccctgtctgcagcagaagggccaggtctggccaaggtagaagggccagcactggcccaggcagaagggccaggcctggccctggcagacatgtcagcactgggcaaggccaacgtgccagccctggctgcagcagaaatgccaagactggccaaggccgaagggctagcactggctgaggcacaagggctgggcgtggccctggcagaaatatcagcagtggctgaggccaaagtgcctgccctgtctgcaacagatgtgccaggtctggccaaggcagaagggtcagcactggccaaggcagaagggtcagcactggccgaggcagaagggccaggcctggccctggcagaaatgtcagctctgactggagcagaagtgccaggtctggccaaggccagaggaccaggcacaggccaggcactggcaaaagggccaggcctggccctggcagacatgtcagccctggctggagcagaag

SEQ ID NO: 15 - DNA, 1302 bp, Bos taurus
gaagttccagttctggccaaggcagaagggtcagcactggctgagacagaagggccagccctgtctgcagcagaagtgccaggtctggccaaggcagaagggccagcactggcccaggcagaagggccagcactggcccaggcagaaggcccaggcctggccctggcagacatgtcagcactggctgtgggagaagtgccaggactagccaaggcagagggccagcactggcttagccgaagggccaggcctgggcctggcagacatgtcagcactggccaaggccaaagtgccagccctggctgcagcagaaatgccaggactggccaaggacgaagggccaggcctggccctggcagatatgtcagcactggccaaggccaaagtgccagccttggttgcagcagaagtgccaagactggccaaggcagaagggccagccctgactgcatcagaagagccaggactggccaaggtagaaggtccagcagtagccaaggcagaagggccgggcctggccctggccgaggccgaagtaccagccctgggtggaacagaagtgccaggtctggccaaggcagaagggccagcactggcccaggcagacatgtcagccctggctgcagcagaagtgccagtactggctgaggcagaagggccaggcttggccctggcagacatgtcagccctggctggagcacaagtgccaggtctggccaaggcagaagggccagcactggcccaggcagacatgtcagcactggccgagcccgaagtaccagccctgggaggaacagaagtgccagatctggccaaggcagaagagccaggcctggccctggcagacatgtaagcactggccaaggccaaagggccagccctggctgcaggagaagtgccagtactggctgaggcagaagggccaggcctggccctggcagccatgtcagccctggctggagcagaagtgccaggtctggccaaggccgaagggttagcattggccaaggcagaaggaccatcactgaccctggcagacatgtcagccctggctcaggccgaaatgccagccctggctgcagcagaagtgccaggtctggccaagccagaagggccagaactggccgagtcagaagggccaggcctggccaaggcagaagggccagcactggatgaggccaatgtgccagccctggctgcagcagaagtgccaggtctggccaaggcagaagggtcagcactggccaagtccgaaatgccagccctggctgcatcagaagtgccagttctggccaaggcaga

SEQ ID NO: 16 - DNA, 370 bp, Bos taurus
gtcaccacattctggtatttcttgaccagcgccacgatcagggccttctgcttgggcgtcacgcggcagcagatgaccgcctggcatcgggaggccagctccacgaaggcccgctcccgccgctcctcagcactccggagggtcttggagtcttcgtgctggggcgccagccccgagctccgcagctggatccccagtgtccgccacatgagggacaggcgcctggcctgcaggaagtccaccctctcctctcccggctcctgccaggactccaacgggtccacgttcacgttctggacaggggctcggggttccttgcgcagggacagcagtagctggtcctggctcacaggtgacaggggtgggtcac

SEQ ID NO: 17 - DNA, 400 bp, Bos taurus
acaggggactcgtccagggagctagtgtgtctaggacatggggagcaggcatctcgctgctagagcacagacgcaggaagggagaggcgcgcgtgagccctgtgctgtgggactggcaccgaagagacaggcatgagatgcaccatgggagtcggctcagcgtagccagagcccacgggcccaagcgccgtcagctccccagtctgccctgaggaaccgggggtggtgggggtccctgggacgcgctggcactggcctcggggcagaagagcctaagacaagcccaggatgctctgtttcgccaaaagctaccaggaggtcacgactggtggcaagagcggagagggcaccaaggccagcccggccggcggccactggtcacagtgggcggcgtgggaac

SEQ ID NO: 18 - DNA, 66 bp, Bos taurus
actgtggcacaggaatcaagacccagatttgagctaaggagaatctaggggagggggaaggaggga

SEQ ID NO: 19 - DNA, 178 bp, Bos taurus
acttgcacttcctgcccttggaagtgaccccctgttggagcctcccactcattttatttaccagctggggtctgcagggcccttcagcctggtggtgctgtttctgttctttgtcctctgttaagactttgaagttcctaagagctgagcccctaaacactgtgtccttcatgcccgc

SEQ ID NO: 20 - DNA, 36 bp, Bos taurus
atctgacgcacacgctctaaagcagctctcgatcct

SEQ ID NO: 21 - DNA, 106 bp, Bos taurus
caagcttagtaaagttgcttgcaggagtcaggtctaagggcagagtcagcacggaaacctcaacacctgactccagagctctttacacagggcgaccgcccgaggc

SEQ ID NO: 22 - DNA, 165 bp, Bos taurus
tagtctgccaggctccactgcccatgggattctccaggcaagaatactggagtgggttgccacttccttctccaggggattttccccacccagggattgaaccactgtcttttatgtctcctgcattggcaggcaggttcttttaacactactctacttctgctt

SEQ ID NO: 23 - DNA, 34 bp, Bos taurus
agacagatactcaagtgaaaacgaaagcatgtaa

SEQ ID NO: 24 - DNA, 2490 bp, Bos taurus
ggctgcagcagaagtgccagatctggccaaggcagaagagccaggcctggccctggcagacatgtcagcactggccaaggccaaagggccagccctggctgcaggagaagtgccagtactggctgaggcagaagggccaggcctggccctggcagccatgtcagccctggctggagcagaagtgccagatctggccaaggcagaagggcccgcactggctggggccgaagggctgggcctggatatggcagacatgtcagcgctggctgcagcagaagtgccagtactggctgaggcagaagggccaggcttggccctggcagacatgtcagccctggctggagcacaagtgccaggtctggccaaggcagaagggccagcactggcccaggcagacatgtcagcactggccgagcccgaagtaccagccctgggtggaacagaagtgccagatctggccaaggcagaagagccaggcctggccctggcagacatgtcagcactggccaaggccaaagggccagccctggctgcaggagaagtgccagtactggctgaggcagaagggccaggcctggccctggcagccatgtcagccctggctggagcagaagtgccaggtctggccaaggcagaagggttagcattggccgaggcagaaggaccatcactgaccctggcagacatgtcagccctggctcaggccgaaatgccagccctggctgcagcagaagtgccaggtctggccaagccagaagggccagaactggccgaggcagaagggccaggcctggccaaggcagaagggccagcactggatgaggccaatgtgccagccctggctgcagcagaagtgccaggtctggccaaggcagaagggccagcactggatgaggccaaagtgccagccctggctgcagcagaagggcctggtctggccaaggcagaagggtcagcactggccaagtccgaaatgccagccctggctgcatcagaagtgccagttctggccaaggcagaagggccagcactggctgaggcagaagggccaggcctggccctggcagatatgtcagtgctcgtcggagcagaagttccagttctggccaaggcagaagggtcagcactggctgagacagaagggccagccctgtctgcagcagaagggccaggtctggccaaggcagaagggccagcagtagcccaggcagaagggccaggcctggccctggcagacatgtcagcactggctgcaggcagaagtgccaggactagccaaggcagaagggccagcactggcttagccgaagggccaggcctgggcctggcagacatgtcagcactggccaaggccaaagtgccagccctggctgcagcagaaatgccaggactggccaaggcagaagggccagcactggatgaggccaaagtgccagccctggctgcagcagaagggcctggtctggccaaggcagaagggtcagcactggccaagtccgaaatgccagccctggctgcatcagaagtgccagttctggccaaggcagaagggccagcactggctgaggcagaagggccaggcctggccctggcagatatgtcagtgctcgtcggaacagaagttccagttctggccaaggcagaagggtcagcactggctgagacagaagggccagccctgtctgcagcagaagggccaggtctggccaaggcagaagggccaggcctggccctggcagatatgtcagcactggccaaggccaaagtgccagccttggttgaagcagaagtgccaagactggccaaggccaaagggccagcactggctgaggcagaagggccagcactggctgaggcagaagggccagcactggctgaggcagaagggagaagggccaggcctggccctggcagatatgtcagtgctcgtcggagcagaagttccagttctggccaaggcagaagggtcagcactggctgagacagaagggccagccctgtctgcagcagaagggccaggtctggccaaggcagaagggccagcactggcccaggcagaagggccaggcctggccctggcagacatgtcagcactggctgcaggagaagtgccaggactagccaaggcagaagggccagcactggcttagccgaagggccaggcctgggcctggcagacatgtcagcactggccaaggccaacgtgccagccctggctgcagcagaaatgccaggactggccaaggacgaagggccaggcctggccctggcagatatgtcagcactggccaaggccaaagtgccagccttggttgcagcacaagtgccaagactggccaaggccaaagggccagcactggctgaggcagaagagccaggcctggccatggcagacatgtcagcactggccaaggccaaagtgccagccctggctgcagcagaaatgccaagactggccaaggccgaagggccagcactggctgaggcaca

SEQ ID NO: 25 - DNA, 343 bp, Bos taurus
tgctgagggcctccccacaggctgacccccacagcttagcacggctgcaggcccgcctctgaggctgacacggacttcagcataggtgacaaagcctgggacctgggcctcttgagtgtaagcttcccaagggcagggactacgggatgtactggatgcctgctaggtgctcagtaaatcctcaatgagcgatgagcgaataaatggagtgtgacctctgaccagagactaagtctcttcacaggcctgcggcaaggctgtgtggccctctgcttcctgcccggcccagtgttaactcagtgtggaccccgctccctccctggacacaactggctcccaggaa

SEQ ID NO: 26 - DNA, 428 bp, Bos taurus
gtagggaccttcaggacagtgcatgagcccagcggagcccccacggcccagatcagccacgggcagggcgaccagcagaggggtgcagggcttgaccggtgtgcattcattctggtgtgtgtgagcacgtgtgtgtgtgcacgtgtgtgcacaattccaccagggctgtgctgggccctacactggggagtgggggagacctgggagctgactcatggggcgggatccagggtggtgtccagggctggggggctggaagagtggacatacaggctgaccacctccagtccagggggccgtgcctgggtgccctccaggtgccaaggggtccagtctccctgcccttcctgccttggtccaggccgagaaacggtggccccacaactcagcgggccagggccagggccgtggaggcagctgtcagagac

SEQ ID NO: 27 - DNA, 1025 bp, Bos taurus
tgaccccacgggcaaccctgaatgtggagacaggaccacctcccacctttccctgggaaccctgctggccctgaggaaccgggcctgggtgaggtctgtgtccctgccaccccagcctctcggctgactttcactctcccctggccagcgccctacagcaccgcggggccgggccccccgccgcccccaggggatggggcagcaggctgccggcgggagcgcaggcaggagcggggccggtcccaggagcggaggcagccctcctcctcttcctctgagaagcagcgcttctactcctgtgaccgctttgggggccgtgagcccccgcagcccaagccctctctcagcagccatcccacgtcgcccacggccgggcaggagccaggacccccgagacaggtacatgggccccgggaccccccatgctcccacccgcatcccattccctcgggccctgggagggagccctctcctgtcctggttcccaggccagagcctgctcctggcttcctggcctgtccatcctgcctggcggggccttggccttcctgctccccagttccttctttctctcctccccgcctgagatgggctgggctgtgctctcctcttccctggccctgtgcccagccccttctggcactgccctcccaccctcctggggcctcccgctccatctcacctacctgcgctcccagcctccctgccctttgatttgggtttgctctgttccgctttcttgcagggtagcggctccgtgaacgggagccccctgctgtcgacatctggtgctagcacccctggccgcggcgggcggaggcagctcccccagaccccgctgaccccccgtcccagcatcacctataagacggccaactcctcacctgtccactttgccggggctcagaccagcctccctgccttttcccccggccggcttagccgtggcctttcggaacacaacgccctgctccagagagaccccctcagccagccgctggcccccagctctcggattggctccg

SEQ ID NO: 28 - DNA, 368 bp, Bos taurus
cctctgccgggcgaggcagctctgctgacaaaggacgggcctcctttggagctggccctccgggcggggtgccggtgcgagccacggcggacggcggggggctgcagagtgcagctggtactgggcatgcagaccggggccgggagcccacgggctctacctgcctcgggggcccagggcagcacgtgttctggagacagaggccgcctggctactgaggcgctagaggtgatctccgccccggcttccggtgcgaccgggaaactgcggctcagagacttagggacgcacagcacagccagacggaggccagctagaccccaggccctgcccctgcctggcgtggcccagttcacccctgcgccact

SEQ ID NO: 29 - DNA, 1899 bp, Bos taurus
ctggctcttctgccttggccagatctggcacttctgctgcagccagggctgacatgtctgcctgggccagtgctggcccttctgccttggccagacctggcacttctgttccacccagggctggtacttcggcctcggccagggccaggcccggcctttctgccttggctactgctggaccttctaccttggccagtcctggctcttctgatgcagtcaaggctggcactttggccttggccagtgctgacatgtctgccagggccaggcctggctcttctgcctcagccagtgctggcccttcgggcttggccagtcctggcacctctcctacagccagggctgacatgtctgccagggcgaggactgacccctctgcctcagccagtactggcacttctcctgcagccagggctaacacttcggccttgcccaaggctgacatgtctgccagggacaggccctgcccttctgcctgagccagtgctggcccctctaccttggccagacctggcacttctgctccagccagggctgacatgtctgccagggccaggcctggcccttttgccagtgcctggcctgtgcctggtcctctggccttggccagacctggcacttctgctccagtcagagctgacatttctgccagggccaggcctggcccttctgcctcggccagtgctgacccttctgccttggccagtgctgacccttctgccttggccagacctggcacatctgttgcagacagggcaggcactttggcctcagccactgctgatatttctgccagggccacgcccagcccttgtgcctcagccagtgctagcccttcggccttggccagtcttggcatttctgctgcagccagggctggcacgttggccttgcccagtgctgacatgtctgccagggccaggcctggcccttcggctaagccagtgctggcccttctgccttggctagtcctggcacttctcctgcagccagtgctgacatgtctgccagggccaggcctggcccttctgcctgggccagtgctggcccttctgccttggccagacctggcccttctgctgcagacagggctggcccttctgtctcagccagtgctgacccttctgccttggccagaactggaacttctgctccgacgagcactgacatatctgccagggccaggcctggcccttctgcctcagccagtgctggcccttcggccttggccagtcttggcatttctgctgcagccagggctggcactttggccttggccagtgctgacatgtctgccatggccaggcctggctcttttgcttcagccagtgctggccctttgggcttggccagtcttggcacttctgctgcaaccaaggctggcactttggccttggccagtgctgacatatctgccagggccaggcctggcccttcgtccttggccagtcctggcatttctgctgcagccagggctggcacgttggccttggccagtgctgacatgtctgccaggcccaggcctggcccttcggctaagccagtgctggcccttctgccttggctagtcctggcacttctcctgcagccagtgctgacatgtctgccagggccaggcctggcccttctgcctgggccagtgctggcccttctgccttggccagacctggcccttctgctgcagacagggctggcccttctgtctcagccagtgctgacccttctgccttggccagaactggaacttctgctccgacgagcactgacatatctgccagggccaggcctggctcttctggcttggccagatctggcacttctgttccacccagggctggtacttcgggctcggccagtgctgacatgtctgcctgggccagtgctggcccttctgccttg

SEQ ID NO: 30 - DNA, 637 bp, Bos taurus
atggccggcaggtgagtgggcaaaggagcgtgccagggtgcacccagctcagcagcctggcatccagagaccactccccgcgagggctccctgtgccaaccagcagggcagcccgcagacgtttattacacttttgcgatgaaaacacaggttcaggaaccacgactggagccccgaggcccggggaggccacaggccccacctcgaggggccagggtcctgatgcatccgaggggggagatccacggggaggccccgggtacgcaacggcaccctggccagttgggggtgggtctttcagggcgtttgggccaggctcagctgaactgcttcccagagtgcggggctcaggtcaagagccaggctggcctggctcgctgggcgctgaccctgccaggacaggatgaggacaggtgcagacacaacgctttgctgccccggctctggggctgcagaccccaggcgtcaggggccttgttggttcagggcgagaggcgggaggcccggctgctgcgggatgtggacgcccggcctcgagcgtccacttggcctgtgggtccgtccctgtgccagtgccatccggccaggaggccccgctggcccgagaggaggctcccttggcccctgcttcagagac

SEQ ID NO: 31 - DNA, 117 bp, Bos taurus
agcaaaattgagaggaagatacagagttatcctgtatccccctgcccccacacatgcatagcctcccctaccattaacacccccaccagatggtacatttattacaactgatggacc

SEQ ID NO: 32 - DNA, 1138 bp, Bos taurus
gggctgacatgtctgccagggcgaggactgacccctctgcctcagccagtactggcacttctcctgcagccagggctgacacttcggccttgcccaaggctgacatgtctgccagggacaggccctgcccttctgcctgagccagtgctggcccctctaccttggccagacctggcacttctgctccagccagggctgacatgtctgccagggccaggcctggcccttttgccagtgcctggcctgtgcctggtcctctggccttggccagacctggcacttctgctccagtcagagctgacatttctgccagggccaggcctggcccttctgcctcggccagtgctgacccttctgccttggccagtgctgacccttctgccttggccagacctggcacatctgttgcagacagggcaggcactttggcctcagccactgctgatatttctgccagggccacgcccagcccttgtgcctcagccagtgctagcccttcggccttggccagtcttggcatttctgctgcagccagggctggcacgttggccttgcccagtgctgacatgtctgccagggccaggcctggcccttcggctaagccagtgctggcccttctgccttggctagtcctggcacttctcctgcagccagtgctgacatgtctgccagggccaggcctggcccttctgcctgggccagtgctggcccttctgccttggccagtcttggcacttctgctgcaaaaaaggctggcactttggccttggccagtgctgacatgtctgccaggggcaggcctggcccttctgcctcggctagtgctggcccttctgccttggccagtgcaggcccttctgccttggccagacctggcacttctttccagccagggctggcacttcgacctcggccagggctgacatgtctgccagggccaggcccggcccttctgcctcagccagtgctggcccttctgccttggccagacctggcactttttctgcaggcagggctgacccttctgtctcagccagtgccggcccttctgcctcggtcagtgctgacccttctgctttggccagtgctgacccttctgccttggccagacctgccacatctgttgcagacagggcag

SEQ ID NO: 33 - DNA, 102 bp, Bos taurus
cacgggcagcaggccaagatctgcctttcctctcccacccccagctccccacatcccatcccatttgagaagagaagggcaacgctgtctttccatcaattt

SEQ ID NO: 34 - DNA, 487 bp, Bos taurus
cttctcctgcagccagtgctgacatgtctgccagggccaggcctggcccttctgcctgggccagtgctggcccttctgccttggccagacctggcccttctgctgcagacagggctggcccttctgtctcagccagtgctgacccttctgccttggccagaactggaacttctgctccgacgagcactgacatatctgccagggccaggcctggcccttctgcctcagccagtgctggcccttctgcctcagccagtgctggcccttctgcctcagccagtgctggccctttggccttggccagtcttggcacttctgctgcaaccaaggctggcactttggccttggccagtgctgacatatctgccagggccaggcctggcccttctgccttggccagacctggcccttctgctgcagacagggctggcccttctgtctcagccagtgctgacccttctgccttggccagaactggaacttctgt

SEQ ID NO: 35 - DNA, 836 bp, Bos taurus
acttctcctgcagccagtgctgacatgtctgccagggccaggcctggcccttctgcctgggccagtgctggcccttctgccttggccagacctggcccttctgctgcagacagggctggcccttctgtctcagccagtgctgacccttctgccttggccagaactggaacttctgctccgacgagcactgacatatctgccagggccaggcctggcccttctgcctcagccagtgctggcccttctgcctcagccagtgctggccctttggccttggccaggcttggcacttctgctgcaaccaaggctggcactttggccttggccagtgctgacatatctgccagggccaggcctggcccttctgccttggccagacctggcccttctgctgcagacagggctggcccttctgtctcatccagtgctggcccttctgccttggccagacctggcacttctgctgcagccagggctggcacattggcctcatccagtgctggccccttctgccttggccaggcctggcccttctgcctcggccagttctggccctttctggcttggccagacctggcacttctgctgcagccagggctggcatttcggcctgagccagggctgacatgtctgccagggtcagtgatggtccttctgcctcggccaatgctaacccttctgccttggccagacctggcacttctgctccagccagggctgatatggctgctaggtccaggcctagcccttctgcctcagccagtactggcacttctcctgcagccagggctggccctttggccttggccagtgctgacatgtctgccagggccagg

SEQ ID NO: 36 - DNA, 120 bp, Bos taurus
caaaggaaaccccacatgcagacagaggtagaggctgtggaccagggagggccatgcaggccccagggagaagctggacttaggagaacgagaggctgtggacgcgggtccagcagaagg

SEQ ID NO: 37 - DNA, 786 bp, Bos taurus
accgtccgctcagtctctcctccctactgcctccaagtgcccagtgggccaggcaacgttggctgggctgcagaggcctggggggacagggacaacctccagctgcgccgccagccaggcccctgatggaagcaggcgctgtgccgggggctgggccccgcaggagggcaccgtcccgagcacggtggcccttggctaatggccgccgcaccggactggggcccctgcctccaagccctcgtgagcagcttccttcctgtgggggcagatgttcctcccggccccaggctcagagggcagggccacgtgggtagcaggagcccatgaccccagacccagctgtccccctactccccccagagttgctggcaggcggtccccgacagcaagagctctgggaccctcctggcctgggcaggcctctgaactcgccctgatgctctggacacctcagcctcctccaatccttctcagcccttggagcctgtccgtggattcccaccaggcgctggccctggagacccctgtgagccctgcctccttgggccaccgggaaggtgcagcctggctatgtttcgaggccaaggcaccatggtgcgcctgcgtgcggcccccgcccaccccgaggtgtcactgctggcccggtcaccggcccgtctggccacgccgacagctcggggaggcagccggacgtggcttcccatgacaagccgacaaccccggcccagcggggatccatgctcgtggggcctgtggacctcgtacaggccacaaacagcctctgtg

SEQ ID NO: 38 - DNA, 339 bp, Bos taurus
acaccctagtgcacccctgcctccatgtgtgagctggaacacatccctaaccctccaggccttggtttttccatctgggaaatggaagggtcaggtcccttccagctgagagggctctggccaaggtgctgagtcactagggacagtggggagggggcaggagaacagcctttcccaccggaaagggacctctgggagtggggacgcaagaggaacaggacaggctgtgcacaggggtgaaaggtggggaaggggtgcaggtgagtgacccaggccaggccagggagctccaggtaggtgctcataggggctgcagggccacctgccaggcaaggggag

SEQ ID NO: 39 - DNA, 34 bp, Bos taurus
ctaaaagctgtgcccactccctcctcaccctcct

SEQ ID NO: 40 - DNA, 891 bp, Bos taurus
gggaggtggcgctgccatggacgatggtgtccgcagtcaggtgcgagaaccgggtcagggccgccacgaggccggaggcgtctgctcggggaggacgacacaccctcaagctgcctcggagcgaggcctggtgctgcctccccgcccaagagccccaagggccccagcagcccagctcccagagcgcagcggggagcggggcaggcccagggcacacagctacaaagcagcagagcaggaatcaaaccggggtctgtgggaccccaggcccgcagggtgggtgggctccagggtgtggctcctcgcaggcggcaatggccggtgggcaccacggcctgcagcctctgccctgtcccatgcacatggcactcacctgccctggaggtcaggtactgggcatggcccttctccagggcgctcaccgcgtccagggcggcctgggccctgctcactaggtaatctgccagacaaggaggccaggtcaggggtggggcctgggctgaggccccagcacgcagcccacctctctcagggtgtagggcgggagggacagtgggcaggcgggcggccctaccgggggagctggtgcagcgcaggtgcagggggtcgtccagcttgcccaccgcgtcctgcaggatgcgttcagcctccgcggcagtagcccggagcatcgcgaactgctcgtccagcagcctctgctgtagcccctgctcctgagactcctggagggcgggggagacagaggggtggcagagaccccggccaggtgcccaagaccctcgctggggagggggctctggatagaggtcctgctccacccctcagtgcctgggtggctcctgccagagcagggcagccagctcagtgctaagaagggctcctgtccccagg

SEQ ID NO: 41 - DNA, 417 bp, Bos taurus
gtgattccgcgtgggcctccccaacctgctccccctctagagatgaccctgccactcccctgattgtggtcgggcaggacccaggtggcacgtatgccccccaaccagccccaggcccgggcctggaaccagagcaccgcggagctcctttgtagcccagccctgccgctctcctttgtctggtctccaccctttcaggaggttcgtggcccaggggaacccttgccaccgcctcccgcctcgggccttctcccacagagcagagcccctgctccacgaggcgatgggtgagcaggtgccgggccgagggcgtctgagtccacctcaccgttccgtggcagccgggccaaggacgtgaactccgtggatgtcactgcccaccagggggcccgaggaggcaggcggtgcttggggtcc

SEQ ID NO: 42 - DNA, 1145 bp, Bos taurus
cacccggggaggggagctcagaggaaactttggaagcctggagagggagtgaggctgccagggaggttgaggagggtcctgttcaaaggaccctgttcaaaaaatccctgcaatgattccggcaccttccagaatgcggaagggctgggaaggccaggctgtggggcagtgagggccgtgcaaccggggcagcagtggccaccccagggtatggatcatgctgctccgtggcacacggttccaccggtggaaggacatggtcccatggcttgtggacaaagagcgtggccagggaaggggcaggggtctgcaggccaccctgtgtcccagccaataggggaggcgcagggctgcagctgggggatgcaggggaggtggggaggggcccgggatgctgcctgcgggtggcggcaggcacagggaggaccagcgtgctccggcctggcctcggggagggctcccaggtgtctggggtggtttgaggcagccggtcttgtgctagagcagtctgaggggacggcttgccctggacccaccgacctgatggctctgctgtgggcagccagtgggggctgggagacagaccttggccatctctgagctctgctctctgtcccagcagaccctggccctccggggggctcccgggaggggcttcccagctgcaggtcccctgcctaggcgtcggggacttctctgtcaccacagacagaaaaatccccgcttcccttcgtcaccttgtgaagcgagaaccgcccctcccaccccctccgccctgggtctcagtttcattctgaaaggtcaggacgtgcaccgtgccagggggtggatgttgggggcacccagctcctgggcagggggtgtctctggactggtcctccctccaagcgcccggcagaccccgagccccagggacaggccccggcaagccgcccagcccggggctagtggccattgcctccgttgccccctctcctccgcgacagctgaggagaggggtcaaggatggggccaagaagtgggcaggcccctgccaggccgcacaagtcgtgcccatctatgggcatgcggcggccggaggcgcatgggagcccgggctgggtggcattgacgtgaagagccgttcccgagcgaggacacagctg

SEQ ID NO: 43 - DNA, 592 bp, Bos taurus
ccgtcggagtgagcggcaacgtggtgactgtgctgctgatcgggcgctaccgggacatgcggaccaccaccaacttatacctgggcagcatggccgtgtccgacctgctcatcctgctcgggcttcccttcgacttgtaccgcctgtggcgctcgcggccctgggtgtttgggcagcttctctgccgcctctcactctacgtgggcgagggctgcacctatgccacgctgctgcacatgacggcgctcgccgtggagcgctacctggccgtctgccgcccgctccgggcgcgcgtcctggtcacccgacgccgcgtccgcgcgctcatcgctgcgctctgggccgtggcgctgctctccgccgggcccttcttctttctggtgggcgtcgagcaggacgccggccacgatgcgctccgggacctaaactgcactgcgcagccctccgccttgccgccgcccttcctggggtcctctctggcttccccgcgatccccgccgtcggggcccgaagcagacgccgccgcgctgttcagccgggagtgccggccgagcccggcgcagctgggcgcgctgcgcgtaatgctgtgggt

SEQ ID NO: 44 - DNA, 580 bp, Bos taurus
ggaagacagaaaggaggctctcggaacctgggctcccccaccccctcccagggctttcacctgcgcccaggtgtatgcgggggaggggaacctgatcaacaacatccccccttcagccctatccccgccattcccgcatcccctggctccggcttatcctaagaacgtgagggtgccctgaccgtggtggggaacccggagaccctcacctgcgtgctctccactccctgccagactctagcggggcggggggcggacgtgtgagtctgggggagggaacgagctagatagagaaggggagtcagctcctagcggcccccggagttctccgagtcccgctggggggggaggggccgtatccccacccccgaggtttccatgggaagcgagaggggggcgtcgttgcctcatccccacccccagtctgacactccgggcgcgaggagggagagagggggaggcgcagagccccaaacaacaaagagcaggagccgcgagcggcagcccctcccccgggaggttgggggtcgcggccgcccggccctctggtcccctctcgaggggcctcgccatttgctcc

SEQ ID NO: 45 - DNA, 952 bp, Bos taurus
gtccccagaccctgggctgcgtgaagtgcttgtggcctgcaggatctcgacagaggaccccccgcaacggcccccctcctgtgaccagcgaagattccggggcaggacgctctgaagggcccagggaccgggaggccaaggagcagccacgggacggcctgggtgcacgggggtgccggggctggggtctgcctcctgcccggacacggatgagcgccccatgctgcgccagcccctgcccaccgctggcctctcggggggtcattcgggacaccgcgggcacggtgcgtcccggcctccccgggcagggcagcggcgcttctgtcatcaaagccccagcagaggcgtcgggctggggggactggggcccgcgcggcaggcccccccgtgctgaccgtcccacggacagcgccctttgtcccccagctgtcctcctggccagaggggagaggccctgtgggggccggcagggccgcggccgtgccccggacaccgagtcccccaggagccgccctgtgccgagcagtcttgcgtgagcggccccgccatctaaaaggctaatttgtgcaaatgagtgttccactcaccggcctccgcctaggaatgcaccttcccaccacgccgcacacattcctctcccggaaactttccatcctcagccggcctgctgggggcccagagccggcctcagggaagccggctccccgcgtggcatctgtctgcctttgggtccaggcggtaccagggccgatcagggccacttgaccaggcgctcgggggacggagcagttccgaggcccagggaagcagggctccagggttcaggactcagactctgctcatcggttcttccggcgagtcccaccctgagcccacagggggatgcccccacagcaaagcccgtggcgcccctgctgatggtcagtcccacaggtggaggtcccaaattagc

SEQ ID NO: 46 - DNA, 497 bp, Bos taurus
cgctctggctgcaccacctggagaagacttcccaggagggtgggagtttgcctcagcctttggtagcagcaacatctcccgcagcccaggggcagagtcaggaatgatgaagaccacctggcagctctgagacttacggcaacgcagacacagcgctgaagccaggctgctcatggcgcaggggcagtcccggtcacccggggagggggtcacccaggaagggggtcatctggggaggggggtcacccgggagcagggtcacctggggagtggggtcctccggggaagaaagtcacccggggaggggtcacctggggaaggaggtcaccagggaagggagtcacctgggagagaggtcacacggggagggggctcactggggagggctcacctggggagatgaagaaggtcctccacagcttcttgtccctcgtgacgtcccggatctccttggtcatgttgactagcctgccggccacgggaggaacccggcggaa

SEQ ID NO: 47 - DNA, 144 bp, Bos taurus
gatctgccaggtcccggcctttgggctgggcgacatcaacctgcttcagaacctgctgttcgggagcctcatctcggccgtggacccggtggccgtcctggccgtgttcgaggaggcgtgtgtgaacgagcagctatacatgat

SEQ ID NO: 48 - DNA, 105 bp, Bos taurus
gaggaaatggagcagactcaacagtaaacacaatccaaaagagaaaaatgcaaagagaaacaacagaagtgtttatacgtcaaaaagcagaaaactgaattcaca

SEQ ID NO: 49 - DNA, 44 bp, Bos taurus
aggcacattaaaggtcttatttctcagccttttaacttgcagaa

SEQ ID NO: 50 - DNA, 309 bp, Bos taurus
acagaagtgcccggtctggccaaagcagaagggccagcactggccgaggccgaagtgccaaccctggctgcaacacaagtgccaggtctggccaaggcagaagggtcagcactggccaagacagaagggccagcactggcccaggcagacatgtcagccctggccgaggccgaagtaccagccctgggtggaacagaagtgcccggtctggccaaagcagaagggccagcactggccgaggccgaagtgccaaccctggctgcaacagaagtgccaggtctggccaaggcagaagggccagcactggcc

SEQ ID NO: 51 - DNA, 381 bp, Bos taurus
cctgggtggaacagaagtgccacgtctggccaaagcagaagggccagcactggccgaggccgaagtgccaaccctggctgcaacacaagtgccaggtctggccaaggcagaagggccagcactggccgaggccgaagtaccagccctgggtggaacagaagtgccacgtctggccaaagcagaagggccagcactggccgaggccgaagtgccaaccctggctgcaacacaagtgccaggtctggcctaggcagaagggtcagcactggccaaggcagaagggtcagcactggcccaggcagaagggccaggcctggcccaggcagacatgtcagccctagctgcatgagaagtgccaggactggccaaggctgaagggcc

SEQ ID NO: 52 - DNA, 407 bp, Bos taurus
agcactggcccaggcagacatgtcagcactcctcggagcagaagtgccagttctggccaaggcagaagggtcagcactggcccaggcagaagggccaggcctggccctggcagacatatcagccctagctgaagcagaagtgccaggtctggccaaggcagaagggtcagcactggcccaggcagaaaggccaggcctggccctggcagacatgtcagccctcgtcggagcagaagtgccagttctggccaaggcagaagggtcagcactggcccaggcagaagggtcagcactggcccaggcagaaggccaggcctggccctggcagacatgtcagtactggacaaggccaaagtgccagccctgtctgcagcagaagtgccaggtctggccaaggcaaaagggcc

SEQ ID NO: 53 - DNA, 590 bp, Bos taurus
aacttctcctgagtcactggagcacgcccacgggacgtgagaggctgctgcaggcggctgtgccggtggggagcccctggccttgagaccgcagtctgcccgtgcccgcagggcctcccctgaccagcctccgtgtgagctcaccctcccaggcgcggcccgagctcgggggatccagggctcagcacctcttctcagggccgtatctctctcagctccgaaacacaagtggaaagtgcccccaggaggcctctttcgctgccagtgaggtcgcaaggaaggtcatcttactgctctggcctgggctgggtcggcctggctgggcagtggccttcttgtcctggtacctggggtcagggatgtgtcccagagccctgtctggttacaaggagggagccggccccccaggccaagggtctggttacagaagacaagactcagccaggcagtgtgtggacgagggggtgacagggcaggcctggggggccaggtggaggggtgtggtttgcagtggggcatggaaggaaaatgagggccggttgggtgggggccctggtcgcacacctgtgtcttccacctggaccaagctg

SEQ ID NO: 54 - DNA, 468 bp, Bos taurus
cagcactggcccaggcagacatgtcagccctggctgcagcagaagtgccagatctggccaaggcagaagagccaggcctggccctggcagacatgtcagcactggccaaggccaaagggccagccctggctgcaggcagaagtgccagtactggctgaggcagaagggctaggcctggacctagcagccatgtcagccctggctggagcagaagtgccaggtctggccaaggcagaagggttagcattggccgaggcagaaggaccatcactgaccctggcagacatgtcagccctggctcaggccgaaatgccagccctggctgcagcagaagtgccaggtctggatgaggccagaagggccagaactggccgaggcagaagggccaggcctggccaaggcagaagggccagcactggatgaggccaatgtgccagccctggctgcagcagaagtgccaggtctggc

SEQ ID NO: 55 - DNA, 1891 bp, Bos taurus
cccacacggccagctgcccctgcgcgctgctcaggaggaccctcagctcgccctggtctgggagcagggtgtgaagacgctcaggcagagtggggcggccccggcaccccctgccgcaccccggggctctggctcactcacagaagtgctcggggaagcccatgtggtaccagaagcgcagacaagaggccgtggtggggggcagaggctggctgatgagcccggccgctcggccccctgggcccagcacgctggtctcgaagagggcgaagtggcctggggatgagagagcgggtgcccagcggacccgatggagccggcgccccgatctgtggcctctgaccccgctgggcgctgggcgggccaggctggggtgaggcgtgtgtggctctgggtgcccttcctgccttggacccacctgcctctgtgcccagggtgtggtccacggggggctgcgggtagcgggagggcgaggctccgctgctccagtcccagctgtacccgcccaggccgggccggggcacatggttccagccacaaagaccagcctcgaagtcacaggaagctgcaggggccagccgtgagggtgatgccccgcgccctccacccctggggcaccgttgctgcgggtgtggaggtcaggccgggcgttgtgggggtggcagggagccaggaccaccaggagccaggccacaggcgcccacctggcagggggcagggcccatcctggaggagcaggtcgtccagtgccacgtaggagtgctccacgccggcggccactgcctcgaacaccacctggggggcaccgggcaaaggtcgtgcctgctgcgccctagacagcccttggggtgaggagggccccctattccactcaccctccaggcctgcccggcctgcaggtccacgctgcccaggcgccaggcggcccctccgcgggagctgacgctgagtacttgctgcctgccggcctcctccacgtggaccctcagggtgcctgccgccgagcagccagccccgtcagcgtggcaggggtgcctgctcaggcccctgtggtgggggggctcccaagggcctcctgaggggaccccacggccccggctcccagcagcccctcacctgggttgcggaggctcaggtggtaccagaaggtcaggcaggtgggctgcaccaggggccgctgctcctccgaggtcagcaaggccgcgtggccacggggcagggcctgcgggctcatgtccacgaccatgtagtgccctggggggtggcagggtcagcccactggccgccgccccgccctccccgccccacctgtctgcccactgccccaccccagtgggcaccctgagctgtctccgtggtgtggtcagcgtgggggccccacgaggcgttggcctggcgtgtccagaggccccggccccccacggagaagccacaggctgagtcctcaaaggagcagcgcctgggggcccagcagggcccgggccgcagggtcacatcgtccagtgccatgctgccgtggtacccatcccggaggccctcaaacagcagctggggggcagagagaggtcagcggctggggcctcgcctcacccaccgcacccacgccctgggcctcacctggtacttggcgcccgagtccggctggtggtggagggtggcccaggcttcgtgccagcagttgccgtgggtgccagaccgcgaccacaagtgcgtttctgcctccccttctcgcctcatcgccaggcgtagggtccctagagacagggggttggtggggagggaataaggccttaacccctgcctctcatcccctcccagggcccctgagggagcaagaggcaggacttggggccccggtgaggctcccttgaggccctgcccagctgctggc

SEQ ID NO: 56 - DNA, 306 bp, Bos taurus
tcgggcgaggactcagcctctctccacgggcccaaccccctgctcccactgacactcctctgccgccaagcgcttcccctgctgatgaccacccctcctgcagggacagggttctgggcacaggaagcagatgggctatgctgcccaggctgggcccacctccaggggctgctgatcccgggggagacctggctccactccaggctgcttggtccctctggcctcagcttccctatctatacaatgggcgcattgatggttttcacctgcagggctgcgcagagcgtcagagtcctgtggatgggc

SEQ ID NO: 57 - DNA, 332 bp, Bos taurus
agcgccgcctctgcgcccgcccgcgagggtctacgccccccaccgcagccctgtagacgcggttatccgtcttctgcccgctctccaaagcgcgtcctcctcagatgtccacgagcttcagacttcagctaagtaaacgctttaaagaagcggctgccttgtctgtgtagcagggcccagggccagtggtctgggtgacagtacccgagaaagtgccccctctcccgctgcacctgccactgagagctggaggccgagtgggaacatggcagggcgctcctgacagaccccagggtccttgtgctggacgcacagcagtgaccacgatgccc

SEQ ID NO: 58 - DNA, 1196 bp, Bos taurus
gacacagtggatccccagcttcaccaggcctggggggacagcaggcctcagaccccatccagccgggccacggctcgcaccctgcccttgggacccctcactcagcccctggggccctgggctcctgccctgcctggcgagtcctgagccagaccccaggaactctctgcccctactcctcaggcaccggccagaggctgcccccagcatggtccctgccccacgccacacggcggtctcggcctcctccagcctggcctcagcatggccctcactccctcgctagtgacacctggagtcgccccccagacctcagcggaaacccagcccctcagtggggagccagggtgacacccgtcccaccccacccacccatgctgggcctcaccgaccctgcaccactacccatcaccctccttctggcctgaggacctctggacagaggggcctcgggcacccgctcaccccaggacaccggtgcagggcccctcctgggcaggacgccatcaacacacctctgctgtggctgcagggaccctcggtcctgctcgctgcctcttggccacgtgggcacagctgggcacctgcccccctcccaatcccgcagagattcactgacagagcccctggcagcctctctccctccccagcctggttcctgtcctcctgtgccctctgtggcctcagacccaaacaccctctgccctccccgccccagcctgggccccgcctggacattccccgatgggcacggaggtgaccaccacagttggagccccctccagcacgtgggagcccagcggcagccgcgggggagacgccgggctccgcagctctgcagcctgtgcttggcctgggcggacgcccgtctcattcatgctgtgctgctctaactgtgacctcgacgcggctcgcagctccgaccagcggggtgtgcacttgcccctttggatgctgctgcggggcagctctcagaaaactgcaaagccctttgcttgagaaacaaatcttgccacatgcagcttcccctaaggggcatgcgtctctttatctgaccgttggtgtcgtttcatgaacgctcacaccttcccagcagctcgcgtcctcctccttgccccctgccccgcagcctcgcccatgggcagcaagctcgcgggtggctgggaggtgatccgtgctccccagaggcaggtggg

SEQ ID NO: 59 - DNA, 1106 bp, Bos taurus
tggtgggcacactggggtggacaggtgctccactggccgaagccccagctctgcccctgcgccccacgacatgtccggttgctatgctttgaatccctatgccaccctgcatgccaccctggccaagaatcaccccttacccgggccaagggccacccccactttgcgcaaaatgggaattcccacagcagcgaactatgtggcaagaccaccctggagtgccagcaacagggctgggtggggaggggcccagaaggacagcctttgatgggcacagggtctgggcagtgccacggccggctggaacgggacgggacagtcaggatcgccgggccggctgcccaggctgaggggctggtgtctcaggcccagcaatgcagccttgtttccagctcaggacccggcgggaggggtgggagccccgggacctcgcttggcctccccaaaccttcctggctggggcccagcagagagtcactgggtgcggccaactcagggctccaggccccggtttcccgccagctccccccacccctcccagcccaggccctttcttccaccccggaaggttcagaggagggagaggccggcagtctaggctgaaaaggaagagacaaaggcggcccagagggagcctggcagacactggtgtgccagcctgccccagccccccccagcccttgaggcccaagtgaagggggcgcagggacgcaggcctggcccccgtgctcatgccaagggcccagaccagcctggggagagccaatgggaggaagtgggggcatggaacagccagggcaagacaggaacgggagtgaattccccccaccaggggccggttggaactgcctcttggcccagagacatccttcgaccacgaccaccaccggactctggaaggcgggctggggggcatgcaacagggaggccctggcaggggcccactgggctgaaggcgagccggcctcctccgggaacctcggcggaggtggggctccagatgttctgcacatgcttggccctggctccaggcagaggaatgtggctgcagagaggtgccaggagaggccctgggggagggcctgcaacccacccccctcgaagga

SEQ ID NO: 60 - DNA, 1412 bp, Bos taurus
agacagccagctgacagctggaaaggggcgtctggggagtctgaatgtagctgccagcctggccctgatcaccctccttgccttagcatttacaggaagcgctggggctgcctgctaggaggggagatcaaaatcccgagggaaactctgaaggcaggggggcctgggcgaccgaggagaacgggcagggggaggaaggcgaggaggaacccacaccgacccccggcgtcagccccagcccctctcccaccccggaggacgccatcacttatatccgtaagtaatccttagctccgccctgcgacccccaaccccggcctccgtggtcattttcacaaaaaagaaccgtcctttccctgccccaggatttttcagttgcccaccgcccagctcggagccctcacacccaggagctccgggtccccggctttgcctttcaagagaccggggtggccaccggcccctaccttccagaaatcttgagggcccccacccctcaccccaactccgccccagaagccgtaggtcccgggtcccccccacgtcctccagtaccctggctccgcccccagggtccccgtccagcgtgcgccctgtccacccgcagtgggtcgcctggccggcctggacgcaggcctgcaccaggtgcacgtccgtctgcacgcgctggacacccgcgtggccgagcttactcgggggctgcggcagctgagggaagtggcgggcgacacccgcgacgccgtgcaagccctgcaggaggcgcagagccgcgccgagcgcgagcatggccgcttagagggtgagtcgcatcccgccgggcggggaaggagggacgatccaggtcaggccgcggaccggtggggagaggggagccccgaagggtgtctgagacttgaggaccctaggtgttaggggcgaggggagagcggccctcgaggatttggggtgtgctggctgatggggagggtgaacatggtcccaaccgtgctagtgagccgaccctgctccggccgtaccacgccccctacggtcccgcccccacgtcctggccacgccccctggcggctctgtcactggctccgggtgactctggtcccgccctcgacccgcccccgaggcactaaccccgcctccgctcggccccgccccgtcccgcccgcaggctgcctgaagggggcgcgtcttggacacaagtgcttcctgctctcgcgcgacttcgaggctcaggcggcggcgcaggcgcggtgcgtggcgcggggcgggagcctggctcagccagctgacagtcggcagatggagacgctcacccgctacctgcgcgcggcgctcgccccgtacaactggccggtgtggctgggcgtgcacgaccggcgcgccgagggcctgtacctcttcgagaacggccagcgcgt

SEQ ID NO: 61 - DNA, 849 bp, Bos taurus
tgccagcaccgtgcgctggactgcggggttgagatgccaccgttagccccagacacccaggtgtcctggagcccctgggcagacgcggtcagggcgtgggctacaggctcccccctcaccttgtctgctgcccatgagcacctccaccagctggaagccctcggccccctcggcctgcgggggtacatttcagggtatcaggcctggatgggaccctgccaccccaagactttggcagggagggcacatgggtggggcggggcgggcacacacctgccgtccgagcagcgggaggacctccagcaccacgctgcgggcagtgctctgcggggtcacacggatggacacgtaggccacacccaccctgcaggggacacgctcaggagctgcggaggcaaagcccgcactccttcccccctacccgccacggccctgctgggacctggtctcacttgagccaggccgggtagatcttcaggatctcctgggtgcggggcagagcacggaggacccaggcctcgggagccgctctgccgccctcctcctcagcgggcccagcgctccagacccttcccgcggccgcggtgtcgccaagctgcacaggcgtggggagtgcctgcagctggaagccctgagggtcctcggagatgtagtaagcccgcagagccgcctcctgtgagaggggagcaaaggtaaggcctgctggggccaggcctgtgggtgcccgcggtcccccacagccgggcgccctcttaccagcacctcctggctccgggccaggcgcgggaccgtgactgcacggaagtggtttcgctgcatcgcatcgctgccgtcaaagatcttcagagt

SEQ ID NO: 62 - DNA, 790 bp, Bos taurus
ggtgtcctggagcccctgggcagacgcggtcagggcgtgggctacaggctcccccctcaccttgtctgctgcccatgagcacctccaccagctggaagccctcggccccctcggcctgcgggggtacatttcagggtatcaggcctggatgggaccctgccaccccaagactttggcagggagggcacatgggtggggcggggcgggcacacacctgccgtccgagcagcgggaggacctccagcaccacgctgcgggcagtgctctgcggggtcacacggatggacacgtaggccacacccaccctgcaggggacacgctcaggagctgcggaggcaaagcccgcactccttcccccctacccgccacggccctgctgggacctggtctcacttgagccaggccgggtagatcttcaggatctcctgggtgcggggcagagcacggaggacccaggcctcgggagccgctctgccgccctcctcctcagcgggcccagcgctccagacccttcccgcggccgcggtgtcgccaagctgcacaggcgtggggagtgcctgcagctggaagccctgagggtcctcggagatgtagtaagcccgcagagccgcctcctgtgagaggggagcaaaggtaaggcctgctggggccaggcctgtgggtgcccgcggtcccccacagccgggcgccctcttaccagcacctcctggctccgggccaggcgcgggaccgtgactgcacggaagtggtttcgctgcatcgcatcgctgccgtcaaagatcttcagagt

SEQ ID NO: 63 - DNA, 341 bp, Bos taurus
ctgtgcccgcctctcctcctctgcccgcctcctgcacagtcgcatggctgcaggctcagctctgagggccccgggcagccctcttctggggtctacctgcggtgggctggcaccaacaggtcaggagctggcctggggggcctcgggcccctgccgccccggtccagggaagcaggacccctcgctccacgaggaaggccttcccgctcaggggaaacagccaggaggtggacatgctgggcccagaggctccgctcagggccacggggcagtgcccaaagatgcggcccgcacccggcagactctggttcccgacccagatggggcaaggggcctccctc

SEQ ID NO: 64 - DNA, 535 bp, Bos taurus
ggctcctgctgtactcttcaccccagacgcgggacccacgcggctcctgggggtccgggctctgatgctggcaggtgggggcaccgcaggtcaggcctacccaccacatcgccgtccctagagctgtccaggcaggccaggcagactcccaccagccacccggacacccgaatggtgagggtgtcctgtcctgatgggaatcgggcaccccaggtgctcaggctcccgtcgaagggtgtctgtgaccttccctgtgggggcttcacccacccctacagcttggccacacgtgaccctggaagaccctgccaagagtgggaagtgggcactcgggccctgagcctccagatgccaggcttcccctctgacccccaggccgggctccccggcgcccagcccgtcacctcccctggctgcacgccctacctctcgtgatgcgctccatgcccagcctccgtgccacggtgctcccacgcctctgcccctgtcggctctcaccagcctgcccgtctgggctgggggtgcgcggcagggc

SEQ ID NO: 65 - DNA, 403 bp, Bos taurus
gaaaagcgggtggagggggacaagaggctcctctccgccggcttgaggggagacacggtcccctgtctgagcagggccacttgaggcggagctggcaccagagcctgattgcacatagggtctctggctgcgggaccttcttgggggtataggggtgccaggataaagcgtttagttcctgagcggggaaggaggggtcctgcccgggaaaggagggggccggcgacactgaggatgccgcctttgtctgcaggcacactttgccaaggtagcgcccgtcccccctccccccaagaggtgagtggggcctggtccgactgattccttgcattttccgtttcttcccggctgggtcgtgggaaacgggcacgtgggtgtggctggcaccgccttcgttagaaag

SEQ ID NO: 66 - DNA, 505 bp, Bos taurus
ggcctggacttgcccacaggtccccaggagagcctggctcctccctaggtgggcagtggtcgaggagggcgtctggagagggttgccctggttttccctccctttatcccttctgcgggtctgcgtgagacgcaaacgtgcagaggtcggggccagatcacagctactccatgacagtcccttcccctccgggcacccgctgtcagtgggggggtgggggtgggaggggaggctggggctccagctgtcaacaggagaggtccggacaccccaccccctcgcatttgcccccagctcttgctaaagatcctccttcctttccctgaccccaggagctccgctgtgtggcctagcaaccagtacttcacacactcctccccatctttcccccagtatctgagactgcagccccgtgcgatctgtatctagagtgggatgtggtgcctggagagatgaattccagaggcggggatgggaaaagcctccttggaagagataagattta

SEQ ID NO: 67 - DNA, 304 bp, Bos taurus
aggactgggaaccagagtacccggggagtggccctgaactgggctgtacgccacagctgcctgcaggaaatagtcaccctcttaagtagaaaatgctcccaggaagagggagggggtctcatcctacctcaggggaggggacgtagccccccggggggagaaaggagcaagttagaggtgtgtacacaacctcagaacccatgaaggcgggcgggaggcagggtccaggcagcgctgtgactggagatgctgtgatggttgaacagggcccacctccgatatggggggcagagggccagttggg

SEQ ID NO: 68 - DNA, 649 bp, Bos taurus
ctttggccttgtccagtactgacatgtctgccagggccaggcctggccttctgcctgggccagtgctgacccttctgcctgggccagtgctgacccttctgccttggccagaactggcacttctgctccgacgagggctgacatgtctgccagggccaggcctggcccttctgcctgggccagtgctggcccttctgctttggccagacctggcacgtctgttccacccagggctggtacttcggcctcggccagggctgacatgtctgccagggccagtgatggcccttctgtcttggccagtgctgacccttctgccttggccagacctggcacttctgcttcagctagggctgacatgtctgccagggccaggcctggcccttctgccttggctagtgctggcccttctgccttggctagtgctggcccttctgccttggccactcctggctcttctgctgcatccagggctggcgctttggccttggccagtgctgacatgtctgccaggagcaggcctggcccttctgcctcagccagtgctggccgttcggccttggccagtcctggcacttctgctgcagccagggctggcactttggccttgtccagtactgacatgtctgccagggccaggcctggccct

SEQ ID NO: 69 - DNA, 350 bp, Bos taurus
tctctttaaaactgtgaagtactaactttccaaggcctgccctctcctctctctctgcctaaccactgggcaaggacagtttcctggcaacccctggaaggagtggctcaccctgggatggggctgggggtagagcaccggggcctcaggacctccaggcgtgtgtttctccgccctgtgtcctctgcttccccatcagaggccacgtcatcgaagccctccagcgctcccctccccatgaaggggctggccagggggtccgagctcccctgccctccagcctcatggccaccagcaccgcccacagggcccccggacacacacagacacacccacacacacacacagtg

SEQ ID NO: 70 - DNA, 547 bp, Bos taurus
accggcccttgggtggcatcaccgggggcctctccgcgctacgcatgagcacccgcccttgggtgacatcaccgggggcctctctgcgctacgcatgagcacccgcccttgggtggcatcaccgggggcctctccacgctacacatgagcacccgcccttgggtggcatcaccgggggcctctccgcgcctttagagtctctgtcttggccagagtgctgtccttggctcctcccagagccccccaccttgtcctgtctacccctccccatttggcttcttgtttccaatctggccagcatcaccaggctgttggggggatgggtgcggtctggggtgcagggctggctctctctgcctccttggccccggggcagcgagcccacagtgggagcaggggagctttaaagtcggttcatgattagggcgcccggcagccctggccaatgggcagaggggagggggctctgggcaggtgggtcttgggggtcctaggaccccgcagggggtgctgaagctggatttgggtgtcacagccagaggggtgc

SEQ ID NO: 71 - DNA, 80 bp, Bos taurus
ccagacattctggaaaggacaggatagcaccccttctctgtcccgccctggccccatgccgcccagaatgatctagccta

SEQ ID NO: 72 - DNA, 22 bp, Bos taurus
ctgtcagcctctctccttggag

SEQ ID NO: 73 - DNA, 308 bp, Bos taurus
gggggccagccgcgtcgttaggacgatctcgagcgggactagtcgttacctccgtccagaaactgctccttaaagtaaacggtgcgatcagcggcggccaggccgagaaggccgagcagcagcggcacgggtagcagcatggcgggccgagggggcggtggcgcgcgggccctttaagttgctcgtccggcagcagctctgcagtggggacggacgccgccgccggccgtgcccttttataccccgcccgctctcaaaccagcctggtccggcctcttggcccgcctccctcactttatcattggtct

SEQ ID NO: 74 - DNA, 338 bp, Bos taurus
ggtgggagttggcgctgcgggccgggtagggaccgcggagctgagatgcggacggggccgcgccggtgaccggagctgccgcccgacatgaactcgctggagcaggcggaaggtagggtgcgcctcctgggtccttccctaactcctcccggaagcttgcggggctctcgaggcgccggaatcgtgggaactggtcgggagagaggaggccgcgaaggatggactgacggggcccggagctgggggcgcgggggccggcccaggggccaaggggcggccggacacggccaggcactgcttccggcgcgcctggcctccgccggcgctggagaggcgct

SEQ ID NO: 75 - DNA, 405 bp, Bos taurus
ccgtcccgcttccccgcccagcgcttagtgtagaggccgctcccttaacagcctcgacactcgtaaggaatagaggcgcgccagaggccagtgaaacgagcttgagttcttgagggctgctcagagacttgcccgtatggctcgtgaggtctcgagcgtctacccttagtccgcctctactgctcaggctgcgcctgccgccctgggagccgccatcttgagactagctcccccttcccccctattctcttcctcccgggtcagttcttccactgcaaaccaaacgcaaggcagcgtccgatcctcgctttcctctttatgactcctttccacaggagccgcttcaaaaaggcagagttaggccccgaagtggcaactgtacggcaggaggagcggtaacggcag

SEQ ID NO: 76 - DNA, 310 bp, Bos taurus
tctgcagcagaagtgccaggtctggccaaggcaaaagggccagcactggctgaggcagaagggccaggcctggccctggcagacatgtcagccctggccgaggccgaagtaccagccctgggtggaacagaagtgccacgtctggccaaagcagaagggccagcactggccgaggccgaagtgccaaccctggctgcaacacaagtgccaggtctggcctaggcagaagggtcagcactggccaaggcagaagggtcagcactggcccaggcagacatgtcagccctagctgcatgagaagtgccaggac

SEQ ID NO: 77 - DNA, 596 bp, Bos taurus
atctcgccgatgggtgtgaagttgtccacgttccagaccttgacaacgcccgcacggcaggcgctcagcagcatggggcggcccggaacgaaggccagggcgcatacccagtccttgtgtgccgtggggatttgctagggaaggagggagccgtaagggtgggcagtggggaccccgtcccggggacgctggagtcccctggtgggcggggggcaggcagcatcgcaggcccaggagccaggctctgcaaggctccgagatgggcccttcagcccccttcccgggctcgaacctgaggccccgcaccactgaagaggccccgcacgacctcagaagggtcagaggaaaagggggtaactgctcagacggggctgagagcagatccttccttagagggcctgcctgaggagggtggggggctctggagagggtgcttggggcaccaggggaagagggtgcagactgagcaggcgtggggggctctggctctgggtgcaagggccacaagccctccctggggccagcctctccctccctcagcacctgcacgagctcctggtgctccaggtcccacttcttgacgccgttgtcccggg

SEQ ID NO: 78 - DNA, 838 bp, Bos taurus
gttccgtgacccttagcccccagcaaccccgcacccctacagacgccggaagatgatggtctataacgggtcacagctggaatcggtggaagagggagaggccgtggcggtcccagggccacccccagaaccccgcgctcccgagcccggagccccggtgccggagcccggcctggacctgagcctgagcccacggtccgagagtcccggacgcggacggcccaactgcagcccgggacggcggaaggggcgggcggaccggcggggcggggcccgcaaggggcggcaggtgagctggggaaagcttggccgccagctcgctgacgaccccccggctcaccccttcaccgccccgttctctgctcccaggtccgcttcctcctggcgccgccttccccggtccggtccgagccgccgccggccgccgcctcatcgagcgagaagcccgaggcgcctcaggacttggggacgccggtgcagcagagtagcctggccctgagcctcgagctgcaggccgcgagggccgcagccgggggccaattcgatgccgcgaaggccgtggaagaacagctgagaaagtcatttcagacccgctgcggcctggaggagagcgtgaccgagggtgaggggggcaggcggccggccgggcgcgggagcgggggtgcgcggcgccgctcacctccgcccctcatgcggtctcagggctgaacgtgccgcgctccaagcggctcttccgagatctggtgagtctgcaggtgccggaggaacaggttctgaacgccgcgctgagggagaaactggcgctcctgccgccgcaggcccgagccccacctccaaa

SEQ ID NO: 79 - DNA, 1124 bp, Bos taurus
tttatacatagacttaaaacattatggggtttcaaatggatagtaaacaatatacttgccgtgttacatggtcagcagaatataatgagtatgttgggttgtgtgctgagtttccatcattgtcttggcttgatgcggatcagtctaaagcattctcaggaatagaggagctcgttgctgatgttgtcgctgatatgattagcaataatgaaaaagttcctacaccactctccgggaaaatatagtaggcagtttgttgtccttgttctcagtggttcaacaaaaactagccctcgaagccgcagaacgtggcgtggacataaattgtttagtatcagtaaaaagagcgatgtaattttagtacgagtgagtgatagagcaggaacctatgcttcatatattcagtaaatggtatgtttttgaattaataggtatttttaaataaagctttctttgatttagtgctttttatttaataaattaattttaaaaacaacttaggctaattgataaacactatctggaaagattaaaagaaaatattttttaatttctgctaattcatttaaggcaggttccctttcaagcagttgaaatgtccaaataagtacagcctgatgattatcatagcaagtatttactccaagaaaattagccattccataacttctattgatgttcatttctttaattagttgatttcccaagtcacgagcttgctggctgatcaataactcatttggtaacacggcggtcatctccacaaaataatctaatgcaggttcaatccttatatttaaaaagttataatttagttgaaatggataaaatatactttctataagttgtaataacctgctgtaggttaattttagcaaagtattactcagtcaacaacttcaaataacccacagcaagttattttttgtaaattaatttaaatatgattacttgtaagttatctagcctcatgggcgatagaaaaattttaaaattaagtgaagtggtgcatgcaacaggtatcaatcgcaacacgctcacaagtatgtattatgaccgtgctgtacgtattgaattaccagtcgccgataagttgtgtaagtactttaactgtacgatgaatg

SEQ ID NO: 80 - DNA, 559 bp, Bos taurus
ctgactggcccaaccggactgactgtataccctgtgggctaaatgcccctttctgtgatgaaggtcccacagtccccaggctgacagcctaccaccctgcagcctgggtcctctccgggctgactgccccttcctgggataagaatcccctgtccccccaccctgggaatgactgccccccttctgggacctgggtcccccacccctcttggggatgactgctcccttccttgggcaaaagtctccttcccaccccagggcaaagctctccttgtctcctcctggtagactgcccctctccctgggacatgggttgtccccccaggactcagtgccccatctccgaggtgtgcgtacccccaggctgactgcctccctcctcgggcgaaggtccccgtgtcctcccacgctgactgcccctctcttggggcatggtccctcatctctcctggttcactgagtctacctggggtgtgggtcccttctccccctaagctgactggcccccttagggctgtggtccctctatcccccaggctgactgtcctaccaggactca

SEQ ID NO: 81 - DNA, 764 bp, Bos taurus
actggctgaggcagaagggccaggcttggccctggcagacatgtcagccctggctggagcacaagtgccaggtctggccaaggcagaagggccagcactggcccaggcagacatgtcagcactggccgagcccgaagtaccagccctgggtggaacagaagtgccagatctggccaagccagaagagccaggcctggccctggcagatatgtcagtgctcgtcggagcagaagttccagttctggccaaggcagaagggtcagcactggctgagacagaagggccagccctgtctgcagcagaagggccaggtctggccaaggcagaagggccagcactggcccaggcagaagggccaggcctggccctggcagacatgtcagcactggctgcaggagaagtgccaggactagccaaggcagaagggccagcactggcttagccgaagggccaggcctgggcctggcagacatgtcagcactggccaaggccaacgtgccagccctggctgcagcagaaatgccaggactggccaaagacgaagggccaggcctggccctggcagatatgtcagcactggccaaggccaaagtgccagccttggttgcagcacaagtgccaagactggccaaggccaaagggccagcactggctgaggcagaagagccaggcctggccatggcagacatgtcagcactggccaaggccaaagtgccagccctggctgcagcagaaatgccaagactggccaaggccgaagggcc

SEQ ID NO: 82 - DNA, 519 bp, Bos taurus
tatgctggagacccatgaggggccctagtgccgcctccttagagagccccagaacagtgtcacccctgggggacaggtttgaccctgcttgtgccgccccggctcccagcagtgagtcttcctgtgtctcaggcaggtgagccgaggaccacgggcctgagacaccatatgggaaagaaggagccactagggcctggggtggccggcgggacaggcccattcctgtgatcacctgagcaccgtcagacacacaggcagggaggctggggagggccagggaaggccggggaggggcaggcacatccacctggcctgggctttgggggaggatgtggagaggcctcctgttggggttggagcccgggagggcaggagggcgtggagccctgtgggtgggcaggagggcgtgggggtcactgcccagcagggagtgtcccatcctagttggctgggctggtggggatggggcgatgacgcccagcgtccactcgggggtgaggcttgtctctggagccgagc

SEQ ID NO: 83 - DNA, 704 bp, Bos taurus
ccctttatacagggaagaagttagtcccagagaggccctgatccggctgagtcactcagggagtccggactgggccagctcagctccccgccaaaggcctgggcctccggggctgaaaagagagccgtctccccccactcaccccagggttccttccaggatgcccacctccacccaagtctgggctcaaggatgagggatccccgaggatgcggagaaggccccgcccggtctccccgctccctccctctctctctgaaccctcgggctgtacgcccaccgctacccccctttcctcggctcccgcgggtccctccagaagcccaaagcgggggtgtgggtgcgggggcgggtggagaactccggagggggtgcggccagagctcgggggtggggcatctgtgcgggcaacaccccctccccagcctattcggagcatccctcccgcccacccccagcgcccaggaattccgctgagtcacttccgaggccgccgctgcgcccacgcggagaacaataaatgtcattcaattcactaggcagcggccgggcgctcacctgcgaaacccggcctctggcctgtcgaaacgcccctctttggccagcaaggccgagcctccctcctgccctgcgttctcccccagccatcccgctggccacgcagcctcttgcctcggggcctttgcaccctctgttctctctgc

SEQ ID NO: 84 - DNA, 330 bp, Bos taurus
atgccctactccctgtgctcaaagctctgctgagctccccagggtggtgctcactgtggggacagccacctccccttgaccatgtcaggctgccacacgcccggagcagaccatgcccatgatggaggcctcagcttctgccacctcctgctccacctccggtgctcgcttgcccccaaccaaccccagcacctcactgccccctgagaaagtctgctgaggcagatctttagagaacatcccctaagcccttcacttgcccacctcctggcactcccaccactgctgtccccactgtgggaacctcctggaggctctgctgccatgctg

SEQ ID NO: 85 - DNA, 408 bp, Bos taurus
cgaagccttcgggggtcggtcccttccccgagatccccacctcttactctccctctcgagagtggtgagagcggaagtgcttgtgtctccgagctctgagtccgcaggcccctccgttctctccacgctcgtattcctcatcccctgcacgatcgcacctctgcgtaccctccggtccgtcctccccctcctccgcctccactttccccgcaagtctgcgtccctacctcctctccacgcgcccccttgtccccttctgattccacaagctccgtgtccctgttctcatcaaaacccatgtccttggtccaacccacatcggaatctctctgcgtcccccatgtctccacttcccctgtagtttcgcgtccttagttcctccctgagcttccaagtacccccgtgtgc

SEQ ID NO: 86 - DNA, 432 bp, Bos taurus
cagctgcaggtgctccacggtgctaaacaggcggtgggccccggtccagaggcgcccatggcttgagggctccatgtaggaatagggcacctggaggcaggcagaggctcggcaacgcccttcccgctctctcctgactcccacccccagctctgaaggactacccaccaggctgatcaggaacaaagccaccgtggcagtgccaaagacggacagctgcacggcagcacagagcttccgcagcgaccctcgcacctgcacccacctgggggcggagaacagaacccagtcaggcccgccccgcacgtctccacccaccctcttgcaccacacacctccagagggcggtcagcagttcccaggccagagaggccgcgcccagccacatggtggggagggtcaccgtcttcagccactgggagagctggtgga

SEQ ID NO: 87 - DNA, 396 bp, Bos taurus
accccaggcctcaggggctcaggggccggtggagccagagtctcgggccccgaaggggacctgctcccgtcactgccccgcgtccggccccagcacagggccactgaggctcacgagttgcccttctgccaggtctatgaaacccccttctacgtggcggtggaccacgacaagaagaaggtggtgatcagtatccggggaaccctgtcccccaaggtacgccgcccagcggccccgcccggccccggcccctcacagccccgggactgtgaagaagaagcccacctccttctccctgttctcagtcccatgcctcactgtcctccctgccccccaggacgccctgacagacctgaccggtgatgccgagcgcctccccgtggagggacaccacgg

SEQ ID NO: 88 - DNA, 718 bp, Bos taurus
tcctctgcgtcccggcgcgtggagcatcacggtcccggtcactctgagagcagcgggcagcaggcagtccagtgtggatgtggggaggcgaggcttgggcaagaagcgaccgagagctggaagcccagcaggggaggcgcgggagagccgggtggcccccagggagctgctggttcactggggtgggcgtgagcaggcaggaccagcgggggaccccggggacgccttaccaagagggggagactgtgtgagaggcagaggaggcctgccggccccccgagctctggggctgcggtcaacatgggcaccccacccctgttgagctcttccaggaccacaggccgccgggagcccacaggtggcctctctgctgcccggaccccgtccgccacccctctgcagctgtgggcgtccagcaggctccccagagggaggtggacagggagcaccaccagctcctcacccaggacaacccaggggccccgcccgcaccgccccctcctgcagtgccagctatctggtggtcatggccgccccgcctgttccaggtctcaagactgcaagcaggagaccccttggctgggggcgtccacagacaggcggcctcgccaatgcgtgtctggaggggtcccaggaccagctgccccgaagacacccccctctgtttccaagagacacccaacttttgggaagaagcacccgccatgggaggcccact

SEQ ID NO: 89 - DNA, 305 bp, Bos taurus
ttctgctgcaagtctgcttggcttgatggccaagctgaaactttagacctgagcctagtcacactcccttcccctgccccccacaacctccagccttttatgcaatcctgttctggcacctgctgttcaccccaccctccctacccacatcactttccggaatgtccaagcggggagggccccactgagctgccaatcaaggaaacagaaactgcaaaaactccacctttggctccccaaggtccaggactcagccagtcaacacctcctctccgcccctggtcctgtccaacaagcccagacct

SEQ ID NO: 90 - DNA, 718 bp, Bos taurus
cagcctgacctccccgccacatcccccgttctggatccagcagccaagagcccagcccctatgatgagagcgaagtgcatgactccttccaccagctcatccaggagcagagccgctgggtggccgaggaggggctggagctgcagcagaggcagccggagactggcgccctgggggcctcaggtgagcccacctgtccaggtgctgggatgcggtgggcagggcactgagcctctcaccctctgaggaagcccctcctagcggcctggggagggggctgggcggggagatctccggccggcaggatccgtagaacctctggacaagtcttaactttgctggacaggcagcgaccacgagaccatgctggggcctgagggtgcccccgtctacagcatggccacgctccgcatcttggccagcatgcccagtcgcaccattggtgagtgggacccttttccttcggaccctgggctgggctggggtttctggctaaagatctacagatcccagagctgggccccttctgctcccacctcaccctatctgctgcctggaggcccagcccagccccaggcagcctcgctgggttctaaaaaaatgactccagagtcatcatggggataaaaataggctcttgggaaccggtggagcttgaggtctcttatgaagggactttcctgggtcctgggaagcagtgtgtgtgtggggggtggtg

SEQ ID NO: 91 - DNA, 330 bp, Bos taurus
ctgacctccccagatccagggccttgggcttctgccctgatccctggcaccaacggctgagagagaaaaccctctgcaagaggtctgcagtctgatgggggaggcacagttcttcgactgctgggggaagacaggcccagagacaggaatgcacagggtcctgaggtgggccagggtctgagcggtagaggggacagtacaggaccccacgcggttgccacggcggctctggggctggagttatcagcctggaatgctgggcagtgagctcagcctcgcggcctcccgcgctggactgtcagcagggcggtctcactggagaagctcccc

SEQ ID NO: 92 - DNA, 712 bp, Bos taurus
cattcacggggaagggagggagcgagcgagcgaaggagagtggcaggctcggcgaggccatgtgatgctgggcgagagagccgccgccgccgaagccttctgctttcctccctcggatttcgggctgtcatggcgacccccaacaacctgacccccaccaactgcagctggtggcccatctcggcgctggagagcgatgcggcgaagccggcggaggcccccgacgcacccgaagcggccagccacgcccattggcccaaggagagcctggttctgtaccactggactcagtccttcagctcgcagaaggtagcctggagggagccggggaacccgatgcgcctcgcggctaggagacttgggggaggggacccgggagggtttaaggatccgaagatctgctcagagactggagggtagaatctggggttggggggcccggaggacgggggacgctgggctgctctccggggaggtgactggccctgtcttgggccccaggagcaccgtccctcatcctaggcacagccattctgcctctttccctcccagaaagacctgatctgttggagggctgagaagagagacaaaggcaaggggaagggaatgggcactgctgggaaggcggcctgttggagggggcggctgctgcggggagggggaggagcgcttcctggggtccagcctccagccccagtcccatcgcaggcct

SEQ ID NO: 93 - DNA, 620 bp, Bos taurus
gccagcacgtcaccaggaagcgcagcggagccgctgatggttcagcagaggggcgggagatggtccagatgtggcttctgaaaggggtagactggaggccgcaggttgggcggcagcgggggtctggaaaaggggcggcggggcttagcagaggctgagtgagtgggggtggacgtggggcaccagagtgggttccctggggtccactcccagctcctcctccgaggctgtgtgctgatggcgccttaagtccctgcaccttgctctttgcccctaaaaggaaagtgaggtcagaactgggccaaaacagaacccctccccccctctctctggatgccccagcatcagtgccccggacagaggatgcctagaccagggctacaggagcccaaggaagcagggaacgtgcacaggatggaggcagggctctggcctggacagcagaggggctgggaggaggcggtgtcccctggggcaaggactgagaagccgaggccatccttgcgagcagaaccttagctgggggtggggggcctgggtgccagtccgtgtcactagggagccagccgtgcagctgggaccctttgctgcggaaaatgaaagtcaggaaggtggctgga

SEQ ID NO: 94 - DNA, 325 bp, Bos taurus
tctggggcttcttcttccagcagccgttgtcttcctacttgttactgagaccggagtttggagagtccagcagcaggaagcaccaaggcgggatttcagcccaggccgggtccttccagggcccctgatccgggggcctccctccccgccaccccctgtcaggacggggtcctctctgggcctctaagggcccccgtctctgggaccatcagcgctctggggccgggtggatgcagcctggatgggcagaagcctgctgggtctccatggctggccagggagggcgtctgcttcccgggagaggtcctggcagaagatggaggga

SEQ ID NO: 95 - DNA, 387 bp, Bos taurus
ccgatgactggctgcgatatgggaacccctgggagaaggcgcggccggagtacatgctgcctgtgcacttctatgggcgcgtggagcacagccccgaaggcgtgcgctggctggacacgcaggtaccacaggcgcttcctggggaggggggctgagcacagggaccctcaacttgcctcatgtcaggggcttcccttggggcttccctgtgggagctgactccccacacacttggtcgggacggttcagaccccaaccatgaagctacatctgtagttaaacaggagctgtgtctcagggtgttttgtggtgagcaggttggtgcctggagggacagggtactggggggtgggtggtgagaacagctctgcatcccagggtccagcg

SEQ ID NO: 96 - DNA, 354 bp, Bos taurus
gcctgcgccccctcacccacaaatgccccgttgccatgcgccccctcacctcctcttccacggtggctgcggtctccgcgtcccccagccggctccgacaggggaaggctctgagcaccgtgagcactgcacggaggcgggagaaggggtggacattgaccctggcccccaaacaccctctctgcccatgggcccccaatacccactctgactgttgatgtcccagcggccccccaggtaccccacgacctggctgcgggtcaggtggctgtggaagtcctggggagggggagggacatgggaagacccgctcagactgtgcctggtcccaaggccgtcctgggggtcagccca

SEQ ID NO: 97 - DNA, 1858 bp, Bos taurus
cttcctctgcagcttcatcacctcgctcattagcagctgcgtcaggcctgactcgccagaggcgtctgcagagcgggcgggatcagatggcgcccgcactcgaggacctagtggctccggggcggcttggccaaggggtcagctccccgcctgggggacaaactgaggcgcggactcacctccgggccctgttcccacccaccccgccgaccccaccagggcaggacagcccggggtctgggcgtccatccctctctaattctgtcttggttgagaggtgggaggatgtgaccctgctggcttctcgggcagggcttcggcgaggtcagagggccttctgggtagaaggggctctgggccaagcatcagcctatgcctcccccctcccacaggccccaccctggggctcgggctccctttcctgcagcctctgcccctccacgagctcagccggcgggatgggagggacgcccacacagggacagaagctggcagccccacgtgaggctcactcctttcctttcaccggcggtttacgcaacaggggaccaggttcccctgcccctcgcgcccccaggcctgcacccaccgctcaccgatgatcacggagaagacgcgggtgggctccttgcccgtgactttcctgtagagctgcgggtagtacagctccaggctctcgaggaaggccacgtagcccttgtggcccgtccgctgcaggatgtccaggagcactcctgtgactcagagaggacgcatgtggtggcccacgaggctgcagagggggccagcctggccccaccttggggcgctcccaggggcgtctgccgtgtggggagcgggcatagtggcaggcagacggggcggaggcagggtgagggtcgggggaccagggtgggggcagagtgcggccgcccgcggcactgacccactttccgcttgcgggtgaccaggctggggtcgctgagcacctgctcctcgtcgtcggggctcagcacgttgcactggcgcaggtagggcgtcacgcgggcggggtcgatggcagagatgagcttcacccggaagccctccagggcgctccagcatgcgtcctcgttctcatagtcggacatggctggggtggggggcggccgctgctgagccgcgggcccgcgggggcccaggccagccgtctcggccgtggcgggaggagccccctgctgccgtcgactcacccagcatctcccctcgcctccccaggccctgctgccgaccctccaggcgccctgggctttctcttccaagggaccctggtaagcgtcaggggtggcgtggagatgctccagggtcagggtggtgtccaggaggacacagtcgccagccccgcggccctcctctggccctgcccgaccccccaggacaccccaagtgctgtgggggctggcggaaggcacgtgagggagggggccagtgccgggcacccgggccccaggctgtgcttcgggacttcctccttgcccgcgggacagcagccctcacgtccaatcctcccagcctcccttcttccggtgatgcaacagttgcccagcagctactgggccgttcccgcccgggtcaggcttggcgacgcggtgccctccaggaagggtgcctgtggacggcccactggagggtgcaggcccgggtgtcctgggaggggtgggctcccgggccctgggccctagctgtcagggctcctctacacctcccggacgaggcctcgctcttgagggctgcccagccagctctccaccaggagaccccccctacgccggaacggcccactgcacctccctcctgctccatgtccagaagcccctctctccagggctaggctgagctg

SEQ ID NO: 98 - DNA, 842 bp, Bos taurus
gcaggcgctgggagccctggatgcgaagtggccacatctgtttgctctcggcctccgcccgctgccgggctttctcacccgctgtgggcggagggttccgggaactcgctctgccctgaccgacagcagaggagaaactgcagcctgcgactccgactccagggcgcccgggttggtcgcgggaggagaagattcctttaaacagctcctgtggatggaggccaggcaggatgaggtcgtgggttatttccagtcctggcggggactgagtagccgcacagcaacgctgggggccgggctgggagtgaggccggcagatgctggggggagtaaagggggcgtccagagcgggaagcccggctccagccgggaccctcctggaccccagctccctcccatgtgaaacgggcgcactcacaaagcccgctgggtcagaaccggttctgagccgccctggggctctgtgccctgccgacatcagcgggtgaaaccagggccctctctagagcccccacactcggggggtgcttgggagcgggacgcggctctgagggtccaggccggagggcccagcgaccccagtgaccacagaggccccggctcagctgggcggcgggggtgcctaggagctccggtctctgtgctttccctgctcttgggaacggccggcccccctggctgcagactcctgttcccatggcgaccacggggtcgcagggacctggcaccagcagtgggggcgcctggcctgggtcgcctgccgctcccttccgccgtgtccagcacccccacagctgacatcagctcctggtgtggggcctcctccaggctggcctcctggc

SEQ ID NO: 99 - DNA, 1220 bp, Bos taurus
gcagaagtgccaggtctggccaaggcagaagggtcagcactggccaagtccgaaatgccagccctggctgcatcagaagtgccagttctggccaaggcagaagggccagcactggctgaggcagaagggccaggcttggccctggcagacatgtcagccctggctggagcacaagtgccagttctggccaaggcaaaagggccagcactggcccaggcagacatgtcagcactggccgagcccgaagtaccagccctgggtggaacagaagtgccagatctggccaaggcagaagagccaggcctggccctggcagacatgtcagcactggccaaggccaaagggccagccctggctgcaggagaagtgccagtactggctgaggcagaagggccaggcctggccctggcagccatgtcagccctggctggagcagaagtgccaggtctggccaaggcagaagggttagcattggccgaggcagaaggaccatcactgaccctggcagacatgtcagccctggctcaggccgaaatgccagccctggctgcagcagaagtgccaggtctggccaagccagaagggccagaactggccgaggcagaagggccaggcctggccaaggcagaagggccagcactggatgaggccaatgtgccagccctggctgcagcagaagtgccaggtctggccaaggcagaagggccagcactggatgaggccaaagtgccagccctggctgcagcagaagggccaggcctggccctggcaaatatgtcagcactggccaaggccaaagtgccagccctggctgctgcagaaatgccaagactggccaaggccgaagggccagcactggctgaggcacaagggctgggcgtggccctggcagaaatatcagcagtggctgaggccaaagtgcctgccctgtctgcaacagatgtggcaggtctggccaaggcagaagggtcagcactggccaaagcagaagggtcagcactgaccgaggcagaagggccggcactggctgagacagaagggtcagccctgcctgcagaaaaagtgccaggtctggccaaggcagaagggccagcactggctgaggcagaagggccgggcctggcccttgcagacatgtcagccctggccgaggtcgaagtgccagccctggctggaaagaagtgccaggtctggccaaggcagaagggcctgcactggcc

SEQ ID NO: 100 - DNA, 647 bp, Bos taurus
ggtgtagaccttggggttcccagcccagacagggggacagcgagtcagggctcctcccaagacccagcagggccaggcatgtgggggccaggcaggacacagggcgcactggccagggggagggagcggacccctctccactctgaccactatgctccactccccccgggggacctgtccctgcatgcagcctctcccttggcccctccttggcccgcatggcatctcccatccttcggttacgcctcgccctgtgctggggccagggagccagagcctgagaagaaggaattcctgggctgagggctcgggcatcctgcgggcagatccacaagcaccagtggggctgaggcaaaggcacctggaagcccctgtgcctgaggaggcccacccagagggggagactttgctgctgatgctgaggctggaagggcgttcaaggcagagggaacagcctgggtggctttggagcgacctgaggtctcaggggcagagccgccaaggtgcagttctggagaccaggaggagcttgagggtgggggtggagatgaggatggaggcgacagagctccagggctccagggaggtgtaggacggggaagagctgcgagtggtggcccagttctcctgcgccaggcggggccggg

SEQ ID NO: 101 - DNA, 561 bp, Bos taurus
tcacagcctggcgtggcaggtggtccacacaggtagcagaggtcagacctgcctgaccgagctgaactccatccctttgctaagccggggtggctctgcctgcttctccgcccctcccctgcccccagcccagcaagcaggagcaagacagcaagtggtcctctggttaagaaaaaacaggctgggcttcagccttccagggcagcaaggtgatgagtgaaagttttctgagagaatcaaagaaatgtgctctccagccttttccccttcccccaactcctctccccctccccactcttcaaacactacatggcccccggccttcaccagccccctttgcccttgattaaagacagaatcactgatcagagaagcccagcagggtccagctggaggagcctgggagaccagctaataatcctgggtgcccacagcggggcaaagcagtctgggggcggggcttcagccccaccttgccatccaactctgggcccctactggtccagccctaggtgaacacggtagagctggggaaactgaggcctggtgaggcctaccggg

SEQ ID NO: 102 - DNA, 493 bp, Bos taurus
ccagggcccctgggtctcccaggcagcctcaccatcaggactggtggtggtgccccagccggaaacggtgcacatggtcccggggggatcgcagtgggaaggcaggttgactttcttcacactggacgacagcttggcccggccattcagtttcaccagcatgaggtcattggcgtgggtctgtgtggagtatcctgggtggcggaaggaccgcgtggccttgatcttctggccgcccaccagccgaacactgcccatgtgcacgttgtactcactgcaggaggcagcagtgttcagagagatgctatgagtgaaacggggcggggggcagggacccagagagagggggacagggaccagagagagggggacagggacccagagagagagggggacggggaccagagagagggggacagggacccagagagagagggggatgagaacccagagagagagggggacagggacccagaggaagggctggaagtgc

SEQ ID NO: 103 - DNA, 335 bp, Bos taurus
ggcgtgcacgtccccagggtccctcagcccctgaaggggcctcatgcagcttcctgcagactgagcctggcttccaaacacgcagccacataggacggtgcccacctgatgcccccagcatcactgctcaccgggcccagccgggcctcccctcaccaagccaggcgatgggacctgggcaggcccaggagaggccgcggccagccgggtgaccccccaccccagcagagggcgcgtctcaccaggggcaggagaaggcgtcgcgacgaatccaaagccgcccactcggggggactcatgggggatgagctccttgccgtccgggcccaccttgc

SEQ ID NO: 104 - DNA, 354 bp, Bos taurus
tgggggacccaggccctcccccacccacagggctgccccagtgcctgggcccttgcacgcatcaagctcgcactggaggagtgtgcaagaaagggcacaggctgggccatggggaccccaaaaggctcctgaggggcctggaataaagtctgaacagaagcctgcatgcggtcagatcaatgccctctacccgcaaccttccaccagctgccccctcccctcacggccccacccctcctagtcccgccccggctccccaggcacatggaggctgccgttggctgctggaaggtgtttaccagctacagggaccaagggcaagccgcagcacccagcctcttggggccaggtgag

SEQ ID NO: 105 - DNA, 337 bp, Bos taurus
cttgcacgggtgtgagggcacagggcggggaggccttcctaccgtgggcaaacccccttcctgagtgtgaaggggaggcagcgagggccagggcccagagtggccccaaggccacgggaaagggcagccctgcatccggcctcgctgcctctgggggatggggaagcctggaggacaggagccccaggggagcccccacggcacaatgggcgtagctgtgtaccccctccccccagggaaacctgtttgacttcctccggctgacagaatggcgcggcccccgagtgctctacttcggggaccacctctacagtgacctggcggtaagggggccggg

SEQ ID NO: 106 - DNA, 878 bp, Bos taurus
tctggcgtctgggcacctggtgaccactggccctcctgtgacaggaagcagccagtttccaggtgcaactgctgccctggggctgatctcaagcacggcttcatcacaggacatgcgctgttggcttcacctcgccctgccggctccagtcccacgagatggacatggacagggactgggcaggtggggtccaccctcagggttcctgactctcatcctgggatccccaggctggcagctgtgctccagaggcctgtgggacagaaggggcagaggcatcacggggcgggaagctgggaccctagggtgcagataagtgacccgccccaggggctgctcagatgctcagaaaaacagtttgtgcagggttggagaaattggggcaggggacttcccagaggggaggggagggggccctggggagggtgagggaccccttgggcagagaggggaaatgagaataggccacttcggggccccaatcaccttggttgtgggtcagagcctgagacatggtccccagcatcccccccgccgccccccgccaaccaggcagatgagggagggggcaggagggtttggtctgagccttttgggacaccaggcagctgggcctcaactctgccctggccttcagcccctaacccctctctaacctgtcctgggagagctggagggcccgagggtgagggctgggctcctctggctcaggtggttcccagaccaagaagcctgtggggctcgggctccagccgcgttgaaggactcaccctcttcgtttcctggggtgtgtgggaactggggctggcaggcgtcctgggcggggaggagcatgctgggtggggtgatgggaaaggcaggtgtgcgtgggggtaact

SEQ ID NO: 107 - DNA, 973 bp, Bos taurus
ccaggccgctctcctcacccgaaacggccggcggcgagccccgagaaggaccctgtccaccttccagcccccagcggtggtgcccagggcctgccacaccagctcgcggcggctgccctccaccacgagcagctccaggaagcctggcaagggatcactgtgggcgcccgtcctgcccctgtccttcgcccgggggctctgaccccccaccccaggacggggcagagggcgcaaagggatggtggtacctcgggggtgactctgcagatagtaaaccaggcggagctcgcagcgggggcccgaggggccaagggtgggtgtgagcgcccgggcctcctcggccagctgcccccaggccctctgcacagccaggaagtgccctggggagagcaggcctcagagctggttcctcccagggaccccgactcagtgccctccctaaggagagggggcagctggaaggcaggccccaggccgtcccgggcggagcctcaccagcagccccgtgggcgtccgggctgggcatccccctgtccggggccgggaggcgcacccactgcagccgccccacactggcgtcctcccagcccccgcctgagagtgactcgaagtccgtggtgcctgcaagggcagggggcatggatgggcctggggcctcgccagccactcccagaggggaggctgggggaagggaggggaggcccctggccggctcaccacattcctgctcgtcctcaccccccgggcattgctgctggaagtcacacagctgctccggggggacacacaggtccccacagaagaagtgtccgggctcgcagaagctccgaggccgcaggctgggtggctggggccaggggcctggggcccagcaccctggaggtggacccgccagctctgtgggccagcgcgggggtcaggtgggttggcggggcctgtgcttctcccagggtggggcgtgagggtgcccagc

SEQ ID NO: 108 - DNA, 317 bp, Bos taurus
gaggcttcgcttcacgtggcatctcagtggaccccgcagctccgggcgctccgtgctagcggactgccaagagatgtgagcaccgtggagatgctcgtggcgagcgacccggagccggcagagacggcgctactcttgaggatgggctgcgccgagaggccgatacagccccggggtgtccgggggaagggacggggaccgcagcccgggaccgggagcatcccggcgctggagcgggcgcaaaagtcaagagccacagcctctgtccagtgcttgcgaagccgccgcccctggggtgcgccccgcggtttgtgagt

SEQ ID NO: 109 - DNA, 302 bp, Bos taurus
tgagaggcccccagggtccctctgctcccgcttactgtgagcccggggcccggaactccaggcgcggcaccggcggctcggcgctgcgggcagtgggatccgagcgaggggcgtccgcgggcagcgccttgcatattgttatggtgaccgccgcggcagccgcagccggagggcagtgaactgtctgcgacccgggcggccgggccggccctcggctctctccgccgggagtgtcgtggagggggcacgaaagccgctgcgcccgcccgggctccggggcgcgcggggctgcatcctctctg

SEQ ID NO: 110 - DNA, 1464 bp, Bos taurus
attgatccacagcgggctctgggcggggtagtcggcaggcacgctgagctccagcggtggcacgctggggagatccttgtcatctgcaacacacgggggcacctgtgacccggcagcgcccaccaggcctggaggccctgcagaggccagtgcggagcccaggcactgactcccgcgcttcccgtctcctcagagacccggagctgcagctggacgggagggaaggggcttccgtgcccggggtgggggcagggatgagagcaggcaggctcagggcccgcccccaggcaagtgcgcctgctctcacgcgtgggcccagaagacagcgggcagattccaggagggcggggctgggcgagcaggcgtcccgccagcccggccccgcgctcacccagcttgcatatcaggtggacggcgccgttgctgctgcagtgcgaaggatctaagttcaccaggaacttggggtccagccgcgccacctccccctggagcacgttggggatggtctgccgctcatcctcttcaaacttccgctttcgggtggacaccaccggggccctggaggaggcagccatgctgagcccctggctgggccacggcctggcctggcctggccgcccaccaagccccgcgggtacgcacgtgatgggcgggccgtggatggccgtcatggccggcacgaacgtgcggtacagggagtggttgaagacgggtgagcggatgttggccagaacagcatccagaagcggctggcacaagtactgctgcttggtggggggcactgggggcggcgggggcgtgggctgcagcaggacggtggggtcagcagcccactcgtggaccccggccctcgtcctcgcccagccggccttcctggcttccgcacaagctccacaccacagacggggcgggcgggcccggacactccacggcagcccggccaggcccgtgggccacgagagccgtgcactgcgagccgcagccagagcttcccatcacgtcctcacctcagcccactgcaccgggtacggcccccgccccctcaggctgcaggctccagctcgtggttagaccgtggtgagccctgaccaccagggatgcccacgaccctacgaacagcgccaggcacggcagcccactcaccaccgccatgtcgttcttaagcttctccagggcgatctcacatttctgcagcgtcttcagcgggcacctgagggaggacaacggggctggccctctgtgcgggggacggggagccgagagctcccaggtctctgcccagccacggacagtctggccttctctggacggagcgggctggggcctgggccaccggcaccactcccaggagcccgccagcccacgcgctgttcccaggcccgaagtctgaggcagggcagccccacgcccaggacaaggcacaccgtgatgggccaagggaggatgcaggccaggcccctggcc

SEQ ID NO: 111 - DNA, 386 bp, Bos taurus
gctggggcccaggctgggcgggcaggcctaagccctctgcccacagcccacaccatagccccctggcctgggcctccgggggtccccagggctcagccagagcagtgagtggtggcccggggcatcgccctcccctagggctgcgtccccccagcccaaggacggccctctgttggggggcagctgcacaggactctgcacaggggcccccggcccagcctcaggttagccccacctgcgcccgggctgagtgactgtagagggagcatggacaggagtgaccatccgtagcccagtgcccagggagagaccggggaggggcggggatggactgctgacacagatggtgacagaggagagcctgcagctcggggcggcctgcgccc

SEQ ID NO: 112 - DNA, 198 bp, Bos taurus
gcttctgctcccaccttgagcccaggggccttctttaaccctcttccccacaacacacactgaagacccagcttcctgacttctcgatcgcccaggtctctaccctaccttcttcgagatcctggttgctcttttgcatccatcctcgagctctggagaccccacgtctcccttgacccctcccggggtacctcctta

SEQ ID NO: 113 - DNA, 139 bp, Bos taurus
gggcttctccccacctcccccatggtcccctgccccgcccccctgaccttctctccagcagccccagcctcagtcctggctccattcaacaagccgcctgatcctcaagttcttttctattccctcgctcttattctta

SEQ ID NO: 114 - DNA, 687 bp, Bos taurus
ctgggacgtggagaatatgggcggcgccgtggtggccactgcgatgctctggcctccgtcgctgaccacggtcacttcggcggccccctcctgaatcagggcgttcaggccttcgaagtcagtgcacgtgatgcccgagctggtgatgaaagtctggttgccagagccttcctgggggccgcccggggtggtggggatgagggtctggtgcagagtggcgccctccgtctggtcgtgggtggtcagcaggacagccggctgggggagcgccgcggcctcgctggagcagccagagggcggagggggcgcgatcaggttgacctggcggaggatctgcagccggctgttgccccgcagctcctcggggttcggcgccaccaaggtgggctgcacgatgttcacggcggccgccgccgcctggacgatggtgctggcctggggcacttggtggccgacgatgattttgaccctgccctcgctgaactggggcgccggggtgggcgggaggggcacctggaggtgccccacggccaggggcgcggcgggcggcttgccggcgtccacctggaactgcagcaccgtcacgtcggcgcccttgccgtcgccgtactcactgtgctgccgcttgtggctgcggagcgagtcctcgcgcatgaacgaggcgtcgcagcggtcgcagtggaaggt

SEQ ID NO: 115 - DNA, 635 bp, Bos taurus
gattccagtgcagacgggccctgggaaaagggggtccccacgaggtgcgtgggcaccgtgcggcgggtggctgaggagttctgagccatggaccaagtggccacggttgggccaggtgggggggctacacttgccgccccagtcgaccagagtcagggtcgcaagtgctcggcctgcacccccgccctgtgtccaggcagcggcccggtgcggaccaggcctcctgccgcccaaggctgccctcatcgcccatgctctcgggaagtgcaggcgtgagacccccacgagacggtcagacgtgccgctcccgccgctctccctgccgtgggcaccccccgaggtgtacagcacagacccccagagggccccggtgggcgctaggccgcacccccataatgcaggcgcctcccaggtcctccggcgttgctggaccgaggccgtcccccagccccagcccttcgcgtgcacaacctcagcccgcacagccctcctcccggcctcgtggctgcagggtgagcgccctgtgcctcccaggctgtcgggttgcaggcttggcgtctgctcaccgggtgccaggagcaccccacccccggggtgacagccggtggacaccgccatcgttgtcggtcccgctt

SEQ ID NO: 116 - DNA, 826 bp, Bos taurus
  misc_feature (317)..(366): n is a, c, g, or t
gaaggcatgcaccttttaaaacatgagtattcctccatcctctatccaggcttgtttctccagaagcgtttgtctctggaacgcgggtggctccatgggcagagaggggccgggctccagggaggactccccactgcccaccctccccaagcttctgaagttcttgttgctttcccagggtctggtggggactggggcgtgggggtcagctcagaacggcagagtctggacaccttcttcagcaaggggagggcaactgcggccagggggtgggggtgcggggggccggggttgcccaggcagttagaggggtcagnnnnnnnnnnnnnnnnnnnnnnnnnnnnnnnnnnnnnnnnnnnnnnnnnncggcgggttagaacctcccaaggggggccaacctgcccagcccctggccggccctctgcggtgctgtgagtgaggacgggtcctggcacttagagcacagagtggctgctccccgggaggcccccgtctgtccctgggccccgggaatggggccggggaggaggggcgcgtgggagggggatgggggacggctctggctcaggccgcttccctcccggcctcccacggagccagcctcctctcgccctgctctctggcctccctgcctccctctcaacccccagcagaacccctcctggaacggggcctcagggagcaggggagggtctcgcacccctgactggcaggcccctgtgtcccccaacccagtgctgggtcaggacagactgcctcgcagggagggaggtgtaggtgagggggtcatccctttaggtgggtgatggaagcttctagaagcttt

SEQ ID NO: 117 - DNA, 313 bp, Bos taurus
ccactcccccaggaggcctcatggcggggggcttcccaaggcactgggggagcagtcagggcccacctccagggcggggcaggcaggagcccccttagctgggtccagtcagctgccccgtaaggaaagagggtgctggggcttccttctggggtgcagagccgtgcaggtgaggctgtgagaaggacaaggcgggtggttctgcaagagctggggggcacgtcctgtccttggaaggctgagggcccagagggcttggacctggggacccctccccagtcctctgtgagacttcctcctgacccaccagggg

SEQ ID NO: 118 - DNA, 344 bp, Bos taurus
caggccggctcactgggtgcccgcggcggccgcgcacgtggagagcgcccagccggggaggcagtaataagggtaatagtaggagggctgcaggggcaacagcgagggtggtcgcagcacctctccgggcaggtactgtctctgatcgtcgcggaccagcaccttcaccgccaccttcttggcggcgggcgccgaggccagcaggtcggcagccatctgccggcgcttggtcttgtagcgacggttctggaaccagatcttcacctgcgtctcagtgagcttcagcgacgcggccaggtctgcgcgctccggcccggacaggtagcgctggtggttgaagcggc

SEQ ID NO: 119 - DNA, 314 bp, Bos taurus
gggagctgaaggggcctttccaggaagggcctgaccctcagggcggggtgctcccggggcctcgcagccagcgagggacgggtgcagacccccactgcgcccaggcggtcttggacagaaacagacctggcctgggcgcttaggaggaaggcaggcctgcggggaggggcctgagtgtcagcaactgcctgtgggctcttagccactgaggtcagcagggcaggggccagggtagccgccttacccccaggctctgattctgaaggtctcgggaggcctggatcgggtgcttctcgtagctggtggtgctgcct

SEQ ID NO: 120 - DNA, 1187 bp, Bos taurus
ccaggccgagccatcactcgagggtctcagctcctcctgccgggcggcctggggggcacccagggtcacagcacctggagcccaaggccaccctcagggcccgtgagggagatggcacgtcctggtctcacaggctccaggaacgacaggatcctggctccacctccagccggagactccgtccccacactctgcggcttttctgtgggcccagccccacagaacggacagcaccaggacaaggggcggctcccagcgtccccctcccctgccccatcccccaggctgcagcacccggggctcgctcaccatctcggccagcgcagagatgaccttgcccagggtggtcagggacttgttgatgttggctccctcctgcacggggatgatgtggccgggtgtcaggtggcctcggaggccctcgctcccgtgggaccccgacctgagctgagctgccctgatgggaggcccagcgcccagccccccagcctgggggacctgtgaaccctcccgccctccccatcctatgaaggcaggaccaaagtgggtgcaggaaatcaactccccttccgtccaccctttgcggccgcccccactgggtgttggtccattttcccctgcgctgcggtctccccctcccctcagaccacccgggcatcaggccccgctgggcagtcggtggtggtaggtgaggaccctcgtggggcggccctaccttcaggcgcgtgcccttggcccccgtggagtcggcccgctcactcccagccaggtccaccaggctgactttgctcacctgtgaggacaggggcgtcacacgcaggagccgcgatgcccgatggggcctgccccccgggtgcagccggcagggcccaccctcccgggccagtgtcgtccgtcagctcctcctgctctgccgtgcgagcccagcgcccggggaggctgccccccagcccccgccccccgctgcccgctgcgtgctggctgctcttcacccacacatcaccgccactgtcacccactccaaggccaggctcactgtatggtgggatctgagcccagttcccagagcccctccgccactggtcaactgccctgtgggggtgcagctgccccagcaatgggtagggaccccggcactcgcccagcctgggcaggccgccacatgctcctgggggaggctcacgccc

SEQ ID NO: 121 - DNA, 501 bp, Bos taurus
cccggctgcaagcctggtcgatcccagtcaggggctctgagctcacggggtgcttcctccctggctgagcacatggggcattgtcacgggtcccctctcctgcccagggctcccccaaatccacaccccaaagccctgcactcaaactctcctccctcctacctgcaggcccccagcttcaaggcagccagagcccacctggaatgggcagcagcagcctgagaccacacccccccctcgccaagaccgccccacagttttcctcggctctcgagacggctggcctggcccacacccccctgagaccctcagcccgtccccctgcctctcacccctgccaggccctgccgccctcctctcccttcactctttgcccagattccctctgccgtccactcctctgctcacgttgtcccttcttctgagctgcccaaaccctcctcataagcagacgggagacgaatgcactgaactgacttagatgctcctcctggtcttgac

SEQ ID NO: 122 - DNA, 529 bp, Bos taurus
cagagcttcctgctcccacgggatcccggctggagattccaaaaggaggctcctggcccgggcaccgtgcccgcccagcctatataggtgttgccagaggcttatataggacttccgcaagccctccactttggaatcattgccccgtctgcacacctcactctcagcaccctcctccacctgtctctcctcctctccttctctctttctggttccccccagatttcttcttcctcctccccccctccacctcctctcacgccttcctcacccccgctccctggggcacagccctcctctccaaccccggcgccaatgccatcgccccccacccctcgttccctcccgcttgccccaagagttctctgcacctgtgactgatctggcccccacccctgtctgcatcgcacaccattagtgatgggtagttgagcggcacgtccacggtggggagggggcatgcgggaggctggcagactgcggcactgagaacgtaactgaactcatgtctctgtatgtgtgtgtgtgt

SEQ ID NO: 123 - DNA, 479 bp, Bos taurus
aggccctgcactccccttctctgccctgaggatcttatggctcccgggcagctgccactctggctgaggtctcagctcagtgtcaccttcttggagagctgtccccagaccacaccagctggggagcttccctacccttccacccctgacccctgccattgacttacatgagctgagaggtggtctggtcgcttcccggccctcctcccacctggatcgtaaccccgtgaggatcctgagcctgtcggtgccctgcgcctggtgttcggtaggcacttaggcgttgttgaaatgaatgaacaaatgaagggccttgcccaaggccctgtgagccgcccctctgctcacctcatctgcccgcccccccaacaccctgcaggaggcatgatcagctttgacgtcttccctgagggctgggacaagcgctactgcctggacagcctggaccaggactgcttcgacaccatccatttctttgg

SEQ ID NO: 124 - DNA, 361 bp, Bos taurus
gctggtccaaggcctggtcctcatccagcttccgggtgatgatggcgcctgaggccctggggctggcggccagccgggccagcacaagtgtgcccaggccccaggccagggtgggcactgcccatcagcctggccttatcccaccctgaacctggactccaccgtgacacctggccccaccctgacaccgggccccctgcctgtctgctcaagttgccttccccccgcctctcccattcctcgccctgcccgcccccccacaaccccagcctcaggttccttccgagcaggtgctgagctggagacaggtggtttcccaggtcctttccctccacagctgggagtttgcacagactcgggt

SEQ ID NO: 125 - DNA, 335 bp, Bos taurus
ggcagaagggtcagcactggcccaggcagaaggccaggcctggccctggcagacatgtcagtactggacaaggccaaagtgccagccctgtctgcagcagaagtgccaggtctggccaaggcaaaagggccagcactggccgaggccgaagtgccaaccctggctgcaacacaagtgccaggtctggcctaggcagaagggtcagcactggccaaggcagaagggtcagcactggcccaggcagaagggtcagcactggcccaggcagaagggccaggcctggccctggcagacatgtcagtactggacaaggccaaagtgccagccctgtctgc

SEQ ID NO: 126 - DNA, 776 bp, Bos taurus
aatttcatactcacctgaagcaggtaaaatataaataccattatcatcaagctctggagaatattctccaatattgatggagtcttttaattgattaccccataagtaagcttcaacatttcctccagtcaacttcacttttaacttttgcccttttttagcataaaatttgtaggaatcatacttgtatccctctatccttccgtaataatttgcagagtaagctttttttgcaaacttaactacaacagtctttcgtatatcattgttatcatttgcaagtacggctgggctaagcatagttaaagttagggccataaaagcaccagctagtaatttcgtattcgctattttcataaggttaccttaattcttgtatcgcccctgacatagtaacataagtaataagtaaccatgaatcatggaacagaactgtgaatagtcataacatagagttgcacttcaaatgctaatttaagacttgcgaaatcaatggttaattatttggtcactaaggaatagccaatctagatataaactttatttaacaaatacttataaaaatatgtgatgcgtatcatacgcatcatatatattttcttctattttgatataatggatctcgtcaatccgctgataggtttcatttaaatttcatgagtcttactgaaattaaagttcgccaagctcattgcaaggaaaaaacttgttttttatccgatgaagatggacttagcctgaagattgagccgactggaagaaaatcttggtgctat

SEQ ID NO: 127 - DNA, 440 bp, Bos taurus
ctgttcagatttctacgccagcgccagaggacgcccctccaaagaaaggaggggggcagagctccccaccctacttccccagctccggacaaagccccttccttccagctgccggcagcccaggccgggcggcaacgccagacgcccggggcagaaggaagcagcaggccaggaggaagccgctgggggcgggggcgacgctcccagccctcaatccccatcaccttccagaggccccctcagcggccccggactctgggcatacagcagccggggtcccaaggagaaatgtgggtgccaccgcctccgcaggtctggcggggcgaaggggaggggacaagggagggtccccggatctggcagctggaaggcgcgggccggtgccgcgctgcgaagcgcagtgggcctggccctccggcccccacgacctccattttgcc

SEQ ID NO: 128 - DNA, 319 bp, Bos taurus
acccagatgctgcttgtgggtgcagaccttacggggggccacccttggccggtgcctcctccctctgagccagagtgcccgtcctcagccctgagcctcccggcctcagtggctccaggagcccggatcacagcggggccccagggctctccctaacggcctctccaaagaatccagggccctcaaaactgcagaatctgtttattgagcagaagggggaggccccaggagatgcccgcgaagggtgggggacgggaaggcaaggctggagggccgtgggtcaggacacttggggtgacaggacccgctgctccggctc

SEQ ID NO: 129 - DNA, 600 bp, Bos taurus
ttaccgtcctcctctccgtcctcctcaccgtcctcctttccgtcctcctatccatcctcctccttgccatcctcctcactgttctcctctccgtcctcctcaccgtcctcctccctgtcctcctctccatccacctcgccatcctcctcaccgtcctcctttccatcctcctatccatcctcctcctcttcgtcctcttcaccgtcctccgccctgtcctcctctccatcctgctccccgtcctcctctccatcctcctccttaccgtcctcctctccgtcctcctctccatcctcctcatctccgtcctcctcactgtcctcctctccatcctcctcctctccatcctcctcaccgtcctccaccccatcctcctctccatcctcctcctcaccgtcctcctctctgtcctcctctccatcctcctctccatcctcctcctctccatcctcctcaccgtcctaccccccgtcctcctctccatcctcctccctgtcctcctctccatcctcctcatctccgtcctcctctctgtcctcctcactgtcctcctctcaatcttcctcctcaccatcctcctctccatcctcctcatctccg

SEQ ID NO: 130 - DNA, 700 bp, Bos taurus
atccccgggggacgagcaccccccgccacccccaggccagccttgagcccaggtgcgtaactgtcaccaagataaaagagacgtaattaagttccaatcagcggagtgatttgttatcataatgcaatatttccctccagccccgggccgacagatccatctccgcaggcctgctccggccggggtccccaggtcctcctgccacccggggcccagctgcccgaggagcggcgggggtggggtccacaccgcctgccccctctcagccacacacactcatcccccagctccaagctgccgaaggtgataatgaagtcatttttctaaggcatgctcacctgggaggcagccctgtccccgggccagctaaccgagctggaaggcgagcccgggggagggcaggccaggcgggccaggaggggcattgggcacgccgggagctgccgccaccgtggggggcggggggtgactgctgtgggcgcccctgggagccaacccggacgcggccatggagctggaaagaaaaaaccccgggggcagacctggcctccagggggagggcggtggccagagctccggctgtggccccctgggtccaggagctgcccagccacccctagaaaggcccccagggtcaccgtggcccccgaccctgagagcccagcctggctggcccactggccccaccggcctctgggag

SEQ ID NO: 131 - DNA, 1180 bp, Bos taurus
cgtgacttctgcagcctcggggctcagggactatttaaaatcctggtgagctgagccctggctgcttcgctgcgaggacacgattgcatttgctgtctccctcttctctggagacagagcggccctgatggaaggctgtccagtgggggcacacgtggagcgggtcctggggtctcggggaggagggcagacagagtctcccttcagccacgtgcactcaggggggcccgttgcctgaggccccccgaccttctgcccccacccctcccgctgtccgtccagggctgtctcggggcgcacccggcctcgccccacaggggcccacgaggccccgaggaagcttttgccagtgactgccggacagcaggggtgctgggcctggggccaggccttgggcgggcgggagcccctgactcacagccacatcaggggctggcacccggggctcttccccaggagaggaccctcatcaccctccactgctgggctgcaggctctgccgtaggagacccaccgagcgtccccgcgggccagagtggaaggctggcactattcttagacggccggcctgacagcacgtgccggcttggcagcgggcagaagaggcgccccgggcatgggtgcctgtggctgggccctgccggcccctcccccgggaggcagccgatggcctcacgttccgtggtgggcagagctgggcgggtgggccgggcacaagctgctgtgcattgctgtgaagatgggaccctagagaaaggagcacttgcagccgtggggctctgcgcccttccctcccccgggccactttcggagggaccgccacgccggccccagggatgctaggatgccctgaggggcgggagtggccggcactgctggagaggctgcccgaaggtcccagaaccttggctgcacgttcgtcgccgcctctgggctcacatcttctggggccgcgagtgtggggtgagtaaccatcccaccccacgccctcccggacacttggaaggttgaggggtgcgcacaggcccggggaccagggctgtggggtggcaggagcccccatcctgggctctaatcccgacgcacccatgctcaacctgcgtgacctcgggcagagccggcgccacgcgggtgtcagtctgctctcccagagatggagtgagcaatcggccctcgtaga

SEQ ID NO: 132 - DNA, 717 bp, Bos taurus
gtgttccaagagaggcccccgaggggcacatggctgtttgcaggtcgggaggtgtctgagataaagcggctgtttttccagtgggctcggagccagagggggccgtggggccgccccagccaaggccaggccgctgcctcaggcgcccctctgtgtggagccccggggggagcaggaaggggtggttgcacaagcaggcccacgcttgccaaccagggggaccagtcctgcctcccacagtagcagccaggccttcccccggctcccaggaagcccccgccccgcagccggctaagagggtctcagagtattagcctcccacatgtgggagcctgtcatgcgacgacttaattaaggacaaatttcacgggtcctggatttggtgggcacggcccctgcccaccggggctgacaccccgggccaggggagggggctgaaggggctggggagtgggaggctccaggcccgcaggagggaggccgggacgctgggattaagggctgacatgatgtgcctgtcacggataagtgcttgtgtagaggaggggcggtggggggctgggtgggaagcgggcaggaggggtctgcaggaaatgggctcaagagccaaccctgcccacccggccggccggcctgggagcctctggagtggcaagaaggcctcgggtctggaagcagacccccaccacaggcctccacagccatcatgcgggggcc

SEQ ID NO: 133 - DNA, 530 bp, Bos taurus
cccgattgccttctgagccctgcggctcgcccagggtctcccaggaagggaggcagcgccccgattgccttctgagccctgcggctcgcccagggtctcccaggaaggaggcagcgccccgattgccttctgagccctgcggctcgcccagggtctcccaggaagggaggcagcgccccgattgccttctgagccctgcggctcgcccagggtctcccaggaagggaggcagcgccccgattgccttccgagccctgcggctcgcccagggtctcccaggaaggaggcagtgccccaattgccttctgagccctgcggctcgcccagggtctcccaggaaggaggcagtgccccgattgccttctgagccctgcggctcgcccagggtctcctaggaagggaggcagcgccccgattgccttctgagccctgcggctcgcccagggtctcccaggaaggaggcagtgccccgattgccttctgagccctgcggctcgcccagggtctcccaggaaggaggcagtggaggc

SEQ ID NO: 134 - DNA, 344 bp, Bos taurus
atgagtcttactgaaattaaagttcgccaagctcattgcaaggaaaaaacttgttttttatccgatgaagatggacttagcctgaagattgagccgactggaagaaaatcttggtgctatcgctatacagatccacaaacaaaaaaacgtcgccgcattcagttggggctttatcctgatttatcgctgaaaaaggcacgacaagtcaaagatgactttaaagacaataattattgttttgagcatgatactgtctctaatgtcatcacctttggtaaggtcggggaggagtggctgcagttcaaactgaaaaatgcatttaatgattcaccccgctgcggtgt

SEQ ID NO: 135 - DNA, 340 bp, Bos taurus
tcccctgccccctcctgcagctctgcctctgttgccccgggctcccaccttccactcacccacttcctcttcctgcttcccccgactctagccctgacactctgtccccaactctggccctttcttctctcctcctgtttctttacccactgctgccccttgctggcggctacccagctgggctccagcccaacagctcctccgccctcctgctccccacacagcccgggaggcacagaccagggtgaggccatgccgcagggctcagggcacaggatagccagcccgaaccctagctgccaccttctggccttgccacccctccctcctgacatgggcg

SEQ ID NO: 136 - DNA, 1590 bp, Bos taurus
  misc_feature (639)..(688): n is a, c, g, or t
gagatgccacttcgggaggcgcacccccatgccggcacctcaccccagcctgcggctgtactgcgccaaggacctgggctcccggcggccgggaccctggctgcaggccaaggtcccttgcccacgctggggccggaccctccaacctctgcccccaaagagggggagactttgggccctacacctctcccaggagtcgggatcccagagtccgagccctcaccccctcagcctggagacccggcagtctggaccccgagtcccttctctgagagggactcgggggcccgggagccccgctctctgcccccatcccctctcgcccccacaccgggtgccccctcccttggctcacctgtctgtcctttgcccagcgtcttctccagccgataggggcccacatattgggcgtgctggggtgggtgtgggtgtgggtgtgggaggtggtaacctttaaaaaaacctcttgaattgtcttaacacagaataccctctggatctattcttgttgtgccaaatgccattcttttcaatcttttctttggctaatattctattatatgtgtagtgtgaatgtttatccattcatctataatggacacttgggttgtttccataggtattggtatttgtaggtannnnnnnnnnnnnnnnnnnnnnnnnnnnnnnnnnnnnnnnnnnnnnnnnngggagtaggcggagccccccctccccagcccgtttctggccggtctcccgcttcctgcgcctccccccgccgggggggtctgaggtgcgggccagcggatgctgcaggccgaggtcccccccgcccccccacgcgccctctctcctccgcctcctcctcctctccgcctccccccgccacctcgtcctttctctccggcacgctgacatcaccatccggggactccgggcccttccccaaagttaaccctttcggagggggagggggagcgggggcagggggcgggcctgggcgccggagccgcggagacgcggtgggaggatgctgggagcgacggaaggatgcaggagggaccatggaggcagacgagtggggcacacaggaggggcagagccggggcgacacaaagggagacggagcctgacacgagtgggggaaggagacagacggcacgggagacggggaccgccagaaccggggctggggccggacagggagacgggggaccgcgcaggcgggcagagacaggagagaccagggggagcagagagacagggagacggaggagaggggagaagctgcctgggagagacagagggatggagaaaatcagagatagaaatggagagagataggggaagggaaagagaggtgaagagacagggaatgggccggggtagggggtgggcgggggttagagacacagagatatggagggacagccgggcaaagagatgaaccgaaaacagagccagagacagggagagactgagagtaatggaggagagagacagtggttgagagacagatggggagagaaatggacagaaatagagggacgggggcagaggggacaagatacagaggtggggcaccaggggaaggtgacgggaagggtggag

SEQ ID NO: 137 - DNA, 387 bp, Bos taurus
gaatttattaaagttttgacaaaagttaagaaaagtttgctgtttaactgggggccgaagggggagggaggagaagccctccgaaggggtggagattcttccccagcagtctcccctcactaccatcgagtaacccgagacccacctgggctgaggctggcgcgaggtggggagatagacgcgtccgctcggcaaggcctctgtcgtcaatgaagaaagagagtcggactgaggtcacggtccccgcgcccctgccgctgggagcaggatccagtagcagggtagctccctagagctctgagcgatttcgccgtgggcagccccttgccattttcgaacctggctcagttccgagctaggccgccagggggcgatgcaagatctcgg

SEQ ID NO: 138 - DNA, 344 bp, Bos taurus
tccgcggggagagggggcggaccatgccccagagcgagaaggtgagaaccaaggtgtgggggaggccccggcccagctcgggaccctacctccgaacaggacgctgttgtacctcttctccgctggcatcccgatggctgctgcaaaccagtccaccccgcgctgccaggagccgtagagggggtcctggggacaaggaggcgtggcgaactcgggcgtctctgaggggctgggagccaggagcagccatggcggggtgcaggggcagtggggctggcggggaggggctcacgtgggcacggccgtggctctccagcagcccgggaagtaggggcagcagcagg

SEQ ID NO: 139 - DNA, 309 bp, Bos taurus
tttcccaggtgaggacagaccctcctctctgactctcagcccctagtctgtgccggctgctctcggggaggatctatttcatcttcaggacaggcccatgtgatggacggagcgggagcaacacctgtgggatgggaagaggggcagggagccaggagtgcccgcccgggacccctcccaggccctggacctccaaaccagcatgcccggtgcccactgtcccgccctgggtcctcccacccagcgtcccgtaccctccaccctctgacttttcatactctttcctcgcagaccccaggccctgcaggt

SEQ ID NO: 140 - DNA, 370 bp, Bos taurus
gggtggggcgcggggtcgctcagccgaagctagtggcccaggtctctctggccccgaaagcaacggccccaggtctctccagctcccctgctctgactctgcgtggcgcttgacccgtcacacggcctcacctgcctttctcagtcgcccgcgccaggacagctcctggggcagcagtggggcacgtggtgacgggcccacagtggcttgagctctggagggatgagggctcgccgtcctgcgtcccctcgtcggccccgctgaaggtcctgaaggaggggcacatgatccaggggcgggcctggcccccaggccccccttccctccagggtgctgacgggtgagcagggtgcctccgaccgacgccggc

SEQ ID NO: 141 - DNA, 305 bp, Bos taurus
gtgccggatgcggagctgggtggtcaggccccccgcggcaggctgaccccactctgggcagatggaactcggctctcccgctcactggtcaggccgctccggcagacggagcacctgctctcacccactgagacccccgaggagctgtggaccacccgcccacccttaggatggaccaggggagacagagggtcactgcccagcctgggaccagacatggccttgccctcacaccatgggaagcaagagtgggagccgtgggggaccccaaaggctggagccacgggcagagagcccgctccatt

SEQ ID NO: 142 - DNA, 641 bp, Bos taurus
  misc_feature (290)..(339): n is a, c, g, or t
ggccggccccgcgactctccagctgcgcctcgggctgggatagccaacgagccggcgaggcccagcggcggctacgagaaatttctccaaacacagaagccccagaaaccgcggcgccggggcccaaactaaaaaaaaagtaaagtgtggcggcggcgcgggccgtcgggggcccggccgcggagggcggtgcggggcgcccgcgggaggccgggcgccggcgcgcggggctcgtggcgggcgcgcggggcgggagggcgcccccggggcccggagccgcgcggccggcnnnnnnnnnnnnnnnnnnnnnnnnnnnnnnnnnnnnnnnnnnnnnnnnnngcggcggggaggagaaaggggccgcggttctgggagctgcggccccgcgggcgggggtcgcgccccccgccggctgcccctcccctgcgcgagccgcgtcgggcggggcggcggcggcggcgaagttttgacagctgctccagttgttgttgtgggttccgggctgcgcgagagcgccggcccctcagcgcccgcgcccgctctccggggctgggaaggaggaagttttgcgggtgtgcgtgtaccacacacatacacgcacacacagacacacacacactggaccggggccgtcgcgcgag

SEQ ID NO: 143 - DNA, 849 bp, Bos taurus
gggttttccaagcaggggcggaagccaagtctggctgcttgggcatcgttggccggggggtgcaggggacggacagggaggcggtccacagcctctcaagaaagaagggtctcatgataggggaagaaggggcctggcctggccggaggcctgtgactcaagctctcccgtgagcgtctgtccaggggatccagaggccgggagacccacgagaaggaggcttcaccgtgcgggggggcaggacggggtagaagggcccgtcccctccccagccctgagggcccaagatcacagcatccagggaggaaggggaaggcgcccgccctgcctgcccggggcagggtgcttcctgccttccagaaccaaaggggacccgagggcggtcccagggcctccctgcccctgaggggcccctggccctgcgggtggctacaccttcttgggttttcggccgacttggtaatagtagtaaacgctgatggcgacaaaaaggaggcggctggccagcagcagcaggatcccctgagacgcgagccctgtctcggctagcgtggcagtgaccactgtgggagagaggcgggaggggcaggggtcaggagcgctgttcctcaccccccagcacgggaggccaggggccccggtgcagctgcctggctgaccccacccccgtcctgagctggggcgtccggccggccacccaaccccacaggtacgtgtggcagggacaggagcaagcatgtgggcacggatgggcgtgtgttgtcccccactgtgctggggacacgagggagcagtgggtggtgggtgcatggacgtgcattcaggagggcacaggcgcacgtcggccgt

SEQ ID NO: 144 - DNA, 848 bp, Bos taurus
ggtggggtggggcaagaggccctggatagacaagctctgccccaggccagatgttctggggcggctcctctgttctggggtacttgccctgcaccccagctgctccaccagggaagcaggaagccctggtcccggctctggtggctgtcccccagatggtgggttgggtccacccacggagagctgagggccctgcctggagtagaaggacccatcatctctcctggcccccactgacatccctccccccatccctgcccccgcctcccaccccagcgccatcctggcctccttgctcttaatctaagaactcctttaggggcccagccctgaccatccccaggctggagggggcgcgtagcaagcctgctttgccaggcacctgtctccccacccatcctgggggcagcgagagaacgagggtggggccagaccacggagcagcccgcccaggcagcccaccctgcccgtcagccccccgctctccggccatgtcgcgtgccccccgccccacctcggccccccttccccctgaacacacacggcagagaaacaggccccagcacgcgggcagccagctggcacccagtcaaactagcagccaggcacgctgccagggcagccagccagccagcgctgccactcgccccggccccgcccgcccgccggcctttacgctgggagctcacagctaggcggctgcaccacgtggcctgtcagagcgaggctgcaaggagaggagggcaggggaaccccgtaggaaggggatcccaggccgggcagcgcaactgcaggcagagaaggggagactggggcacaggcctgggaggtggtagggtccccaggac

SEQ ID NO: 145 - DNA, 703 bp, Bos taurus
gtcggctccccggcggaccgaattcacggcccagggttggcagtccccggtcactgagcgccaggacaatggccccgcactgcccccgtcccgctaaacgcgcggctcgggagagagtggggagcgccggtttccacctgtgagtccctaaccccctcccaacaactggggcttaggcgcggctcccgcctctgctgcccccaacccgggagactggggtctcccggtctggctcctcccccctcccatgggtcctttatctcccagctccagacacccacccactttctgaggctcgagtctgcgagccgtggggctccaggcggtagctctgggcggttgggcgcctgcggtgctccccgcccccggccccccgaaccagaagatccggacagacttgtgggcagcgggaactccgccatctccagcctcgacccagcggggtcgaggaaagggtctcgcaagtttgtgccgcgcgcaccccggacccgggaacaatggagggaggaggaggatggggacggggatggagcgggggaggggacagagaatcgagcgcggggaccgagtcagaccccagagcgggagaaactgctttccgctcggcttctccctcgctccgaagcagaaagcccgagcccgagatggggaggccgagggcggaatcaacagtccaggagcggggagacggaggagaaaggga

SEQ ID NO: 146 - DNA, 751 bp, Bos taurus
ggagctgaatgactgctcttcacctgacgctgttgtctcccctcctgtttgaaagacatactttcttggtccgtgagtctgtccacccgtgtcattagtgaccactttttatgtttggaggttgaagcagccagcgttctgacaggaatagaggcccatctgaccgcactgtctgcagaggtgggtgtgggcggggcaggtggcgagtaccctggggagggtcccccggcctgggtcggaggacgggcggtgggcagagagggatgcgcacacagcccgctggctggggaggaggagaaccccgtgggccgacctctcccgcatctctctgcagctcgtcccggtggccgaatccagggacggcagaacggccgggaggcggggtggggatgagaggccagccgggaaggcagcggggtcatccttcccgggaacgcgtcctccgccggcgcctggagacacccccagagcgcccggcctgtgcgcgccgcccggccagtgcagtcccggcggcctcgcgccgtgaacgtccacccgtgtctctgtcacgtttctctgcggcctgcgaggcaactgtcagaaacgccggcgcgaccgcccagcccagtcctctgcccagcggcttagccctccctcacccgccttccgggtctccgcccagcgcccgtggacgtgggccctggggctgccctgctgttccccgggcagctgcatcgcccgctctccgccgcaggctcttgcgcactggagg

SEQ ID NO: 147 - DNA, 1118 bp, Bos taurus
cctccacccgacacgttgagactcacagggcggggggctccctttgggcctgtgtgggaccctcccaatggctcagggaagcacagcaaggcacgcaagcagctttccccatggaggagaggcctgctcatacggagcccagcatgcaggggggggaggcggcaaggggctctgaaggcttccaccgtggccccccaggacccggcccacgaaggccgctgagacagtgtgggagccaagggctgcgggctgcggggccggagcaggtacgacaggccgggcgggaagccacagcccctagcacggctggcggctcctggagggtctccccacagctgctgggcagcctcacagacactcctcccagagcagggtgggcgcccctggggatcccggggccagccagtcgggactggaggtgcccagggagccgccccctcagccagacccccgtcccctggggaagggaggctgcaggtgccaagtcagcgtgggccgtgccccacagcagccacaccccagcctcatgggcgggcgcgggcgcatggacgcagttcctacctcctgtcctccctgcccctgtggcagctctgcagcctgggccggcgccccgcccggctctggccccgagcctgctgccgtgggggcctctccagtgcgcagagtggcccgccctcgcctcgcctggcccagcgggcccccaaggcccccctgcccccaggccggtgcccagcggacacacacgggtccggccagcacccaggatactgttgctcgtgatctcttgccacttgttctcccgcttgagtcttggcgcctccaggtcaatgagggagacggcttcttcgtctgtgatcccctcctccaggtagaattcaaccaggtgcagcacctctgcagacacaggcaagggaggggctgggaggctgtcagaggcagcgctggggggcaagaaggggggtcccagagctccttcccgcccactacaggtctcgctgaaagctggcggggcggcagggggaagcaggcgtatgtgggcaaggggctcctctgcaggggaagagaggcctcttcctggctcctggaaaggggcctctaatccccatccatgtggaagg

SEQ ID NO: 148 - DNA, 301 bp, Bos taurus
ggggaagataggactcagatctagggaaaaacaggagagatgagcatgaaaagacagaggaatgggcaaagatgagaaaatcctgggcagaggtgagcagaagtggagagagatgggctgaggtgcggagatgggaggtgagacagacagagcgggcagaggagggtgaaaactggtcagcacgtgtccagaagcagagagatacagggaaagatggtcggaggtgggcacacaggagcgaagcggaggaggggctgagaagcgggaaagctggaaaggggaaagagggaagagagaacag

SEQ ID NO: 149 - DNA, 942 bp, Bos taurus
cgttaattaaacagcatcgggttgcagagccggccgcggccgggcgggaagcagcgccgcctggcgggcccggctagcatcccgcggaccgaccagtcctcgagctggtcctgcgagccggtccgggggcgctggctgcgtgatgtgttcccaggccgtcaggcaggaaaacaggggtggccggcgggctgcccaggacctgctctgcggctgtttatggggaggggataccccgccccccgtccctccctcccgggcgcagaggcgaaggggcggcgcgcccgggcctggttaaccgcagaacgatggagccgcgcccgccccgcgcccgccgcctaattgtctccgccgcccgtccccatctgtccctctccgcccgtgtgtgttttggcctttgtctcgccgccctgcgctgacaccctctttcaaagcccacttctgtcgcccttcaccgatcgcttctgccccattaacccatcggcacgaggctcccgcaacaaaggcccggagtcgggcggacgcgcgcgcgcccgcccattgtccgccgctgaccccgcggcccccagcctggcccggccggcccgacggggcggggacggggtcggccttgcagataaacacagaaggcgtcctgcccgggccgcgctccgccctcagtgaacgggacgggacaggacggccgccttggcaaggccggcgagcagctgccgccgcgggaaccgcgggcctgcccgccgggcagctaatccgccgcaggcccctttgtccctcgctggacgtgaagctggcagagagagcggctggagagagagcaggcccgccgcctggcgtgcctggtaaacaaagagaagtgttgctgagcggggctgccgtcctccggttccggttcccggtcctgcccggggaaggagagcaggccccgccgacctggcacagggactc

SEQ ID NO: 150 - DNA, 640 bp, Bos taurus
taagcttgctaggtcaagtctggcttggcccttagcgagtccagggccccagaccggctggacaggggagaggaggccgctctcaggtgagctgcgagcccaggaggccctcgccccacccgctctctaggaactccaccgccagcgcgaagtcaaactcgtgcagcggcggcccatctaccgcgatcttcaccacggggccgccccgcccttcccccgccccgcctggcccccaccaccgcagctcccggctgcggccgaccttgtccaggaggccggcgcccgcaggcagcgccaaggccagtgcggccagggcggcgaagtcggggaagagctcgtgcagttctgagcgcgcgcacgccagcttggtgcacagggcccgcgggcccagccgccccaggctgcacaccacgcgcttgaagagcgcgaagtcgcccagcgccctctggcgcaccacagcgggggcgaaggcgcgcagcaggacgcggagtgccccctcgccgtgagcgcccagctcctctggggcctgcgggtagcggcgggggtcgaagatggccgcgaaggcggccacggcgtccagcgagggcccggggtaagagtcccgcagccccctccgcatggagtccaggaaggctccccg

SEQ ID NO: 151 - DNA, 379 bp, Bos taurus
gtttccggcccatggcggccacccaggcaggcagaggaggaggccactgccgggaaggagaggcaggccagcgggcacgagagacagctgctgagagagagcgagagagggctggggccagagagcgagcctggggccagcgatgggcagacggacgacccagacaaacagcaaggaagtcggagggttccagggaatgacgcgtctcccccacccccgcaagcatgcaaacagcagacctaagccaatgagaggccgcctctgcggggcggggcctggtcccagccaatggcaggcggtgcaggcaggggcggggcagttgatgcagctgtgctctgggggagggggggaggctaagtgggggacctgtcaagatgcc

SEQ ID NO: 152 - DNA, 695 bp, Bos taurus
gagaggcacagacctgagagccgccgcgcggtgaaatccgaggagctctcttggcgaggaggggctgtcagaggccatttatctctcgccagagtggaggctcggctcccacaccgcccgcgagtaacaatggcagcgatcagcagctctgcacgcgcttcagtgcatgaatggattgcggaagatgcctgcgctcttcttcgctcaggcggctctcggagcgccccctccccgcccgccgcccccgccccgccggcctcctccggcccgagagcgcgcggtgcgcctcggggttcgcgagtccggccccgcggctcggtcactcttacccggatcccccgccgcccttcccccctgccgcggctcccagggctctggcaccttgccaccgagtgtcattaggggctgaaacttgacacaagccttcccgacgcagacacccccgccagccccctccctccttcgctcccctcgcggcaagtttactttcaaggagttggacggtgtttccgcctcaaaggccggccgagtgagggttccagggggaggggcgggctcgggtgtcttcacctgccccacggcctagaatgttcgcaagcccccggctggcccccagcccgagcgcccaaggcccgctccccctcctcttcgttttgtagcggctaatccaccgagagtcccagcgatccccacac

SEQ ID NO: 153 - DNA, 395 bp, Bos taurus
ctggggaggaaggcagggctccgacccccctcccgaagccctgggtgcagagggggcaccgggcagggtcaccagcactgacgtctgcactcagaggaggccccggggccagggcagggacacgagagcacctgctcaagaaacaggtgggagagacaacccaggctcggccgagcaaggcctcagccagctcttcagggaggggcccccgggggtgacaggagcgggtggacgacaccaccctcccccgtcccccagcggatgatgctggctgtggtctgcctgacctgccgagagtcccagcaggactgaagcctggctgcccgggagcaaaccctcctttcagcgtggaggccaactcatggaagaacctgtcatttcatgtggcttattct

SEQ ID NO: 154 - DNA, 567 bp, Bos taurus
ggtggttggaaccgaggactgcctccatccctccatccctacccagcacagcccggaccgcactagggcttggggctgtagccttggtcacccggctcctgggcctcgatggcctcgtagaggcccagcacccgcctcagcagcacttctgagcccctgtgctcgagcctgagccaggggccgagggcaagggctccttcctgccctgcttcccggcctctgtgctctccatccccccattccccaggtgggccctggggcagggcagaggctggggtcagaggggccaggctggaagcccctggcgtccctgccgctctcggccaatggcctgtcaccgcccaggggtttactcccctttctcccctgggccctgcagggcccccggcagcccaccagccctgtgagtgctgccttccaggccctgagttggggggaggaaggcggggagacacccaggagcactcggacaggcggcttcatctgtttcctgctcctggtctggtgtcacatccctggggacccccgggcccgggcccccacctcgacacggacacactcatcctc

SEQ ID NO: 155 - DNA, 156 bp, Bos taurus
tgcagggcagaaatagagacacagacgtggcgaacagacaagtggacatggggtgggggggaggaggagggtgggaaaaattgggagattagggttgatatatatacacagccatgtgcaaaattgataactagtgagagcctgctgtacagcaca

SEQ ID NO: 156 - DNA, 512 bp, Bos taurus
cgcgaaacacaggaaacctaaaaggaaccgggcttgagggcctgggcgagggggtgttatagagaccaaggggctagacaggtcacggaggcgcccagctggttcatagagaggacttgagaatgatagaaagtcccagggatttggggaggagagatgggaacaagggagactctctgggaccttggtgaggcatagaggggatcctggatgaaggattgcttaggagacgtggcgggcagtggagaaggggctagagggattattgaggggagcacggaggagaagcactgcagatggatagtcttgggagaacccgaaacagggagctggggggcctggaaaggagaggtcattataatgtggggacttgcgggaagtgccagaaggtagagagctcagatctcaaagaaggggcctgggggggccttagacatccatggggaagagatctggaaagctggggggcatggggggaagcctggttccagaggggtcatgagtcggggagc

SEQ ID NO: 157 - DNA, 782 bp, Bos taurus
  misc_feature (110)..(453): n is a, c, g, t or u
ggcaacgtggaggaggtgtgcaggccccggacgcggatgctctgctcccagaacacctacacgctgcccggcccgggcagctcggccacgctgaggctcacggccaccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntccaggcggcgcagctggcggagctcacgcggccgccgccgcacccgcccaagcccaagggcacggcgcccttccccgcgcggccgccgcccgcgctctacacctgcagccagtgcagcggcgcagggggcgcggcgggccggcccgacccgggggcgcagccgggcgccagcctggcgcacgcggccagcacctctccgctgacctcccagtcgtcctacaaccttctgggcgcggccgaaggcgcccgcgagcgtgctgactacgtgaactcggccttcactgaggacgaggcgctggcccagcactgtccagccgacaagccgccg

SEQ ID NO: 158 - DNA, 503 bp, Bos taurus
gagccgtgacccctcccctgacaggacgtgacctgagccgtgacccctctcctgacaggacgtgacctgagccgtgacccctcccctgacaggacgtgacctgagccgtgacccctcccctgacaggatgtgacctgagccgtgactcctcaccttgacagtcacgcccgggaccgtgacccctccccggggggacgtgacccgagccgtggctcctcaccctgacagatgtgcccgggaccccgtcctctcccctgacaggacatgacctgagtggcagctcctcactgtgacagacccgccctggattgtgtcctctcccctggggggacgtgacctgagccgtggcttctcaccctgacagacccgcccgggattgtgtcctctcccctggggggacgtgacctgagccgtggctcctcaccctgacagacccgcccgggattgtgtcctctctcctggggggacgtgacctgagccgtggctcctcaccctgacagacc

SEQ ID NO: 159 - DNA, 944 bp, Bos taurus
  misc_feature (573)..(622): n is a, c, g, or t
ggacagacacctgccagctgcctcctgggccaggccaggccaccctcaggttcctcactgccctgtccacaggggactcgtgggggatgctcgcgtgtctgtgcaccgtgctctggcagctccctgcagtgccagccctcaaccgcacaggagacccggggcccggcccctccatccagaaaacctatgacctcacccgctacctggagcaccagctccgcagcttggctgggacctatgtgagtatcggggctgctcaggggctgggagctgggtgaagaacggggctgagagaagagagaacacctagagagtgggggtcctggtgaatgacggggtgacggggaggggcttgggctctcaggagtccccctggctggcccatctcctgccctccctcttggggaaggtgccccctcctccccatccctggccccaggaccaggcatgtgggcaggccttgcacccgccttggcccactgccgcactggctgccggcccagccgcccgcctccccctgagggccagggaagtctcctctgtttacaccgtgttgtggtgtctcttgcccgnnnnnnnnnnnnnnnnnnnnnnnnnnnnnnnnnnnnnnnnnnnnnnnnnncaccccccatgcctgtgttccagctcgtctctgccccagacctggggccctgctgctcaggacccatgggcctcccttctgtctgcctctcccatcctggctgggcctcctaagggggacttggggagaaaagggggctgtagggaaccccggccaggaggggcagggggttcgggatgtggatgcaagctatgccataaggatctgggtgcggtccggaggtgctcggagagcccaagagagaagggaggagggtcggggttgccgaggaccgtggggaactggcccctcttcccgtgttcctcctccacatcccagaccc

SEQ ID NO: 160 - DNA, 460 bp, Bos taurus
ccggacgccgctgctgccgccgccaccgccgcctccgctcccgtcgcctctggttcttcaagctgtcgtcacctgagagaatctgttccggtgtcggatccggcggggaaagtaagtccaccccttatccgttttctgcctccacactccttctcccggacagcctcccactcccaccctcttcggggggccgtgcggggccgggctcggggggggggtggggggaggccgccacctcaccctggcttggggcctccctcgccgccccctccgctgcgcgccccggggtcttcgggggcacggtcccggcggcggccccgccgcgctcgcttcctccgcccgtcagcgccgggccctgcggtcgccgcgcgtggggctctcgggccccgagggcgcgctgtccggggttggagacacctcgtttaagcgaggagtcagcgtctgcggacgccggctccgg

SEQ ID NO: 161 - DNA, 330 bp, Bos taurus
agcttccgggtcacggggccctcattcagacccagcatgtctcaagctccaccacggctcactgtgcctctgcccccaggtcgtgtggaccatccagggctgtgtccgggggacacagtgcccctagtgtggccagcccagtcccccccaggccctcccacccgcaaggagacgcggcctgccgctcccagtctccgtcccccttgaggatgtgggctaccactgctcacagtgcccggggttcctgactgttagggagcgcggggcaggcagaggtgcttgtttctggggccaggccttcttcatgcccgtgtctgcccccagaggaac

SEQ ID NO: 162 - DNA, 317 bp, Bos taurus
gctgggctgatgggcaactctgtaggtgcgactggccttccagcacggggccctgggtggcagtgggggcctcctctggctcccgccccgctacggagaagaggaggcggggggccctggagctcagcacagtgggcgctccgcgtccccctccatgtcacggaggcaccaccggcctgcctcgtggagggtggggaggcctctgccgcaggcctggctaacaacaactcgggccggagggcctcctgctgtccggggtctcgggtcgacgtccctccatccctgcagcaaggggagaagtcacctggcgtcaactg

SEQ ID NO: 163 - DNA, 607 bp, Bos taurus
gcggatttgcatgaaacacagactgggagcgggcgggagtaggagcgcggcgcacgccccgggccggcccagctagcgagcgagctgggaaggaggagggcagcggggaggcgaggctgccgaggcggagggatctgcgcatcaaagcgagcgaggcgagcaaagtttggctgggggttggactttccttcctggaggcggcacccaaacagccaccccgtgcggaaacccaaactttcttctgccactccggtccccgcggccgccgcctccgccccgcgtccctggccttcccgatcccgcttcgctactgcctgcccgccgccccgccgtccttctccccgccgtgggagacgcccggggcgcagggccgcgcgccgagccccgccggctgcagcgccgcggccgggcccggagaaccagcaagttcgccgagagttgaagcgaagtttgggcggccgcggccggcccgggctgagctcctcgctgcgcccccggcgtcccgccgcgcccagccccgccgggggcgctcctcgcagctcgcgcgccctccccaaccatgtcgtccatcctgcctttcactcccccgatcgtgaagcgcctgctg

SEQ ID NO: 164 - DNA, 1525 bp, Bos taurus
  misc_feature (233)..(1462): n is a, c, g, or t
ggtggtgacccggccgccccgcaggccgcgagttccccagctccttcgaggccctggtgaagaagatctgcaagtacctgttccatgtgctggcgcatatctactcggcgcacttcaaggagacgctggcgctcgagctgcacggacacttgaacacgctctacgcgcacttcatcctcttcgcccgcgagttcaacctgctcgaccccaaagagacggcggtcatggacgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccacccccctcccccatcaaacctgagtggcagtgatgggaccctgggctctggtcttgactc

SEQ ID NO: 165 - DNA, 474 bp, Bos taurus
gtcaccctgagggagagcctcgcagagaccctgcatgggggactgtcgaggaggccaggatggcaggagcaggaatgaggtcagggaggaagtgcatgcctccgggggtcacaatgagtccgacttggccatggagcagaggcctggtctgacggaggctgcagagaaccaccggcggctgtggaggcaggcctgagggcagggctcgggggctgtaacggaggcggcgggggggggtggcccaggtgggcgctgcgggcagctggggagaagcacctcctagagagatgggggaggaggtaccagtgggggcgggggcggctccatggcgtctggctgggcagtcaccgccctggtcactggctacagaggcttggcagggagttgcctgggcctggatcactccagctctgcactcgtaaatgccgggctctgtttgcagtcacaggggaggtggtcagtgtgtagtggaca

SEQ ID NO: 166 - DNA, 727 bp, Bos taurus
ctcggaaggctgcctgacaggggggcccaaggtccggggccttcactctgtgagaggaggtgaccggggctggaaccggtgggcctcattccgggtccccgcggcccactcagctgcgcccatgtgcccatttcgcagatgcaaacactgagtcgccgacggggcggggctttctaggatacggcgacgggttgctttggaaaatcaggaccaggaagcctagcctcccaggatccggcccttctctcatcctcgggagggtggtcagtggcggggaatcttgacccttttctcctcccctccacaattcccgcagagtggttccctctacggtccccgtgtggcctcagtggttcagccctccggcccccgagtccccccgggtggagggtcggtggtaccgcccgcaggcccccagggtcagtggcttgctcccccctcccctggagcccaggcggagggggctgacagagacggttccggggcctggcgactaggggctgtccccggcgctgcctgagctccaggcccgacgccatcttgtggcggcgccgggtttccgctcaggcaagtgcgagagcgactaaagggcctgggaccccggggccctgttcggacagccccgccggcccgggacctcgccgccgggtctccccgcagtgtggccccgcttcctagcagggtgcggtgtggcgacctgactctccccgaagctcacctgacgctt

SEQ ID NO: 167 - DNA, 401 bp, Bos taurus
aggaagtgagccgcatgggtgtcaggctggtgggtgaagagggccatggcctcgagctggggcgggagcaacagagcccaggacttgctgatggggggcccagaggcacagtcccggggtgcctcaactggctcagctccagtatgggaaggggccccagagagaggagcctggcagcatcaggccggaccatgggccctgccggagccagaaggaaggcgagggggatggggcaggcggcctggggaagggtactcacgggcatgatggagaactgcatgaagaggcaggtgagctccagggccgccagcacgtacgtgagcagcacgacccccgagcggcccagggcgcccattccgctgggggcctggccctggcccccgggggccccgactttccgc

SEQ ID NO: 168 - DNA, 383 bp, Bos taurus
cttctccttgtagcgcctttgcagctccgccagctgcacgcgcatctccacctcctgcgtgtccagccgatcgatggcagccttcagtgggcacacctagtgggcaggggcacaggggcctgtgagcccagctaggggaccacgatggggtgtgcgggcagccagcctgggcactcgaggccccgcccaccgtcacggcagtgggcaatgggggcgaccagggacccctaccactcacaggcttggtcttgggggtccaggtgtccttgcgccgcaggatctgcatgcggggggtgagggtgggagccctgcaggggtcagcagtggggggctgggccgaggaggcggggctgtccaggttagcggcttcgaccagggaccag

SEQ ID NO: 169 - DNA, 397 bp, Bos taurus
ctccacgacccccatcaaggagccaagcccgccccacgctcagcactgaggtcgcggccccaggaggtgagggctcggaaccgggtggcctttttcctcaagtctctggctccatctgctgtttctgacggtcacgaggagccggaagcagtgtccagagggtggccctcgggtgagcagcccccgccccggcagggcggccctcccgctcagaggacaggcggggcgggggggcgctgctcaggagactgtttgcttgacccctgagcccgccgtggtctgcttccaattaagtaaaaagtgaacctggttactaggttaccctacttgccacagctcgtccagaaagccagaaatcacttcactcaggctgacctgggcttccatcgatggtgcg

SEQ ID NO: 170 - DNA, 696 bp, Bos taurus
ccgggcacacgggggtgacggtaccccaggagccctggaccccaccatgtgattctccagcttagcctcctctcatcctcctgcacccccttgtctctcctgcgtgtgctcacctgcctgtgttcacgtctcgtctgtctgcccagcccccagggtccctgacatcctgagctccgtgaggaggggctcggggaccccagaagccgagggctccccgccctgcccgtctccggctcccattagccccctgcccaccccatgtaagtagccgggccctgctgctgtgggggtctcctggaggggccagcagctcctctgagggttcactcgagggccaagggccagctgagctcgggcaccagcctgcccctcacagcccctcaacgcaggtccccagcacctctacccctcaagctgggccttctccactggcctggggggggtctcaggaccggggctccttggtcctgagatacaccctggggcgggagagagagggaggcctgcttcctctccaccagcctggcccgccgcctcctcacgtgtctgctcctgtccaccccagcccccaggatctctgacatcctgagctccgtgaggaggggctcggggaccccagaagccgaggggcccctgcccaccccatgtaagtagtgccccaggcctgctgcctctgctgccaagtggctcccgcat

SEQ ID NO: 171 - DNA, 385 bp, Bos taurus
tactggccgaggggtctttctggagtgccccccctttctggaagctcctggccccacttcctgcatccagtggggtccctgatgtcaactcctcagcctggttcctggccctctcctcgctctcggtctcttctgctcccccagcatctcaggggctcccctctgtcaccccccatctcctcaccctctgcaaccccgccgaggcacctgacgaggctcctcgccccccatgaggtgccatgtccctcgcagaccttctgtgttcagattccacctcccgtcccccttctgcccagtgatggtggtggcagaggggctcctcttcctgcctcagcagcaccccagccccatggctgacttggagccagtggtctctgcacattgg

SEQ ID NO: 172 - DNA, 449 bp, Bos taurus
acacctggtctcaggcgaggcagagaagaccactggtccagaacaggccggcaggcggcgtctgcgatgatcccagaccctcatcttgggggagctgccgggggcgggaccagtgcatccctgacgggtgtcctggtgctcagggttgggtgtgggaggctctcatgtattctggggccatggccggtggggtaggggagccaaccctcctggggcagttctgtacccctgggggttcactctcccactcggaaggtggggctcacgtccaccagggtggtgctgggcgggaactcagacactcacgggagtgaggcagtggttggagctggggagggctgactcatcctggctggactgccctggggcagatccctgcggctcaatggggggggctcccagctggtggggatggaagcgtgggtcccgaagggctggggaccctcagc

SEQ ID NO: 173 - DNA, 708 bp, Bos taurus
ccagcacttagaacagcatagcagcagctgactgagtgcacgacccgcgggttccgtggagtgaagcgctagctgggcagccgggagggaagggtctgttctggtcccccacccagtctgccttctatgcccgagcccccagctcaccttgctgaccccggtccccacccccaccttctgcacccaagctcctggctcaccttgctgatcccggtccccgcccagcccaccttctgcatccgagcccccactcaccttgctgaccccggtccccacccctgcttcctctgcccaagtcccccgctcaccttgctaagctgaatctccataaggacgtggtgctgcctcccactgcccccctgtgcacgccaggagttctggggccggttggggccggagctgcgagaaggggaactccgggggcctgagaggctgacccttgccctgggcagaggaacaagggacgagttgactcaaagggaacccccttcccacgttccctccggtcagagtcggggccagagaccctccccctgggaagttggcggggcatgggaaagaggaagccttaagaccagaagatgaggcttgggttcaaggtctccagctgccgaaggtcagaactggaccccagcccgggccccatctcctgggggccccactgccacccgcacccgctccttctcacctgtggccagggtgggggcc

SEQ ID NO: 174 - DNA, 350 bp, Bos taurus
ccatatcagctggctgtgcccaggactgccagccaactggccctgggaaggtgtggggcaggcccacggggtgccccttgagcagtggacggacaggacagggcaagcagaggcctgcggccaccacccccgacctgccgtgacctggagccccgctggggccctgggaagtggctgcagaaggcacaggtgggccacggcatccaggctgctcaggggcaaagggcacagcagagcagaagggacagggcagcaggccaggtcctcgggggctcacttgcccgctgggcactgccgggacacaggccggccccccaggggagtggctggcgggggcgaggggcgtggcc

SEQ ID NO: 175 - DNA, 445 bp, Bos taurus
cggtgctgcgatggacgcgcatactttcccacttgcgtcttcagattccgcgcctcgtgcgcctgtcgccctggctgggcagctcacatctggagaagcgcgaggtgtgtggggagcaggctgcccagccaggcctggccccccgccagcgcttcgggggccgcaggcagggtcccggagcctcgggctgaggtcgctgagcggggcccccaggctcggggcgagacagacataactccggggagcggggtgcagagcccccccggcccgtggtggtgggcgtcagatgcgtagccgccggccagctgctgagcgtctctggcctgtttcctcgcctgttagatggggggtggttccagcagggcaggcttcttggggcgatcctgagggggtccccgtcagcaccagaggccggtgacacggtgtgggaagtccaggctcgtaa

SEQ ID NO: 176 - DNA, 1389 bp, Bos taurus
ctcgggccattgtgtcggaagaggcctgcacggtcacgggcatggtcggggtggaaggccctgtcccggggggtggggcgcacgtcccccacggccgctctgtgtccccccaggggcagaagagccggcgggaggcgcagagggcccgggcttcccgccccaggagccgcaggacagcgagcatgtgaagacggcccccgagggccctgcggcagaggccgccccccggactggcgcccagcccgcctgcagccagggtaaggcccagcatgaccccggggccagcaggcggcgggggtgtcccagcagagggacctggagacggcgggggcgggggcagatgagaaaggcaggggcacccgagtcccctgctccacactgccgcgcagatgggagtggttcgggtggtgcgaggcccaagccgaggccacttggcccagatggagctgaggggaggccggggcggccgggaggccgggctgggcaggggacatggcgtcgcgtccacggccctgcctgagtcagagccagttcccagcagccaggcccccggggactccgtggggcggggcctctctcagacccctcttgggacagcagggccgctcccgctccttttaaagccacagggcggtggacgggcgtggacatcagccagcctgcggctcctgggcgcacaggggtgtggacgtgggccagggcgggctctgggacgtgagagctgacgaggtctgggaggctcggggctccccggcgcccccagcggacacactgctcctgagccccccacccccacccccaccccgtctccgcttccgccagccagagccaggccctcagggaggcagggagacaggaagcggcctgggagccagcaccctgcggtctaacacggccagaggggagcagggtgacgaggcttgcaaaggacaggctggcctgggaggctgcggaccctgctcagccttctgggtccgtcccgcagtggccagctccaaggcgcccggcggcgggagtgccccaccccccgagggccccctgagcaaggctgagcctggccgggccaagtcacgcctgctgcccaacatgccgaagttggttatcccctcgtccaccaccaagttcccccccgagatcaccgtcacgccgcccaccccgaccctgctgtcacccaaaggcagcatctcggaggagaccaagcagaagctgaaggtgacagcaggtcctggggggccccgggtcgggggcgcgggtgggctgaggctgggccgccactgacccctgcccgcccgcccggcgcagtcggccatcctgtctgcacagtcggccgccacggtgcggaaggagagcctgtgccagccggcgctggaggtgctggagacgtcgagc

SEQ ID NO: 177 - DNA, 607 bp, Bos taurus
gcccacctgaagcccaccatcaccaccccccgaatggttccaaagcctgagctgggggctgacaggcattggaccaggcgcagaccctcctcctctgcacgagggacccagaacagaccaggcctctccgcagcccccccaggccctgcttggagtcctgctcaggccgggtcccagaggcatgacccaggggcctgcaccatccctgctactgcggggagggtgccctgagcagccttgcgtccggcggcccctccctcccagctgggcggcccctggcctctgcacccccggccccctactgtggtgtctggctccacacctctgggctcaggcccgagcccctccccggggcagaggcaggctggccgaggtcaccgcaggaaggaaggaggtaccctcgtgggctttacctcagcccgggcagagcgcccctcccctggcccccagctgccccttctgatgggggtggtggtgggccggggcccccagcctcgtgtgcaggccaggctccgtgctcacgtgttctctggctcattcacctgttcattcatttgtaaattctccttcaggagatggtagctgcaggcaagccttgcagcaagat

SEQ ID NO: 178 - DNA, 828 bp, Bos taurus
cagtgaggtgaaggggcctccgaggaggccgggatcccggcggcaggcggggccgaggcagccgctggggcgacctgatcaggaagccgcaggacgggcgggcgcccaggcttacaacgtgtggccaccgcgcagtgggcagacggggctgggggcccgaaggaactcgtggcggcgggagaagccacaggccgcagcagccacctgccaggcagcgtgggagcccatgcgcagggcgctggctccccggggcccgggcgtgggctgggcaggggcacagcgatcctgacgcgcctgtggaattgaaggaggtgacatttgggacgcaggatgacactgggcccaggggactctggccaaggcagcctgcaggggtctgggggaggggcagtcccgggctttccgggtcaggaccagggcctcagggcgtatctgggggctccctgggcaaccatatgggtggacgggcgcaggcacaggggccggcagcctctctggagcacccccatacccctggggagcccagtcacgtctccccaacaactcccgcctctgtaaggagcctgatgccttgacccagggcctcagggagggtgggcagcaccaaggaaagaccatgtgggcaggtgcccgcgctgacgcccgccaaggcgccccctgccaccccgaggccgggcgggcgagcccctggccctgccagggctgaccccgcaggcccagctgcctttctcactcacccaggcccctcctcccctcctccagcagacaaacacaggaacattcccagtagacttttgggggctgggacccaataatagatttaaccat

SEQ ID NO: 179 - DNA, 301 bp, Bos taurus
gagacgcttgtctcctctctggggctgcaagatctggggctgcaagatctggggcgtgtttatgtccctagggcctaccgtgaggttgggtaaatggtggggggcgtggaaaagtctcccaagtcgtcccttctccaccgcggggctggtgaggcccaccttgctccctcccccacccagcatctcacggtcccctgggagcggaccccctggaactcaggctgacggcccggggactggctggcagctctccgtgtctcaggcccgtccagcccctgggcagcccctcggctgactctgc

SEQ ID NO: 180 - DNA, 305 bp, Bos taurus
gccctctggccgcccctcagggcctcctgggccaggcttcctcaggcgggagagccgggcgtgggaatgaccggcgcgtccgagacggatgtgcgctgagcgcagcggacagcccgccccacgggtctccctgggcagactgggagccacccggccctgtatcagctcagcctcgccttacaaggccaggccctggatggctgcatggcccacaggatgccctgagtccaagagtcctctggaagtgcatgggcccagggaccctggctgtccctgacggtctaccgcatgggccaggcctgaga

SEQ ID NO: 181 - DNA, 470 bp, Bos taurus
tcagcccttccggaactgggccagcgtgtcctcagtgactgcagggcctcgtgcttccacaccggatggccgtggctgggctaagcagagggcccgtgttccatgcagaagcgccggccgtgggcggggggagctgggtctgggcaggagttcctgccggagtggaccccccgtccccaaggccaggactgaggtcccagccactgcagcacactggaccggctgccctcgtagacatgggtccagaggggtttccgcagacgggcctagctggctcctgcagcaggggcagctccccaccccaaggggggcctggaccctgcagtcagctgcacgcagtcccaggcatcccccgcacgccctcggcggcgatcgtgtaggagcaggatgaggccggcaggtggggggcgaagactaccctgagggtgcggccaggcggggtgagaggcgcctcctcagggcgctgccgg

SEQ ID NO: 182 - DNA, 340 bp, Bos taurus
gatgaaggggctgagagtcttgagcgcagcctgggggtgagggcattgcccaactagcccttcaacctcccctgactccgctgtgcctgcctcccgggtggggaaactgaggccccaagatgcagaaggagccaccactggaccctgggggccgagcccagagcccgtgcccgagtgtgggaaagaggaagcagagagagagggacgtccgccgggggccggcggtttcctctcggaggcaggagtgcgaggaggaagaggagggctgggcaggaagcggccatgcaggcgggaggggcagcctcctgggggctcggacagacctgggcccgagtcccgt

SEQ ID NO: 183 - DNA, 564 bp, Bos taurus
gatggaccagggtcacagccaggcgtccgggtctgggtccccctgggaatgggcccagccaccaccctgccctgcggggcctccagggagggcgaggcctgcctcccacaccatccccgtgagcagggtcacgcgcagctggtttccagtggtcgcatggcacccctgccaggccctagtggcttacaggtctctggtggacagtgctctcccgacattggccgaaagcgccccgtcccagctcccctccgtgtctctcgccccctccagcaagtgctctgagccccgtctgccccccccgcccccagcccctacctgggtgctgggacaagtgtgaccctggggaggcagtcttgcttctagaaaaacctgcacacaccattgcagacgcacagtcacgggcggccaggccagccgggcgctgtgactaaggagcagtgactcacccccctccagccccgtgtgacagaggcagccgcccaccgtctgggtgcagctgtgatgagataacatcttcagggaattttcttatggggtccctccggccgcccccgcccctgcggc

SEQ ID NO: 184 - DNA, 688 bp, Bos taurus
ccacgcaggctgcctgctccagacccccactcccacctgtgtagcccctgccctgccggcaccaccgccctccaaccttttccgtccctgggtcctcactttccttcccaatcatcagactcctggtgaccccctacccccagtcactcttggtaaaacctcaaccccaagtaaatcttgaggctcctacatgctgggggagggggcgggtaggggttgactgtcccaactgccgcctttcccccctccctgtctggggggcccttccgctctgcctctgacccccagcccctctcccctcctctcagttgtgacttgggctgtgtccccgtaagaccagaaggtcccagagaaatggctgtgcgctcagctcgcccagccccgcttctgccgccccttctccggtctcctcagcaccgccttccggagcccggtgctcccagcgagttctctgctgccttgacaggccccccttgacaggccctcccagccgcccggccacccaggggcccgtctggctcttcagcgcggccctcagtgcggagcagagctcccatcgcgcaccccgcgccccatcttttgggtcctgacagctgggctcctcgtctcctctgccagcccgtcagcagcagcagtcttccttagatacgttggagctgcggcctgcctcagagtagacagcggcttc

SEQ ID NO: 185 - DNA, 835 bp, Bos taurus
ctgtggtgaggagaatgggttgaggggcctggtaaggccgtagaccgtcctcgagtggccccttgggacctcagcaggcgctggtgacggcggctccagtgggagtggggtggcgggcaggcactgcgctgagggtccgtgacccccccatgcccctgccacccgtgacctgcccgtgctcctcccacccgtgacccctgtgctcctgcctgtgacccccccaccgcgctcctcctgcccgtgacccccccctactcctcctgcccatgaccccccgctccacccacccgtgatcccccctgttcctcccgctcgtgacccccccaccgtgctccacccgcccgtgacccccctgctcctcccacccgtgacccccctgctcctcccgcccgtgacccccgcgctcctcccacccgtgatccccctgcttctcccgcccgtaacccccctgctcctcctgcccgtgaccccccccaccgtgctcctcccgcccgtgacccccgcgctcctcccatgcaggggacttcgtgcagctgccggtgcccatcatccagcagctctaccactgggactgcggcctggcctgctccagaatggtgctccggtgagtgcgccgggccagccctgatggaggggtgggggtgcacggggagagccccgggcgggattcgttgtccaggcggctgtctccagctggggcttgggggaggcccagcatctacccaggctccaccccacctgtggaagcccgcggcggtggaccaggccccagctctgcctccggaggatgcccagagtctggaacggccaggaagtcacaagtgcccaggcctgc

SEQ ID NO: 186 - DNA, 1477 bp, Bos taurus
agacgcccaggtctgtgggcaccgcctgcccttgccccccaagagggtctgcctgggccacccggggcagtctcagccctctcactgggctccacccatgcagctgagtcctcgaggggccgggaactcggggggctggccagaataggctgaccacagggctccccgctgagccgctgtgtccccaccctgccacccactcccgggggtccccccagccctctcctgccatgggctccccccgaagtcaagcgctgcctccatgcatagcgacccccagccgaggccaaaccccaaactccctcagcccctcacagtccaagcaagtggggactgctgcacagccaggcagggccacgagccctccacgccgcccgccctggggaagaaccctctgggggcatctgtgcccgtgtgacccgggtgtgccacccagagttccctctctgccctgggctgccgcaggtgcagggctcgcattcctgtctggggtgggggggcccagaggggagggcccagcggcacccccgtcatggggagacaggagctggtggggagcagccggagccatcgcctgggcaaggccagagcaaggccctcctgccttagtgggggcttgtctgggggctgcctgggctgcgtgggtgtgtcccctgggctgcggggggtgtctccggggctgtgtgggggtgtgtcctcgggggctgtgtgggggtgtgtcctcggtgctgtgtgggggtgtgtcctcgggggctgtgtgggggtgtgtcctcggtgctgcgtgggggtgtcctcgggggctgtgtgggggtgtgtcccggggctgcaggcagaaggcacacccgcggggccatccgtgtcgggacaggcatgcccttcctcgccctggcacccggtcccgggtctcgggcccccgtgtgcccctctggccgcagctctcctgacccccaggtacccagccccccatgcaccaccagggttgctgtggccacttgggagtcgggagctcaggtgacctgcagggggcactgttgggccagcctgagaccctgtgggcagtccgcactgcatccagggtccagccccggcccctgccccccggggtctctgggaccctcaccacagggagtcaaggggccacctccctggtgccctgcccagctgtctggggcatggtcctgccctcccacactcccctctgttcttcagacccctgtgactggcccagccccttgtttgtggaaggcaggcccacccactacacaggggccagctggcaggcccaggcccagggcaggtggggagctgacgggcaccatcgccactgagcaagcccggggcggggccatgggacccgtccgcagcaggaccccccgccttcctccccagccccatccttgtccagtgacttctgagtgccctctcagtggggtgggtgctgcccccagcctgacacacgggcgcagcaaatgctaga

SEQ ID NO: 187 - DNA, 502 bp, Bos taurus
tggccgtggaggtcggtctcccaggccagaccaccagcacgcctggggccctgggcaggaaacgaggctcatctcccactggctttcccagcagtgcgatgttgcctcctgcttccgtcccttccgggccctccccaccccaccaggctcctctgcgggcgtcctgacgcccctccaggcgcacttgtcttccaggctgtagttccttcggctgctcccctctgcatctccctgacctcagtgcccatggctctgggcacctctgctatctgggcctgacttccccgtggccctgggcacctccgctgtgcgggctgggcttccccatggccccgggcacctccgctgtccaggtctggcttcctgtggccccgggcacctccgctgtccaggtctggcttccccgtggccccgggcgcctccgctgtgcgggctgggcttccccatggccccgggcacctccgctgtgtgggctgggcttccccatggccccgggcacctc

SEQ ID NO: 188 - DNA, 653 bp, Bos taurus
cccagaagaacttcccgcgcgccgacagaactacagcgcctgctctttagtgactccgggagccctatcggcggctgggctctgcgcgcccttttgcaagaatcagagtaacttgttaacagtcccgagccaagctggcgaggctccgcgctccggacccgaacaatagccagtgcgctcagcgctcggaggcgcgcggttcctcccgaatgcagcgcgcggttcccgcggggttccgaaagcgcacggcaggaggcgacatggggcacaaggcgccgagggctcggggcttgcagcctgttccctcaccgctgctgctcctcgccgcggccgccgcgctgctggtcgtgccgggcgcgcacgggcgcccagccgaggaggacgaggagctggtgctgccggccctggagcgcgacccgggactcaggaccgcgcacctccgcctggacgccttcggtcggcagctgagcctggagctgcacctggaccgcggcttcctggcacccggcttcacgctccagaccgtgggacgcagacccgagccggacgtgcagcgttctgatccggtgggcgacctggcgcactgcttctactctggcacggtcaacggcgaccccagctccgccgcggcgctcagcctctgcgacggcgtg

SEQ ID NO: 189 - DNA, 786 bp, Bos taurus
gggtgcggtgagtgctgggggtgggcaccgcctggcagctttccagagcagacgtgcagtttctgcccgacggggagggcgtgagggctgcgggcactccacactcaccgtgctgagggccgcctcctggtaaatcctcccagcaggcgggttttagaccctgtcgagttgttactagaaacggcctgagaccaccgccccagtcatccgttcccccgggcgctccctgaaacccccaccccggtcacccatcccctgggcgcttcctgagactcccgccccggtcgcccgtcccctgggcactccctgagacccccgccctggtcacccaccgggcactccctgagacccccgctgtggtcagccgtcccctgagaccccggccctgatcttccatcccccagacactccctgagacccccaccccggttgcccgtcccctgggcactctctgggacccccaccccggtcacctgttccccgggcgctccctgagaccactgccccggtcacccatcccccagccgctccctgagaccaccgccccggtcacccgtcccccagccgctccctgagacctccgccccagtcacccgtcccccggccactccctgagaccaccgccctggtcgcctgtcctccgggcactccttgagacctccaccgtggtcacccattccccggccgctccctgagaccaccaccctggtcacccatcccctgggcactccctgagaccactgccacggtcgcctgtcccccggccgctccctctgccgtcactcg

SEQ ID NO: 190 - DNA, 835 bp, Bos taurus
ctgatgctccgcccgcccccaggacgacgaggtggtcctgcagtgcaacgccacggtgctcaaggagcagctcaagctctgcctggccgccgagggcttcggcaaccgcctctgcttcctggagcccaccagcaacgcccaggtctgtgcgggacaaagggagagggggtctgacgagcagagaggctgggacctgaagaaaggggtctgggggggggggtctggagagggggtgctctccccacgagggaaaggggcgaggtctggggtgggcggaggtctgaaaagagcggggcggagggtcttcaatccaaggcagaagggctgggagggtcagggggtgctagcagggggctgactagggagggggctggaggttggagaagtggagggagtttggggtctgagcctgaggtgtactcaggatctaagggaggaggtgacctggggggatggggttggggctgggagagggaggggatctggagactgacggaggacgcggccgggggcctgaggtgcctggggtgggaggctcaggggggcggcgtgaatccgtgaccgccgttgtctgagggaggggcaggagctaggcctgtctgctggtgatggcgggaggtgggggttctctgcggggagggccaggaggaggctccagggttggaatgggcgcagtgtgtctgccggggccggggcggggcggggggatgaggcctcctatgagattaggtggggtgtggaaacttctgtgaggttcgggcgggataaaagagtgtgcaggaggaggaaaacaggtatttcccaggatgttggcgcggatgtggggtgtctgaag

SEQ ID NO: 191 - DNA, 332 bp, Bos taurus
ggttcagatgctacggctttagcagcgtcaccccccgggtgtggtccgcccccagtgaccctctggagctcctggtggcaggtgaggaagtaccgtcctgacctcccaacttgctgaggcaccagggtgggcattcgatgcttttctactggagctgtcccagaggagagggtggggtgagggggtggggctcatgggacagacacacagactgcagacggtgaggcctggggaccagaacaggagaaggggctcatgggaggagccgtccctacagtctagtctccccaggggtgtctggcaatgagatggggagggcagcttggagctca

SEQ ID NO: 192 - DNA, 322 bp, Bos taurus
tgctcctgctcggcgagttgcagaccacagcgccctcctgcaggcgtgcggggtgaggcggcgatcctccgaagaggcacagcagcgtgtcgtcgtcacgcaaggccgggagggggctgaccgtcagctgcacctggggaagggccgtgccgctcgcaccaggtctcttctgccccaacctggcgagtgggcctcctcccagcccggccccacccggggcacctgtcccctggtttgctctatggccgccctcctgaatgtggacccctgctccctggaaagctctctagaacccaactccacttcccctaaatgggacccc

SEQ ID NO: 193 - DNA, 372 bp, Bos taurus
acagctccccagacgggcccgggagaggcccccgggggacccggggccggggcagcgcagcacctggtagcctggcctcgatccgaggcctccagagccgctccatgtccgtcccagacgacgcccacttcagcatgatggtttttaggatcggcatccctgaccttcaccagacagtgagcaatagcctgctggccggcggatggggggcgggggggtggggtggtggtgggagacagggaccaccggagtctgcacggggagagaggaaggggaggggggctctggttctggatgccctcacacatggcccccactgccagaccctcatgggtttcccccagccctccgctgtcccgggaatcacccgta

SEQ ID NO: 194 - DNA, 325 bp, Bos taurus
accgggtccgggtatgcctgacatgggcctgcagtgcttcctgaatgcccctctgccagtcccgggccagctgcactggggtcactgaagcctggaaggagggcgctggctcagtgcccagtctcgtccctgagccgcccgccccgcccagccgcccgccggcactcaccgcgttgtgcacgcccagcccagctccgtagagcagcagcagcttcatggctctgtagcgcccgtgccgcaccgcctcgtgcagagccgtgtcaccttcctgtcgagagggatggggtgtcatcctccgcagccccgctccccgggaggggcaggc

SEQ ID NO: 195 - DNA, 398 bp, Bos taurus
ccccttcttcccagccccaggcggcacccacgcccgggccagtacagcatctgccactcacccattctcccccttcccaaggccgcccccctgctcccctcctgagcgcaggtgacagctgctcagactgtctgattcaccgacagctgaactagctacgagtatagcgggtgggcgcttcgtgaaccatctccttcccagcccctcaccccagagaggagtgggctggtgggtcaggaaaggcgagatgggtggataacagccgagaaccacttgcttactcttccctctctcccgtcccctcccagtttcatttccacacatacagccacccaccggggaccggaggcggctggtgttctctgaacgcagggaactggctctctctgtggtcttgt

SEQ ID NO: 196 - DNA, 486 bp, Bos taurus
gctggctgggagaggtggggctggatgcggactccgacccccggcagcacccacagtgaacaggcctgcgccagagagcctgccagagagcctgtgtgtcggggtggagaagagcagctctgggggcttaaataaagggcagggggtgagggatgcagtgagaacagagggtgaccgtctggccagtgggcccggcctcgggggccccggaagcagtgctgagcccgcggggtggctgtagagtcctcggtgggaggagggaagggctcgcagccagggtctcgggcccccctctctctgggctcacacggcgatcctcgctggagggcctggccgggggagagcggacggggcagggagacgggcgccggggccctgcggggcttagcgcaggtcttcctcgtcaccctggatggtcttcttgatgcgcagcagcatggtggtgagccactggtccagtcgggagatggagtcgtattccttcac

SEQ ID NO: 197 - DNA, 615 bp, Bos taurus
gagctgaccgtgcccccgcccccagcctgcagagtctgccgtttgcactcacgcacctcaacctgaaggccttgtggctagcggagaaccaggcgcagcccatgcttcggttccagaccgaggatgatgcccaaacgggcgagaaggtgctcacctgctacctgctgccacagcagcccctgcccagtcttggtaggtcgctgggggcggggcctggggcggggcggggcggggcctggggcggggcggcgttccccctgacaagcgcttccacagaggaccctgagctgcagagcagcccttcggaaagctggggagacgccccgctcagccgtgtcagcgtcatccagttcctggaggcccccgcgggcgacgacgacccggaggaagccgctgccgagaaacgggtatggcggggtggggtccccgggggctcccgcctccccgcccccgcctgacccctgccccacctgcccctcacccctgtgcccactgcagggcctgcagcgtcgggctacgccacaccccagcgagctcaaggtgatgaagaggggtgtggagcgccggagtgaagccacctcctgcaggcctgaccctgcgccaccctcgccctcc

SEQ ID NO: 198 - DNA, 1115 bp, Bos taurus
gatccgcctcagtgtgaagctgccctgaagccctgaggccccaaaccaggcccagccacaccctcttctccaggcgccggccgggtcctctgtgggaaccccaccaaaggcctgcgggagcgtcggcacgggtgccaggtacagcctgcccccacggcgggcagcacccagcccccagccccggcaggccgtgccctgagccagtcctctcccttcccaggctggggtgcacccagagaagctgccctctcccaggctggaagaactggcccccagggcctccgcctcggagcctgaccctgccgacgatcatgacctcctggcctgggccgggcttcaggctttgagggagctgcgcctgcggtgggtgccacctgccccacaccccagccctgccaccctatgtgctctgaccagggcggggtaggggggccttgtggctgggacctgccttggctgagtggttgctggtccccgggtggctggagggtcgggtcacgggagcccaaggctgccccaagtcacactcctctgggatctgctccaaaggaggagcaggctgcaacctcccctgcccacaccccagcccccttcccagtaggtgcccagagtcctgggaggagggggccgcaggcccctggggcccacagaggggccctgaagagcaagaggacgaggctgggcccaagcctcaccgcagccccccaagcctggccccaggtagcctgcctgacagagccagcaccgcagagcagcaccgggctgaggcgggaggggcagggagggcctctcctgggggtgggggctcaagttcccctcccctgagcccaggggccaggcagccgggtcagctggctgaaagagggagagggcaggccctcccgacaagggcggtgcatgcggagccagaggggccctgaatgagagtgggtggcccggggaggggccagggccaacacgtggggcagaccagcagaggttccccggagcacagcgcaagcccagcgaagggaacaaggcccccaggtgggtgggcaggagaccacggtgaggggtggcgggtggcagctactgcctttcctctgcagacagggcctgtcctgggctggggcaga

SEQ ID NO: 199 - DNA, 729 bp, Bos taurus
cgctcgtaggcaaaggccctgtaggaggacgtggctgccggctccagctgctcggacaccacgctgtatcggtcgtctgggcccccggcccccagcctcagtgagcgcagggacaaggtgtcgtaatctgcccggctgcctgccccaccacgctcgtggaaggacacaggccgggcgggcactggtgcagtgggcggggcaccccggtaccctgcagccccaaaggcaccacccccgttgtggacggagcttagcctccgactggagctcaggtccacggccgagcgggaagaccaggaggcggggctgtaggtgggctggcgatgggccggaaggtctgtggggcagggaggggaggggacggcagtgaggggccatgggggctcagcgggcccctcttcccgaccacggagcccagcccccctcggaacctccacctcaggggtccgggctagaggggcgtcactcaccgaggtttccccgcccaggccctgggagcgagagctgaagccggcctgcaaggtgtggtactgagccctggatgcacctgtagacggggcaggggcagcacagcccgctggggcgacggcaggcgctggcctcctgcctcccctccaccctgaggctggcagggcagccccccggcccccaggggggacagacgcgctcaccctgctgacctcctggcagcctggggagtgtgcctggccccctcgcccaggaagccgc

SEQ ID NO: 200 - DNA, 694 bp, Bos taurus
gtctctgcccgcgcttgtcggcgacccagagggcaggcctcctgtgtgcggggttcgaccccttgccttcccccgccctgcggcgtctcctgacaccgggagccataagtctgccttttccctccctctctctgccaccactcctcagctcccctctccctggccctttctccagagtcctcggtccctagttcctgctcctttgatccctggtcacagaacgacctccctatcccgccgccaaaggcccctgtggccccctcccagagcagctcggcccccaccgtccacacccacaccccgagcttgagtcccctctgccccagggtcctattcaccccccgtctcctcatcacctcacgtcgcctctcggggtgccttgctgagtcccaggagcacacactagtttgactcagaggggtcgggggttgggagagctgtgggcggggctggccttgcgggtggggttttataactgatgccgtggaaaggggccctgtgtaagcccccccgcatgccccacagccgggccagcctccctgaaccacctgatgggcagggggctctggtgggggaggccgcccccgcaccactgacctgctcagggtggccccccagcgcagccgctccttgccctctggtctgtgccccccgcctccgggggaactgccagcaggagcctgtcgctgctttg

SEQ ID NO: 201 - DNA, 742 bp, Bos taurus
tcctcgtccattggggcaaggtggccgacctggccatctgcaacaaccagctggtgagagagccaccgcccaccttgcatctttctgccgggccgccggcctcctcccgctcaggtccccaactcccttctggccctgtggggaccctaccctcagagccccatgaagggtcccgccccagctcttccatttcctgcagataggcgtggccttctcccagagcaacgtctcctcttacgtcgtggacctgacgcgggtcactaggacaggcacggtggcccgggaccccgtgcaagacagccggcctccgacgcagcagccgccccaccccagtgccccgcttcgccgcatctacgagcggcccagcaccgcctgcagcaagcctcagaggtggggtgctcggggccttggaggacactggggggtgcaagaggtgagccccccagcctcaccgccccccccctccccgcctccctttgctcggccggccccccagggtgaaacagaactcggagagtgagcgccgcagccccagcagcgaggacgaccgggacgagcgggagtctcgggcggagatccagaacgcagaggactacaatgagatcttccagcccaagaacagcatcagtgaggctctcccctcccttcccatccccgcctagccccaccattcccctctgggacgcagaggcagatggtcctttccaggatggtgtccacgttgctgcgccccccttagcct

SEQ ID NO: 202 - DNA, 448 bp, Bos taurus
gttcacgggagcagctggacctgcaggaaaaaccagatgagagatagcgaaaagccactgttactgccgtttattgagcacctactgggtgcccacaatcgtcctaagatcggtaaggggagggtcaggagcggcggcacgtagctaaagcgtggattgaaggacttctaagtgaggattagatttgcgaatacgcggaagacagcttttagagttggagctgagaatgggaagatacagtgaggaatgcgaggtcggaactttagaaagtccagccggggctctccgggaactccgggatggaggggagggaggggtgagtggaatccgaaccctgggagagggatggaatttaggcggcggcagacctggggatggggagataagagaagacaatgagggaatgaaatagacaatgaaggggtcgctgggtaaatatttggaattg

SEQ ID NO: 203 - DNA, 860 bp, Bos taurus
actcaagtcagtgccctgggtggttggaggggcccggaccggttccccggggctgcctgggcctctgatcctcagtcagcggcagaccccccagtgctgggagtcctgccgctccccatttgctctgaggagcccccgcagccccccggccacccccacccccgcccgctctgggccacactcagggtccccccgggcatcctgggtgcgcgcctggagggctgggggctctcaagggaccctggcaacgtgggagtttggggacgtgaggccaccttccctgagctggcgttcctgccgagcgaccctgagcccccgtggcaggccaggccccagtgtgggggtgaggtgggcggtggagacgtggatgcctcggcctctgaggtctgcagcttggggacctaggggcacgaggcagagcagactgtcggggtgtcctgggggcagcgggtgtggagtggacccacaggatgacgggggcacctgggccaagggggctggccagtgtgacagcagcgagaagtgattgacgcaggagggtgggggcaagcgtggcatttcagcaagcggggacagggggcagcgactggagtttaattttcaaaagatccttcacgctgcctggtgaggcacctggtggctggggagaagccggcgcggggagctcgtggggctcaggctgcgggcacggacggccggggggcctgggaggtgggcagggctgcccccaccccaggaacacttggcatcggtggccggcagggaagtatgcggccctggacgcggaggccacccgcccgctgctgcagctgctgacctcgtctctgagcaaggcgcgcctgaacgccatcaaggccctc

SEQ ID NO: 204 - DNA, 310 bp, Bos taurus
tgcggtagggcacgtcgggaatgtcgtagtatggctggtccttgtggatgtcctggaagcacagggcccgcctcacaccacatccccacgggcgcctccctccctgcccctgcccatctgtgacgcgcgccccccgccccccacttggggcgtgaggcaggctcggcggggcgggcggggcactcacagcactcagcgacagggacagggtctgcaggatgtccagcatggtcttcagcaccgtcccgctccagagcaagtgggggaacctgcacccagagccccctgctcaaggcccagagccccccgc

SEQ ID NO: 205 - DNA, 444 bp, Bos taurus
accgccctcttgtgtgagtggcgttgggatcccgcgctggttttcctggcggccaggccgtgtcccggtgttttctggtccccggacggcccccgctgtgcgtgcttgtcggcccctccccgccgctgccggccttcctcctcgtcccgcgcgagaccgccagcgttcccctgtctgggaccgagccggcctcgcctcacgtgggtggcgtccctgggcctccgtccgccggtggcgtccccgggcagagcagtgccctcggagccctgagaaagggagcggggcggggagcctggcacagcaggattgcgcgtgtgagggccatgggggacgccggtacgcggcgggagagcgttctccctggccggctgtggctctgggtgtgtaagtggcggtgggcgcgagccccgtgaggggggcgccagtccgtcgtcccgggtgtgc

SEQ ID NO: 206 - DNA, 331 bp, Bos taurus
gcatctgcatgcgctcttccctgtgcctagaatgccattccccaccacctccccatctgacagaagcctgcttgtccttccagacctggcaccaatgcctcctccttttagagcctctctgcccctcctcatgccctcctcccaggcaggggtgatccccactccaggacttcctgaggcactgcaccccacccccagcagtgccggacgagaggcagctgtagctctcctcgcaataagctcacagcccagcatgggccttagcccagagggaccctgctccacagcagaggaagtcactaccccatcccattttcccagcgagagctgt

SEQ ID NO: 207 - DNA, 849 bp, Bos taurus
gccccacttggattcctggctctctgaccatggccacagtctccaccctccactgtcctctctgttcacgagcactgcactcccaggctgccccctgaagccccaaaccgggcaccgagcccaggacatgcctggcgaagcgggtggtgaggatccctccccgtgtctctggccagccctctgggaacccccccaccactggcctgaccccctctggaaccccctctcccccttggctctgagccgtggagcctgaagtcacagaggacctgtggccgcacgaggtgaggaggcatcctgcgaggggctgcccggtggggcgggagggggatggctgccccaggacagaggggcggggccagggtgtctgagaccctcctggggcagcactgggaaggcggccagtggggctgggggtcaggagggcactgctggccggggaggagggctgatcagggacccacgcctgcgggaactgccaggcagtggggctgaaggaatggttcttggggtgggagtgggcagtccttccaggaggagaggggcacagctgctcagtgaagtccaggggccgacaagggccccagggctgaaggcactgggcagcgacactggctgctggggggagcgatgagctgggggacagagaggccaggggtggccacagccacacggccggggggcctggttgtgaggctgggcccagcagagcgtccccgggacggtgactgctaaggtggcctcatgggcctgggctgggctggccgggtggacagcggggctgctgagggcccccggtgcccctgcccccggccggtcatccgtccgtaaccactgatgaaaagggac

SEQ ID NO: 208 - DNA, 778 bp, Bos taurus
ccagggactcaggccagatctgcagtcagacgggaccgtgggggacctcagggagtgtcatcacgtcctctggcagaagaggctttggtgtggaactaggggagccggtgggtgcagggagcaggcctgaagggccccggggctgggggagagtccaggaagcagagggcggaaggcggcctggaggaggaggagcctgggggccacagaccgctagggcctgacacaagcccactggtgcggcacggcctgcaccccgtgccctgggggacggagggcccatctgcccggctcagccctgctgcctgacggtcggccgccagaggggacacggggcagagagcagcagggaataaccggggcaacgcgacgcggccggcttcctccccagctctccacacacatccagcctgggcagctgggggctgggcgggacgcgggctccctggactctcctccccggtccagctccggctcgtcaccccaggggccgccgggctccctggactcccactctgggcacacacctcctcctggtccagctccggctcgtcactccaggggccactggccccagaggctgcgcctgcgctccgtggcctcacccctgaaggctctgctcacgtgcgcactgctgtctgggtgaagctgaccatgtgctgccgggcccccgacccctcctctctcagggagggtcaggtcgtgggggaggccactggggttcagggtgcttgggagtgggtggctgagccctgaccctgaccccaggaggaccggt

SEQ ID NO: 209 - DNA, 324 bp, Bos taurus
gaggagggcaagaaggagactggcggggttggtggaaagagggttccggggtgggggcagcgaggagcccctggatgggcaaggccagcagtggagcccaggagatccagtcactgggccatagtggggtcgctggtcttgtccctctgatcccacagaagtcccagcctagcgacacacttcaggatctctgcggtactttgttcaaaggctgatcagacagtatactgaaacacgagttcagactgttctgctgggcagggggcggggaggtgcgagggccatttagcaaagtcctgccagccgtatgcctgctcttcccca

SEQ ID NO: 210 - DNA, 647 bp, Bos taurus
tgactgccagcaggagctgagcctggtccggactgtgacccaggggcagcacaccttcctgagccgagaggaggtgcagcacttcgtcaaagagtgagccccgacccgttccaggccccctcagccccgcaccccacccccatcccttgccctggctctgcacacacacccctcgccccacggctccagccctgttgcgcggtacccccttcctgcaggtgtggcctcctcaactgtgaggctgtgctggagctgctcatccgccacctggctgccaccagtgagcgtgttcagatggtgaggaggggagtgggcggggctggggtgtgccggggcacggggagtggggagagtgagcacaggacacccagtcgattgtctggtcgattgtctgctgagggctgagaggagggagggagggagggagccggccacacagacctcccttggatggggaaccagctgacatcaagcccacccaccctgcagagagccctgggtgccattgcctccctcgggtgcaccgacctgctctcccaggagcgagtcctgctcctcacccggcctcggctgcaggagctcagtgcgggcagccccggacccgtgaccaacaaggccaccaaggtgggcagcctccaaggcccaga

SEQ ID NO: 211 - DNA, 1100 bp, Bos taurus
cttctcttgggggccacggatgaggacctgcccccccacggggctcccttccacttccagttgagccccagggtcccagagctggcccggaactggagcctcagccaggttaatggtgagccccccacacacacccctggggttcagagcctgggggaggttcccgctggtgcaccctcaggcccggcagggattggttatcagacgcagaggacggatggggaaaccgaggcctagcatgccaggagcgccgcccagtcggctgctgcccagggcacagacacgggagggggtacggccgcccccggcccctgcttgaccgcaagcgccctccgcagtaagccacgcgcgcctgcgaccgaagctccgggtcccggaggggctgcaccgcctcagcctgctgctccaggactccggacagccgccccagcagcgcgagcagcccctgaacgtgaccgtgtgccgctgcggccaggacggcgcctgcctgccgggagccgcggcactgcgggcgggtggcgcgggcattagcctaggtgccctggtcatcatgctggccagcgtcatcctgctgctctgtgagtgccggcgcccgcaggcccttgcccttggaccccagcctcttggcagccccacccgccctctagccccgcctcctggcagccccacccgccctccggccccgccctccaccctcgcgctccacccccacccgaccccgacctccgccccgacccccacccagtgctcgccctgctggtggccctcctggcgcggttccggcagcagtcctgggacaagaggcttctgcatggactgcaggacgacctccgggacaacatccttaactacgacgagcaggggggcggggaggaggaccaggtgagggggccggggcggcttacaagtgctgcgggtagggccaagggccccagctggcctgcatcctcccgacccgcgacgatagcggctgaccggaacgcgctccatctgccctcaggacgcctacgacatagaccagctgcgccacccagcagaactggcagccttcggcgcccccttgggacggccgccgctgcgcagagacgccccgttcggcc

SEQ ID NO: 212 - DNA, 569 bp, Bos taurus
tgggcttattcccaggggcccattctccccgcctcagacccagacccgaacctggggagtctcggggctctatgctcaggggagacagcccaccccagggcactttgagattctctgggaggacaaggcctttcagatcgtcaaaggctggtggggagtcgggcggagatggctcgggagccacaggctgcaggggcctgaggctgctggatgggggtcagtctgggcagcagcagacccgccccaacccatcctacccccggaggctccagggatcaccccatcccccgggcagagtccagcaggcagatgcagagggctgagtgagcgtctgtgggggggcagtggtggatccgtgggaactgtggttgggtcccaccctgggaccccgaagaccaccgctcagaggcggctcccaccatgggaccccgcagaccacagctcaggggcgcccccccccacccggggaccccgcaaaccacagctaagggacgggccccaccctgtgctgtctgagctccggcagctcagggtcctgagcaggaatgtctgagacagtaagagaggcc

SEQ ID NO: 213 - DNA, 868 bp, Bos taurus
cgcttactaatactcctgacagtgtggaggccagcagcccaaggtcagggcgccggttgatccagcattttcggtgggtagacactggctcttagccgcgttctctgtggctgagagcctggtctcaccctcatgagcctctgaagtgggtcatccctaaggctcatctccaacatgggcaagatgggattacacttcaccacgtggatttgggggtcaggacatggggaagtgggtggggtggatgtcagctgggcctccgggaacagctaggaaggggtcaggaggaaaggtgggtgccaggggtcttgagggacagggaggactgtgatcagtcctggacctgacctgccaagcactggcaaggggcaggtggggtcgggaggggcagtggggggaggggtagtgttaggggaggggtgggggaggggcagtgtgggggaggggcagtgttaggggaggggtgggggaggggcagtgtgggggaggggcagtgttaggtagggcaggggagggtaagtgttgggggaagggtggggagggctgtggggggaggggcaggtgggggagggaagtgttgggggaggggcggaaaaagtggggagggggtggggaaggctgtgtgggtgagggcggggaggggcagtgtgggggagggcagaacccaggagggttgggggcaggaagacctctggagggaagggtggggtctcagatgagagtggtgacccggggtggggctgtgggctgggatcagggagagcctgcccctggtgggcagcagggcggctggcctgcctgggaaggggctggcactcccctgggcctgggctggtggagcgccgtgtagcccacaagggaagggctg

SEQ ID NO: 214 - DNA, 379 bp, Bos taurus
ggactgctcgacccggcttgacatctagcccgagcgtccccagtcctagcaaggctatttgcatcctgaaccatccggattgcaggcactgccgctccggtcccatcctcgtcctccccaggcccttttcgaactcagctcggccaggcctcgaaaaacttggagtggggttcccactgcagagacgctccccggagcgcggggtcactggttgggtggggaggggacccagttctcgtctccttccccgatcctggggggagcgaagaggagagggatcaggggagcggggagggtccttggagcgcgaagaccgacccttcaccaggaggtgccctgcgtccacggcttgggagacagacaagagtcacagcggcgc

SEQ ID NO: 215 - DNA, 310 bp, Bos taurus
gccctgcgacacccagaataaggattcagtgaagcggacgctggagcagattgatgtcatccagcgcatgtgccagctctaccccgagaccttcctgtgtgtcacggacagcgcaggtgggtgccagcgccctgggcaggggggcgggggccgtcctgagcagcgccgctgggctgtcccttcacattgcagcccccacccccaggcatccagcaggccttccaggaggggaaagtggccagtctggttggcgtggagggcggccactccatcgacagcagtctgggtgtcctgcgggcgctctaccacc

SEQ ID NO: 216 - DNA, 446 bp, Bos taurus
gagctgaaggacgagattgagcgcaagtttgacaagtggcaggagccgcctcccgtgaagcaggtgaagccactgcctgcgccccttgatgggcagcgcaagaagagaggcggccgcaggtggggcccggggaccaggcggggcgggaagggctgaggccccccttcccggctgacccccctggtccgccccaggtaccgcaagatgaaggagcggctggggctgacagagatccggaagcaggccaaccgcatgagcttcggagaggtcacacccccctacctcgcgcccctgcatcccctccactcctatggccatggcaacacctcagcctcttccctggtcccctggcttccactctctcccagccccagagggatctccgtgaccgcacaggcccccaggctcacctcccaccttccccaccccaccaccctgctgtccag

SEQ ID NO: 217 - DNA, 310 bp, Bos taurus
gacactgggacccctcaaggagaagcaggcgtgcaggtgaccagcaacggtccccaggaaggtaacccccgccccaccaagcgggcagtggacagctgacccatgaaggtgtcccaggcgggaagggagccgggcaggcacggatggacaggggtgggtaaggcctgtggggagaggaggggctccgcgggcccatcacggtgcggggagcagcaggtgagacaggggcccgggcagatgccctctcaggcccagggagcggaagaccgccatcggggtagcccagggcggcccagctcccatccaggcc

SEQ ID NO: 218 - DNA, 2006 bp, Bos taurus
  misc_feature (643)..(1456): n is a, c, g, or t
tcccctgaggaccgggctgcagtggggcgtgcaaggagtggggtgctgtcagaccctggggcctcctcaccaccctcgcctccgttccacctgtgagagcccggacagtgcctccttccctgtcgccccccttcccttcctaccccctcctggtggtccttccctcccagcctcttgacccccactctcactgatgccccagggcggtgggctgggcgggtggcccagactcggcaggctgaaaggggcccttgactgcaggagggttggggtgggaggggaccgagggaggaaggggacacagacactggaggcagagagagggtacctggagcctggggagggcaagaccagcaggacaactgggccagagcctggggaggggccctgggaagtgagggaaggggtgggaatcccagggcctctgggtgccgtccccacaggccagctcccgctgcgtatcaacaagctgtccccacacacaccccgtcccccaccatttcccccatcgtccacctgtagggctccccgcaccatcctgtccccaacagtgtcccccgacacaccatccccacagactgtccccccacacagcgtccccacacagcatccccacacagcgtccccccatgcagcgtccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatccccccacacacaccgtccccacacactgtcccacacacaccctccccacacacacaccgtccccacacacaccgtccccgcacacggtccctcacacagcctcccccctgtccccacccttcactgggacaggtcaggctccctgggtggagggagtgacggtcaggccccatctgcccctccctgcatcctgccggctgggctgtcctctgggccctgtgacctggtcacaccccttcaggtttgggacatttcagtcgggctcactgcggggctgaccgtctgaccccacatgtgaccctctcacaagcccaggcatggggctgggaccgtcaggccacacggggcccccagtgcagcggggcctccggggaggcggtcactgcggagcgctccaggcgtgacggtggcgggccggcatcccgcatggccacctgctggcacaccggggctgccgtggctgcggcccaccgaggggtgcggcgcgtgcgtttggtgctgtacggcgtgcccgtgacggcctgcctcctgcccccgaggtgccgc

SEQ ID NO: 219 - DNA, 330 bp, Bos taurus
aaccgaaaggacaaacccagcctcgagcacgtggtcacacgcagagccagcagccgagggcagctgtgccccagtcctgctccacactcgtggaggggctgcccgcagtgaccccatgaagacatgagggcccaggaggagggtgggccagctggggcacccccaggtgttcctgcactttgggtctgaacatccagaccctgcaggcctggtcgacagcaaggcccgggctgccgagcaggagggaacacacctactcacacgtgctggaggggtctgagggttgggggggagcggtaaggggctcgagaaggaaccccaagcctggag

SEQ ID NO: 220 - DNA, 346 bp, Bos taurus
gaaaactttctttcaagtcgctttcggcgtcgccaaacttttcgaggctggttggctatggctgccagctgtcggaagctggggagatccgcgttcaggggcacggcccccagggaggggatgggcatccctttctccgggctgggaaggtaggagcggccgcccgcggcaccggcgccacggaccccgctcctccccggccgcccggcagcgccgcagttgtccccctggcatccgcgcgccggcgggggcgacgcggccgcacaaaggcagcggggcgccaggtagggcgagcacctggcggtgggcaggcggctgcgggcgagggtgcctcggggcagctgag

SEQ ID NO: 221 - DNA, 1115 bp, Bos taurus
  misc_feature (495)..(848): n is a, c, g, or t
tgctcagtgcacgcgcagtgtgggttgtgtctcagcgctcacgtttgggggactctgctctgggcctgtgggaattctcactgctgttcttggagcttttctctcagtacagtgttatttccagtttgagagttaaagaaggagaaaggccaggaatgggaagaagtcggggcgggaagggggcacctgaagcccccttaaaggctgggagccctgggcgggcgggccgcgcggcccttctagacgacagcggcggcgaccaacccccggggagggacgcccccctcccggcccgagcagaagggcggccggttcctcccttttgaaagggtggctcctcctgcccgcgtctcagggccgcttcatgctggagacgctgctggccctgaagaacaacgacgtgcggaagatcccaggctacgaccccgagcccgcggagaggctcgcggaagctgcagagaggcctggtgagcccacccccctcctcactggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccctcctccctgggagccctccttcctccctgaggagcgagccccccctccttcctatgggcctccttctcctccctggggagcacccccttctccctggggagccctcctttcagctcctatgctccccagctgcactatcccatgagacttctccaacaaatagaggagccacaggtcaggtgcccgtccagtcgccagggtccccaggtcagggggaatctgcaggtttctgtggtgagacccccccccaccttgtgagctgtg

SEQ ID NO: 222 - DNA, 959 bp, Bos taurus
ccctctccctgcagggtgggggctgagcactcggaggtgcgttgtgggtgctggggccaccaggccttccctgggactgaccctgacgtccttgattgacaggcactgagtcctcccacagcgatggtgagcaggagacaggtgagccctgggccacgtgccagggacatgaggggccccagcatctggaaccccaggatgggggagagggaatgggcctgtgccccaaggggagggcagtgccagggagggccgggaagaaagaggccagtcccccgtgacccagagggttcctaataggagaggcaggggcgggcggctgggggctgagggaggagccactaagcagagtgggtgggggctgcctgcctgagggcggggctcagggttaccgcttccacgtccagactcagccccggaggaggcccccgggcactgctcctacgacccaccttgccgttggctggaggacgcagacctacagacccacgtgccggccacgctggccaatggcacccggctgcagctggcgggcgtcccggcgggcgtgctgcggcggcccgggctgcggcatttctactgctgccccggctgtggcaaagtcttctgggaaggtacccacctggcccgagtcaccagcaacttccaagagatcctggagggcgccccccgcccagccagcggcccctcctaagagcggggcctaggctgcccggactaaaagtgcagaagctgcacaggtggtggccgtgggctccttcctctggtctcccctcacgccccgccctgcgctcgccagcccctcgcagcctgagggttggacccaggtggccccccaaacctgctggagctcagtaggtgggaggtgggggtccccccactgtgctgtggccgttcccgtcgccccctccaccatgcaccccccaggatgagggccaggcccctaggtcccgggacag

SEQ ID NO: 223 - DNA, 1091 bp, Bos taurus
gctgcccgtggaggacctgtactaccaggcctgcgtcttcgacctgctcaccacgggggacgtgaacttcacactggccgcctactacgccctggaggacgtcaagatgcttcactccaacaaggacaagctgcacctgtacgcgaggactcgggagccgccaggccgggcgggggccgcccctgggccacccccggccccctggcccctcctcggcaccctcctgctcctggccttgctccccgcgctcctgggggccgcctaggacagaccgcgggggcggcccctcactgcacggggtctggtggcttcctgctggccccgtgggccccacgggggacagttacgtccccgccgccttgggctgcctgctggcgagggctgggggcataggatgtcccctctctgcccccccgggggaccggctactgggggtggtggcgatgggtgctgtgacgtgtgtcacctccagctgtgcagagaacaagctgcccccccccgcccccgtctccgcgcgccccaccgtgtgaatgtgcagatcagagccccagcccccacgcgaagcccccaccccacgccagagaccctggacggggcagcatgtcaagctgctcccacccctgcgatgcactgagagaggcccggctgactgctgcacacgtgccccccggggccgcctgcgcccacgcacgcacacacactacacactcgcacacacggaggcgggggcgggggcgggggcgggggcgggcccaggctggacccccgagcagggcatcccttcctgctgggcctttaggacacgccccagggcgctgcttggcgtctgtagaaaactgggggaatcctttggccaaaacgttgcaccctccccatgccttcctgcccgtggcctccctgtgagcccagcagagaccaccaggcgccccccaactgggcacgggttcaggcccccgtgaccaggccccctgcgctctgagcaatgacctgctctggccccttactcccaccacctgtccttccgtcccccttcccgtctaccgtccttaggaagtcgaacctttgtttttaaattgtctggtcctccccag

SEQ ID NO: 224 - DNA, 2291 bp, Bos taurus
atgagatttggaagaggtgaggggtgcctgcaccccctcgcatccctggcccagctcctccggaccctcgattgcagaccacccccgtggtttgcatccgcgccgtctccccgacccagggtctctacctcctggagctcaggatcctccctgcttcccccccacgacgggcggggccgggggtccccgaagcttgaggtgggggcaaggagaacctcggctgaagaccgctcaggcgcgcaggggtgggcgagacgggagtggggacgtctccgagacgcgggaagagggtcgggggcgcgggaagagggtcgggggcgcagagcccgggggcgcagccgagtgggcccggccctctgctccgcagctcgtcctgggggacacagggcggaagctgccccctcttcccgccccaaacccgcagagcagtggctccgtgccgctcgagtccggctccccagacgcctcatctggacgcgcgcaaactcccccaaaggacggtgcgggcacccgggcccctcggcctcaaagctccgcgaccgcccgcccccgggagcggaccccacccgcggcccctgggtgaggagcagaaacttctgaggaggcggtcggcgcccctcaccgcggcggtacgactcaccggggtggccagccaagagggacccgcgtccccgaagtgagggcaagcgggctgccggcgacggccgagggggcgtccgctcggcccggagtcccgcggcgccccggccccaactcggccggagtgtctttgccgcaagacttgctgagcttgcagcctgcgccgcggcgagggaggcgcgcgacccgcggagcgcgctgcagtgcagggaggtgatgggggcggggccggggcggggtccggaggaagtggggcggagccggggcggagaccggcaggaggcagggaggcgcgcgaccggccgagagcgctgcagtgccgggagggggtgggggcgggggcgggggcggggcccggtggaggggcgtgaggaagtgggggaggagcccggatgaggggcggggccgagggcggggcccgggttgggggggcacgaggcggggccggagaggtggggcggggccgggcggagacgggcagaaggcgagggaggttcgcaacacgcggagcgcgctgcagtgcggggagggggtaggggcggggcaggggcgtggcccgaaggaggggcggggcgggaagtgggggcggagccaggagggcggggcccggatggtcggggaggggcggagcccggcaggaggtgggaacctgtcaagaaggggcggagtccggggagagggcggggcccggaggggcgagaggaagtggagtcggggcccgcgaagcgggcggtgaccggctggaggtgggaacctgccagaagcgggcagagtccgggggagggggagggaaccatgtggggcggggcggggtggggcggggcgggagaggaggggcgataggggcggggcggggcggggccgggggcgggggcgggggcgggcctgccccgggcggacccgggtgccctacacgcagcatctctgggctctcagagcccactccggactcctgctcaaggtcaggaagggtcggaccgggaaccgggcctcgcggtcctcacccgagagcggctgtctctctcgtgccagctccccctcaacgtcccaaacccactgcagctcgaggcgccatcactggccgcaggagccctggtagccgtttggggtctctgagcccctctcctcgccgtcctcctcctcggaaggtcacttccacctgcagttgtcacctggagggaggggtttctgagcctcgagctttggtgagaaagaggaggacccttgggaggtattaaaggtgcctggagcgggtcagagaacagcgtgggtgagggagaagcacggggaggtgggaggaacaaagcagaggaccaattatcacacgacagggctttccacacggactggcctcagaactgaggtgggtttggggtggtttgcagggagcaaacagcccccaaatggtcctatggccaagtagcggcagatagtcagtggaggcatggaaggggcggggtgggaagcaccagctgctgctctcccttccagggcttggaggagcggctccgcacctggaactcctgcggcatctggaatatctgaatcttcatgagctctcctggagtcaacttgatcaccagagggcagtatctgcctctcccagagatgcccagagaggttcccgccagaatgcgcctgacctgacctca

SEQ ID NO: 225 - DNA, 485 bp, Bos taurus
gcgggcgcggcgcggcgcggccggagggaccgcagctcttttctgcagaaacctcattccgcttgtttctgaagccatcggggctcacgagggactcccccgtccaaggcccagaaccccgaccgttaacaaggtaacgtccaggccctctggacccctcctccgcctcccccagaggcctcccaggtgacagctcccgccgggccctgagcctggtcccgggaggcggctcccaggtgcgggggaggcagggagggcggctgggcctctgcttcctaacaccctctggagtgtccctgccattaggcggtaacaggcggggcggggcgagaagggggcgggatggggtgggcgtggcgaggagagactgcagttcgctgcctttccttctgatagtcacctactcctcttttggcgccagcccgacccaggggactcgggttcccaacatcctgactctaaaaaccgcccaggaggagcatgga

SEQ ID NO: 226 - DNA, 428 bp, Bos taurus
aactggggtcctcaaacctggggactccctgccctggatcccgggacaggtgcaagggcctgcccaaggagatggcatgagggtgtggttccccgggagctctaagaggggtcctccctggaagccctgttccaggtggtatcctggggcctcctgaggaggggtgaggtgtctggaggtgggtgtgaggttgctggggcctgggatccggggggtggagtgaccgggggggcctgggtgtgacgggcagggcctgcagtgacctgggtgtcaccccaggtcctggtggggagtggggcccctggtctccatgctctgtgccctgtgggggcggctaccggaatcgcacccgagggagcagcgggcccagccccgtggacttctccacctgtggtctgcagccctgtgcgggtgagggcacccacctt

SEQ ID NO: 227 - DNA, 1015 bp, Bos taurus
cagtgctggcccttctgccttggccagacctggcacttctgctgcagccagggctggcacattggcctcatccagtgctggcccttctgccttggccaggcctggcccttctgcctcggccagttctggcccttctggcttggccagacctggcacttctgctgcagccagggctggcatttcggcctgagccagggctgacatgtctgccagggtcagtgatggtccttctgcctcagcaaatgctaacccttctgccttggccagacctggcacttctgctccagccagggctgacatggctgctaggtccaggcctggcccttctgcctcagccagtactggcacttctcctgcagccagggctggccctttggccttggccagtgctgacatatctgccagggccaggcctggcccttctgcctcagccagtgctggcccttctgccttggccaggcctggcccttctgcctcggccagacctggcacttctcctgcagccagggctggccctttggccttggccagtgcttacatgtctgccagggccaggcctggctcttctgccttggccagatctggcacttctcctccacccagggctggtacttcgggctcggccagtgctgacatgtctgcctgggccagtgctggcccttctgccttggccagacctggcacttgtgctccagccagggctgacatgtctgccagggccaagcctggcccttctgcctcagccagtgctggcccttctgccttggccagaactggcacttctgatgcagccagggctggcatttcggacttggccagtgctgacccttctgccttggccagacctggcacttctgctgcagccagggctggcactttggcctcatccagtgctggcccttctgccttggccagacctggcacttgtgctccagccagggctgacatgtctgccagggccaagcctggcccttctgcctcagccagtactggcacttctgctgcagccagcgct

SEQ ID NO: 228 - DNA, 514 bp, Bos taurus
ctctccctggggtgggcgtgggtatgcacaggccccaagtcaatgtgacacctgcaaccgtgcttctgaggccacaaagaggttagtcagagcctgcacgtgagaagtcagcatcgggaaggcaggagggaaaggacacacgaggccggagcccggcagcctccctgtacctcagagcaggaacggccgcgtcaggcctcacagcgtgctcagcaagagctggggagccggcgccgtccctcccacccaccacggccagcggtgggtgggccagtcctccggaggctcagagccacctcggagcaggcccctcagtgccctctctggtctcccagaccacccacccaggccgcaaccctctccaggactcacgtttgaggaacacagatgtgcggcgtctgcctgcgtcccccgctgcccgcatccactccagacaagctagaccgcccgctctctctgcccatgccccctcagcactgcgcagcagagaaccagccatctttctgccggggaa

SEQ ID NO: 229 - DNA, 332 bp, Bos taurus
gcccaccgccccacagtagcccaggatgcccagcggctccaggaaggcctcaaagaggccctgctcgcctggctctgagctctgcagcagcactggggaggaacgggagccgtcaggtggagggcccaggtcttcctcccacacctcctaactctgggccctgccctcccagctccaccctcttccccatcagatcctcctgtctgcagggactcaggagcctggggcccgcctcacctcggggccgggccttggtgagctggtaggtggcgcgcagagccctgagcacctgcacggactcctgaacccgggagaagcgcctctccagctca

SEQ ID NO: 230 - DNA, 448 bp, Bos taurus
cctcgcttctgctcccaggtctgctcccgccctgccgggcccctcaggcgagtcccgttgtggagggaagggcctgcagccagcgcagcagacatggggcgcccaggacgctcccaggcccgcaggcgagacgggcagaggagcccagcaggcgtctggctgcagcaggccgccctccaccacgagaaggtgccggccgctctgtcccccttggagcttctgctggaacgcaggtgactcccctccgctccggcccccgcccccaccctcctctcgcggtctcccgccagctctcagacagaccctgcactggggcaggcagtgagtgcgctgccaggccctgcagcaccgcgggcccagggcacggtaccttcgtgggtggctgcgtctgctgcggcagtgggggctgctcggtggttcccatcggcagttcaaatcgagggctggt

SEQ ID NO: 231 - DNA, 462 bp, Bos taurus
ctacacaccgaccctcgctggagggagagccccacccttcaagctccaggtccccagtactgggttctcagcccccactgcctggaagggggtctcagagtccacagccccctccccaggggcttgagtcacccacggggaaaagccgcgaggggtgagtcacggggtggagaagggggagcgaggtctgctgtggccaggggaccctcaggctggcctgagtcacacacgtcccggcctcgctgaccccagccccttccccggagattaggaacgggaagcccattatcggtggactgtggagttaaaggaagagccgggacgggggaggaagcacgcgcccaggcgggcgggaagctccggttctcatggtaactggggggggggggggtctgtcagctcggtggcggggttccctccctcgtcagggacggtccccgagcactggggaagcccccgcgc

SEQ ID NO: 232 - DNA, 765 bp, Bos taurus
caacggtcaatgtcagccagactcccccatccccagcccccagacctgctctgtagaatccacccggcgaactgctcccctgtggtccaggactccacctcggccgagtagcactcctctgcaaggacgaggggcaggggtgagagagagggacagcagggtcctccctgatggggaaaggggtgctgtcccgtgggggtcccaccagcaggacggcagggaggtcaaggtcacatcgggaggtcaggatcacattgggagggctgcggtgggaagggggtctggggagggtgagggtgccgggcagactctgccgtgggagccctgagcaggacgcgcgcctcaccattgaaggtggacacgtccagcgccatgcggccccgcctccatccggccgtccactcgagctgggtgggtgggtgggcccgggcagacccaggggccagctgcatctgctccagggcccccagcagcttgtgttggcacagcgctgccatgcccctgggggcctggtcggagacaaacctgcgggcgcagcacagggccgctgtgagcctggggtggcctgctgaccccgtccgcggggccccccacccacggagccccctacttgagcagtggcttctgcagggtgtgcatcggcgggaaggcactgagcaggatggccatgagggcccagccgcgctggctctgctgctcgtcggggttgcgccagagctgggccaccagctggctgaagagctcatctcgcagccccggctggcg

SEQ ID NO: 233 - DNA, 1492 bp, Bos taurus
  misc_feature (425)..(1356): n is a, c, g, or t
gctggcggcagatcgggcaggcgctgagcgccgcgccacagggcgcgcacgcgccgtggccgcactggaacacgaggcggatgtggctgtcgatgcaaatcggacaggtgatgcgctcctccatctgcctgtagcggctctgcagctcctccaccaactgccgcggtgggccgggcgcgggggctgcgctgaccacctccgtgccgtctgcggaagaggtccaggtcaggggcgggggctcggggctgaggtcttgcgagagaacccgaacagagatgtgagccacgacagggggcggggccgtggacagcgggcggggccgcgggcgggcgcagcggcccacacctgggggcagcggggcggggcgggcgccgggccccacctgggcgcagcttcttgctgatgaccacctggcacctgatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccctccccacccggcactcaccctcgcacactgtgcgatgctggcagggcgagaacagcaccagcagcgccagctccgagcacactaggcactcagcggcctcggggcccgtcggcgcggccacatgcaggttgg

SEQ ID NO: 234 - DNA, 20 bp, Bos taurus
agacaaaggattatatgtca

SEQ ID NO: 235 - DNA, 635 bp, Bos taurus
tttgcacccccacacagaggggacccgcctatctcagagctcactccttggcaaaacccacaccctgggcttgaaacaacaggaattcattctcccggggctgggaggctagaaatcctagatcaagatgcaggcagggccagcgaggttttagaggaggagcccaccagactcgctcagcttctggtggtctagacagtgctaggcttcacagaatcaggctagtctctgcctcatctccccgtccctgccccgtccccctccctcccctgtcccccgtcccaccccatccctgcggcacccccacccggtccccatgatccctgacccctgagggggtgagacaggccagacaggcctgcccttgcccgctccctcgcgagggggcccaacctcacaacgtgcagcccctctgaggcacaaggcaacccccccccgacccagtggcagccagtcaccagcctcagatgccacttcagccccgagagccttgaaacaaaactgaccaggcctgacggcccctcctcccccaccctcacctcacctcctccaaggccttccagacaggagtgccttcccttccactcctcccttctagaaccttctatggatctccccccaacccaggaaggatg

SEQ ID NO: 236 - DNA, 587 bp, Bos taurus
ccaccaccagccccctgggccggtccctgctggcggcctcaccccggggaggcagcctccctgacggtgggctccagaccctgcctaggagggcctcggtcctactggaattggtcggctcagtgggcatgagctcgacagcctgggcgctgtgggaaccaggcccaggcggaccccgggatgggggagccaggagcctgcccactcctccttcccccactcacccgcctgaccaggctctgggggtggaggcccaccctgccctcaggcccccagcctggatggggtctcagacacctcaagggacccaggagaccactgtccagggcatccacagccccccgctggtgtctgcccagatctgctgacatctgagcctggggtctgcacagcccctgcagggagtgggcccagctcagatgtcacctgcctacgcccgcacagtgggctgaggccctgccaggtgggggcgcccggccaccggatgggtgaccgtgagcaggttcctcagccgctgggcagccccttcagaacagggggttggaggagccgtcctcccccatggggcagggggacgggccgtcagc

SEQ ID NO: 237 - DNA, 489 bp, Bos taurus
tccctggaggccggggctgttgcagggaaccctctaccgtggagagggtgtgaggaaccttgtgccaggacgagggagggacaggcgtcacggcctgggcttgttccgtgcagccggtcatggccccgtgtgttcttgtggctgtcagtggggaggggggtgtgcggacgggtgggcggggaggtgggccgaccccaggccgaactggctgtgtcgtgggaccctctgtcacttctggagagagacccctcggcggggtggggggcggtgagaggacggtccggtttgcacgcctgggcaccttgctcagaggagcactcttggcaaaaagctggagcagccgctgagaggccgggagagggaggaggcaggcacggaggagtctgctctgggccccctgccgggtcaaggtccgaagccgcccgtcccgcccccgaggacgtagctgcagaaacgggccggaggacagcggcggagggcctggcccgc

SEQ ID NO: 238 - DNA, 793 bp, Bos taurus
gccctttcccgggctgagcgaagacggcggctgagcgactgcgttgcggccccgggctccgtgtgttcggagcggaggaaagatggcagcggctgcacttcctcttgcactttcacccctggggggaggagaaggagggggcgataccaacaacaccccccccctcgcttgcagaaaaaaaaaaagaaacggcgagctcggcgtggggggccctcggtgcccccggccctgcacgtgcgcacccggatccctggcccggacctccccgcgctgcaaactttccactttctttgtgccgagcgccccccccccctcactcagccgaggatgcacgtccccccttccctatttatacacccccccctcgcagctccgctcccccccgcttcctgcccccccctccccagtcccctgctccgccaatgctagcgcagattgagcccccgtccggcccccgcctccccaccccgcccacggggccgggggctgcagtctcacccctcccggaccaatgcaaacgaagcgcctagccagcaaaccagctcccactgcccctccctccccgccaagagtggcggctagcggacagggccgggggaggggggagaccaagggggtgcagatccgggccccccagcctgccgtgtgcgcccgtaagtccagcgggcgtcggctacgcttcggcacccccctttgatgcgcgaaatccccagaagtcgcttgcgaccgcctcccccactcccaaacccagagagggccagggtcctcgctgggggcaggccgaaccgccgga

SEQ ID NO: 239 - DNA, 500 bp, Bos taurus
ctacacctggcccagcggaaacacctaccagggctactgggcgcagggcaagcggcacgggctgggggtggagacgaagggcaagtggatgtaccggggggagtggtcacatggcttcaaggggcgctacggggtccggcagagcctgtgcacccccgcccgctacgaggggacctggagtaacgggctgcaggacgggtacggcgtggagacctacggggacggaggtgagctgcgtcgctggcggctctgctgccccgcgagctgggcgctgggggagggctttgcccctgactgcggggccgcggggaggacgcgaggcgcacgtgtccggaaacccgccaaaacacctgggggagcctcctaccctcctcccgcggccactctccctgggagggggcgttaggacgcggcgggtgcgctgggggttggatctgaccgggttctgggggcgggcggtgtgtccacacccgggggaacaaggcgtgaagacacctggc

SEQ ID NO: 240 - DNA, 408 bp, Bos taurus
ccgcagggtctggagctggcccagaagctggctgactcagagggcctgggcttgctgaatgtgggcagcgggccggaggagccccgtggggaggagcctgaagtgccagaagtggcttccgctgagctgtgaggaggagggaatgagggggaggagaggggagcaggaggggtccctgaaatgaggctgagaccccaccctccctccagcagctccatctgagctgtggttgccccaccttccatgggttaggggaacagcagccctggcctggctcaggccaatgcccacctcttctccctggtagtggtaccagcgaagggagcgtccaacagtccccgctggagctggggcctggagagtacagggtgttgttgtgtgtggacgtcggcgagaccaaggggtgag

SEQ ID NO: 241 - DNA, 306 bp, Bos taurus
cccccagagggtcggggcagggctgtgctctctttcctgagtcccgggaggtggggggcccgttggccatgcccggcctggcctctgtccagcgcccacgccgatgagctgctgcccttcggtgctgaccggactgccaagccgggctgcctgccggcgtcgggctgtgccacacacggagcggggctgtcgcagagcctggacgccgggccgctcggccgctggacggccctcggctccgccgccccaggacatggacgagttcctaaaaccgctcacactgcagctgcctttacttctaggcct

SEQ ID NO: 242 - DNA, 680 bp, Bos taurus
tcaatagtgtggctgtcgcccccaatgacaagctgctggccacgggctcacaggaccgtacggccaaactctgggccctgccacggtgccagctgctgggcaccttctcgggccaccggcgcggcctctggtgcgtccagttctcgcccatggaccaggtgctggccacggcctcggccgatggcaccatcaagctctgggccctgcaggacttcagctgcctcaaggtgagccgcccctggcccaggcctggcaccccctccaccccagacccggccccccacccgacgcctcaccccccaccccccccacagactttcgaggggcatgatgcttctgtgctgaaggtggcctttgtaagccgcggcacgcagctgctgtccaggtgagtaggggttgggcagggccggggtgcgccgggtgggcccaggctgaacccctccccacccccaccccagtggctctgacgggctcctgaagctctggaccatcaagaacaacgagtgtgtgcggacgctggacgctcacgaggacaaggtctgggggctgcactgtagccggctggacgaccgcgccctcaccggtgccagtgactcccgcgtcgtcctctggaaggtgtgtgggccagggagggcatcacagtgggcagggcagcgtgggggccagtctgagcccaatgt

SEQ ID NO: 243 - DNA, 447 bp, Bos taurus
ctcagggctctggccacagctcgcagcttcctggggctcctgggaggatgaggacaaggagaagtggggggcggggggcctccctgagctggtcactgagcacccccagaaaccccaggcagcagaccccagctcccctctatgtcctctgggcccgtcccccagccagcagcaattcgaaacggccatccatcccttttaatctcagtcggggaggaagcccagggaggcacagaaagcggaccctcagcctcatcactgcgtctcacctgtgtccacggggcccagactggcctctcaaggggctgagggcagaacaggctgtggggccgacacctcgtttctcagaggggcctggggttaagcccggaagcagggagggaagtggtggatgggggccaggatggcaggacgctgagggcagaggcccaggcccacaggggtggg

SEQ ID NO: 244 - DNA, 975 bp, Bos taurus
gccccaccctgggaacgggacgaggctcacctttcccagatgccgggctcagcacagcccacagaggcgggggcgggcctgtgggcttgctcaccacctgcccacctgtggccccgcaatctgcccagggctcgtggtaaccgctttcctcttgaccttctgtgcactctgcctgatttctttttacctcctgattttctgaagttgctggtcttaatgttctgcccgcatgtctttacccggcctggggtcagtaggaaagagccttgtgtgagggggtgggaatcaggcccgccccaggagtgctgcccccgccccaggaggctgggtgagggctgtggcctccaggctgcctgccccttgctgaccttggctccccccgccaccaagagcaggtccagccaagccgtgcacccggagtcgtgatggtgacaccccagactgggcgggccccactgggaggagcagcgtgtgggaagcgggcacggggagggcagccttggagggcgagatgggcagtcaccgcctccacgtgaccagtgggcggcaggcgactccttggtgccatccggcaagtgtcctcatcgaggccctcactgcctgcaatgaccccatgggctggcagtggcgggaggcccaaccccaccccccaccccgccgtgactctgtgccctctgcggaacatctgtgtgcaccctggctcacctcgcacacccctctccccaccccaagtgtgtctccatcactggccctgtgcagactgtggcagggaccacggggatgccaacaggggcacgggggagcatgggtccctcctgggtctcactcggctgcccacgatgcccttctagcccctgcggttgcacctcaggtgggctgcacccccgagctgctgcagaagccgcgcccgccggtaggagatgcagcattgggaccccggccccatgttggccggatggtgcttgtcgggcagcg

SEQ ID NO: 245 - DNA, 336 bp, Bos taurus
cctctgcgcccggggactgcggggcaggcaaggtgcctgcggtgacccccgctgtcccccagcctgatcctgagccgggcgcccgtggcggagcatgccatcctgccggcactgaagcagagcctcaagaccagctttgcggagcggcagcggcggctgcaggccgtgcagggccggcggctgcaccgctcggtgctctgagtgccgtgctggccaccaccctgcgggactgcgccccacggacccccggcactcacacgccaagccagccaggcccacggtgcataccgcacgaggtctcggggctgggaactgtttattttcttcctcagtatc

SEQ ID NO: 246 - DNA, 531 bp, Bos taurus
caggcgcttctgtcttgaagccagtggtccgctgctgggaccaatgggttaagagtcgctgggtggggcagcctcttgctcccatctggttagacgggttgaggccggctcagccagcagcctgcatctttggggtgggggggggaggggtggaggccagggaccctgggtggagccggcagcagggaggtggaggggcagtggacagcagaggccgagcctgcctcgggccgcgagggttaaggggagcctggaactcccacccagggggctgcagagggcgcctgctggggtgagtgtgggcatgctggggagggggcaagtccccatcctgcctcaggagctcactgccatggagacgaaagctgagggtcttcaagtggcggaatgggaggggtggtgtgcacagtctccatgggacgtgcttctgcacgtccgaggaattcacggataacacgggggagcgcagcacgtgccgaccttctcctgatgattcccttgagggatccagcacccctgttctgtgggggg

SEQ ID NO: 247 - DNA, 586 bp, Bos taurus
cactgtgagcccttggggccagcagcccagcctggggctggtggaggggaggacacggcagaggggccctccgcccaatgctgcccttcctgtgaagtcagcagagccaggctagagaacagagggcacgtgctgagggctggcgggcagccggggtgggggcttgcaggagcggaccagcgccgccccgggcccaggcgcccacgggcactgaggcctcctgcctcccaccccttccatgcagatcccttcggaggacgacccccagcaactgcctgaccccttcagtgacctctctgtgggtgggtgggaggtcacagacgagcccgtgggccgcctctccgtgtgggccgtgaccctgcagggaaaggtaaggccaccccagagctgcgggtgggaggtggcggaccgggccgcccacggagcgggtgggggagaggctgcccccgccggcctcctgggtggacgaaggagggcgctccctgggccctcctcatgtcctgtcccagcagtgggggctgcctgagcaggtggcatgttgctgggggccccacagtcccccgtcctgcgaccctgctgtcctggg

SEQ ID NO: 248 - DNA, 367 bp, Bos taurus
ccgtgtgacggtctgagggcagaggcagggccccttgctggacacccggccgcccctgggggttcctgcggcctgggctgccctgagccctgggggggcgcacacaggcatctccccacggtccactgtgcgccccgcccggtcagacccagctgactgcagactcccctccagctgccccagcccctccactgcggacctggggccggcagtgggcgtgtcttgggccgccgcgccctctctggagcggaggcgtccgcagagggctggcgtctcccagcccactgcccccggaagcgggcttctcttctgtgcacccagttactccccggaagaatggtctgggcgtggggacggcagggcggca

SEQ ID NO: 249 - DNA, 591 bp, Bos taurus
tccacatcacactgtccttccaggtcggaggagcggcctgtgaagaagcgcaaggggcagggcggccacctgcagagaaagaagctgcggctgctggaggccctgctggaggagcagcgccggctgagccgcgccctggaggagacatgccgcgaggtgcgccgcgcgctggaccagcacagcgtcttgcaggcccagagcctgcagctgcaggagcgaatgatgagtctgctcgagcgcatcatcgccaagtccggcgtctaggccgcccgggccccgcccccggaacggcccgggccacgcccccagcctgggccccgcccctgacccgggcctcgcccaggaaaggcccaggcctgggccccgccccccggcctgggccacgccacagtctgggccacgccccggagctgccccgcctctggcctgggccccgcccctggcctgggccccgcccccggaacagctccggcctgggccacgccccctgaacggccccgagcccccgggcgtgggaccccccggcgtaaagcccctccctggacaccaggcccctggactcagggtcactgctgggcggctccccagtaa

SEQ ID NO: 250 - DNA, 408 bp, Bos taurus
ccagctccccggcccagcctcagctgggtctcctgcccgcagggagcaggcatcgacacggccttccagtggctggacgagggcgtggagtgggtgagcacgtggccgcggtccttcccagacggtccctgcctcccccagcttcacatcacccccctcatcgcccttctgagcgtgacacgggtaggcaggaaactctgctgcctggggggcggcggggagggaaccctgggctcttagagctcccccaggatgcgccaggccccactgccacaggggcccgcctgtgctcaggtgggctgggtgggcagaagaaagggggcgcccttccccgccaaaggcagattcctaaagccccatctaggtggcatctgggcagagccttcaggacggcagctctgggcacag

SEQ ID NO: 251 - DNA, 336 bp, Bos taurus
gccctcctgggagcggaggtctgaagggtgagccaggagacatggggcctcaggtatgtaccccggggccgccttctctctgggaagatggagccctgtcaccgcctctggtcagggaagcggagtgtggggagctgccctctctgatgcaaagctggaggcggagcagccgtggggtccggagcgggactgcgcgggctgcttcccatcctgccctggctcagggccgcggaccctgcgcctgtctgcgtgacgccacgtgctgacagctgggcggccagggggatgagcacggggtccaggcttggctttgtcctccaagaagcggacacgcta

SEQ ID NO: 252 - DNA, 1093 bp, Bos taurus
ttttctgttggacccttaccttgtcccaggcctgggcccgggctgcggcgcacggcactcccgggaggcagcaggactcgagttaggcccaacgcggcgccacggcgtttcctggccgggaatggcccgtgcccgtgaggtgggggtgggggacagaaaggcggagcgagccaaaggcggggagggggaagggccagggaaggaggggggccggcactacagtgttggcggactggcgggactggggctgcgtgagtctctgagcgcaggcgggcggcggccgcccctcccccggcggcggcggcggcggtagcagctcactcagcccgctgcccgagcggaaacgccactgaccgcacggggattcccagcgccggcgccaggggcacccgggacacgccccctcccgccgcgccattggcccctccgcccaccgccccgcacccattggccaactcgctgccaatcagcggaagccgccggggccacctagagaagaggctgtgctctggggctccggctcctcagagagcctcggctaggtaggggagcggaactctggtgggagggggtccggcggattggtggggggatgggtggctgaggtcgtctggccggtacctgggggtcgctttccccggtgggaagaggggagaatagcgtttgttacgctgaaagggagagaggtggtcagaggcaggcgggagtgcggcccgccttgcggcagccgaagggggagggagaagggagcggaaaaggctcgaatccggacggagccattgctcctgcagagggaggagcgcttccggctcttatcttgtcactgattggttgctgctgctcccgccgtgtgtgaaaacacaaatggcgtgttttggttggagtaaagcgcctgtcagttacagcctcgggagtgcgcagccgcctagggactctcgcattgcccactgggtgggtgcttaggtaggtagggtggagagagacttggatgagcaggcgcggtcggcctcccacggggaggtagggctgggggtgggaagggagggtcagtgaaagtggcttcgcgcgggcgtcctaccacccaccccttcct

SEQ ID NO: 253 - DNA, 357 bp, Bos taurus
gcgggtctgcttgaactttcctcctaggaggtgggaatggaggagccttctctgcacatctctgcccaagtggggctaagggaggaatcagaaataccaggaggctccacccccactgaagtttgaggggaagcggtagggaggaagagaaggcagggtgaggcccagggagcaagaaggggagggagaaggagaggaaggacctcaggaggggtggctgggagggctgccagcagtgttacaagcaaagggttttatcgaacacaatatgcccagaaataaaagggagtgaaacagaaacccagcagctgataaggcggtggcagtagcagcggcagcagccaggcagcctagtat

SEQ ID NO: 254 - DNA, 323 bp, Bos taurus
aagatatgcagggaaggcgaccgagaaagggcacacgtggtgaggggtgcatgcctcttgccctcgggggtctgcaccaggagctccgcggagggggagccagccccgcagacaccggggaagatggcccacccgcgcggccggtgcggggcctggcggggagaccactcgggccggcccagcggccttgtggtgagtggactttaggggctccggtgtggacacgagagacccgtgaaaaggctgaccctgaaatgtggtggcaggcaccatgctcccgtgtttcaggcgaggacactgaggcccagagagacgcggggtga

SEQ ID NO: 255 - DNA, 542 bp, Bos taurus
gcacgggcagggccagcagggcctcggcggtggcccgcagcttggggtcaggctccagcatcgtgacgaggacagaacgcagctcagaggacagccctgtccccgggcagggccacgtcaggggcagggccttgcacctcccagccccttgccaccagccaccccgacccccagagcctgtcctccccgtggcagacagccccccggcccttgccccccaactcaccagccgtgaactcagggggcagatagccctgacgcagccgctgccagccctccccaccgcggggcagctccatgttgcatgccacctccaggatggtgaggcccaggctgggggacggggacagaagcggggcggggagggccgtgagatgcagaacccagcaactcgggttgaccccacggagagggctgctggcctccaccgtgagccagggccctgggggaggaggggctgatgacagggaccacttcgtggagcgtggatctccgggccagcacggctcacacaccaccgtggtgagtcctcccagcagccc

SEQ ID NO: 256 - DNA, 444 bp, Bos taurus
tgtcggtggcacccgccggcctgggcggggccctgagcacggtcaccatccaggcccagcagtgcctggagggcatgtggagcgtctccagggtcaacagcttcctgccgccaacgtgcctggtgagtgccctgcgtccggtgggctgggggcccccgggacaccgggcagggctggactccgccgggctgagtgcccaggtgcgaatgggggtccctcggtgtggtttccgtgagggcaggggggctgtgcgtgttggtggaggccccagaccgtcccacggcgcacatcaggtgtgcgggcaaggtcacgtgggcagggcccgctcccaccccctggagcctcgctcctgccccgcgcagcgcacctccgaggtggtccggtcaccctgagaccccctgtggcaccggggcttggcggaagggcctgcaggctcctgggagg

SEQ ID NO: 257 - DNA, 475 bp, Bos taurus
cccagggcggcaggagacatgaccgcagcgggaccggcagggggaccgggcaccctgcccctcctgtccccatggctgcagccccaacggcctcacacggccatcccgcgggggccacaggtagtcgggacagcgcccgtccacatgcagcgcggccccactgctttctggacaccaggcggcaggcctgccctgaccccggccaccgcccccaagtgctgggcccaacgagcgcaatgccccagaaaggccagggcgggctctgcccgggaggatggagataaccagggcctcaggtgcccacctgcccaccctgatgcctcggggccagcccagcctgggcgaggcctctcctggacgccaaaccagcaagagggagagaacagaggaggaaccggggtcagcgctgccaggacgctgcccggaggacacgtctttcttgctctggatcatgacccgaaggcaggaactcggt

SEQ ID NO: 258 - DNA, 369 bp, Bos taurus
ctcgaagaggttgtgtgcctgcaggagaggggttaggaagcgctggaggaccggctcccacctcagtttcttcccagcctgtgagtcctgcgcagatgaaggccctggccagcctctgtccggggtgctgcccctgctccccctgcccgccccgctgcttctgagccgcttctgagctgtttctcagcctggagggctgggcctgcaatgtccactatgaccaccaggggtccccagagtccacacaagggaccagtgggcctcgccactcaggatcatccagcatcagccgtggacacaggggttgggcctcctttaggcccccaacccgctccgtccctccatggccccatgtgctaggctctgtgc

SEQ ID NO: 259 - DNA, 421 bp, Bos taurus
cctccttccgggagcgccaggccctgggccttgtgggggaggagcgcacggaggagggggtgcgggtgacggaggtggcatgctcctgcggcccctgaggaggcgcccccgccgcaccctgacctgggcgtgctttgcctggacgctgctggaccctccctccagccccgagcctcgttcctgtgcggtgagcactgccctccccgacagccagcgtttcagagctcgctcgcctgggaccagttccagcccacgggacaaagcggtaaccagggccacaggcccggctgaccctggacgggcagttggctctgctgcctggcagggcccttgccatggttacagcccacccagggctctgctcagaggctggcattgtctgggtgttggagtgtggtgagcgcgcgggagccaggccggc

SEQ ID NO: 260 - DNA, 320 bp, Bos taurus
tttatcgaacaaaagaaacttccagagccgggagtgagcccagacgggctagaagccccttctcttctctgcagcctgtggcctcccggctgtgctctgggctcccacgggcatcgggggcccgcctgcctgtgctccaggtgcctgtgggtctcggggggggtggggcacaggggtcctctggctctgggggctcccctgtcgctctgctctctgtgcaccccatcctcggggcgggctcccctggggtcacaggttcaggggacctctgccccaaggctctcatgggctcctgtatctgagaggcccccagccccggg

SEQ ID NO: 261 - DNA, 447 bp, Bos taurus
taaaagaacttcaagatgcgcgtcctggcccatctccccaccggcctcgggctcctctttcgctggtgcagtgtgccggctgtgtgagtgcatcgtgcaccctcctgctgtctgggggtcggcctacagtccgacctggagggctcccgctgcactgagaggcgccaagggcccagcgcccacccctctgctgacgtgggctcctccccagccacaggctgcagcacacgggaacgctcttggcccaagacccctgtctctgaacagccccgtcctctgggcctgcaggctcaaaggggccatggcagctccccacccccgtctccccacccgacccaagcctcgcccatggcggtccttggtggaggcccagatcccacaagggacacggctcccgccctgccgtaagccccgtctgccatgggagcgcagcagaccctgtgtcca

SEQ ID NO: 262 - DNA, 505 bp, Bos taurus
tggcagctctcctgtcttcaatttccgccatccactcctgtcacctggtggcccccagtccccactccgaggatccacaggtgagggcagggggatggctcgtcactggagatccgcccccccccccacacccaaagactgcccctgctgacctcatgtgggcaccttgtgacccctccattgggtctgtctggtacctgaatgacttccttggtgacattcactggcccccacaatgacctcctcacaaggaccccctagtgacaccacgtggcccccagctctccatccacataatgccccatcagtgactcacccactgacaccccatagcttaccccacccagggtccccagtgaccccagaatgaccactcatcacccctccccaggctcgctgaagtcctccccgtccatgtcccacatggaggccctcggcaaagcctggaaccgtcagctcaggtgagcacatcgaaagggcccaggcagtggagtcagaggggcca

SEQ ID NO: 263 - DNA, 663 bp, Bos taurus
ctgcctgcccactcggtgtcctatacactctggcccgcggagagcacattaccgaccgtccctagataggggcggggcataccgcgtccctgttccccttcccttttattatttattatcatttaaaggggccgcgacttgaaaatttccgtccactctgtccccttccctaacacccctgagctcttggagcgtcttcagacctagcgcggggggcgagggggtgtctgggaagggggagggggccttacgagagccctccccacgtcccgggccgcggcgctgaatcaccggcgaggtatttgcatctgagagcgatttgtcacccggtgatttgtctgcggggcggtgagcgcgccggtgggggggaggggaggggcgggggaggccgcgcgcggggagacttttaccaatcgggtcggtggggggtgtagacgcggactcggctggcgactcggtgagtcggcgcgcggcacaaacccggcggtggccgggagccagcagagcgcagctcgcggcccgggccggaggccaggcgcggccggggcagggccggacgccaggcgtggggcatgcgccccccgagcgggccctgcgccccccgagcgggccccgcgcggtgaggctggggtagtaggtgggccggccttttaaaacgcgggt

SEQ ID NO: 264 - DNA, 927 bp, Bos taurus
acctttgacagggagcactgtccccccagcctcaacggtggtgggggcccgaggctctggaactctggagggaagagtgttctgttccgtgggcccaaccagtatctaccaccggtacccgccacgggtaccccccaccctccccctccaccccttccttctcctctgccgctcccctgctcctcttgctgttgtccctcctccctctggtcctcccctcccctctatctccctgcctctccatcctcctccgctccccctcttcccctctcctcccagcccacccccccccaccccggcccaccgcctttccttccctcctcccttcccctccagagacaccactgcggttgtccacacaaagaggcgattgttactccagccccggctgaagcccgaggagcatggtggaggcctgatttccatgaataatcgctggggccactgcaggcagcaacctgagctggggcggccagccgggtggtttgctccggggctctgtctgcgaatcaagggccctggccgggtgccatgctggcttggcggtgggggtggggcccagggcccaaggctggatgcccagcagttgggggactggaggcggggtggtcagcaggggccagagctccggagcagggatctgcaggtggcgaggggctggacgctccagatggagacccggtctgcctgggtcccgtgcgtgggggcacctgccgggcagccctgttgcaggcgtgtggacaggacagctccatcgtcccgattcacgtggtggctggaggcggggttgggggcaggggggctggaagtgcccagcgtgggtgtggcacccccacaggtcaggcgggcgctttctgggaccaagcctcggcttctcacctgccacagctgggcggtggtggatgcagggggcccagcccacggccg

SEQ ID NO: 265 - DNA, 497 bp, Bos taurus
tctgtcccatgaaactgggtttccctctttatccagatggaactcctgggcccccgggggaccctgacaccagatggtcacggggctcccccagaagaccacagagcctggctcagcccagatggtgggtttggggagggtccctggaaggaaatcagaggtgggctcaccaggagtctcccccaggtcctggctctcagcccagacctcccagactccccctccgtgagccctgagctgccctccccttgcccgcagcctggaggtgaccttgtcctcatgaaaggagggagctgggccctgggagagactcaccggcctgcacctgggtcctcaggcccacactcagctctggaagagagcgccctgtgagaggtgtgccctcagccctgagcagcgcctctccacccggggagtctcctgggccctggggtcccctgacggagtaggctggctggggggtccctccagaccagggctccccctgcccctcccat

SEQ ID NO: 266 - DNA, 421 bp, Bos taurus
cacaagttccgtctgcacagctacagcagccccaccttctgcgaccactgtggctccctgctctacgggctggtgcaccagggcatgaagtgttcttgtgagtctgcgaacccagcgcttcgcggcccctgcaaaggcgcagagtctgggtctcgagtaacgcgcgtggtggtggggggagcgtggaggaggggaagtggggtctggagttaaaggcgggacccacagtcccacctcccagttctgtccccccgccaccaggctgcgagatgaacgtgcaccggcgctgtgtgcgcagcgtgccctctctgtgcggcgtggaccacacggagcgccggggccgcctgcagctggagatccgggcgcccacttccgatgagatccacgttacgggtgagaccccgcctcctcgcctggcccc

SEQ ID NO: 267 - DNA, 412 bp, Bos taurus
gaagggcgtggcctccatcttgccccccatgatcatcaccatctcgtccgtcagcttgatgtccggctcccagccgaggttgccgcccggggagctttcgaacatgaagccgaaatctgcaaagcgccgaaaagtcacggtgctggggtcagctgcgggctgggagagccccgcctccgggccccggcccgcccccacgtggatgagggagggagagccccgccccctgccgccccgccctcccgcgcccgccccgcccaccgatgtggatgaggtggcccttcttatccagcatgatgttgccgttgtgccggtccttgatctgcagcaggaacaggaggaggctgtaggcggccatgctgcggatgaagttgtagcgcgcctgcggaggacagcggctcagggcccgcgc

SEQ ID NO: 268 - DNA, 488 bp, Bos taurus
tgacatgtctgccagggccagtgatggcccttctgtcttggccagtgctgacccttcttccttggccagacctggcacttctgcttcagctagggctgacatgtctgccagggccaggcctggcccttctgccttggctagtgctggcccttctgccttggccactcctggctcttctgctgcagccagggctggcactttggccttggccagtgctgacatgtctgccaggaccaggcctggcccttctgcctcagccagtgctggccgttcggccttggccagtcctggcatttctgctgcagccagggctggcactttggccttgtccagtactgacatgtctgccagggccaggcctggcccttctgcctcagccagtgctggcccttcagctttggccagtcttggcacttctcgtgcagccagggctgacatgtctgccagggccaggcctggcccttctgcctgggccagtgctggccctt

SEQ ID NO: 269 - DNA, 439 bp, Bos taurus
ggggggggcgacgcggcacccgacttctcggagcccaccttcttcttcttgccaaccttcgtgaaggtatgggtggtggccccggccagcagggaggagacagcgaccgtcgtgggcacgctccgcagctcctgggccgtgaggcccgcacggccatcctcctccgcctcctctgtgggcggcggcccaccctttttgcccccctttccccccttgccagacttcctgggggggctggctgcccccgctggtagactgggaccccggggcaggatgcgccaccttcttgctggccccactgttccaggcagagatgaggctggtgggggcactgctgggcttggaggcagagggcaccagggcagggaagtcttcctcctgaaaggtggtcctgcccctggcgggcacagggtaagccagcgccagccccacagggcct

SEQ ID NO: 270 - DNA, 794 bp, Bos taurus
gtctggtcccggtaccagccccatatgcagtggatgcaaaacctgccgcggcgcggtccctgagccccttggtggctcggagctttaatggctcccaagttgcgctgcgtctgttgccatggcgactgggccagcggccggaagcggggcctaggcgcgacccaccccctgccggcttcctggtagccgcgccgctccccaggccaggtgggctccagctagagctggggggattctgggaacctgcgttaggagtcagcgatctgagggagaggagactttgacccggggcggatcctgccgcttgtcggcactagttaccttttcttctgccgaggcgttagttgggggcgggcgtccgggactgggcgggagtagggggctgggagggaagaaggcttcgagggctagagctgagtgtgtgagaacgtgtgtggagccgccctcggagctcgttctgagacagaaagtgccccggggctgcgttgggaaggcgggagtcatcctggatagatgagtggggcggctggccaaggggccttgcccgggccgagcgtgcgtgagctcccggcggagagaccgggcctgtgcgtgggacccagccggtccggacgcggagtccggcggccgtgggggaagagatggggtgcaggggtgttccgagggcgtggaactaggcgccgggcgaggtcttccgtgcgggagacgcgctgcgtgaatgcggacaggctgccgaaccgtcggcggccatgggggagaagacaagcagcgggaaaagcccaggcctcggggga

SEQ ID NO: 271 - DNA, 339 bp, Bos taurus
acgtgcctagagggagctgggccagggacagatgcgcgctcacacacaccctgtaccgccggtggccactcccctcccataactgagactttctgttactgggccaaggttttctgccagactcacttccccttatttataatgagtgaattatccccagaaagttcctccctcctcccaggaagccgacctcgacggcctgcggggacacggtgggcccgggtgggggacccgcccggcccatcaccaggacagagctcgtttaggagaaacgcatcggcgcgcgcgcatgaagggcgtgcaggtatccgaggtttgcgcccagcccagggcggcccg

SEQ ID NO: 272 - DNA, 477 bp, Bos taurus
ttccctcttcacctactgctgcccccaccccagtgcctccctggggtcgccacggcatctcctgagtcctgccccaggaaccacggccccggaatctgggaatgtgactctgaccttgcccgcctgccccagcggagagcccgtccctggtgcccacgcccctgatgaagggccactccccacagtgggcacccagggccttggcggccaaccaccctgccagtcccagccctggacagcgcctcccgctgtccccgtcctcatcctccctgggccccaggcactcctctgccggtgcccctctgtgcctgccccccatgctttcctcacctccctgctttctgcctttccctggcccctgctgctccctccggctgccgggcacccctcaccgagctcacagcaggcggccagcaccagcgctggcagtgccagccgtgtcccccacatggtctggttcctgcagcacgtcggtcc

SEQ ID NO: 273 - DNA, 435 bp, Bos taurus
acgcacctgtgttcacacctgcacctgcccgtccccacgcacctgtgttcacacctgcacctgcccatcctcacgcacctgtgttcacacctgcacctgcctgtccccatgcacctgtgttcacacctgcacctgcccctccccatgcacttgtgttcatacctgccctgcccgtcctcacgcacctgtgttcacacctgcacctgcccgtccccacacacctgtgttcacacctgtcccctcatccccacgcacctgtgttcacacctatccctgcccttccccatgcgtgtgttcacacctgcacctgcccgtccctatgcacctgtgttcacacctgcacctgcccatccccatgcatgtgttcacacctgcacctgcccgtccctatgcacctgtgttcacacctgcacctgcccatccccatgcacctgt

SEQ ID NO: 274 - DNA, 497 bp, Bos taurus
ctcccggccacgagtggcagatctcgtcggaccctgagaccgagcggggctcaggccgcctcccgggctggggagccgtccgccgcccctggctgggactcccctcctcccggtttccacgcggcccgcgtcagcaaaggctcgtcagctgcgcgcgggaccctccacgtcgcagcccgtcccgcttctccgcccctgtcaggagcgccgcccgggcgtcgcgggtcggccgcgacgcgccggcgggagggacgcggactgggcgttccaggtttggagcctgcaggctcctcggcgctggtcagccaagagtcgtccctgtccccagagggtcaaaagaaggcacggactccgaggcgccccactccctgagggaacatgtcgactttcccagtgacggacgctggaacccggaagtaaaaccccacgcccacaggaagaggtgatgtggggggacagaaagtgaaaactgaactgtggctgccccctaggggggg

SEQ ID NO: 275 - DNA, 476 bp, Bos taurus
actacgccttcacgggggtcggagggaggagcacaggaccctttgcaggagtcccagcccggggccggcgcggcctggaccctccctgcgccaacggagcggccgggggactggaggggacgctgcgagaagacgggggaaggaaacctgagcaaagccgcgcccgcatttcgcctcgctgccccctcccctcgccgctgagggttcgagaccaccctctgtgacaaggtgccacccaccccggtcgaaaaagccaccgccctttatactgcgggttccggtgaggatgggagtctgcccctccttcccttccctgccgccccgccccccccagctgcagacccaccgccctcaagggcaacgacccccctcctcccccgcccgcagcaggcccgaggatctcgcggaggagcgggcactggacaaggcgggaaacgcgtggcaaatactggacgcgagggtgctgcccggcaacc

SEQ ID NO: 276 - DNA, 323 bp, Bos taurus
gcctcttccaactcactcgagctgataagagaagctgtagttgtctgggccgactccggcggtggccgcttcctcccagaaacacaccaaatcctccaagcgctcggtgaagcacagaaactcatcaggctcgcgggctgtcagcagggccgctgaggatgggggtcaggggagggggtcaagggacccgagtccctttctcgagccacagcatccttccctccctcctgccgcctgtgcggaccgataagaaaaaaagccccgcccctgttcttccagcggggcccttcgaggggtccgccgaagtagtgccccccaaactt

SEQ ID NO: 277 - DNA, 469 bp, Bos taurus
tgccccatcccgtcccactcctgggtctcaccgcctgcacactgggctgagcacttggtccagggtgcgtagtgccaggaatagggaggcagtgcatcacgggcaatgggtgcattgaagcggtagcggagggcaggcagttcagtttgggtcagtacctggggtcgcagcggtgggggatggggggtgggggagaatcaaatagaagccaagtttgagatcccaaaaggatgctaacgtcctcctgcaccgtgctcccagccctgccgttaccatgacgatgagagatgcattaatgggtcctagggcttctaggctctgggtctgatccggcccttgtcggagctgaaaagtggttccggccaggggcaggcggtggggctggggggtcccgggcagcccctccagcagcagggactcctggtctcccttcagggctgggggacagcaaagaggtcagaattccagc

SEQ ID NO: 278 - DNA, 663 bp, Bos taurus
gcgctggcctcgccttattaacaagttctctggggagcggcggcccgacccggagccggctggtgagcccgagaactccgcctgcgcggcggcgccagcggataaccaggcccggagcgccggagctggccttctgggaaggggcgggaggcgcgcgcggtagagcccgcccggccagggccccggacgctccaagaggccgaccgcgctcccagcacgcccgaagctcatgcccggcggctggccggagctgcggctggaggggtggcccggctctgcatggccccggctgctgacatgacttctttgccactcggtgtcaaagtggaggactccgccttcggcaagccggcggggggcggcggaggccagacccccagcaccaccgcggccactgtggccgctatgggcgcagacgaggagggggccaagcccaaagtgtccccttcgctcctgcccttcagcgtggaggcgctcatggccgatcacaggaagcccggggcgaaggacagcgtcctggcggcctcggagggcgcacaggcggcgggcggctccgcgcagcctctaggtgcccggccggggtcgttgggcgccccggatgcgccgtcctcgccgcgaccgctcggccatttctcggtgggaggactcctcaaactgccagaa

SEQ ID NO: 279 - DNA, 388 bp, Bos taurus
ccagaggtcaaaggtgagaggtcagagaacggaaggacagtgagagagagcaagcagggcggcagagagcaaaccaggacctggaagctgacgggacggaggagcctggggctgatgggcaggctggaggctgtgcggagtgggggtctcgggggtcaccacctcgtggtgttcaggagctggggccaacgtgcccggggtggactcagaaaggagtgaacccagagggctcccatgctgagttctgctacaaagaggagcagagacctgcagggtggcagggcggagttgggaggaggagactgaggataggaaggacccgggggtggcgctggcccctggaagattctcctgggaaggagaggctggggaagcctgagtatgtggc

SEQ ID NO: 280 - DNA, 660 bp, Bos taurus
gctaatctgaggggggcgtgctaacgagctgctggcggcccgcggcccccgcccctgtgcgccctgagcgctcggcgtccaggtggacaaccacggaggctgcggagctgggcgctccggccaccccaagcgagcggcacacgcctgggggccgcgcccccgcaggcggcacggggtccctggccgaccctgggcttcaaggacttgggctcgctgcgtcctggggtccctggggtctgagtgctcaccctgacactccacatgggcctcagattccgaccagcagagcggagctgagagtggcctccacgtggggcccccccaggatctcccagcgcccccaacgccagcccctcccaaccctcccccgcaccccccggggcttgccgaccatgtcctaaacacggagccccccgacaacccagctgtcccgcggggaaccccacggccctccatccacccgagtctgttcttcccacgacccagcccagatcgcactgcctgtccctctggattaagctttcagaggctgactgctgcttataggtgcagaccgggagtcctcctcacgccctgcagagtccacactgggcctgccaggaccctcgtggctccgctccccactgtgtctggccctgcaccggggaagggcttgtgtcc

SEQ ID NO: 281 - DNA, 350 bp, Bos taurus
gggcggaggccacacacaaggctgagatgacccctctgggctctttagatcaagttctgggacggggcagtctgccagagccaaagcagcagtgaccaagtagggggaacggggagggctctgagggaggctcaggctgacatgagagtttccagacagcccccagccgtcgctccccagctagtcctcacagtcccccatcaggcagaggccacatgctagcccctgggtggagggtgggggcagatgaggcaggtggtgtctgggtggatgcacggagggggctggctccatctggtggggtggggtaaggaatggagacaagggtggagccacaggaagtggggagg

SEQ ID NO: 282 - DNA, 651 bp, Bos taurus
ggtggacacacggacgctcgctaagttcctaaatcgtacagggtcatttatatatactcttcggagagacgccgtcactttataaaagtccactccggcggcacgggcgatagcgcctcggacacgcgtgacaggaagcggtccctgccccgctcgactgagcttccggcccggggacgggtttcctcctacgtcacggcccctgcgacccctagcggctcagcgccgcggggctccagcgggctccgccccctcagtctggcgggcgggacttccgggcggcgggacttccggcagctgagtgacgggcgcagcatcagcatcagcatcagcggcggcggctgagccctgaggcgacagcggctgcggcggcgacgacgggccgcggcctccggaccacgagcagccggggcgggcgggcgcgccggcggacagcgggccgcgctggcggctgcggtggccacgatgatggagatccagatggacgagggcggcggcgtggtcgtctaccaggacgactactgctcgggctcggtgatgtcggagcgggtgtcgggcctggcgggctccatctaccgcgagttcgagcgcctcatccactgctacgacgaggaggtggtcaaggagctgatgcctctcgtggtgaacgtt

SEQ ID NO: 283 - DNA, 1976 bp, Bos taurus
  misc_feature (1010)..(1059): n is a, c, g, or t
ggacagtccggttggcgggggctccgatggggtagctcagggccttgtgggcgctggagaggctgctgattccacctgagattggtggggtgagagggggtctcgtggagggctttgaaagatgcttcagaggccccgtgggttgtgagggggtgctggtgtgggcgaagcctgggcagcagcccctgctggccctcctcctctcagccccctcccctcctcccgcagtgctccctggtagagtctcacctctcggaccagctgaccctgcacgtggacgtggccggcaacgtggtgggcgtgtcggtggtgacgtaccccgggggctgccggggccacgaggtggaggacgtggacctggagctgttcaacacctctgtgcagttgcagccaccagtcacagcacccgggtgagcagggggtgtgggaaggggacagagccaggccccccaggccgggccagcctcccaccccacccctcccacgacccttctgggctgcctggggctctgggcagggaggggcccctcatcccatctcctccttctacccctaccttcccaggcctgagacagcggctttcatcgagcgcctggagatggagcaggcccagaaggccaagaacccccaggagcagaagtccttcttcgctaaatatgtgagtggggttctgcgcgcccctggggccctttcccaccagccctctccctcccatcctgagccctgctccgtcctgctcggctcccctcctgtatgcaggtctctgtgctcgtctccctggcccctgggctgagtgctggagccccgcgccctggccctcccctgccggtgtctgctcgtggcggtggtcacggggcatccggtctcagaaaacagcccgccccggagctgggcactgaggggagccgggaccagccccctgggtgcctggccggggtcagaccaagcacaggcagtgtctgtccagtttggagggagttcgggggcggggggggggtggggtgggggtnnnnnnnnnnnnnnnnnnnnnnnnnnnnnnnnnnnnnnnnnnnnnnnnnnagggggcctgagctgcgctggggctgcggggtggaagggagagatccctgtccccccatagggacaggtttagaaaggagggagttggggcggggggtccacccgtcaggctggggaggtgggtggcagggtctggggaggatcctgggagccgagggtccttggtgctggtgacgctggggctggaggaggccgtcccacccgggccagggcaggggtcgcggaagccagcgaggcctcggttagcgtgctggtcgctggaaacgcggcccggagaggccgagggcgcgcccggcccggcccgctctccctgacggccgccggacaccgcagccactgctggccctgacctgcgcccccgcccccctcagtggcatctcatcctggggggggccgtgttgctcacagccctgcgtcctgccgcccccgggcccgcaccgccgccgcacgaggcctgagtgaggacctgccccctcccgtctccccgctccccgcagccccccagccctcccccagcccgtagtgcagtaggggacccgggcccactccccccacgcctcccctcacgccgcgcccacctcccccacgctggctgggccaccgcttacctcacctctcttctctgtctctctgtccatctgcccatccacccccgtgccgccggctcacagtggatgtacatcgtcccagtcgtcctcttcctcatgatgtcgggagcgccggatgccgggggccagggcgggggcggcggcggcggtggcggcggaggcagcggccgatgaggggcacggggccggggtgccgcctgccctctttcacccggagcacgcccttcctgcccagcatgcccgaacccttgatcatcttaggagggactctccggaccgtccatctctgcatcccgtgggggtcagagg

SEQ ID NO: 284 - DNA, 318 bp, Bos taurus
ccacggctggtgaggctggaaaccaggtggaactgcagccagggctcggcgtctccctgccgcgccgtccaggccccgcggtggaggatcggtgtccaagtcggcccggagaccccgagcggccccactccctccccgctgcggccgcgctgtctggaaaggggaagagcgaccggcgaggccgaggcccgcttccgcgagccagagcagagccccctctcctactgacacagactcccccgcgtcccctgtccgtccactcccgggcccagctcacctctctccggctcagcggaccgaactcagcgccccacttgg

SEQ ID NO: 285 - DNA, 518 bp, Bos taurus
gtggcgcgagagcgaagcgattggcccaaggccgcgtcaatcaaggcgccggaggaggcgacaaccccgccccccgccggcgcctcccttcagactgtaaggccgctaagggcctgagaaaaatcagacttcgaaaaagtcaacgcagcctttagggaggccaaacccctgcccgaacccgcagtgcccaagccccaataactctaaattacctataggccgcaaataggcgggaagtagacaagacgtcgaggttggtgcagcagagaaacagaggataatggcgtctgcagtgctttcgcctgaagcgccctcctcctttctcgttcgcgcactcactggctggggggggagggggggaggctctcgcgagaactaaaggctgtcccgttctcgcgctcagggaggggcgggtgtagggggtgcttctggacctgaaggaggctttcaggggtgggaggaggggcgtgagcgcgcggcaaatcgggcgggaagcagcgaaaaagggttagggcgcg

SEQ ID NO: 286 - DNA, 596 bp, Bos taurus
ggagaagggcacccgggctggcgggtcccgtctccagtcctgtccaaggccgtgatgtcaggaggaggaggaggagggccaagcgccaggagggctgggccagcaagcgggtggagtcacattggcctgcggccccccagttcccgggcagcgccttgtcacccaggtcggggccaggctgccctgccccagcccgcccagcaggttcagatgctgctcgtgccccaggccggcccgggacagcagccctatggccctggcacagccccttggagcctggctgggtggccctgcatggtgaggcaccgcgcgatgcccccagcactggccccgcggattccaccccgactcacagggcctgtggctcggtgcccatggcggccccgccgcctccacagagcaggaccccctgtccgtcccggctgctctgctatgccagggccttcccacgcctcacccagttctcctgtctgaaggcaccagactgcaggcccccgggctcctctgcctggtgacccctccacctggggtctgaaggagtttcttctgtcctggaggccctggagacggccccaggggtcaccctggtccccctc

SEQ ID NO: 287 - DNA, 631 bp, Bos taurus
ataacacccgggcgcagctgactgccccaggcccgggccagagcgcaccctaaggaagagggaggccgggcctggaagggcctggaggccaaaggaagtggcccgctcgcaaaaggaaggacccaggcgggggtgccgaggacggctgtccaagcgacgagggccagcacgcgcctcgccctgcgcggagccggccaagccctggaggagctctaagacttcccagccaaaagccagcttcgcgccggcggcggagtgggagggaggaggcccggcgcgcgccccgccctcgccgcccgcggtcacaggggcaattacactcctcgctgcagacggttaccagctcgccagcgcatccctctcccggttcccctcgcctcgcctcccctcccctcctgtgccgaactgctggctccatcttagcgagcgcggccgggaagcgcggctgaggccagctgcctctggggacgtcgcgaggctgctcgcacgccgcgcgccccccaggtcgccggggagaggcctccgcaccgcgcagtgtgtcccactcgcgccacagccccgtaaggggttgtcgcggtctcttgaggaccccctctccacgcgctgcaagacccttgcgaccgcgctttac

SEQ ID NO: 288 - DNA, 1051 bp, Bos taurus
cggggaccgcctgccacttcctcctgcttcactagaaggacggacaatactgacggaagcagcatctcggtctgttataaaacagttctttccccgccctgggtggggagcctgtcggcagcactccggaagcatctggccagtggtgagggggctttccaggagcccctctagtgggagatggacccccaccccacccccggagccgtgggacccggctcactgtgcccggggtggacgctgggtcgtggcgggagcttcaggccgcggtgagccgcggggaagccagccacgcaggggggcgccgctctgtggtccatctgacgttgggccccctccagccccacccccaccaggccccggccaagggacgggtttcagacacggcgcacagatgcgttttccgcattaactcagtatcaagttccccacggaacaggccgttccagtgctttagaggaaccgcggtgagtcccacgcggggtcagcccgcctgcgtcccagccccatcgcctgtcgtggaaacggagacctgcagggcccctgcggccggctgagggtcttgcctgtgggtgggggccggggggctgggctctggcccacctgggcctctgtcacaggtcaggggttttcagtcggcggttgtatcggtgacacggagacagcagagacgcgtgtgaattgttcacaagcccgtcccacgggagggtggcacggtcactgtgcaggtgggtgaggagggccgccgtctgccccctgcccagccccagagccccgccgcggggtgagcaggcagctcccagccggcggcccccctgtcctggaggcgggccccaccaggccctcagacggcggcaggggcccacgtggccatcctggccagaaaggggcgggcggtctcctgacagctcgtctccgcagggtccccagccacctgcggtcccgcgtccccgtcgccccccggcactcagtgtcgagggatggtctcccatgccgggcctggcatccagcctgcagcctgcgggcagccggccgaaccctgaaacagcag

SEQ ID NO: 289 - DNA, 375 bp, Bos taurus
ggggcaactccccagcccatttcctggcctgcagatcaagtcggacaagcagcagctgggtagcgtcatctacagcatccaggggcccggtgtggacgaggagccgcgaggcgtcttctccattgacaagttcacggggaaggtgtccctgaacgcgatgctggaccgggagaagaccgaccgcttcagggtgggccccgccccgccccgcccattcctgcaccattcctgcaccattcccgcaggggccccgcccacacctccgcctgcgagactagcgtccttagttgggggcagcgggcagggtttgggacccgttcccacagaggcaggtgttcagggtcgctcgcgcattggacagatgcgcgtgagcag

SEQ ID NO: 290 - DNA, 313 bp, Bos taurus
cacttcctggcagggccagcaggccatacatcacccgacgacacgacgaagcccgaggcagcctggctaggcaggcgggccggcgcccgcccctgcgggacccaccctggggctgagggtggggtttcaggcacggactcctgcctgtgaacagggagctgaggccagcagaggaggggtctccttggagtcccaaagcccaccccgggagatagccactgcctgtacggggggagacacaagtggccgttgcgggccatggggtgacctgaaggggagcgcgggccagccgacagcccgcaggcggagggca

SEQ ID NO: 291 - DNA, 546 bp, Bos taurus
gaatgccgacacaggggggtccctgcgccgacaggtgcctgacagaggagggcagcgaaggcccaggctcccagagtcgccctttgttctccgccagtcgcccagaggaatacattaagcggggaacaaaaagagcggggaagggggcgggggttcgggaggagggcggagtcggggggccacaggcctgatcccctcgggccaagggggccggcacgggagggcctgaggaccacgctaatgtcccccacccacccccgcgggcctgggggactccagacaaagggtcggccgcgcccccgccccgcccactgtgtgcgcgctgtcacggcaggagggtgcactcagcctgggaagctgggaagcggccatctgtcgctgccagcgtgtcacccccgccggccttgggtgggaggggcagggatgcctctgcagccagcaccaggcgggaacgccagccccggccaccgctgccccttccccccctgcacgtccctcttctgtgtccctctcagtgggcgcatggggcatcaccgtgccacagat

SEQ ID NO: 292 - DNA, 763 bp, Bos taurus
gccgtcggggggacagaggaccgggctccggctactccgagagcaacccaagcccttcagggcgcttcctgtcgggtgctggttcaagggagtgtgcgcgggatccccagggtctccgggcgccagatgtgggagggagctagtccggctgcggaggaggcagaggaggaaagaagggagggagggagagagggaaagggttgcggggggttgggggaggaggtggcgctcagcggcgagcccggggaggaggaaaaagagggtgggggggccgggctcccgtccccatctcagcggaaagatagagaaatcgacacaaagctatcggggggaaggtcagctgcttccagggccggacatggatcagctcctggaagggggaggggaaaggaggagagggagccgggaaactcgcgctctgctgtgaggtggtcgtcgcagcgcgctctcggcgcaggggagatggatgcccgctgccttcctccctgcgtggcccggctgcagctcggggcgcagggcggactccggccccctccctctccttgcctggctgcctgctccccccggtggcaggcgcgcctgtccgctcgcccgccctcctctctccttctccccgcctctcggaagagatggcgggcccccgggcaggggcaccaggggtgctgttttgtgctgctgccgcggagcgctgcgctccgtgcgctccggctggatctggggctctccgctggattgcgctccgactcgggctccggctccggcg

SEQ ID NO: 293 - DNA, 467 bp, Bos taurus
tcagaccccagcatgtagcatatccctcagagaacgggagtgggagggaggcaaccccatgaggccggagggcctgagacccgccttcccatctctcccacttcctggcccacaggccctgccctcttccttaggtgcccacacaggggtgcagcctgcggtccccagcccccgagcccaggcccaacctccccctccacgtacagcactcctgacccctctccccacccaccccagcccctgtccttctcagccctcagacattccaggaagccgtctccccaccccctaccccccgccccccagcaaaagcccttggctgtggcactagccacaccacattgtttatggtctctccccaccacggggacccacattctctgactgcccacccccaccctccgcagggcctggcccctggtaaatgttgactgaacccaggtcaggactgattaacaaggttaaaa

SEQ ID NO: 294 - DNA, 333 bp, Bos taurus
actgccttcaccggctggggaccaggcccggcctctggtcacttgcagggcaccgaggggcacaaacgccatccttgcccctgaccaactgataaagaactccaggtgtggcgtcctggcaaggaagcaggcgtgtgtcctctgcccgggggacccggagcccctgaagcccctccctcaggcctgcaggggggttgggggggttggttcctaagcagcagcttccaagggccacgcccaccaggctcagagccagagtcccctggcctggatccccgggggcccacagcacagcagggaaccctgatggcaagagggtccctatccccccaa

SEQ ID NO: 295 - DNA, 379 bp, Bos taurus
acaggctctctccacctgctcggctccactcggctctcctcagtcccgctcggctcctctcggctccgctcggctctacccgacaggctctgtccgcctgctcggctccgctcggctctcctcagccccgctcggccccgctcggctccgctcggctctcctcagccccgctcggccgcactcggctccgctcggctccgctcggctctacccgacaggctctgtccacctgctcggctccgctcggctctcctcagccccgctcggctccgctcggcccacctgctctgccgctgtcgacccgtggaacaatcgcgattcgcgcgcttccctggctgggcgctacggtccttgctgggtccagctcggccctatttgg

SEQ ID NO: 296 - DNA, 358 bp, Bos taurus
gaccccctggtgcaggccgtggggcaccatgccctgctgcgggggcatgcctgtcctgcccagcaaggacatctgtccagggtggcacatggacatgttgagcccccgttggacaccctgctggcctgggaggttggggggccgggagggggtctgctccgccatcatgttctgcaggttcatgagatgcagattggggggctgggccttgggggggtggctctcgctcttggggaagtactggagggtgctgcttggcttctccgaggggatgatcctggacaagtcgaactcggggatccccgtcggggtgggccggatcacctcgctcagctcggggtcattcagcactgatgcc

SEQ ID NO: 297 - DNA, 447 bp, Bos taurus
ggcctcctgcagctccactacttgcggctccccaaggacatcagtgacgaccacgtgatcctgatggactgcacagtgtccacgggcgcggcagccatgatggctgtgcgggtgctgctggtgagtgagggggagacgaggagtgggtgtgtggtggggccggggcgggtccagggccgcagctcagcccctcaccccacaggaccacgacgtgccggaggacaagatcttccttttgtcactgctcatggcggagatgggcgtccactcggtggcctatgccttcccccgcgttagaatcatcaccacagcagtagacaaacgcgtcaatgacctcttccgcatcatccctggcatcggtgaggctgcctggcctcagtggcgctcaggggtggggtggggtgcgagggggtgggcagatgggcctgcctgagaccccaccacaag

SEQ ID NO: 298 - DNA, 379 bp, Bos taurus
tgcttcaaatcgccaggtcagagacaaaacaagacagaaactcatgcttggctcggggagccaaggagaatatcagtgaaggggacatcctgcttcttcagcaaaccctggcggggcggggggcgggcggtgcgtccctggtctctgggcgcgggggggcgggcatcagccccgcagccaccgagcctcatctgagtccccaggagcacagggtccgtactaaccccacactcagcccttcccacagggtgcctggggcttggagggctacctggggcactggccggcacggggagccactgtggccgcagcccaccgtccagacgtccaggtgccgccagacgaggggacgtggttccagcgaactgcaaggtggggc

SEQ ID NO: 299 - DNA, 533 bp, Bos taurus
  misc_feature (182)..(231): n is a, c, g, or t
cattacctcctccgcacatctgcccacttcacacacgccccagccatgaggcgcacgcgagagggctctgcctaagggtcaggggcgcctcccggagccgcacggacggggcagtacggggtgcgggctctcgcatccacactgacaacccggtgatcaagcctggggtggggggggggggnnnnnnnnnnnnnnnnnnnnnnnnnnnnnnnnnnnnnnnnnnnnnnnnnnccccccaccgggccccggcgcggcgcacggaccggttgaattcggagaagacgtagagggcggcgcccgcatcccggccgccggctgccaccacctgcaggtacacggtgttcgggcccccgggcccccatgacgggccgcgcttctcccgcgtgcgcaggtgccgcagagggtccttgggcggccgcgggcggcgcgccggcccctgtgacatggtgcgcccggccgcgcaccgcagccccagcagcgagcggagtgcccacatgcgcagcctcctcttcccggacctgaacgcagccggc

SEQ ID NO: 300 - DNA, 331 bp, Bos taurus
tccagagcccatggcatccagtggattgaggggctgtgcccagggtcgcatggtcaggcccgggcagcccatctgccaggggcggccaagggcggctgggcgacagatggggccttaggatggaagagaggagccaccgtgcaagaaggtggagcgctggcgggcggggggcgggcgggtgacgtgcaggtgccgtgcacgctgtgcatgcgggagagtccacgtgcgcgtcgaggggctcctcgcacaccgggcccgcgtcgtgaggtccctggagagcatccacggctgagcggaggaacccaggccgggggccctttggggactcccc

SEQ ID NO: 301 - DNA, 307 bp, Bos taurus
ggttgacctcccagggggagctcggccagggcctggtaggtgcccccggccctgcccacgcggcacagccagagtgtccccaggggctggctccccagctcctgccgcgggctgacctcgggaaggcagacttgggcgccctggggcaccttggccagggtgaggtgggggcgtatccccccgagaggctgcaacaccctcagcccctcggcctccagcctctggctcagcctctgggccaggctctccaggggtggggacgggggagcgcagagcacgtgggagcccaggcacaccaggcgaccga

SEQ ID NO: 302 - DNA, 676 bp, Bos taurus
tccaccctctccaagcaggtccccgggaatccggagctggaccgtggaggtcgggcccggcgcaggtgagcggcgctgtcaggacccgggactgcgcttgaccccgcggagcaacgagcccctgagtcgacgagcgtgattgggtccctaactttctaactatcccccacccctagcctggcgctcaccccttctccctccgcatgcccgctctggcgcccacacgtcgcggccttggagccagactcctgcgggagccctagagactgctcccagctctgccgccactctgctccgcgcgcgctcccgccccgacgcgcgcccccgcagcccccacctgcctgcgccacccgtgcaccttgccgttctctctaccagcgcgcgtccgcacccggcctcacttgtgcccctctactcccgccagaacccgtggctcctcgaccccggaggggagtatcctgccgagttggcctcccgcccgctctcccgagttctcgtaaacttccccggagcctgaaggcccaggaatcccgcaaagaccttccgggcagaaaaagccgcccgctcgtgcggccagcgctgggggggaccacggaggccgagggcgctcagcccgggtccaacaaggcgaccgtgcttgggggaagggggtgtccctgcggcggccccagagtcg

SEQ ID NO: 303 - DNA, 517 bp, Bos taurus
tcgtgggcggcccttagtccgggctgcggccgaccctggatgccaggaggagcagccccggggccagctgagactcggctggggtgggacaaccgcgccccaggacggcccacggggccagcttccatctgcccgtttctccagtggccagcccgtccatctcagtgccaggcctctggtcagcggagggaggggggccggggctccccggttgaaggcaggaagagagaggcgcgttctccagcccgcggcagccccgtgggtcctgtgcccattccccgcgcccacaggcaggctggacgccgcggaggagccgtggtaccgtcggcgtggccctcctgcccggtgcccgcctggccctccacggctccgggggccgcgggctgggcggcagcgggacggcggaccctctcacccccgggtgccggcggggtgaggtagtaggttgtgtggtttcagggtagtgatgttgccccctcggaagataactatacaacctactgccttcctcgagggg

SEQ ID NO: 304 - DNA, 608 bp, Bos taurus
tcccgggggaacctgttgccggaggaatgggcgggcggcggcggcgaaccctccatcgcctccgcctgggcctcatggcggccctttcgacctagacgctgcgcggcacggcccctccctgttgcctctgttttttttttttaaaaacgaaagaaagaaaaatcagcccccaaccctcacccgtgacctgcgggcccggcggcggctcgtgcggcttccgggagcggaggcgcggccccgcgggccggaggaggcggaggcggaggaggaggccaacatggcggcgcgcagggctgggccgggccgccgccactcctgagcgccccagcgccgtagctcggctcgcggtgctcacgcccgcgcccaacctggcccggcggccgcggagcgcgcagcccaggagcgcaagggcccgcccgggcgcggggacggcgcagaggcagcgccgctgggcccgccaggccccggacccgcagcttcgctgctcgccggccggcggggcccggaggccggagccatggaatcggaggaggagcagcacatgaccacgctgctgtgcatgggcttctcggaccccgccaccatccgcaaggccctgcgcctggcca

SEQ ID NO: 305 - DNA, 419 bp, Bos taurus
gcctgcgccgcctcgacctgacagtctgccgcctcttcagcgtgcccgagcgccttctggccgagctgcccgccctgcgcgagctcgccgccttcgacaacctgttccgccgcgtgcccggcgcgctgcgcggcctggccaacctgacgcatgcgcacctggagcgcagccgcatcgaggccgtggcctccagctcgctgctgggcctgcgccgcctgcgctcgctcagcctgcagggcaaccgcgtgcgcgccgtgcacggcggcgccttccgcgactgcagcgccctggaacacctgctgctcaacgacaacttgctggctgcgctgccggcagatgccttcctcggcctccgccgcctgcgcacgctcaacctgggcggcaacgcgctgggccgcgtggtgcgcgcctggtttgcc

SEQ ID NO: 306 - DNA, 388 bp, Bos taurus
ccttcccaggaccccggactcggtgctgggtgctctgtgttggggctcccggctcctggcatggacccgtggcttgtgctgaaatcaggaagttaacaggccatttgtctcctgcctctgctggagcagctgcttgccttctcctgagatcgggggtgggggcggctcctggggtcccgggctcccccacacctgcatccgctccctccagggcccaggacctctcagagcccatcctgtgaggggcttgtggccccagagacatcatcacccttggtgaggagcagggtcccagggcagggaggccgcctctgcctgggggccccgcccagcccagcctcctagctccagcctggccttccctctccacattcctggccccccatcc

SEQ ID NO: 307 - DNA, 316 bp, Bos taurus
gttttgtggtgatggtgatggtgatgatggtagtggtggtttgtagtaatagcaggaggaacagcagctagcatgtgtgattttctactgtatcaaacttcgctacacacttcacatgcattatctcaaagactcctcataataacattgaggtaaggtctactgttactcctgtttcacagatgaagaaatggtgaatttattcaaggtttcacagctagaaaacagcaagtccaagattcaaattagggatgtctgtgtgcctgtgaagcttaaacttctcccacttttccaaagaggatataaaaccaatcag

SEQ ID NO: 308 - DNA, 268 bp, Bos taurus
gttggttttggcgaggactaaatgagcatgaaagcatgaaagcaagagtatctcaagaaaattagagataccaagggaacatttcatgcaaagatgggctcaataaaggacagaaatggtatggacctaacagaagcagaagatattaagaagaggtggcaagaatacacagaagaactgtacaaaaaagatcttcataacccaggtaatcacgatggtgtgatcactgacctagagccagacatcctggaacgtgaagtcaagtggg

SEQ ID NO: 309 - DNA, 177 bp, Bos taurus
tttgtgggtttctggaaaaggcacaactataggcagagaaatatgtctgtggttgctagggtataggagtggaggaaggagtctgaaaggaaatagcccaagggagcattttcaaaccaatgggacgttttctacatttttattgtggtagcatttacacaatggtatgcatatgtc

SEQ ID NO: 310 - DNA, 349 bp, Bos taurus
ttgtggttgtatatacgtgtgactgtatgtcgtcgtcagggagttgtgcagatgtgctttgggtatccgtgaatgtttgtgtatatgtgtaagggtatatgtgtctgtgtatacgtgtaagtggtgcgtggacatgttaagcacgtgtgcgcctgtatgtgcatgaataaggcacagttatatgtgtgtgtgaacagccatatgtttgggtatatgcgagcaaaagtgcatttggttgcattcaggcatcttaccccggccggaaggcctggaataagctgggtcctgtccaccctcctgtttcatttttagtcccttctctttccacacacccccaaacacacccaac

SEQ ID NO: 311 - DNA, 308 bp, Bos taurus
tcacagaccatcatctacgcacactcaaccgggcatgcaccaaaacagccgcacactaccccgcaatcagtgcagacgacctgtaccaagactctctactatatattgcggtgcgggaacaataccacaacctagacaacaaacactacaccaaaatgtgttacagggtagccaaacgtttagccaacaaaaccatacaacacctcgaccagccgaaacccttacccgatatcattcatctagccgacaaccaaaccagcaactgaaaggggagaacacacaccatggttaaaaccaccatcgacgac

SEQ ID NO: 312 - DNA, 266 bp, Bos taurus
ttgggtcaatgagtcaactttttcacatcaggtggccaaaatattggagcttcagcttcagcatcagtccttccaatgaatattcagggttgatttcctttagggttgactggtttgatctccttgcagtccaagggactctcaagagtcttctccaacaccacagttcaaaagcatcagtttttcggcacttagccttcttcagggtcaaaagtctcacatccgtacatgactactgaaaaaccaaccccaaacacacccaacac

SEQ ID NO: 313 - DNA, 284 bp, Bos taurus
tgtttggtgaagtgaagtggcttggtcgtgtccgactcttagcgacctcatgcactgcagcctaccaggctcatccgtccatgggattttccaggtaagagcgctggagtggggtgccattgccttctctattgacaagctatttgcatttagtaaattcccttagaaatacaccccggaaagccttcctcttcctctctaaaggtaacggtagtgcaacaaaatcagtaacaattttagaaagatgaaacagataacttcagtaattcgcaagttaatatatg

SEQ ID NO: 314 - DNA, 300 bp, Bos taurus
gtttgtgtttatacgaattcccgcttccaacgaaatcctcaaagcaatccaaatatccacttgcagaatccacaaaaagagtgtttcaaaactgctctatcaatagaaaggttcaactcttttagttgagtacacacatcacgaacaagtttctgagaatgcttctgtctggcttttattggaagacgtttccttttcaccaaaggcatcaaagcgctccaaatgtccacttccagattcttccaaaagagtgtttcaaacgtgctcaaagtaagggaatgttcaactctgtgacttgaa

SEQ ID NO: 315 - DNA, 132 bp, Bos taurus
ggtttgtgtcttgagcatcggatcctccatttagagtctttgaacctttagaaagataccagtgggaaactgttagagctttgaggacatcttaaatactaggaagacccaaattcaagaactaacacacaa

SEQ ID NO: 316 - DNA, 136 bp, Bos taurus
tgtttgggtcacagccattctgactggcgtgaaatggtacctcatagtgggttttgtatttgtcatttcttctgataatagagtgatgttgtagcatcttttctatgtgtttgtttagtccactctgtatgtcttc

SEQ ID NO: 317 - DNA, 181 bp, Bos taurus
aaggtgacagcatgtgtccaaccacctcctagaatccttctcgctggcgtccatcttggctgagcaatgtatgcactactgggaaggactctgagtcagaatgactggccagagacaacccagaaattaatcccatcctctgggaacctgttagaaatgcaaattttcaggccccaccaac

SEQ ID NO: 318 - DNA, 174 bp, Bos taurus
tgaaactccgggagttggtgatggacagggaggcctgaagtgctgcagttcatggggtcgcaaagagtcggacatgactgagcgactgatctgatctgatctgaacacctatcctactcaaattcttccagaaaattgcagagaaagttaaactgccaaacacattctatgagg

SEQ ID NO: 319 - DNA, 270 bp, Bos taurus
tgtggttgtgtgtgcctgaagcaagtggaagattcctgcgccagttcagctaatattcttaaaacatcagtgatatgttaaaatattttctatattgtttctgtaacttctatttgtctataagaggaaagagaatttgatggcagtatgttctaagtccgtcagtttcaaaacattctatacagttcagttgagttcagttcagtcgctcagtcatgtccgactcttgcgacccatgaatcacagcacgccaggcctccctgtccaaac

SEQ ID NO: 320 - DNA, 270 bp, Bos taurus; identical to SEQ ID NO: 319

SEQ ID NO: 321 - DNA, 120 bp, Bos taurus
aacgtgctagtaatttagataaacatggagtgtttgacagattgaaggtctaagtacgattgttttccaatttatttccctcctaaccctctacaagggccaaccaagctcttcaaacaa

SEQ ID NO: 322 - DNA, 275 bp, Bos taurus
tggtttgtggatattgttctgaagaactgggtttaatctcacttaattctaattagcttaaaatttaaatttaatagccacaggtggctagtgactaccattttgtacaatgcagccctagagaaaccactgaaaataataaacagtgggatacctaaaagctagtagggaataagactgaaatcgcaaaacaaaacaaacaaacaaaacccaaaacttcaagtaagctcaaagaaagaagaggaaacaaacccaaacccaaacacacccaacac

SEQ ID NO: 323 - DNA, 364 bp, Bos taurus
ttgttgggtacaaggaattctccagttgccctctcccatgactcgaaaacaattgcaagctttcctggggctaactagttattgtagaatctggatacccaactatggtctaattgcccagcctttatatgaaagcttaaagggacgagatgattcaatcccactgatgtggggaactcctcaaaaggaggcagaggctacactaaaacaggccttaactcaggcacctgccttgaggttgccagacccagaaaaagcattccaactttatgtccatgaaagagagggaatagccttgggagtgttaactcagaggttgggatctgagccccagcctgtagcttacttatccaaagaggctcga

SEQ ID NO: 324 - DNA, 142 bp, Bos taurus
tttgtgtgtcatctctttgcttgagatggtgcattttctgcattgcaggcagagtcttcactgtctgagtcaccagggaagcccattttaaggttaatgaatctttattatagactcagagttgatactggaacagggattt

SEQ ID NO: 325 - DNA, 290 bp, Bos taurus
tttgggtggcattgtgggagaaggtttagtgccaaggtaggttcaagaaaagtgggaatgagcactagtgtgtaaggatgctttctgctcattggccagaagtggcttccacacattaattctttaacatagtccagaagttggaggggcttctggatggctttatccagcaactaaacagtgccagattttcatgtctctgctctgcagttcccagtgtcacgtttaacagtcaggcaatagatttccttgttcatatccaacaaaagaatgattgatttatctttacc

SEQ ID NO: 326 - DNA, 160 bp, Bos taurus
gtgggtttgatccctggtcaggaaactagattccacatgccacagtgaagacctgaagaagccaaataaataatgataaataaagaaaatttaggaggaaaacaacaatatatatttttaacaaatagagtatgggctttgcagtctgagagacctggcc

SEQ ID NO: 327 - DNA, 137 bp, Bos taurus
ttgtgtgggtaagggaataatggggaaaaaaaccaacctgagactgtatcatctatcttaatggcatataaaatgtatgtggagagacaactgtaatgtggaaattgatcagtgtaactttttatatattttagact

SEQ ID NO: 328 - DNA, 311 bp, Bos taurus
tttttggtggggaacacatgtatacctgtggcagattcattttgatatatggcaaaaccaatacaatattgtaaagttaaaaaataaaaaaaataactgttcagcttctttggcattagtgattggggcataaatttggattactgtgatattgatgatttgccttggagatgaacagagatcattctgtcatttttgagattgcacccaagtactgcatttcagactcttttattgactatgagggctactccatttcttctaagggattcttgcccacagtagtatatatagtggtcatctgaatacac

SEQ ID NO: 329 - DNA, 255 bp, Bos taurus
tggtatctcttcacggctaccccaccattcaagtcaggagtggtagcagtgaggagatatacctcttccaaggtaaggagcagtggttgtgctttgctggagtagccgtgaagagataccccaaggctacggtaacagaatcccacgttagatggtaggtattgcaagatggcatcagagggcagaaacactgaaacaataatcacagaaagctagacaaatctaatcacacaacaacccaaacacacccaacac

SEQ ID NO: 330 - DNA, 293 bp, Bos taurus
ggtgtgtgtatacatacatacatatgtggtgtgtgtatatatgtatctaagtggtgtgtgtgtgtgtgtgtgtatgtatatatatagtgtgtgtgtgtgtatatatgtatatacatgctgtatatattggagaaggcaatggccaccccactccagtactcttatcttagaaaactcccatggacggaggagcctgggtgggctgcagtcccgtggggtcgctaggagtcggacacgactgaacgacttcgctttcacttttcactttcatgcattggaggaaggaaaaaccc

SEQ ID NO: 331 - DNA, 309 bp, Bos taurus
ttttgttgggtccataccatttctgtcctttatcaagcccatctttgcatgaaatgttcccttggtatctctaattttcttgaagagatctctagtctttcccattctgttgttttcctctatttctttgcattgatcgctgaggaaggctttcttatctctccttgctattctttggaactctgcattcaaatgggaatatctttccaacatataaatagtacctgctacctctcaggtactattccaagtaaactatattaaattaaaatgtcattaaatgacattcagtcatttaatcctcacaca

SEQ ID NO: 332 - DNA, 376 bp, Bos taurus
tggtgtgttggagatctgcaaggagatccgaaccagtctaccctaaaggagatcagtcctgggtgttcattggaaggactgatgttgaagctgaaactccaatactttggccacctgatgcgaagagctgactcatttgaaaagaccttgatgctgggaaagattgagggcaggaggagaaggggacgaccgaggatgagatggttggatggcatcaccgactcaatggacatgagtttgggtaaactctgggagttggtgatggatagggaggcctagtgctgtggttcatggggtcgtaatgagttggacatgactgagtgactgaactgaactgatgagtaaagctgataatacttatgatcacaatcaacct

SEQ ID NO: 333 - DNA, 307 bp, Bos taurus
tttggtttgtgctcacaccattgtgattatctgggtcatgaagatcctttttgtacagttcttctgtgtattcttgccacctcttcttaataccttctgcttctgttaggtccataccatttctgtcctttatcaagcccatctttgcatgaaatgttcccttggtatctctaattttcttgaagagatctctagtctttcccattctgttgttttcctctatttctctgcattaatcatggaggaaggctttcttatctcttcttgctattctttggacacacccccacccaaacacacccaacac

SEQ ID NO: 334 - DNA, 253 bp, Bos taurus
tggtttggtggttgttgttataatttttgtgttcagtattcatttggatttatctcataatttcattttcatttctctttattttttctttgctcttgtagtagcagaatgacattttcttggattgtttgtcttttatgtgtttctcatgatttacctcattttcctttgaatattatttgggcattaaagtcagtagaaggctaaagtgtacatggttaatattgaaagtcatttaaattatccccaacaa

SEQ ID NO: 335 - DNA, 305 bp, Bos taurus
tgggggacgacagaggacagaatggttggatggcatcaccaactcaatggacatgagtttgagcaagctccaggagatggtgaaggacagggaagcctggcatgctgcagtccatgaggtcacaaagagttggacatgactgagtgactgaaacaacaaaatcaaactaataccaacaaagtgctaattatgtgccaaggatcttacatgaattatttcatttaatcattacaactaaatttaaggtactatttactcaccccttgactacagataagaaaactatgatttacggaatttccttg

(19) United States
(12) Patent Application Publication
(10) Pub. No.: US 2019/0226025 A1
(43) Pub. Date: Jul. 25, 2019

(54) Methods and Systems for Detecting Bovine Spongiform Encephalopathy in Cattle

(71) Applicant: CNA Diagnostics Inc., Calgary (CA)

(72) Inventors: Anne Balkema-Buschmann, Insel Riems (DE); Robert B. Church, Calgary (CA); Paul M. K. Gordon, Calgary (CA); Martin H. Groschup, Insel Riems (DE); Christoph W. Sensen, Calgary (CA); Maria Sensen, Calgary (CA); Deepthi T. Thomas Kannanayakal, Calgary (CA)

(73) Assignee: CNA Diagnostics Inc., Calgary (CA)

(21) Appl. No.: 16/114,060

(22) Filed: Aug. 27, 2018

Related U.S. Application Data
(60) Provisional application No. 62/559,476, filed on Sep. 15, 2017.

(51) Int. Cl. C12Q 1/6883

(52) U.S. Cl. CPC C12Q 1/6883; C12Q 2600/156

(57) ABSTRACT

The present invention provides methods and systems for identify bovine spongiform encephalopathy in bovine, and related uses, agents, and kits. The invention includes and methods for detecting and diagnosing BSE.

This is a United States non-provisional patent application claiming priority to U.S. Provisional Patent Application Ser. No. 62/559,476, the entirety of which is hereby incorporated by reference.

### FIELD OF INVENTION

This invention relates to polynucleotides derived from circulating nucleic acids related to bovine spongiform encephalopathy (BSE) and kits and methods for detecting and diagnosing BSE in bovine.

### BACKGROUND

Transmissible spongiform encephalopathies (TSE) are characterized by the accumulation of misfolded prion protein PrP^{sc}. The misfolding leads to an increased stability against proteinase-K digestion. TSEs have been described in several species, such as Scrapie in sheep, Bovine Spongiform Encephalopathy (BSE) in cattle, Chronic Wasting Disease (CWD) in deer and Creutzfeldt-Jakob Disease (CJD) in humans. Although prion diseases display a substantial inter-species variation, they all are characterized by a spongiform vacuolization of the brain, accompanied by a degeneration of nerve cells and a deposition of PrP^{sc} aggregates.

BSE is a transmissible TSE and is believed to be the cause of variant CJD in humans. The infective agent from BSE-infected cattle can be transmitted to humans through occupational or dietary exposure to infected tissues. The dangers of transferring the disease from animals to human has had a significant economic impact on the cattle industry as cattle exports from various countries, including the United States, Canada, and Great Britain, have been blocked numerous times based on fear of BSE transmission. Therefore, assays and methods capable of accurately detecting BSE in cattle, preferably live cattle, are important for preventing widespread infection of cattle and protecting the public from occupational or dietary contact with meat and/or other infected tissues from BSE infected animals. To date, rapid assays used for screening cattle for BSE are generally immunoassays that detect the presence of PrP^{sc}, the misfolded from of the prion protein, which has been linked to the disease. These assays, however, can have a false positive rate as high as 5% and require a tissue sample from the obex part of the brainstem of the slaughtered animal. A rapid assay that could identify BSE infection in live cattle is desirable and would be useful for early detection and identification of BSE infected cattle so that the infected cattle can be removed from the herd to prevent further transmission within the herd and to prevent BSE infected cattle from entering a processing facility or being exported.

It is well known that nucleic acids (NA) bind to PrP and in turn NA are co-eluted with prion protein in most prion enrichment procedures [Dees et al, 1985, J. Gen. Virol., 66 (pt 4):845-9; German et al., 1985, J. Gen Virol., 66(pt 4):439-44]. The role of the PrP-associated NA in the generation, transmission and propagation of TSEs is currently being debated [Meyer et al., 1991, J. Gen Virol., 72(pt 4):37-49; Oesch et al., 1988, Ciba Found Symp., 135:209-23]. A recent study shows that the destruction of the PrP-associated NA lowers prion infectivity substantially, indicating a supporting role [Safar et al., 2005, J. Virol., 79:10796-806]. Although preliminary data suggests that circulating nucleic acids (CNA) are potentially promising as biomarkers for prion infection [Schutz et al., 2005, Clin. Diag. Lab Immunol., 12:814-20] and other chronic diseases [Beck et al., 2009, Zoonoses Public Health, 384-390] in livestock, CNA based assays and methods for detection of BSE in a broad range of cattle breeds that are capable of discriminating BSE infection from other brain-related neurological diseases or trauma (which could account for an unacceptable incidence of false positives) are not currently available.

### SUMMARY

According to one aspect of the disclosure, there is provided a method of detecting BSE in a bovine, comprising analyzing a diagnostic sample of the bovine for the presence of one or more of the polynucleotides selected from Table 1, 2, or 4. In some embodiments, the step of analyzing the sample includes isolating nucleic acid from the sample; amplifying the isolated nucleic acid using primers that are specific for or capable of amplifying a sequence corresponding to the selected polynucleotides; and sequencing the amplification products. In some embodiments, the step of analyzing the sample includes obtaining the sample from the bovine. In some embodiments, wherein the nucleic acid comprises genomic DNA, mRNA, or cDNA obtained from mRNA. In some embodiments, wherein the step of determining the presence of the one or more polynucleotides includes use of at least one of a PCR-based detection method and a hybridization-based method. In some embodiments, the one or more of the polynucleotides include one or more nucleotide motifs. In some embodiments, the nucleotide motif comprises ATP8B3, CDK5, CSK, FSD9, LOC507825, NOTUM, POLN1, or POLN2. In some embodiments, the sample is obtained from live bovine. In some embodiments, the steps are repeated on a periodic basis. In some embodiments, wherein the periodic basis includes an annual basis. In some embodiments, the nucleotide comprises a circulating nucleic acid.

According to another aspect of the present disclosure, there is provided a use of an oligonucleotide capable of identifying at least one of the polynucleotides selected from Table 1, 2, or 4 from a sample obtained from a bovine to detect bovine spongiform encephalopathy. In some embodiments, the oligonucleotide comprises a DNA or RNA probe. In some embodiments, the oligonucleotide is labelled with a detectable marker. In some embodiments, claim 12, the detectable marker comprises a radioactive marker or fluorescent marker. In some embodiments, the selected polynucleotide comprises a nucleotide motif. In some embodiments, the nucleotide motif comprises ATP8B3, CDK5, CSK, FSD9, LOC507825, NOTUM, POLN1, or POLN2. In some embodiments, the oligonucleotide is mounted in an array. In some embodiments, the sample is obtained from live bovine.

According to another aspect of the present disclosure, there is provided a system for detecting BSE including one or more probes for detecting polynucleotides selected from Table 1, 2, or 4. In some embodiments, the system includes at least four different probes. In some embodiments, the array includes at least eight different probes. In some embodiments, the array includes one or more probes for detecting polynucleotides associated with one or more bovine diseases or disorders other than BSE. In some embodiments, the polynucleotides include one or more nucleotide motif. In some embodiments, the one or more nucleotide motifs include ATP8B3, CDK5, CSK, FSD9, LOC507825, NOTUM, POLN1, or POLN2. In some embodiments, the array is a microarray, gene chip, DNA chip, or film array.

According to another aspect of the present disclosure, there is provided a kit for detecting BSE in bovine comprising at least one primer pair for amplifying a polynucleotide selected from Table 1, 2, or 4. In some embodiments, the selected polynucleotide includes a nucleotide motif. In some embodiments, the kit includes primer pairs for amplifying at least four polynucleotides. In some embodiments, the kit includes primer pairs for amplifying at least 8 polynucleotides. In some embodiments, the polynucleotides comprise of one or more nucleotide motifs. In some embodiments, the one or more nucleotide motifs comprises ATP8B3, CDK5, CSK, FSD9, LOC507825, NOTUM, POLN1, or POLN2. In some embodiments, the bovine is a live animal.

According to another aspect of the present disclosure, there is provided a method of detecting BSE in a bovine, the method including the step of analyzing a biological sample of the bovine for the presence of one or more of the polynucleotides selected from Table 1, 2, or 4, wherein the presence of the one or more polynucleotides in the biological sample is indicative that the bovine has BSE. In some embodiments, the step of analyzing the biological samples includes the steps of obtaining the sample from the bovine; isolating nucleic acid from the sample; amplifying the isolated nucleic acid using primers that are specific for or capable of amplifying a sequence corresponding to the selected polynucleotides; and sequencing the amplification products. In some embodiments, the nucleic acid comprises genomic DNA, mRNA, cDNA obtained from mRNA, or circulating nucleic acid. In some embodiments, the one or more of the polynucleotides comprise one or more nucleotide motifs. In some embodiments, the nucleotide motif comprises ATP8B3, CDK5, CSK, FSD9, LOC507825, NOTUM, POLN1, or POLN2. In some embodiments, the sample is obtained from a live bovine.

Additional aspects of the present invention will be apparent in view of the description which follows.

### BRIEF DESCRIPTION OF THE FIGURES AND TABLES

The invention will now be described in relation to the drawings and tables.

FIG. 1 is a flowchart of illustrating the steps taking to identify the polynucleotides of the disclosure and identifying the BSE-specificity of these sequences across multiple commercial cattle breeds according to an embodiment.

FIG. 2 is a flowchart illustrating the steps taken for the 454 sequence analysis according to an embodiment.

FIG. 3 is a flow chart illustrating the steps taken for the Illumina sequence analysis according to an embodiment.

FIG. 4 illustrates multiple sequence alignments of the reference sequence for ATP8B3 to the 10 main bovine breeds and 3 lesser bovine breed, according to an embodiment.

FIG. 5 illustrates multiple sequence alignments of the reference sequence for CDK5 to the 10 main bovine breeds and 3 lesser bovine breed, according to an embodiment.

FIG. 6 illustrates multiple sequence alignments of the reference sequence for CSK to the 10 main bovine breeds and 3 lesser bovine breed, according to an embodiment.

FIG. 7 illustrates multiple sequence alignments of the reference sequence for FSD9 to the 10 main bovine breeds and 3 lesser bovine breed, according to an embodiment.

FIG. 8 illustrates multiple sequence alignments of the reference sequence for LOC507825 to the 10 main bovine breeds and 3 lesser bovine breed, according to an embodiment.

FIG. 9 illustrates multiple sequence alignments of the reference sequence for NOTUM to the 10 main bovine breeds and 3 lesser bovine breed, according to an embodiment.

FIG. 10 illustrates multiple sequence alignments of the reference sequence for POLN1 to the 10 main bovine breeds and 3 lesser bovine breeds, according to an embodiment.

FIG. 11 illustrates multiple sequence alignments of the reference sequence for POLN2 to the 10 main bovine breeds and 3 lesser bovine breeds, according to an embodiment.

Table 1 illustrates BSE-specific motifs by reference to the nucleic acid sequence numbering of the reference region identified by accession number, according to an embodiment.

Table 2 illustrates the genes to which the BSE-specific sequences identified by 454 sequencing were mapped.

Table 3 shows a listing of the breeds and types of samples used to obtain the chromosomal DNA.

Table 4 shows a listing of 8 regions determined based on Illumina sequencing results.

Table 5 shows a listing of bovine genes to which the BSE-specific motifs in Table 2 were mapped and a description of the genes, including cellular components, biological processes, and molecule functions in which the genes are implicated.

Table 6 shows a listing of bovine genes to which the BSE-specific motifs in Table 13 were mapped and a description of the genes, including cellular components, biological processes, and molecule functions in which the genes are implicated.

Table 7 is a summary of multiple sequence alignment of 8 BSE biomarkers to 10 main breeds and Brahman, Nellore, and Plains bison.

Table 8 illustrates the sampling strategy used to collect samples from control and BSE inoculated animals.

Table 9 is a summary of the sequencing data from ten different runs of the Roche 454 Titanium high-throughput DNA sequencing.

Table 10 illustrates the sampling strategy used to collect blood serum samples from atypical L-type BSE-infected cattle.

Table 11 illustrates the sampling strategy used to collect blood serum samples from atypical H-type BSE-infected cattle.

Table 12 illustrates the sample strategy used to collect blood samples from control animals for atypical BSE-infected cattle.

Table 13 illustrates the genes and Kyoto Encyclopedia of Genes and Genomes (KEGG) pathways to which the BSE-specific sequences identified by Illumina sequencing (i.e., Table 1) were mapped.

Table 14 describes each of the KEGG pathways to which BSE-specific sequences identified by Illumina sequencing (i.e., Table 1) were mapped.

### DETAILED DESCRIPTION OF THE INVENTION

Various embodiments will be described in detail with reference to the drawings and tables. Reference to various embodiments does not limit the scope of the claims attached hereto. Additionally, any examples set forth in this specification are not intended to be limiting and merely set forth some of the many possible embodiments.

In general, the present disclosure describes polynucleotide sequences derived from circulating nucleic acids (CNA) isolated from blood serum of BSE infected cattle. The polynucleotide sequences have been found to exclusively occur in BSE-infected animals and can be used to detect BSE-infected animals before the usual slaughtering age of the animals. Assays and methods comprising the polynucleotide sequences and/or primers and/or probes for amplifying or detecting the polynucleotide sequences in a sample, such as blood serum, from cattle are also described and are useful for screening a broad range of cattle breeds for BSE infection.

The following definitions are presented as an aid to understand the invention.

The term “DNA” means a polymer composed of deoxyribonucleotides.

The terms “sample”, “biological sample”, “diagnostic sample”, and the like refer to a material known or suspected of expressing or containing one or more polynucleotide or polypeptide markers. The diagnostic sample may be any tissue (e.g., blood, bone, brain tissue, endometrial tissue, kidney tissue, mammary tissue, muscle tissue, nervous tissue, soft tissue, etc.).

The terms “polynucleotide” and “nucleic acid”, used interchangeably herein, describe a polymer of any length, e.g., greater than about 10 bases, greater than about 100 bases, greater than about 500 bases, greater than 1000 bases, usually up to about 10,000 or more bases composed of nucleotides, such as deoxyribonucleotides or ribonucleotides, or compounds produced synthetically which can hybridize with naturally occurring nucleic acids in a sequence specific manner analogous to that of two naturally occurring nucleic acids in Watson-Crick base pairing interactions. Polynucleotide and nucleic acid include polynucleotides that encode a native-sequence polypeptide, a polypeptide variant, a portion of a polypeptide, a chimeric polypeptide, or an isoform, precursor, complex, modified form, or derivative of any of the foregoing, and any precursors thereof. Polynucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may be modified after synthesis (e.g., by conjugation with a label, such as a radioactive, chemiluminescent, chemifluorescent, or fluorescent label, and the like). Other types of modifications to polynucleotides known to a person skilled in the art include substitution of one or more naturally-occurring nucleotides with an analog, internucleotide modifications (e.g., uncharged linkages, charged linkages), and the like. Polynucleotides can also include circulating nucleic acids (“CNA”). The term “circulating nucleic acid” or “CNA” refers to free nucleic acid, including RNA and DNA, circulating in the blood. CNA can include gene transcripts, transcription factors or other polynucleotide sequences. CNA can be obtained from any applicable biological sample, including blood, plasma, serum, and the like.

“Oligonucleotides” include short, single-stranded polynucleotides that are at least seven nucleotides in length and less than about 250 nucleotides in length. The term “polynucleotides” includes oligonucleotides.

“Label” refers to a detectable compound or composition and “labelling” refers to the conjugation, fusion, or attachment of a detectable compound or composition to another. In some aspects described herein, the label is conjugated or fused directly or indirectly to a reagent, such as a polynucleotide probe or an antibody, and assists with the detection of the reagent to which it is conjugated or fused. The label itself can also be detectable (such as radioisotope labels or fluorescent labels and the like). In some aspects described herein, the label is an enzymatic label which catalyzes chemical alteration of a substrate compound or composition and results in a detectable product.

The term “diagnosis”, as used herein, refers to the identification or classification of a molecular or pathological state, disease, or condition (e.g., BSE).

“Primer” refers to a polynucleotide capable of acting as a point of initiation of synthesis along a complementary strand when conditions are suitable for synthesis of a primer extension product. The synthesizing conditions include the presence of four different nucleotide bases (adenosine, cytidine, guanosine, thymidine/uridine) and at least one polymerization-inducing agent such as a reverse transcriptase or a DNA polymerase. The primers are present in a suitable buffer, which may include constituents which are co-factors or affect conditions such as pH and the like at various suitable temperatures. Primer includes single-stranded polynucleotide that is capable of hybridizing to nucleic acid and allowing the polymerization of a complementary nucleic acid, generally by providing a free 3′-OH group. Double stranded sequences can also be utilized. Primers are typically at least about 15 nucleotides. In some embodiments, primers can have a length of from about 15 to about 30, about 15 to about 50, about 15 to about 75, about 15 to about 100, or about 15 to about 500 nucleotides.

“Vector” or “expression vector” refers to a nucleic acid molecule that is capable of propagating another nucleic acid to which it is linked. It includes vectors as self-replicating nucleic acid structures, vectors that incorporate into genomes of host cells to which the vectors are introduced, and vectors that can direct expression of nucleic acids to which vectors are linked.

A “motif” or “sequence motif” refers to a nucleotide sequence pattern that is generally conserved across multiple species. Polynucleotides can be derived from the motif. The polynucleotides can correspond to the entire sequence of the motif or a portion or portions of the motif.

“Marker” or “biomarker” refers to an indicator which can be detected in a sample, and includes predictive, diagnostic, and prognostic indicators and the like. The marker can be an indicator of a particular disease or disorder (e.g., BSE) having certain molecular, pathological, histological, and/or clinical features. Exemplary biomarkers include, without limitation, polynucleotides, polypeptides, polypeptide and polynucleotide modifications (such as post-translational modifications and the like), carbohydrates, and/or glycolipid-based molecular markers. The “presence”, “amount”, or “level” of a marker associated with an increased clinical benefit to an individual is a detectable level of the marker in a sample. The presence, amount, or level of a marker can be measured by methods known to a person skilled in the art. The presence, amount, or level of a marker may be measured prior to treatment, during treatment, after treatment, or a combination of any of the foregoing.

“Encode” refers to a polynucleotide “encoding” a polypeptide if, in its native state or when manipulated by methods well known to those skilled in the art, it can be transcribed and/or translated to produce the mRNA for the polypeptide and/or the polypeptide (or a fragment thereof). The anti-sense strand is the complement of such a nucleic acid, and the encoding sequence can be deduced therefrom.

“Array” or “microarray” refers to an ordered arrangement of hybridizable array elements on a substrate, such as solid substrate (e.g., glass slide and the like) or a semi-solid substrate (e.g., nitrocellulose membrane and the like). In some embodiments, the array elements may be polynucleotide probes (e.g. oligonucleotide). Array may include DNA microarrays (including cDNA microarrays, oligonucleotide microarrays, SNP microarrays, and the like), protein microarrays, peptide microarrays, antibody microarrays, and the like.

“Amplification” or “amplifying” refers to the production of one or more copies of a reference nucleic acid sequence or its complement. Amplification may be linear or exponential (e.g., in a polymerase chain reaction (PCR)). A nucleic acid copy produced from amplification may not have perfect sequence complementarity or identity relative to the reference sequence. In some embodiments, the copies can include nucleotide analogs, including deoxyinosine, intentional sequence alterations (such as alterations introduced through a primer that is hybridizable, but not fully complementary, to the template), and/or sequence errors that occur during the amplification process.

The terms “expression” and “expression level”, in general, are used interchangeably and generally refer to the amount of a marker in a sample. “Expression” generally refers to the process by which information (e.g., gene-encoded and/or epigenetic) is converted into the structures present and operating in the cell. Therefore, as used herein, “expression” can refer to transcription into a polynucleotide (such as mRNA and the like), translation into a polypeptide, or even polynucleotide and/or polypeptide modifications (e.g., post-translational modification of a polypeptide and the like). Fragments of the transcribed polynucleotide, the translated polypeptide, or polynucleotide and/or polypeptide modifications (such as post-translational modification of a polypeptide and the like) will also be regarded as expressed whether they originate from a transcript generated by alternative splicing or a degraded transcript, or from a post-translational processing of the polypeptide (e.g., by proteolysis). “Expressed genes” include those that are transcribed into a polynucleotide as mRNA and then translated into a polypeptide, and also those that are transcribed into RNA but not translated into a polypeptide (such as transfer and ribosomal RNAs and the like).

An “isolated” nucleic acid refers to a nucleic acid molecule that has been separated from a component of its natural environment. An isolated nucleic acid includes a nucleic acid molecule contained in cells that ordinarily contain the nucleic acid molecule. The nucleic acid molecule may be present extrachromosomally or at a chromosomal location that is different from its natural location.

An “isolated” antibody is an antibody which has been separated from a component of its natural environment. In some aspects described herein, an antibody is purified to greater than 95% or 99% purity as determined by methods known to a person skilled in the art, such as electrophoretic methods (e.g., SDS-PAGE, isoelectric focusing, capillary electrophoresis), chromatographic methods (e.g., ion exchange chromatography or reverse phase HPLC), and/or the like.

The term “sequencing” and its variants include obtaining sequence information from a strand of a nucleic acid molecule, typically by determining the identity of at least some nucleotides (including their nucleobase components) within the nucleic acid molecule. The term sequencing may also refer to determining the order of nucleotides (base sequences) in a nucleic acid sample (e.g. DNA or RNA). Many techniques are available and known to a person skilled in the art, such as Sanger sequencing, high-throughput sequencing technologies (such as the GS FLX platform offered by Roche Applied Science, Penzberg, Germany, based on pyro sequencing), and the like. High-throughput sequencing technologies refer to sequencing technologies having increased throughput as compared to traditional Sanger- and capillary-electrophoresis-based approaches (e.g., with the ability to generate hundreds of thousands or millions of relatively small sequence reads at a time). These high-through-put sequencing technologies include, but are not limited to, sequencing by synthesis, sequencing by ligation, pyrosequencing, sequencing by hybridization, and/or the like.

In the methods described herein, protein to be assayed can be isolated and purified from a diagnostic sample using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to PrP), and precipitation (e.g., with isopropanol and a reagent such as Trizol). Isolation and purification of a protein can be followed by electrophoresis (e.g., on an SDS-polyacrylamide gel). Nucleic acid can be isolated from a diagnostic sample using standard techniques known to one of skill in the art.

As used herein, “reactive” means the agent has affinity for, binds to, or is directed against specific CNAs. As further used herein, an “agent” includes a protein, polypeptide, peptide, nucleic acid (including DNA or RNA), antibody, Fab fragment, F(ab′)2 fragment, molecule, compound, antibiotic, drug, and any combinations thereof. A Fab fragment is a univalent antigen-binding fragment of an antibody, which is produced by papain digestion. A F(ab′)2 fragment is a divalent antigen-binding fragment of an antibody, which is produced by pepsin digestion. By way of example, the agent of the present invention can be labeled with a detectable marker. Agents that are reactive with CNAs can be identified by contacting the CNA with an agent of interest and assessing the ability of the agent to bind to the CNA.

In one embodiment of the present invention, the agent reactive with a BSE biomarker is an antibody. Antibodies for use herein can be labeled with a detectable marker. Labeling of an antibody can be accomplished using one of a variety of labeling techniques, including peroxidase, chemiluminescent labels known in the art, and radioactive labels known in the art. The detectable marker of the present invention can be, for example, a nonradioactive or fluorescent marker, such as biotin, fluorescein (FITC), acridine, cholesterol, or carboxy-X-rhodamine (ROX), which can be detected using fluorescence and other imaging techniques readily known in the art. Alternatively, the detectable marker can be a radioactive marker, including, for example, a radioisotope. The radioisotope can be any isotope that emits detectable radiation, such as ³⁵S, ³²P, or ³H. Radioactivity emitted by the radioisotope can be detected by techniques well known in the art. For example, gamma emission from the radioisotope can be detected using gamma imaging techniques, particularly scintigraphic imaging. By way of example, the agent of the present invention is a high-affinity antibody labeled with a detectable marker.

Where the agent of the present invention is an antibody reactive with a BSE biomarker, a diagnostic sample taken from the bovine can be purified by passage through an affinity column which contains the antibody having affinity to the BSE biomarker as a ligand attached to a solid support, such as an insoluble organic polymer in the form of a bead, gel, or plate. The antibody attached to the solid support can be used in the form of a column. Examples of suitable solid supports include, without limitation, agarose, cellulose, dextran, polyacrylamide, polystyrene, sepharose, and other insoluble organic polymers. The antibody can be further attached to the solid support through a spacer molecule, if desired. Appropriate binding conditions (e.g., temperature, pH, and salt concentration) can be readily determined by the skilled artisan. By way of example, the antibody can be attached to a sepharose column, such as Sepharose 4B.

Alternatively, a diagnostic sample from a bovine animal can be assayed using hybridization analysis of nucleic acid extracted from the diagnostic sample taken from the bovine to determine the presence of a BSE biomarker, such as a CNA. In this aspect of the invention, the hybridization analysis can be conducted using Northern blot analysis of mRNA. This method also can be conducted by performing a Southern blot analysis of DNA using at least one nucleic acid probe which hybridizes to CNAs (including amplified CNAs). The nucleic acid probes of the present invention can be prepared by a variety of techniques known to those skilled in the art, including, without limitation, the following: restriction enzyme digestion of nucleic acid; and automated synthesis of oligonucleotides having sequences which correspond to selected portions of the nucleotide sequence of the BSE biomarker, using commercially-available oligonucleotide synthesizers.

The nucleic acid probes used herein can be DNA or RNA, and can vary in length from about 8 nucleotides to the entire length of the nucleic acid encoding for a BSE biomarker. In some embodiments, the nucleic acid probes are oligonucleotides. The nucleic acid used in the probes can be derived from mammalian polynucleotide sequence complementary to the BSE biomarker. In addition, the nucleic acid probes of the present invention can be labeled with one or more detectable markers. Labeling of the nucleic acid probes can be accomplished using one of a number of methods known in the art (e.g., nick translation, end labeling, fill-in end labeling, polynucleotide kinase exchange reaction, random priming, SP6 polymerase (for riboprobe preparation)) along with one of a variety of labels (e.g., radioactive labels, such as ³⁵S, ³²P, or ³H, or nonradioactive labels, such as biotin, fluorescein (FITC), acridine, cholesterol, or carboxy-X-rhodamine (ROX)). In some embodiments, these nucleic acid probes are used in an array or microarray.

The detection of BSE CNA biomarker expression in methods of the present invention can be followed by an assay to measure or quantify the extent of expression of a protein encoded by the BSE CNA biomarker in a diagnostic sample of a bovine. Such assays are well known to one of skill in the art, and can include immunohistochemistry/immunocytochemistry, flow cytometry, mass spectroscopy, Western blot analysis, or an ELISA for measuring amounts of the protein encoded by the BSE CNA biomarker. For example, to use an immunohistochemistry assay, histological (paraffin-embedded) sections of tissue can be placed on slides, and then incubated with an antibody against such proteins. The slides can then be incubated with a second antibody (against the primary antibody), which is tagged to a dye or other colorimetric system (e.g., a fluorochrome, a radioactive agent, or an agent having high electron-scanning capacity), to permit visualization of CNA biomarker that is present in the sections. In some embodiments, the proteins encoded by BSE CNA biomarkers can be detected using arrays, including antibody arrays and the like.

It is contemplated that a diagnostic sample of the present invention can be assayed for protein expression by a veterinarian, a laboratory technician, or other clinician. Accordingly, the method of the present invention can further include providing to a veterinarian a report of the results obtained upon assaying a diagnostic sample of the bovine for BSE biomarker expression.

In addition, the present invention provides a method of determining whether a bovine animal has BSE. The method includes analyzing a diagnostic sample of the bovine for the presence of at least one BSE biomarker, and recommending a corroboration test for BSE if the at least one BSE biomarker is present in the diagnostic sample. In some embodiments, the corroboration test includes ELISA, immunohistochemistry, and Western Blot/immunoblot or a combination of more than one of any of the foregoing.

In a further aspect, the present invention provides a method of determining progression of BSE in bovine. The method includes analyzing a diagnostic sample of the bovine for the presence of more than one BSE biomarkers. The detection of the presence of more than one BSE biomarkers can be indicative of BSE progressing in the bovine.

In the methods described herein, the step of analyzing a diagnostic sample can include obtaining the sample from the bovine; isolating nucleic acid from the sample; amplifying the isolated nucleic acid using primers that are specific for or capable of amplifying a sequence corresponding to a BSE CNA biomarker; and sequencing the amplified nucleic acid. In some embodiments, the isolated nucleic acid includes genomic DNA, mRNA, and/or cDNA obtained from mRNA. In some embodiments, the step of determining the presence of the at least one mutation includes use of at least one of a PCR-based detection method and a hybridization-based method. In some embodiments, the step of determining the presence of the at least one mutation includes an immunohistochemical analysis. In some embodiments, an array or a microarray is used for identifying the BSE biomarker.

The diagnostic sample can be assayed for expression of BSE biomarkers in vitro or in vivo. In addition, the diagnostic sample can be assayed for expression of BSE biomarkers using all of the various assays and methods of detection and quantification described above.

The discovery that certain CNAs constitute BSE biomarkers provides a means of identifying a bovine with, and presents the potential for commercial application in the form of a test for the diagnosis of BSE and kits including same. The development of such a test or kit would provide general screening procedures; these procedures could assist in the early detection and diagnosis of BSE. Accordingly, the present invention further provides a kit for use as an assay of BSE, comprising an agent reactive with a BSE biomarker. The agent can be any of those described above, and can be used in any of the above-described assays or methods for detecting BSE biomarkers.

Oligonucleotides antisense to the BSE CNA biomarkers having a mutation can be designed based on the nucleotide sequence of the applicable motif or sequence. A nucleotide sequence complementary to the selected partial sequence of the BSE CNA biomarker, or the selected variation sequence, can then be chemically synthesized using one of a variety of techniques known to those skilled in the art, including, without limitation, automated synthesis of oligonucleotides having sequences which correspond to a partial sequence of the BSE CNA biomarker nucleotide sequence, or a variation sequence thereof, using commercially-available oligonucleotide synthesizers.

The present invention also provides the use of an oligonucleotide capable of identifying at least one BSE CNA biomarker to determine the presence of BSE in the bovine. The oligonucleotide can be labelled with a detectable marker, such as a radioactive marker, fluorescent marker, the like, or a combination of any of the foregoing.

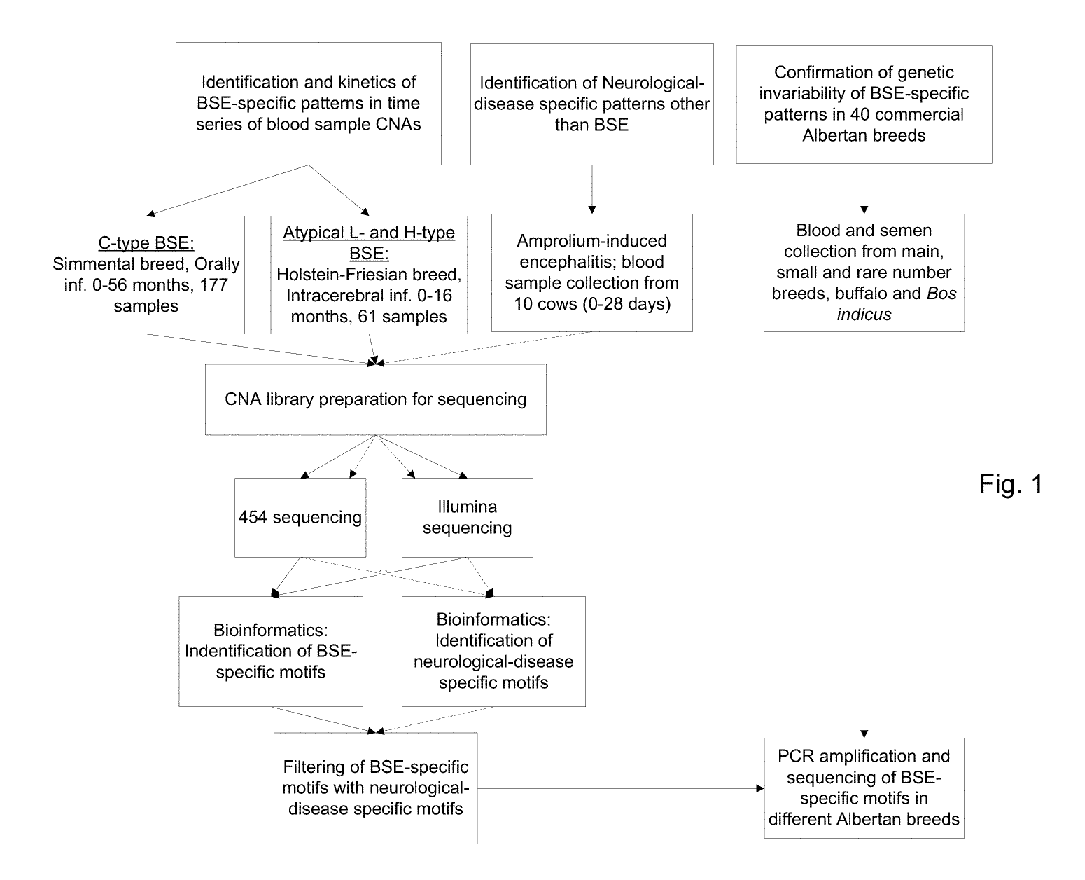

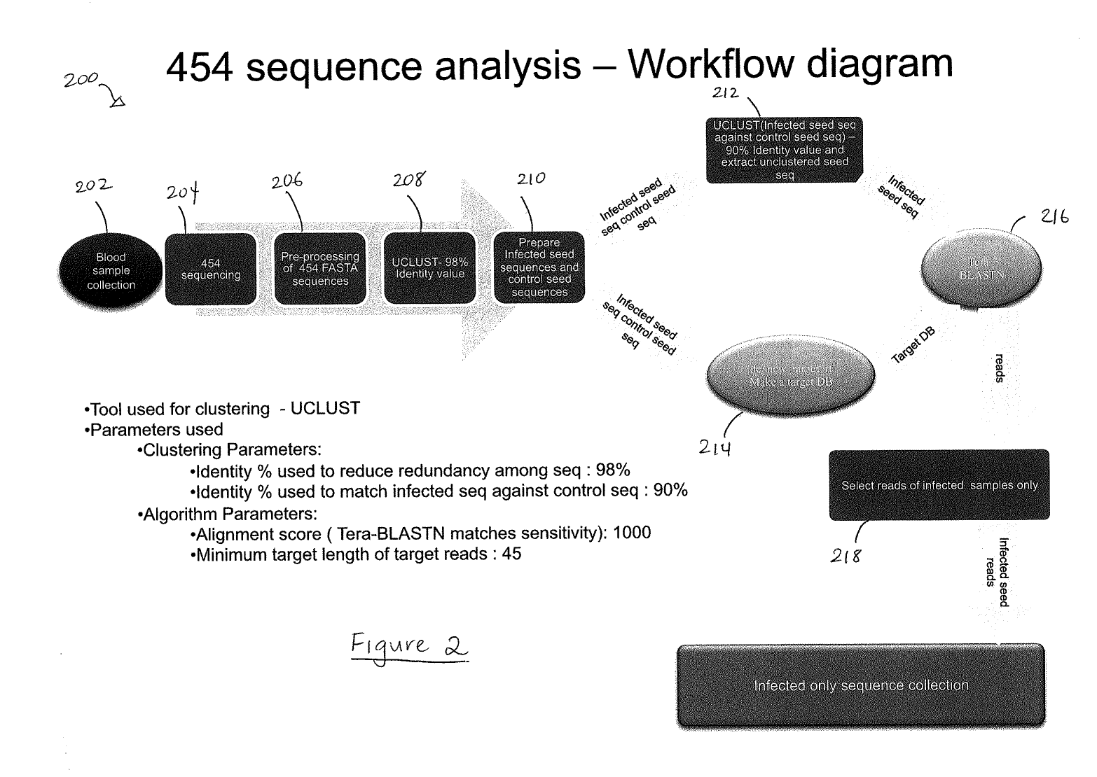

FIG. 1 shows a flowchart of the method for identifying the polynucleotides of the disclosure and identifying the BSE-specificity of these sequences across multiple commercial cattle breeds. Unique BSE-associated signatures in circulating nucleic acids (CNA) from blood serum obtained from cattle infected with the C-type form of BSE were identified and the kinetics of their appearance in BSE infected cattle over the course of the disease was established by direct comparison of normal, non-infected cattle and “classical” (C-type) BSE-infected cattle. Blood samples were obtained from normal, non-infected cattle and cattle infected with C-type BSE or atypical L- or H-type BSE over the timecourse of infection. CNA libraries were prepared from the blood samples and the libraries were sequenced by two different methods, Roche 454 DNA sequencing and Illumina DNA sequencing, respectively. The raw sequencing reads obtained by 454 sequencing and Illumina sequencing for both infected and controls animals were then mapped to the bovine genome using bioinformatics processing and the sequences from infected animals were filtered against sequences from the normal control animals to provide a pool of candidate sequence motifs specific to BSE-infected animals.

The raw sequences from the 454 sequencing and Illumina sequencing are filtered for quality control to remove sequencing artifacts and error-prone reads. The cleaned sequences are then mined using both pattern discovery and assembly/mapping bioinformatics processing methods. The 454 sequences were analyzed using a pattern based method, such as the all-against-all bioinformatics processing method described in Gordon et al. 2009, Nucleic Acids Res., 37(2):550-556.

FIG. 2 shows a flowchart of an embodiment of a 454 sequence analysis method 200. CNA libraries are obtained from the blood sample collection 202 and these libraries are sequenced in a 454 sequencing operation 204. After the sequencing operation 204, the method 200 proceeds to operation 206, where FASTA sequences are outputted from the 454 sequencing during operation 204. The FASTA sequences may be stored in a database for later retrieval and/or review. At operation 206, the FASTA sequences are pre-processed at the processing unit. Pre-processing may include a variety of actions, including, but not limited to, filtering the sequences with quality control filters in order to remove sequencing artifacts and error-prone reads, assigning the filtered data to respective source animals, and the like.

At operation 208, the processing unit divides the set of FASTA sequences into clusters. In some embodiments, as shown in the flow chart, UCLUST software is utilized to conduct the clustering with an identity value, such as 98%, to reduce redundancy among sequences; however, it is understood that the identity value may differ in alternate embodiments. In general, every sequence in the set of FASTA sequences with similarity above the identity value are arranged in a cluster.

After operation 208, the method 200 continues to operation 210 in which the processing unit prepares the infected seed sequences and control seed sequences. The infected seed sequences and the control seed sequences are then further processed at operation 212. In some embodiments, for example, the infected seed sequences are clustered against the control seed sequences utilizing the UCLUST software according to an identity value such as 90%. However, it is understood that this identity value may differ in alternate embodiments. The unclustered seed sequences are extracted from the infected and control seed sequences. Simultaneously, at operation 214, the processing unit may create a target sequence database. The target database and the infected seed sequences are then provided to a Tera-BLASTIN operation 216, a hardware accelerated version of the BLAST algorithm on dedicated TimeLogic® Decypher® hardware (Active Motif, Carlsbad, Calif.), in which reads from infected samples only are selected.

At operation 218, reads of the infected samples are selected and extracted against non-BSE neurological disease specific reads to provide a collection of sequences specific to BSE infection. In some embodiments, these extracted reads are stored in a database for later retrieval, review, and experimentation. For example, these reads may be later retrieved for further clustering with other sequences.

In an embodiment, the creation of the 454 sequencing reads shown in FIG. 2 was conducted at the McGill DNA Sequencing Center [Genome Canada Platform] and the analysis of the reads at the University of Calgary (Calgary, Canada, University of Calgary) using a TimeLogic® Decypher® biocomputing platform (Active Motif, Carlsbad, Calif.) and multiple CPU servers at the Visual Genomics Centre at the University of Calgary.

The Illumina sequences were analyzed using a genome assembly/mapping bioinformatics processing method. The cleaned sequence reads were mapped to the bovine genome using a fast mapper, genomic hotspots for infection were identified, the reads were comprehensively mapped to the identified hotspots, and a gene search was conducted in the hotspot region to correlate the reads with a gene. Reads that did not map to the genome were assembled to generate clusters and then analyzed using the same procedures are for the hotspots.

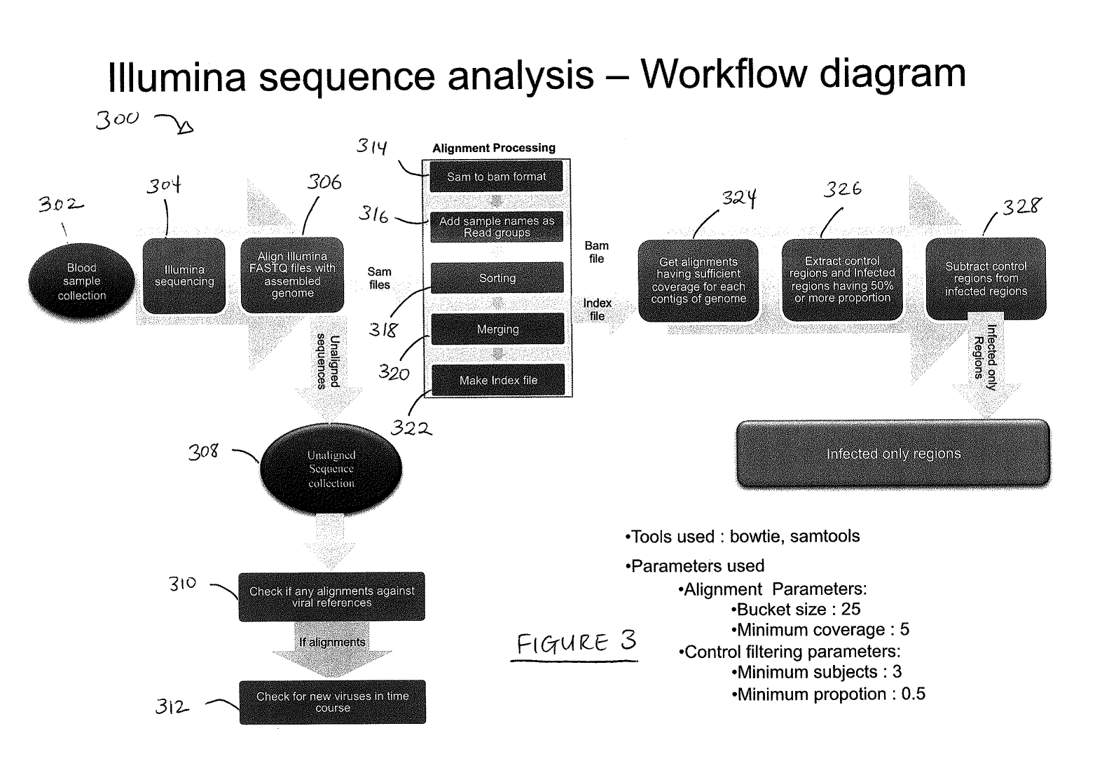

FIG. 3 shows a flowchart of an embodiment of an Illumina sequence analysis method 300. CNA libraries are obtained from the blood sample collection 302 and these libraries are sequenced in an Illumina sequencing operation 304. After the sequencing operation 304, the method 300 proceeds to operation 306, where the processing unit stores the output of the Illumina sequencing in a text-based file format, for example, a FASTQ format. It is understood that the output of the Illumina sequencing can also be stored in other file formats, such as, for example, SAM or BAM formats. The sequencing files are assembled with a reference genome to determine genome locations with significant counts of exclusively infected or uninfected animal reads.

During operations 314 to 322, the results of the alignment are processed. In some embodiments, the results of the alignment process are outputted in a text-based file format, such as a SAM format. A SAM formatted file is a tab-delimited text file that contains sequence alignment data. In such embodiments, the processing unit converts the SAM format to a BAM format, which is a binary version of the SAM formatted file. In other examples, the alignment process outputs a BAM formatted file, and thus, the conversion step 314 is skipped. In operations 316 to 322, preparation for and creation of an index file associated with the BAM formatted file is created. The index file creation process may include categorizing by adding sample names as read groups, sorting, and/or merging, as shown in the method 300. It is understood that each step of the method 300 is not necessary, but an optional step that may be chosen by the system or a user of the system.

The created index file and the BAM formatted file is further processed by the processing unit in operation 324. In particular, operation 324 includes reviewing the alignments and extracting the alignments, which have sufficient coverage for each contiguous sequence (=contig) of the reference genome. In an embodiment, the alignment parameters comprise a bucket size of 25 and a minimum coverage of 5, although it is understood that the bucket size and minimum coverage value may differ in alternate embodiments. At operation 326, control regions and infection regions having 50% or more proportion are extracted and compared to determine overlaps. In some embodiments, these regions are stored in a database associated with the processing unit for later retrieval and/or review. The method 300 continues with operation 328 in which the extracted control regions are subtracted from the infected regions so that only the infected regions remain thereby providing sequence reads that are present in only infected regions. In an embodiment, the control filtering parameters comprise a minimum subjects value of 3 and a minimum proportion value of 0.5, although it is understood that these values may differ in alternate embodiments.

In some embodiments, at operation 306, not all of the sequences are aligned. In such examples, the unaligned sequences are collected, as shown in operation 308. The unaligned sequences may be stored in the database for later retrieval and/or review. In operation 310, the unaligned sequences may then be aligned against other references, such as viral references, to determine if any alignments exist. If alignments exist, the sequences may be stored in the database for later retrieval and alignment with new viruses, as desired by a user of the system, as shown in operation 312.

In an embodiment, the analysis of the Illumina sequencing reads shown in FIG. 3 was conducted at the University of Calgary (Calgary, Canada) using a TimeLogic® Decypher® biocomputing platform (Active Motif, Carlsbad, Calif.) and multiple CPU servers at the sequencing center.

Specificity of the BSE-specific CNA expression patterns identified by the 454 sequencing analysis or Illumina sequencing analysis were confirmed by analyzing blood sample CNA expression patterns from cattle with brain-related neurological diseases other than BSE or trauma. As shown in FIG. 1, bloods samples were obtained from control bovine overdosed with amprolium to induce polioencephalomalacia (PEM) in the animals over the timecourse of treatment. CNA libraries were prepared from the blood samples and the libraries were sequenced by Roche 454 DNA sequencing or Illumina DNA sequencing as described above. The raw sequencing reads obtained by 454 sequencing and Illumina sequencing for PEM control animals were then mapped, similar to the BSE animals and normal animals, to the bovine genome using bioinformatics processing and the sequences from infected animals were filtered against sequences from the normal control animals and PEM control animals to provide a pool of candidate sequence motifs specific to BSE-infected animals.

Sequence variability of the identified CNA motifs specific to BSE infection was then analyzed in a sampling of cattle breeds representative of most commercial cattle breeds worldwide through PCR and Sanger sequencing of the PCR products to determine if the identified CNA motifs are capable of detecting BSE-infection in cattle breeds destined for the human food chain (FIG. 1). Previous studies have indicated the origin of disease specific motifs is endogenous in CWD [Gordon et al., 2009, Nucleic Acids Res., 37:550-6]. In healthy humans, it has been shown that approximately 97% of CNA sequences are of genomic origin [Beck et al., 2009, Clin Chem., 55(4):730-8]. The same is believed to be true in cattle. The candidate CNA motifs were therefore characterized at the bovine genomic level to determine the genetic stability of the motifs in a broad range of cattle breeds and to ensure that there were no major variances in the BSE-specific CNA motifs in the various cattle breeds used commercially.

Blood and/or semen samples were obtained from approximately 40 bovine breeds and 2 different types of bison, which accounted for all cattle breeds in Alberta, Canada. Table 3 shows a listing of the cattle breeds tested as well as the type of sample collected for testing. For each sample, regions containing candidate BSE-specific motifs were amplified via PCR from chromosomal genomic DNA obtained from the blood and semen samples. More than 300 BSE-specific CNA motifs unique to animals infected with the C-type form of BSE were identified. Eight of the most prevalent CNA motifs were frequent among the infected animals. The region and polynucleotide derived from each of the eight CNA motifs are shown in Table 4. The nucleotide sequence of each motif is shown in Table 4 by reference to the nucleic acid sequence numbering of the reference region identified by accession number.

The PCR products were sequenced at the University of Calgary's DNA sequence laboratory (Calgary, Canada) and bioinformatics analysis of the polynucleotides was performed using the massively parallel TimeLogic® infrastructure at the Visual Genomics Center at the University of Calgary (Calgary, Canada). The copy number of the BSE-specific motifs was screened against the taurine bovine genome [The Bovine Genome Sequencing Consortium et al., 2009, Science, 324:522] as shown, for example, in FIG. 3 and surrounding genomic sequences to identify genomic hotspots. The BSE-specific motifs were comprehensively mapped to the hotspots to correlate the motifs with a gene or gene and the gene functions of the motifs were mapped on metabolic pathways using sequential functional analysis. Table 13 shows the genes and KEGG pathways to which the BSE-specific sequences identified by Illumina sequencing (Table 1) were mapped. Table 2 shows the genes to which the BSE-specific sequences identified by 454 sequencing were mapped. Tables 5 and 6 show a listing of bovine genes to which the BSE-specific motifs in Tables 2 and 13 were mapped and a description of the genes including cellular components, biological processes, and molecular functions in which the genes are implicated. Table 14 shows a listing and description of each of the KEGG pathways to which sequences were mapped. The functional mapping of the BSE-specific motifs to metabolic pathways provides insight into the biological mechanism underlying the host response in BSE infection.

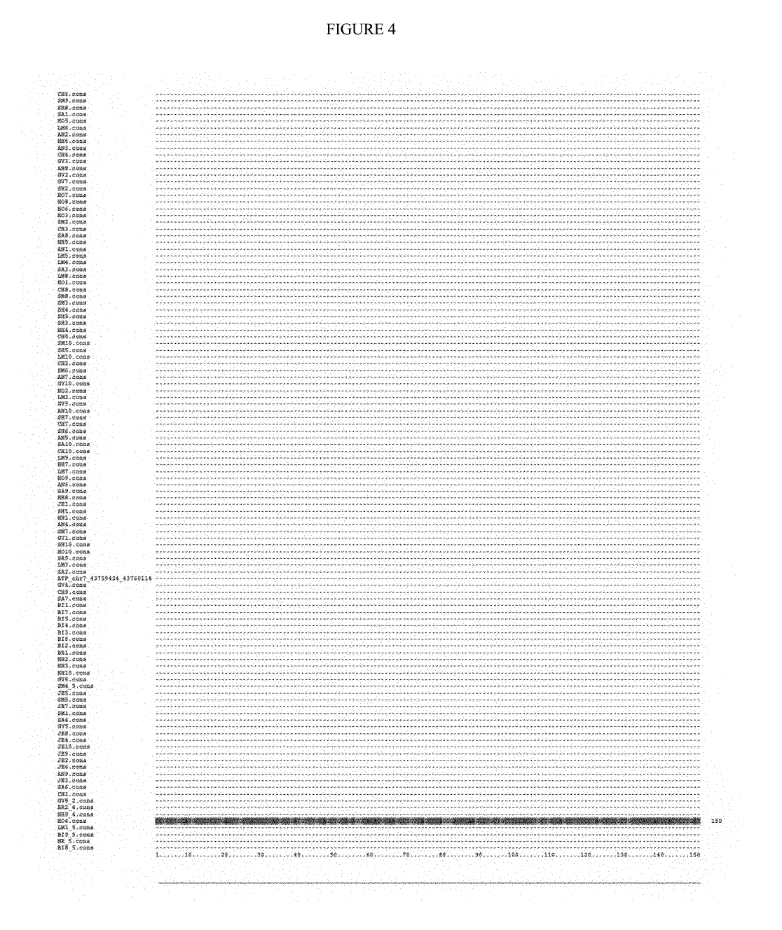

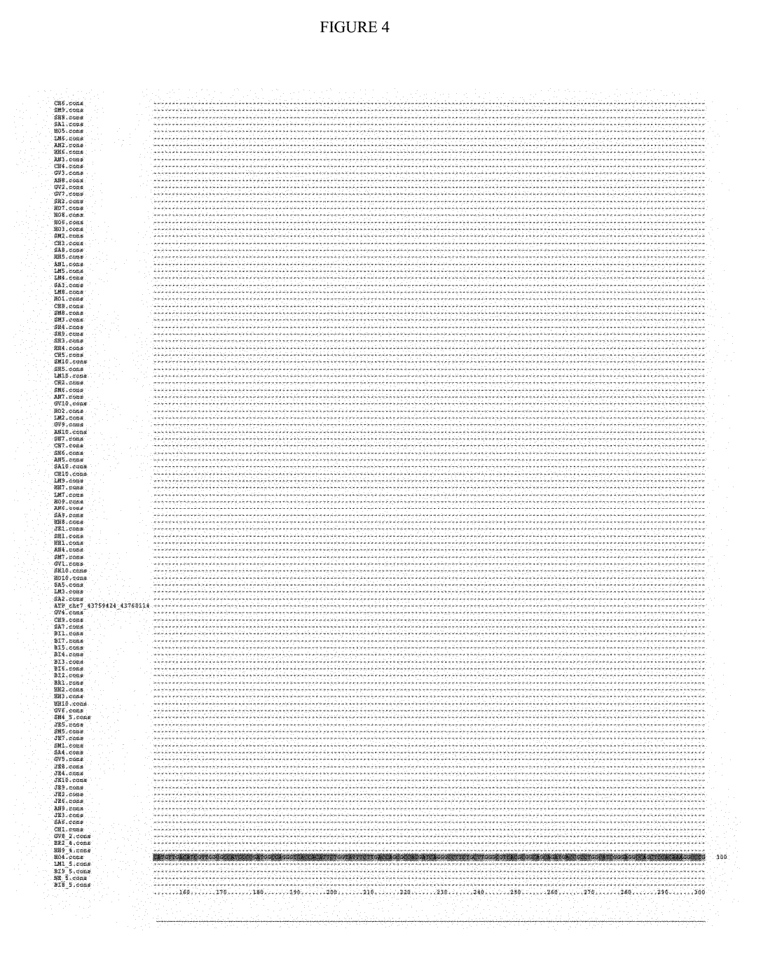

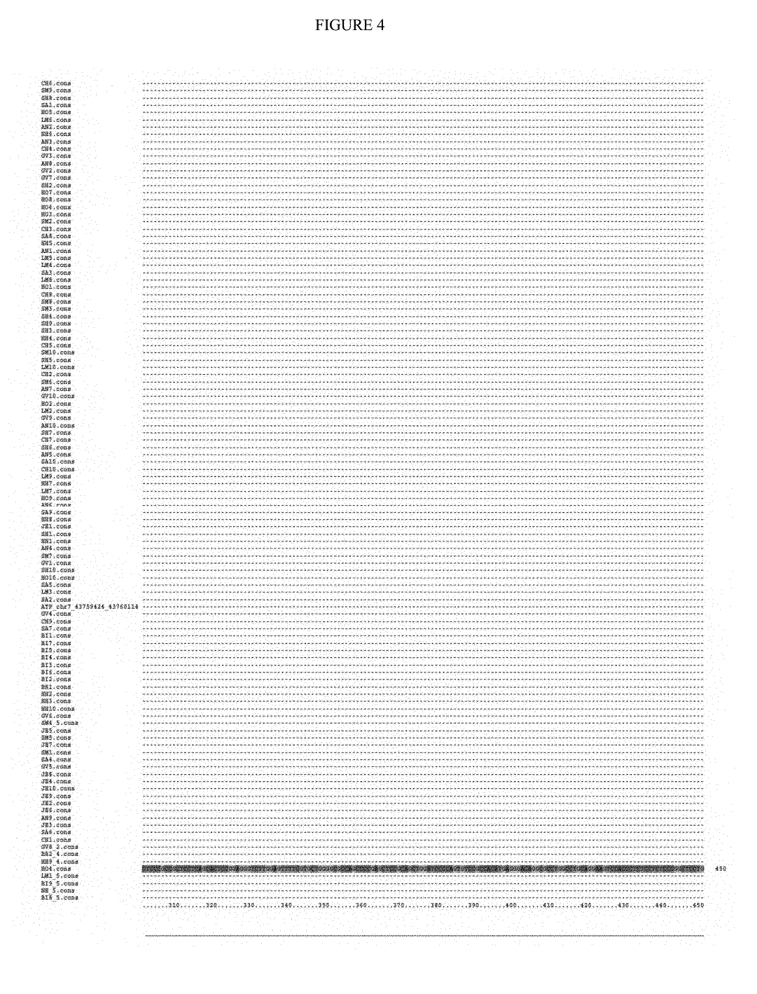

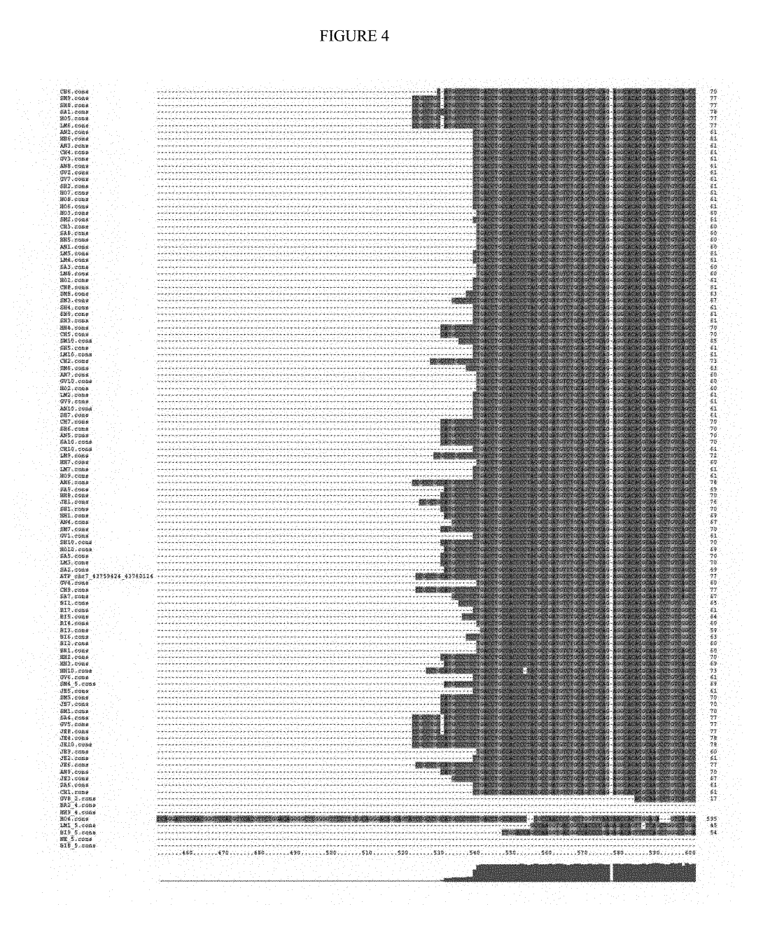

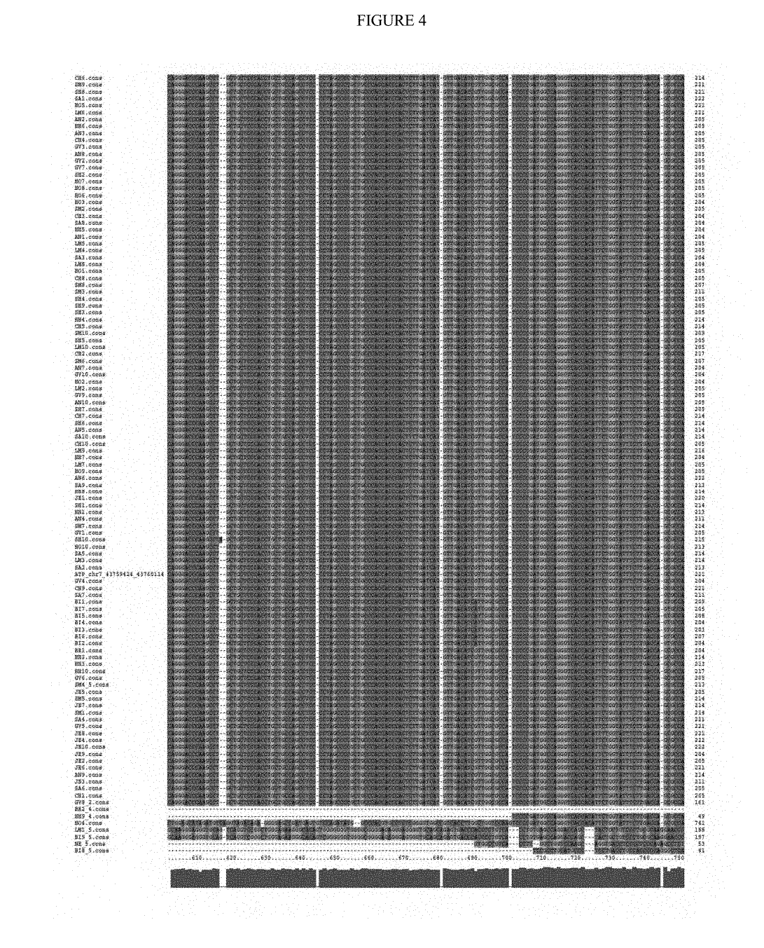

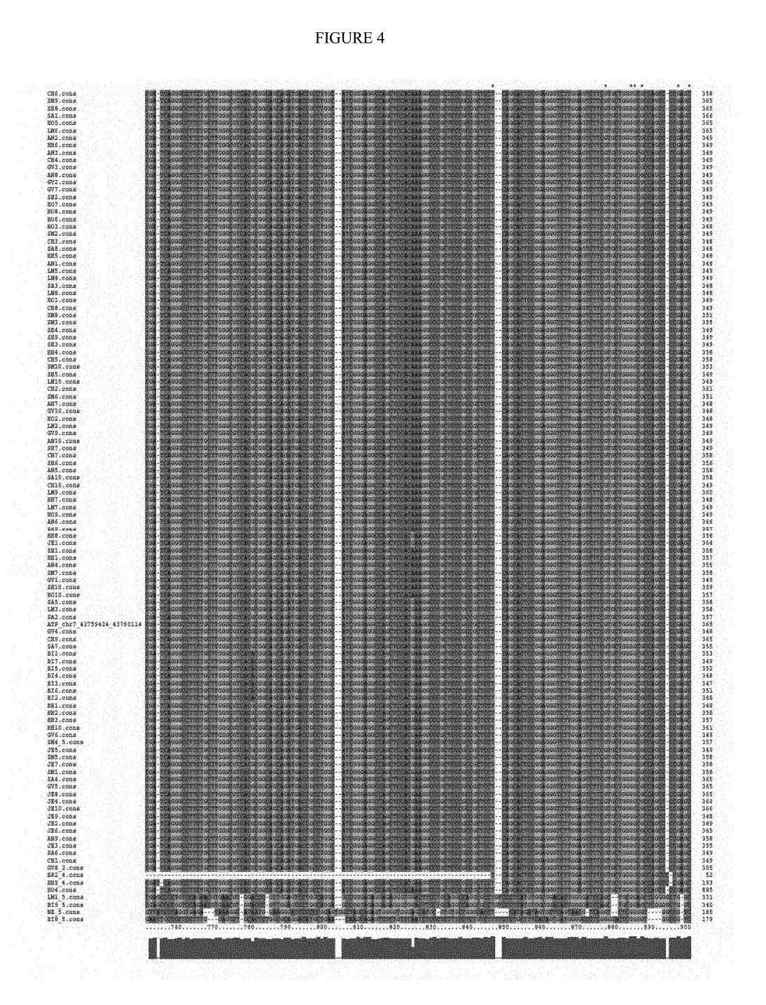

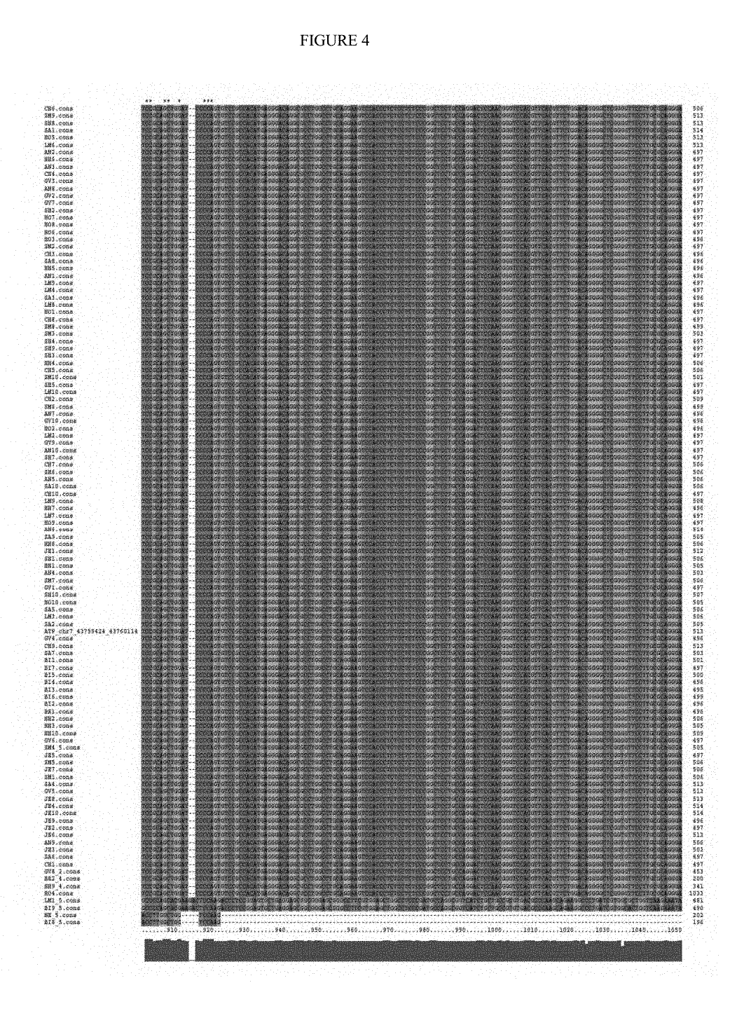

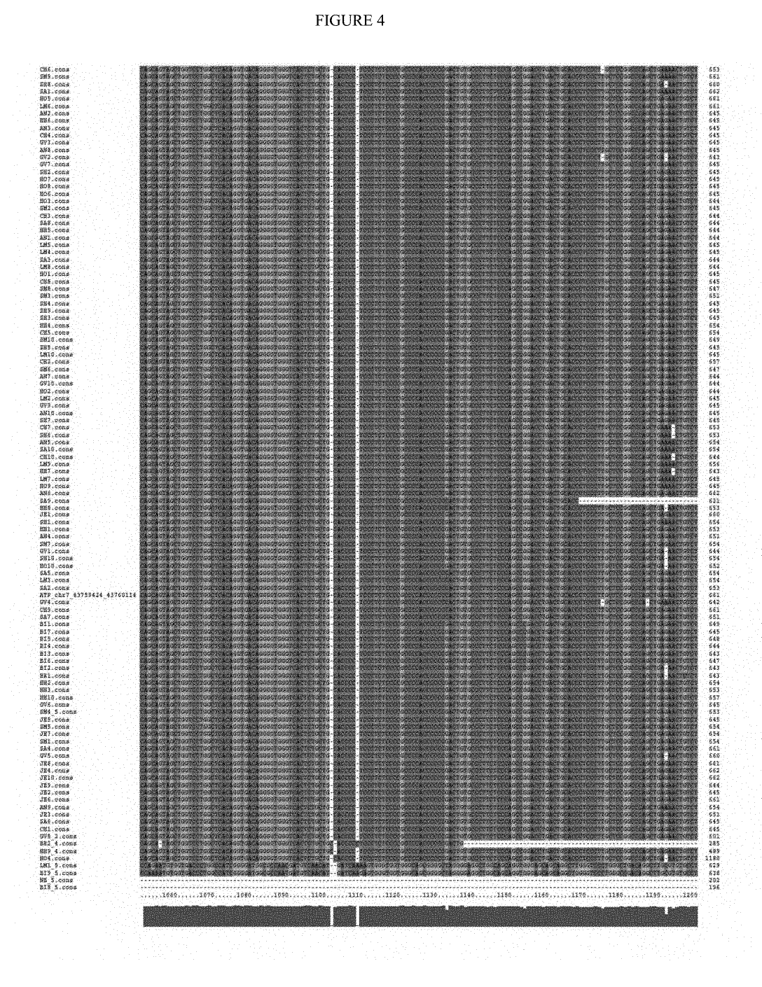

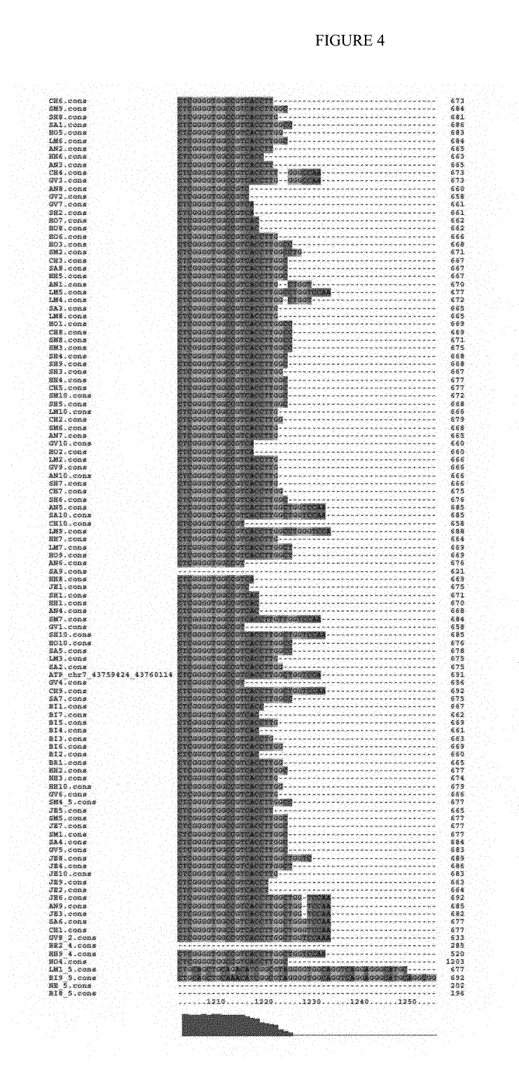

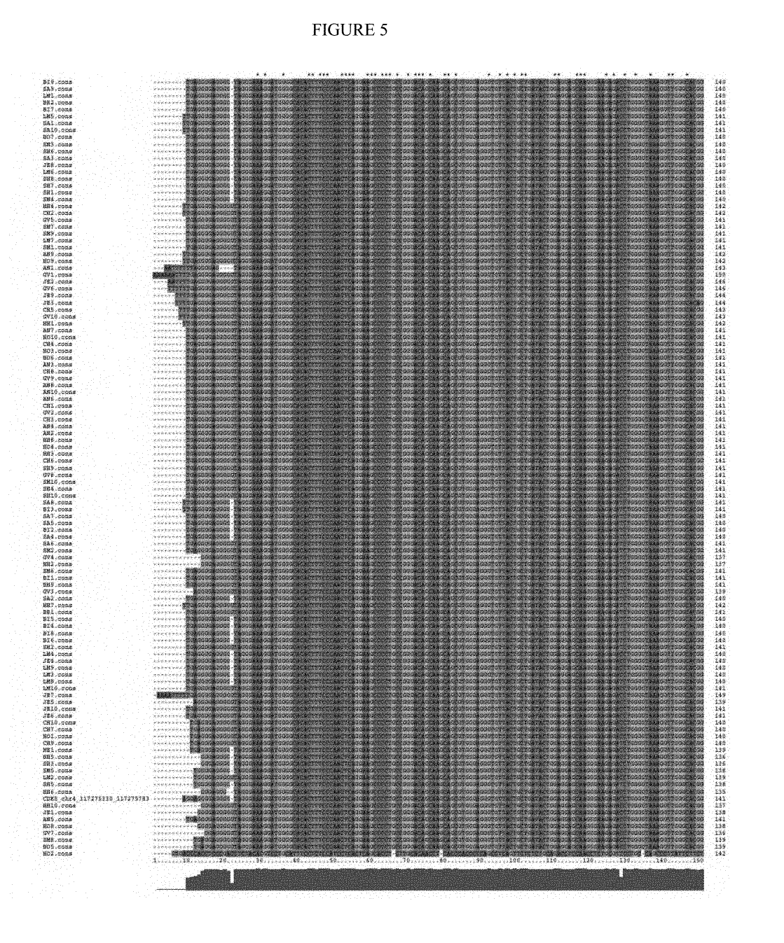

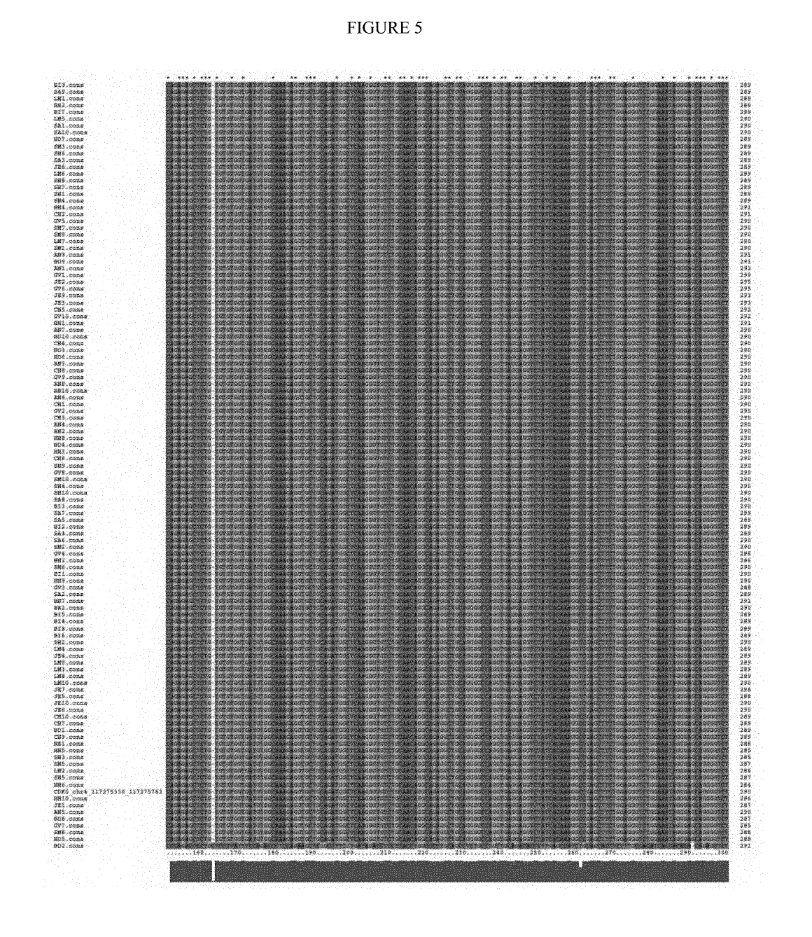

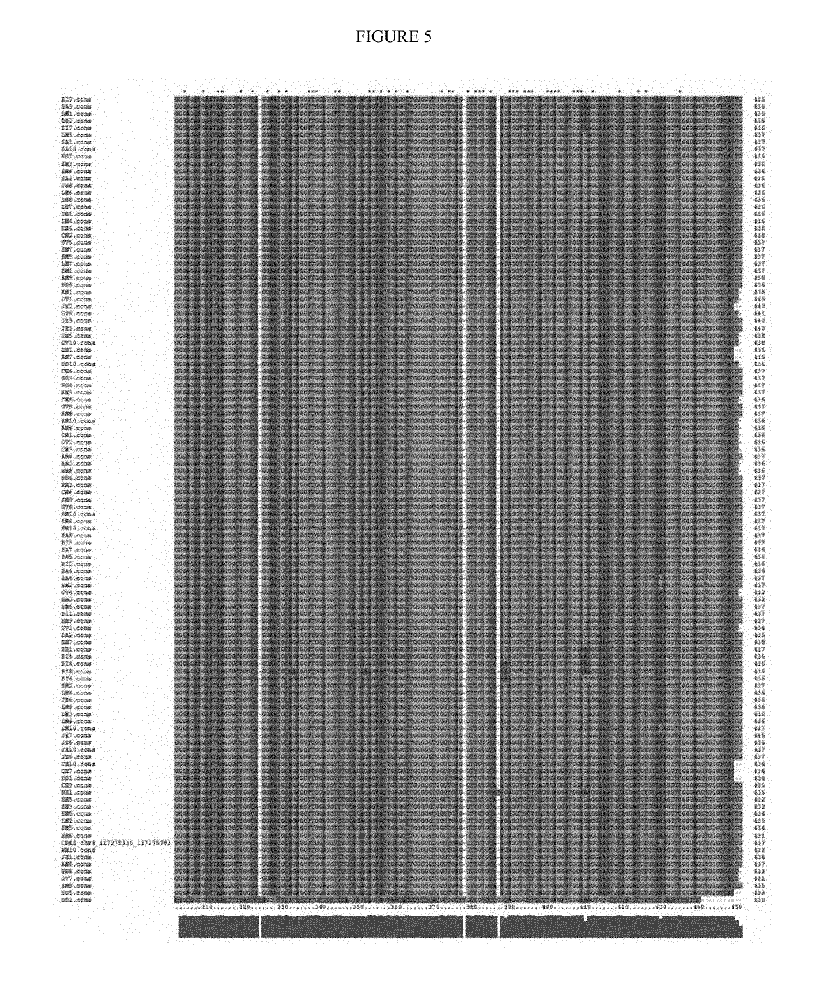

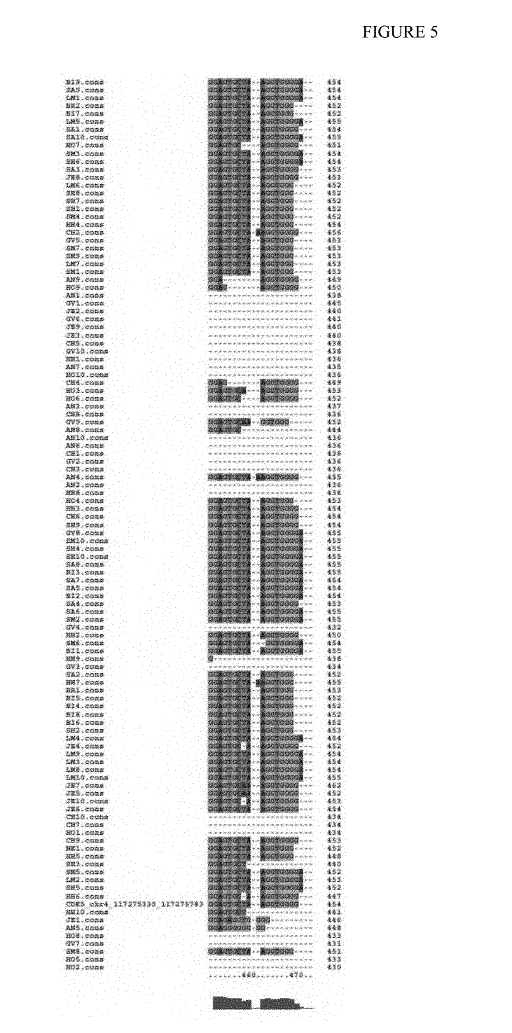

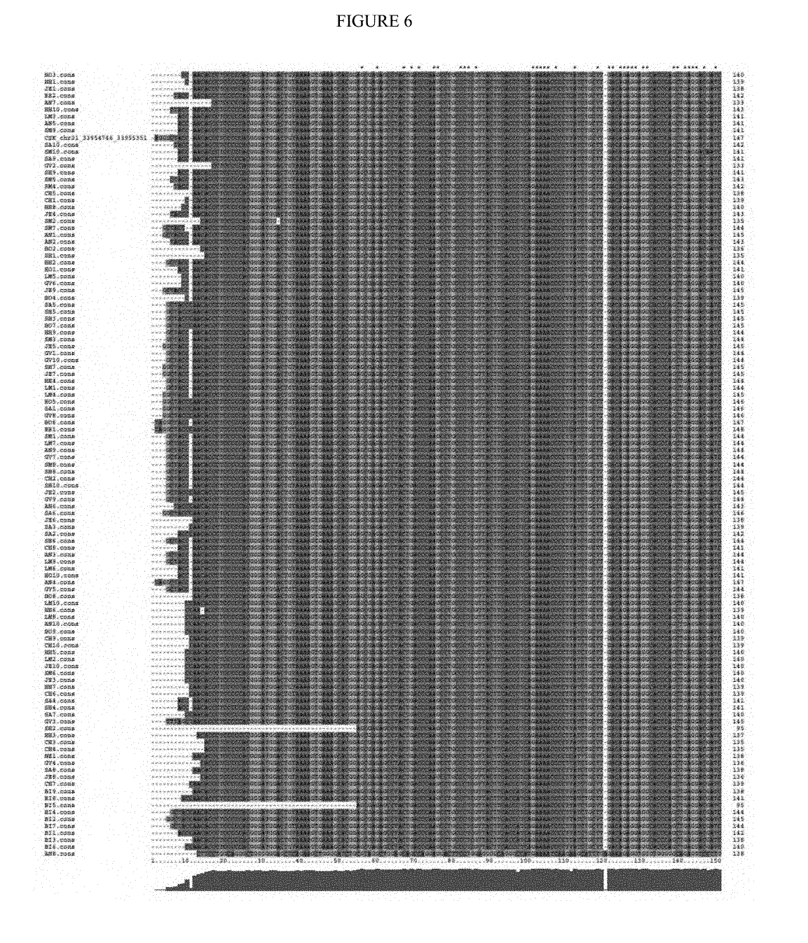

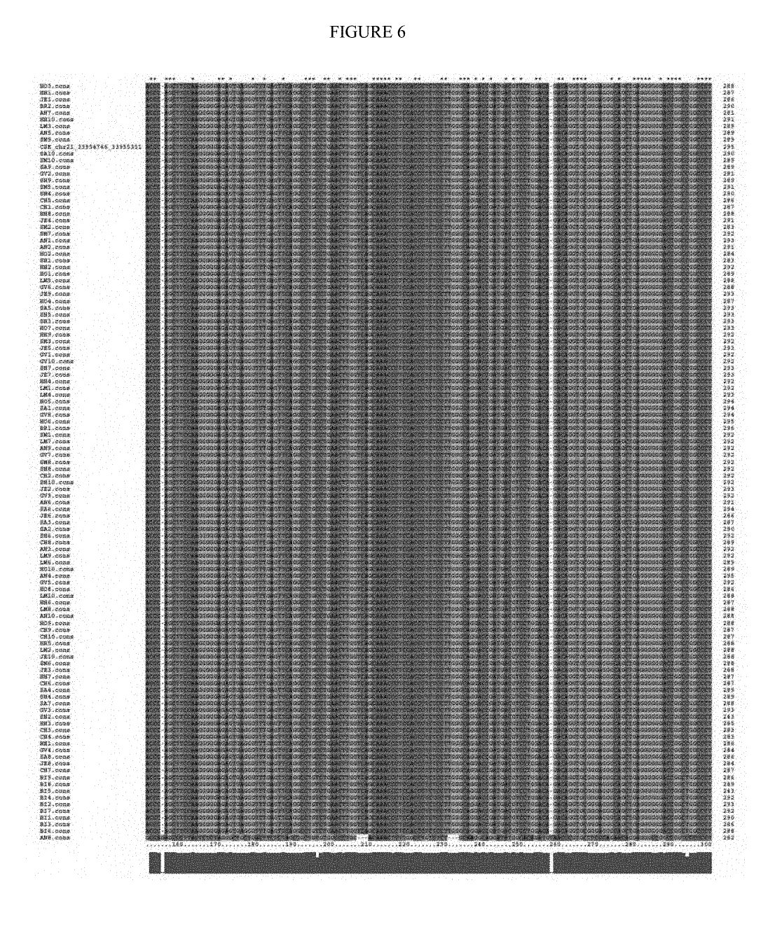

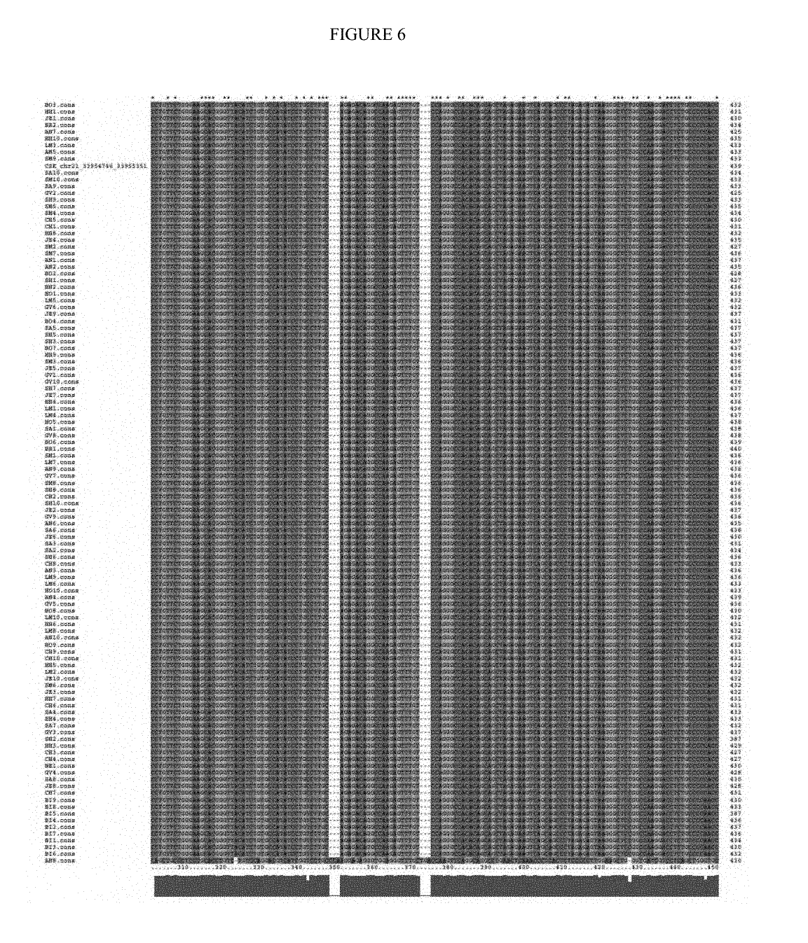

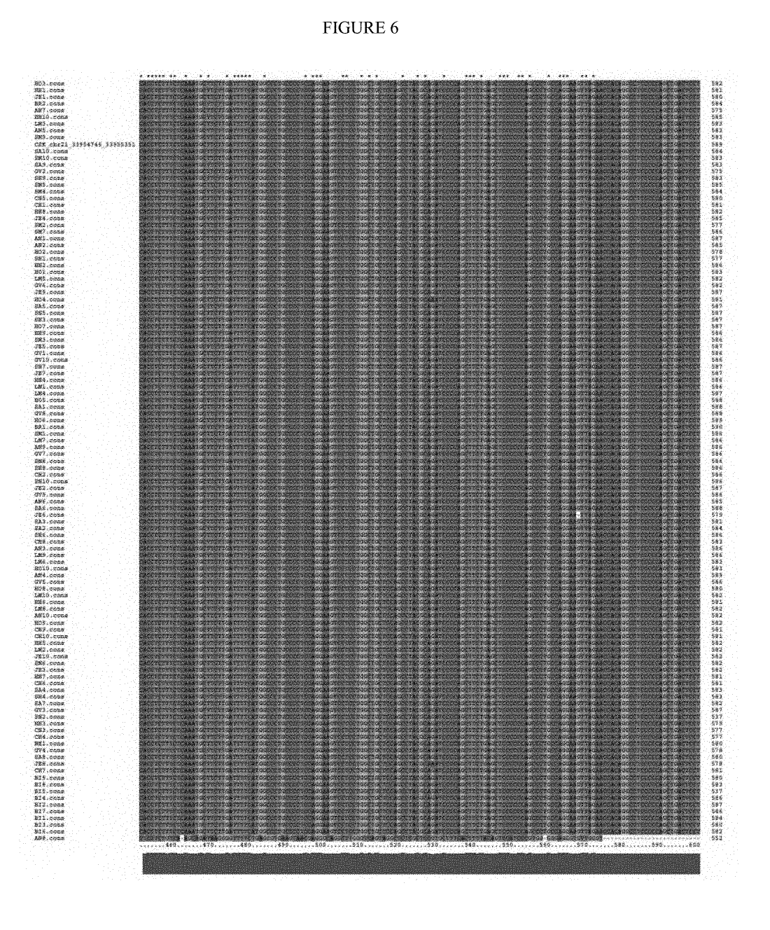

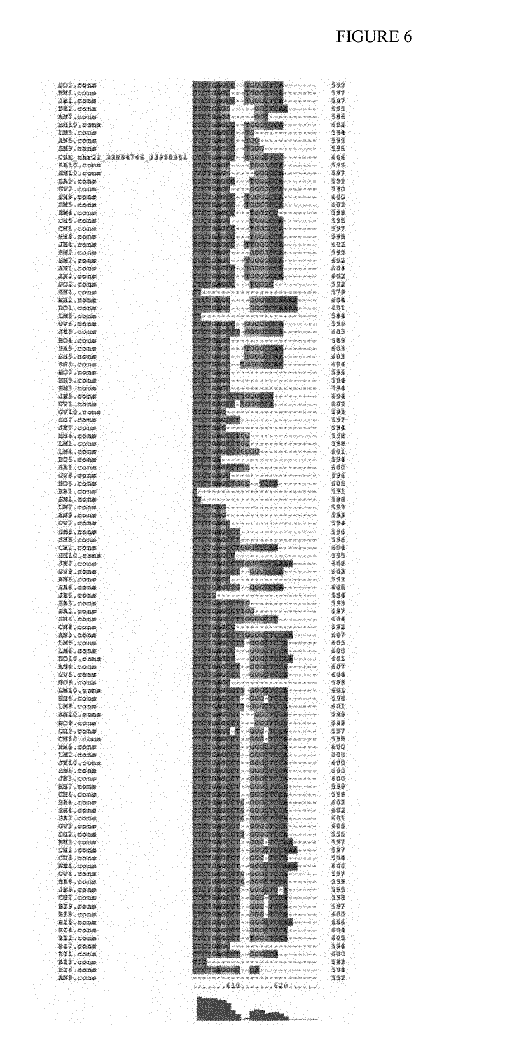

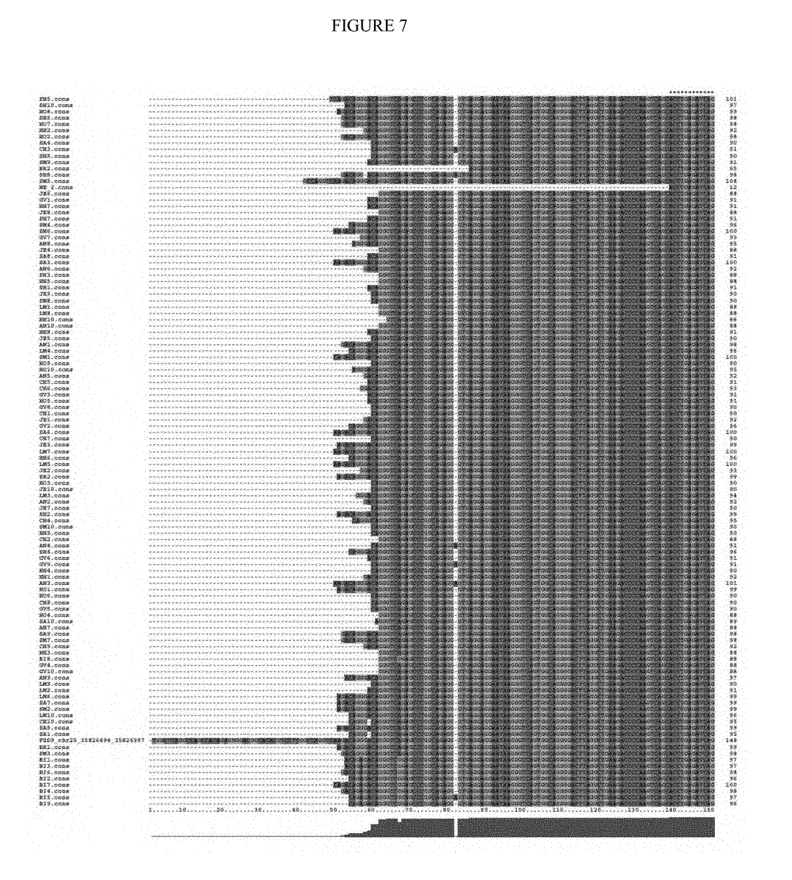

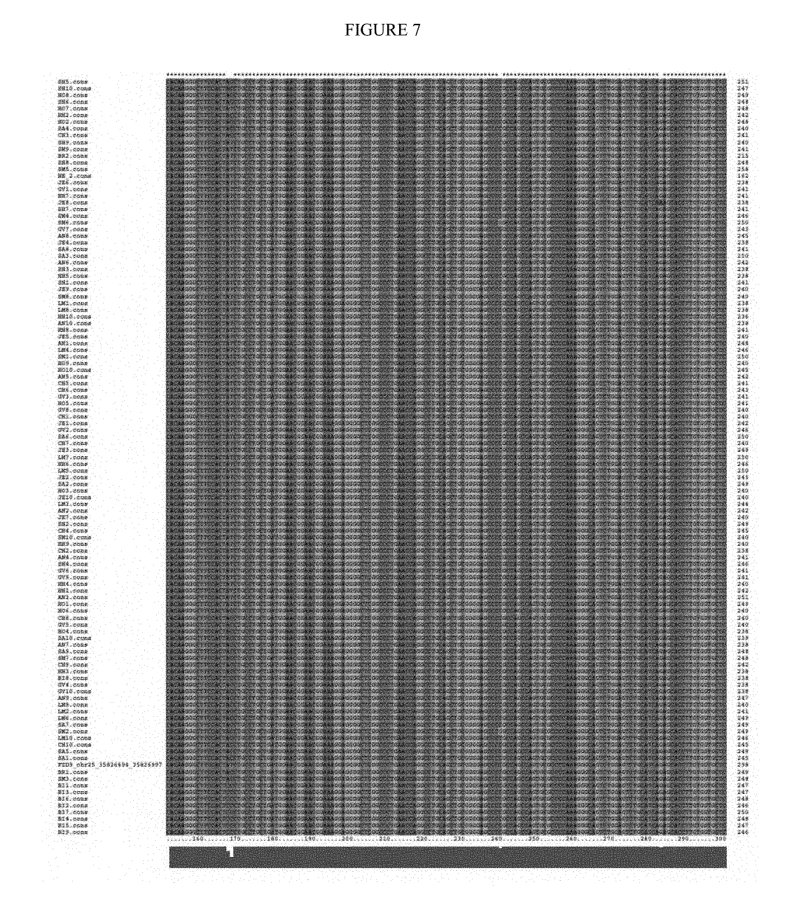

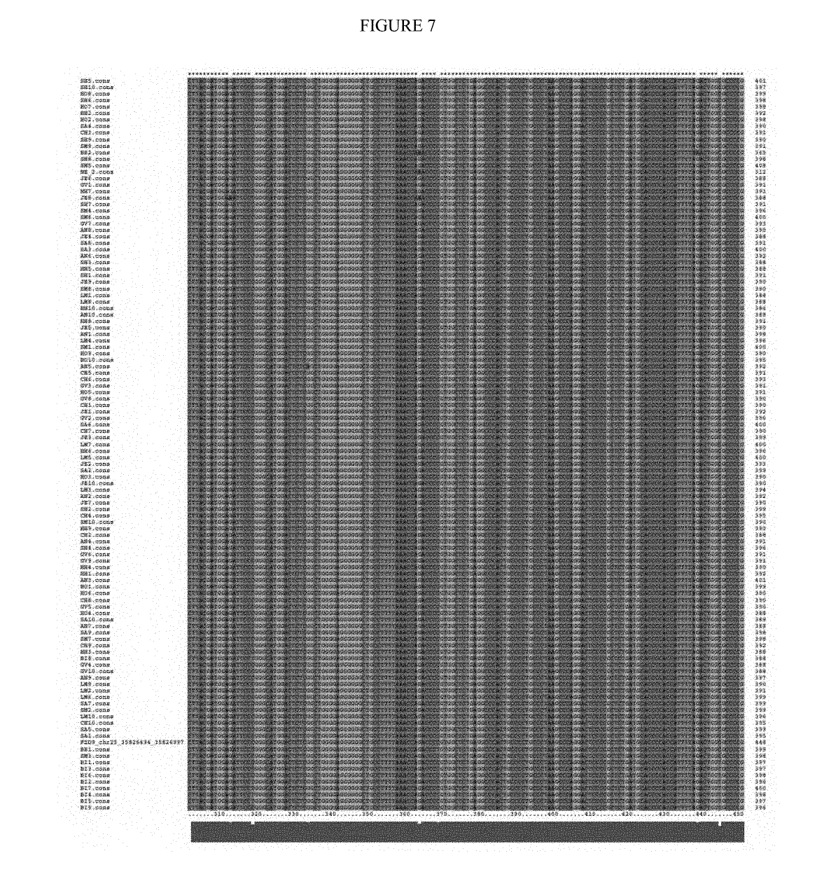

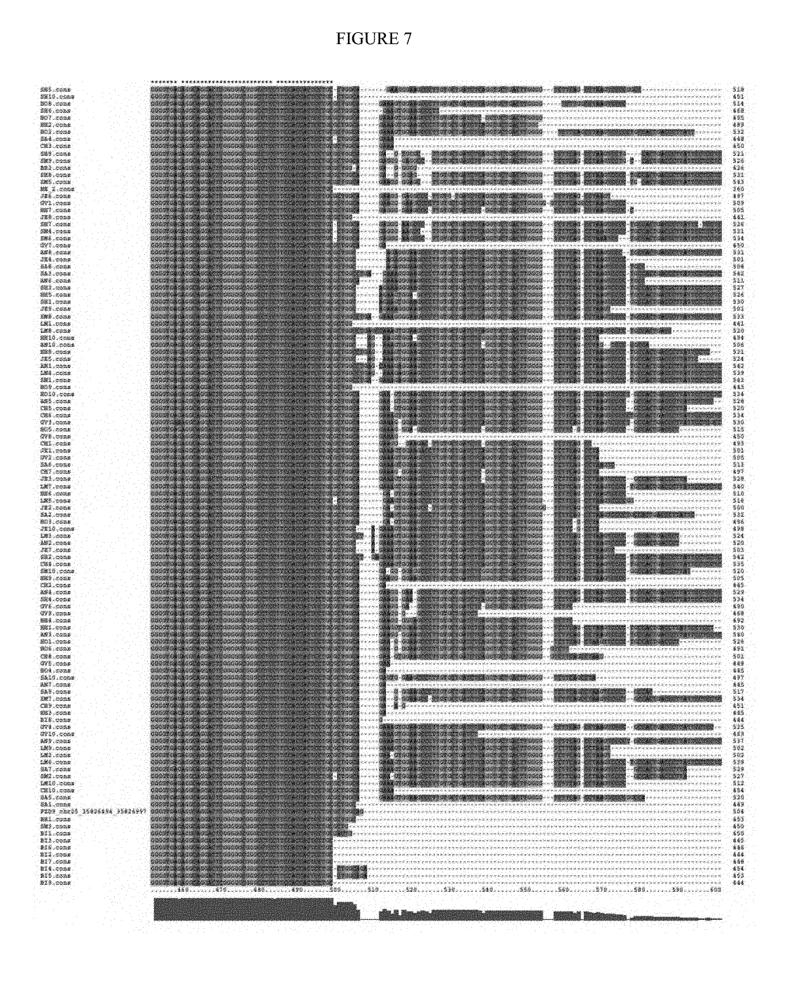

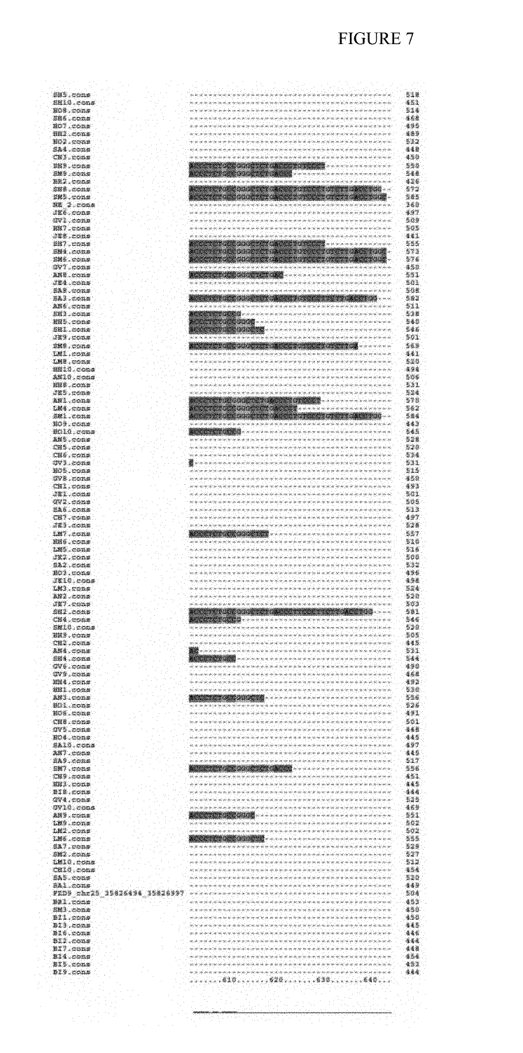

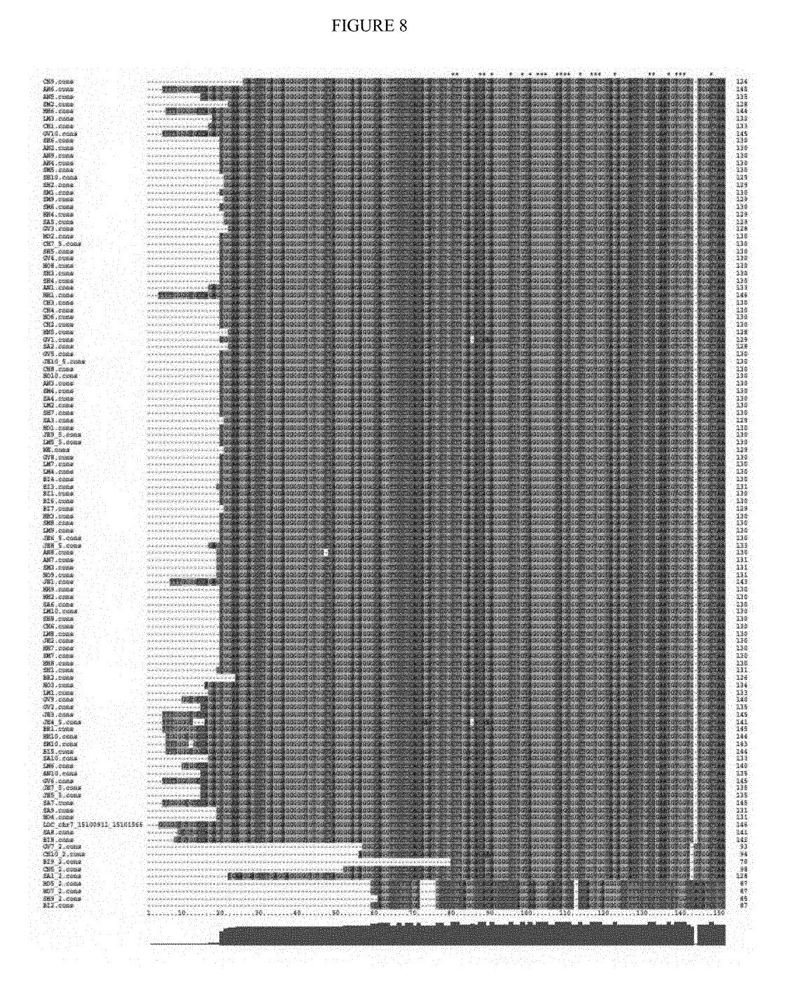

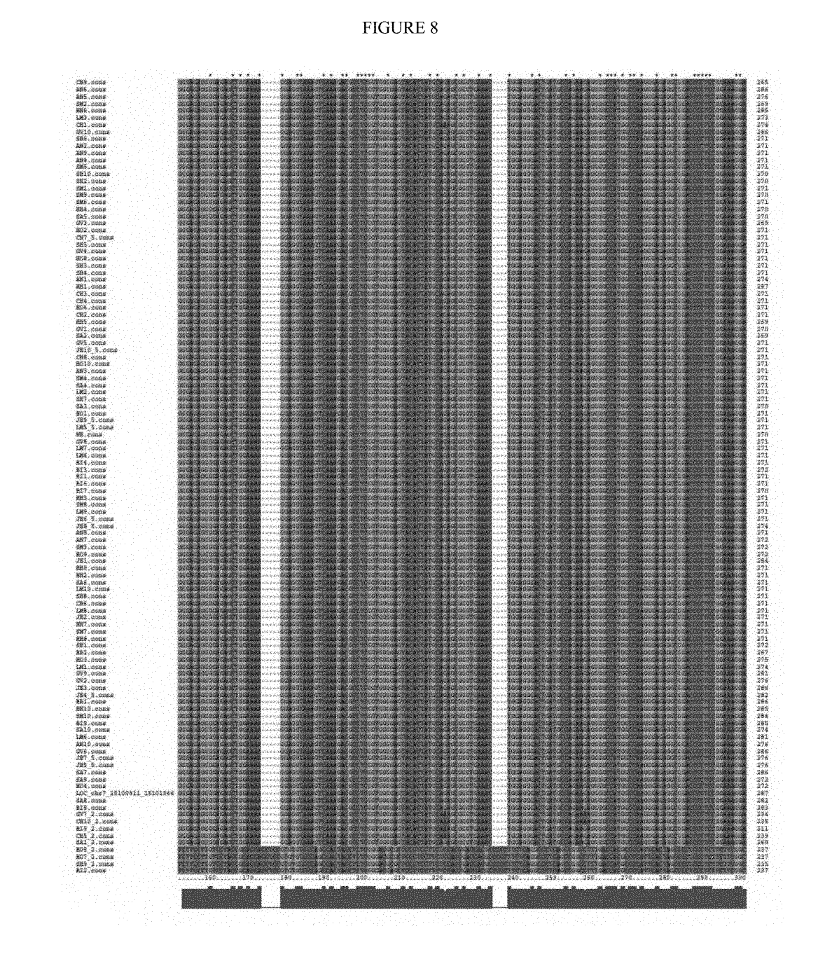

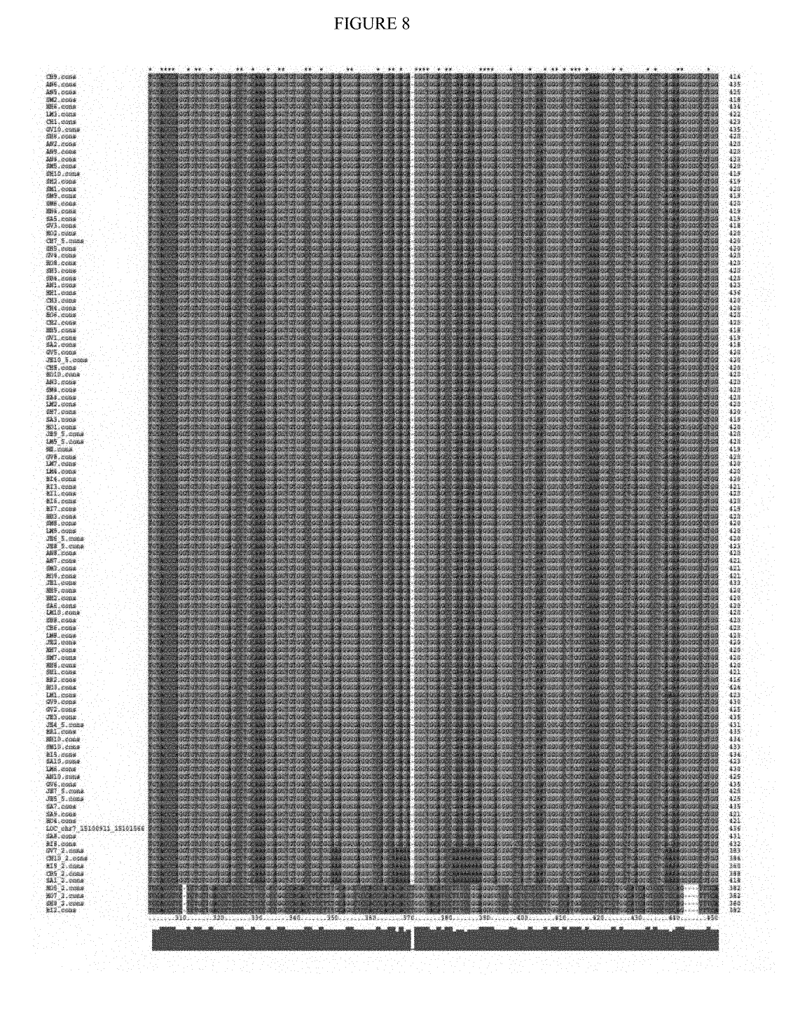

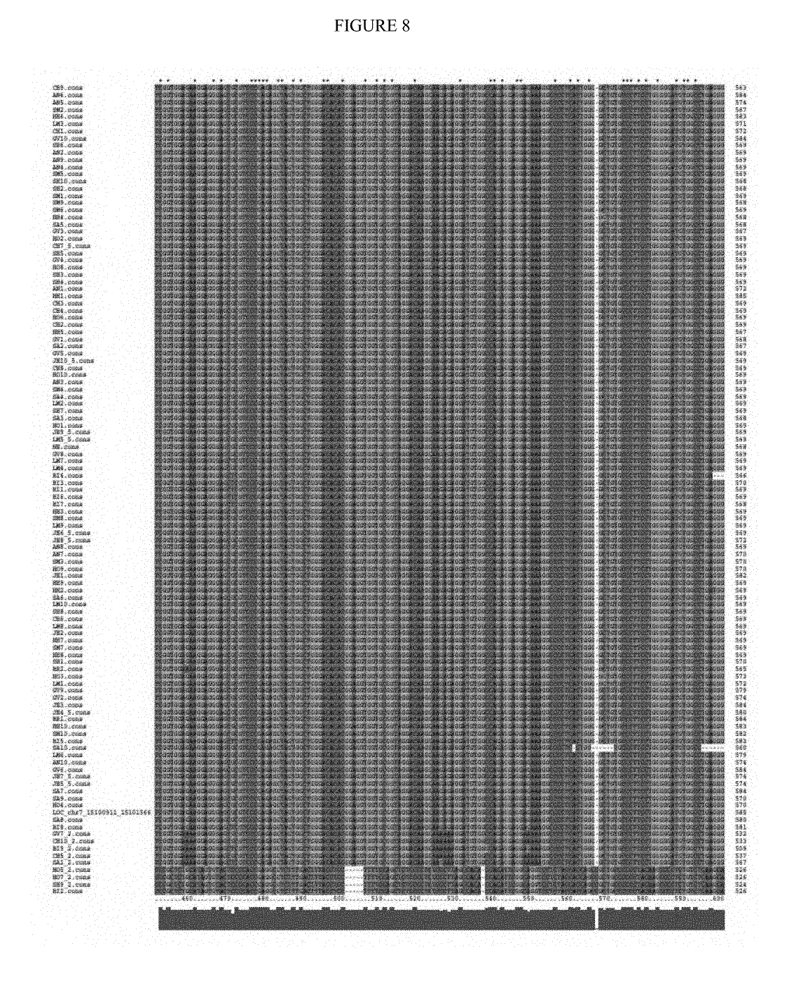

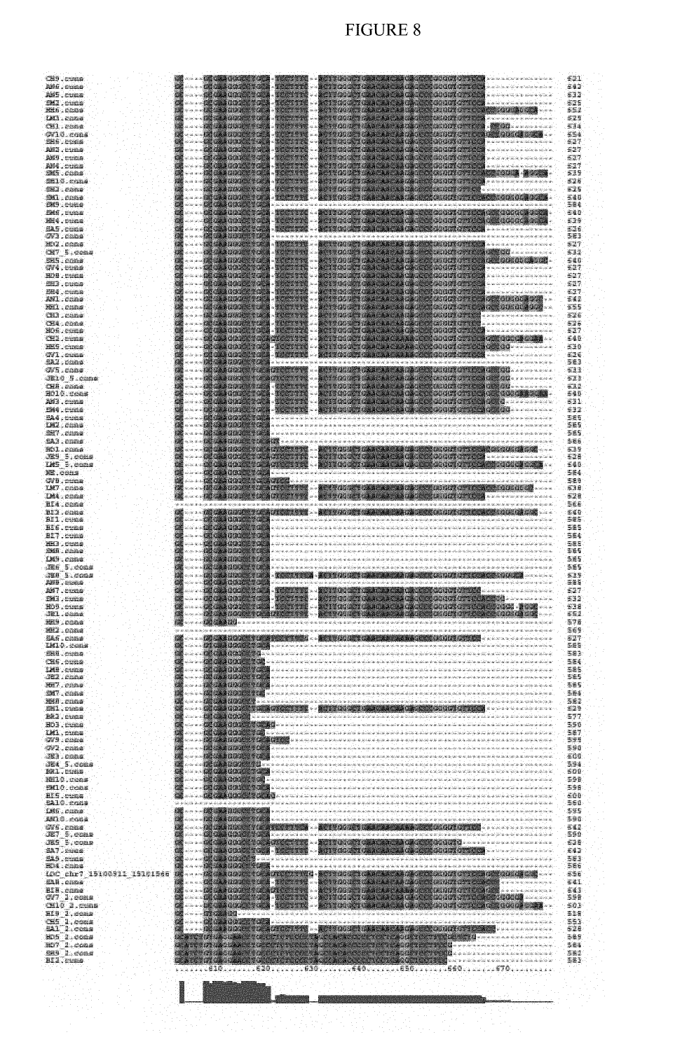

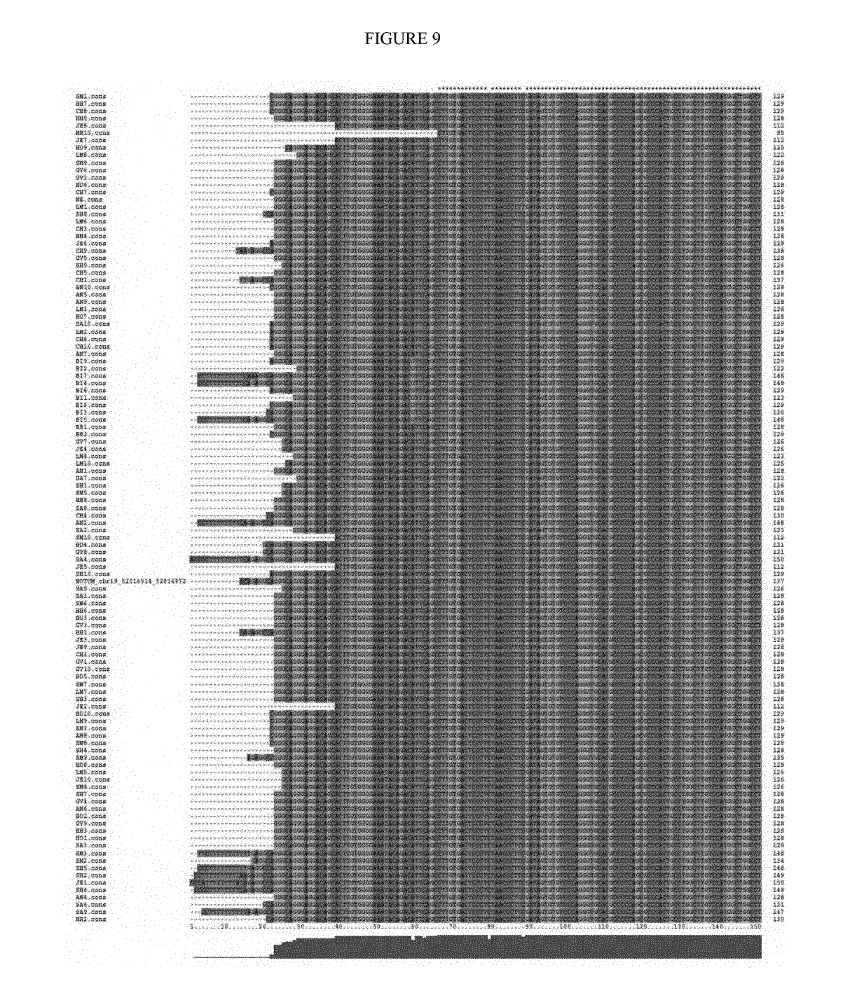

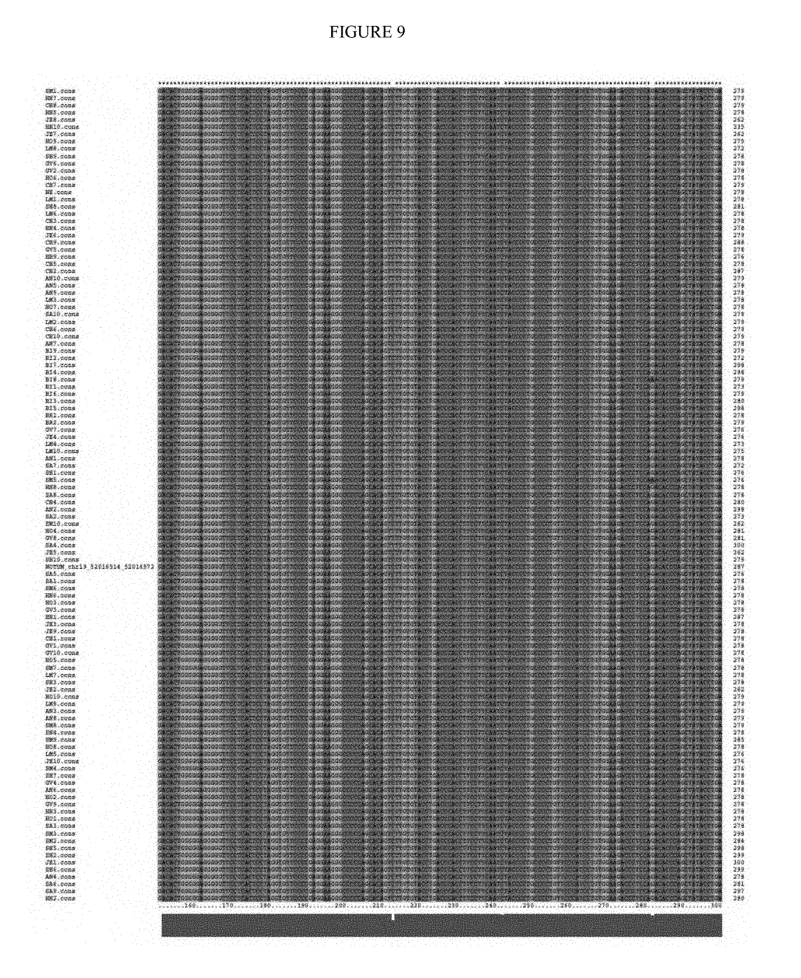

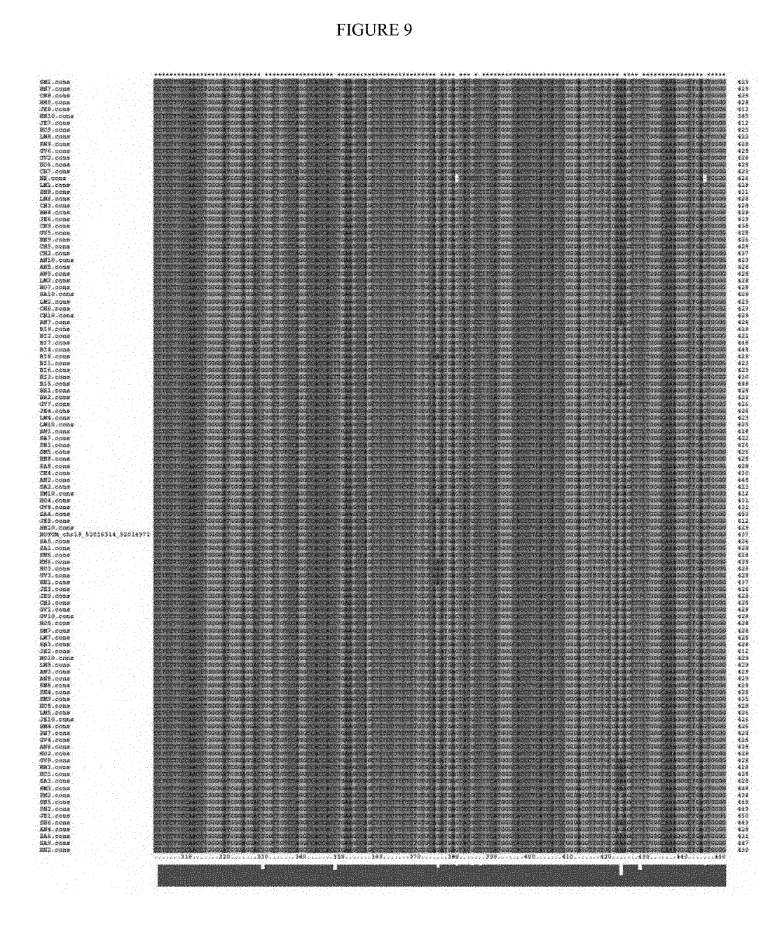

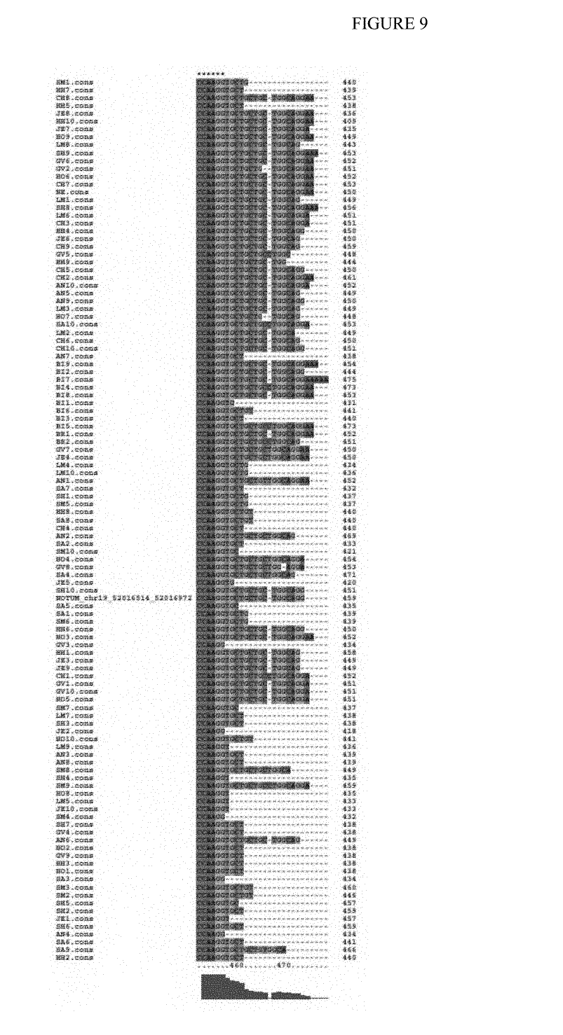

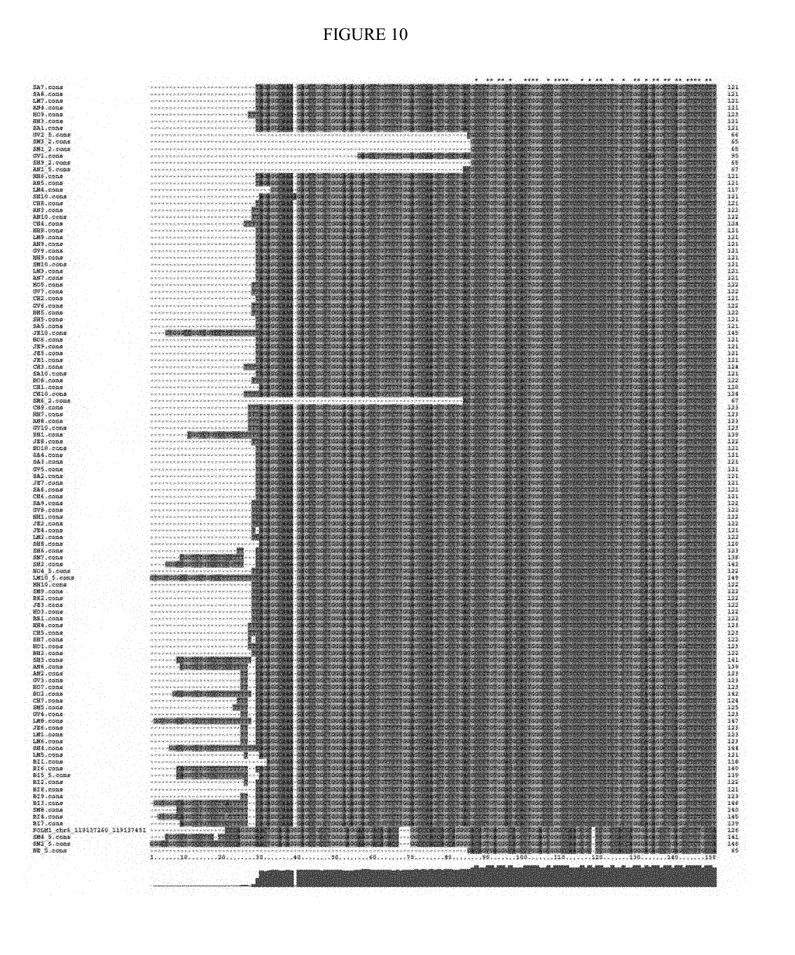

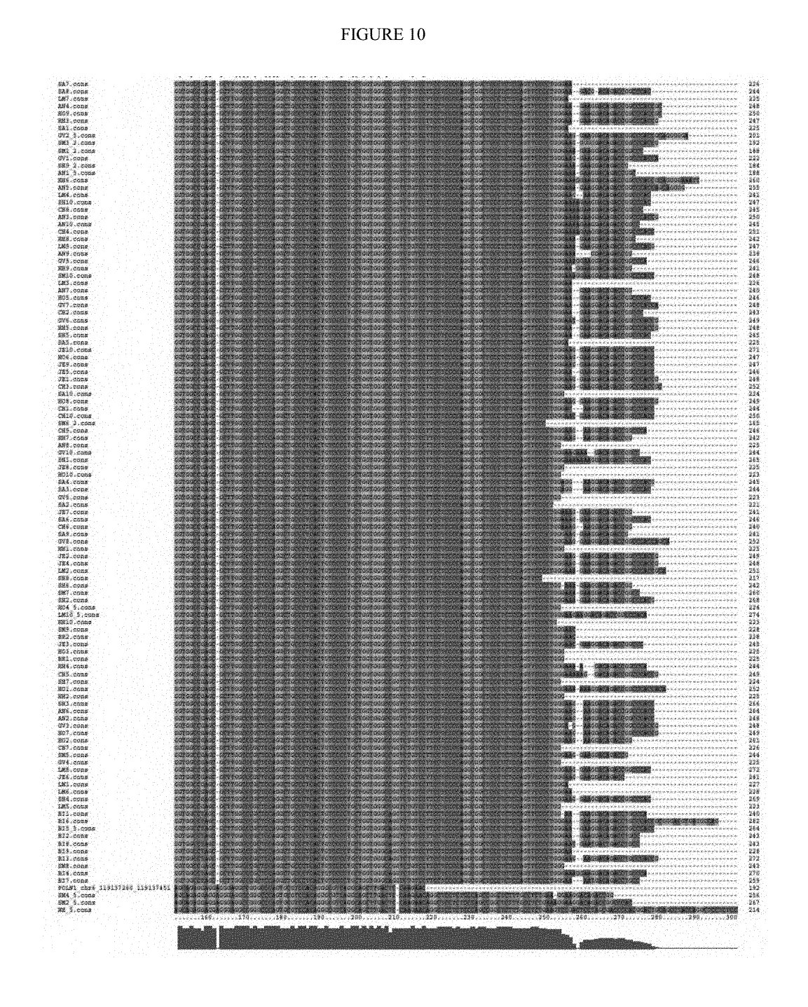

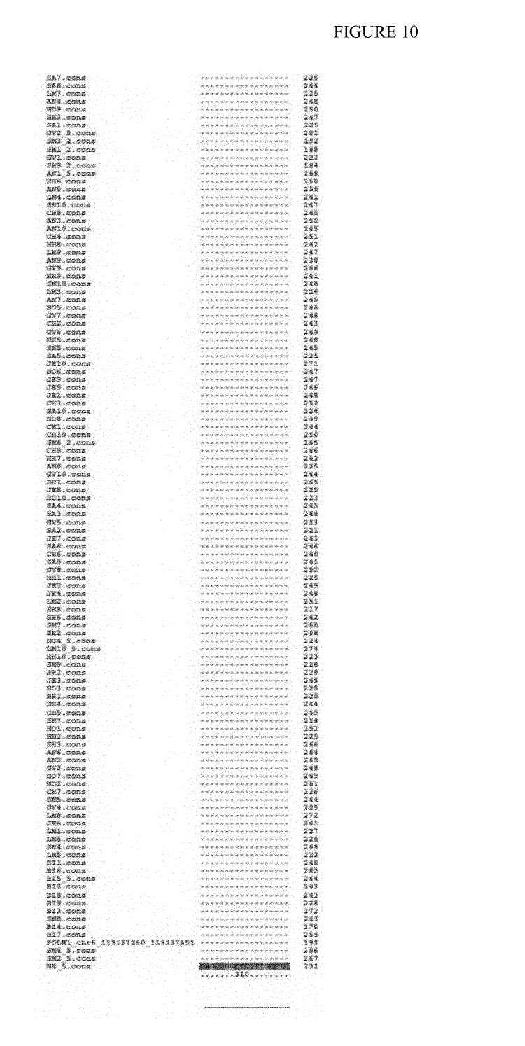

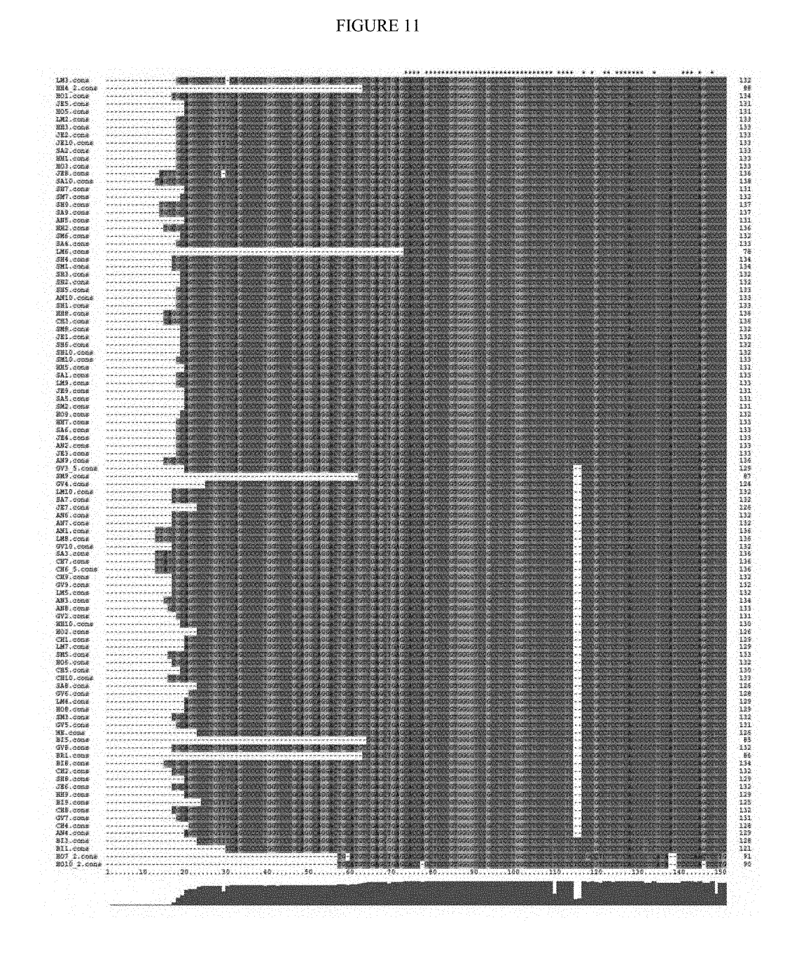

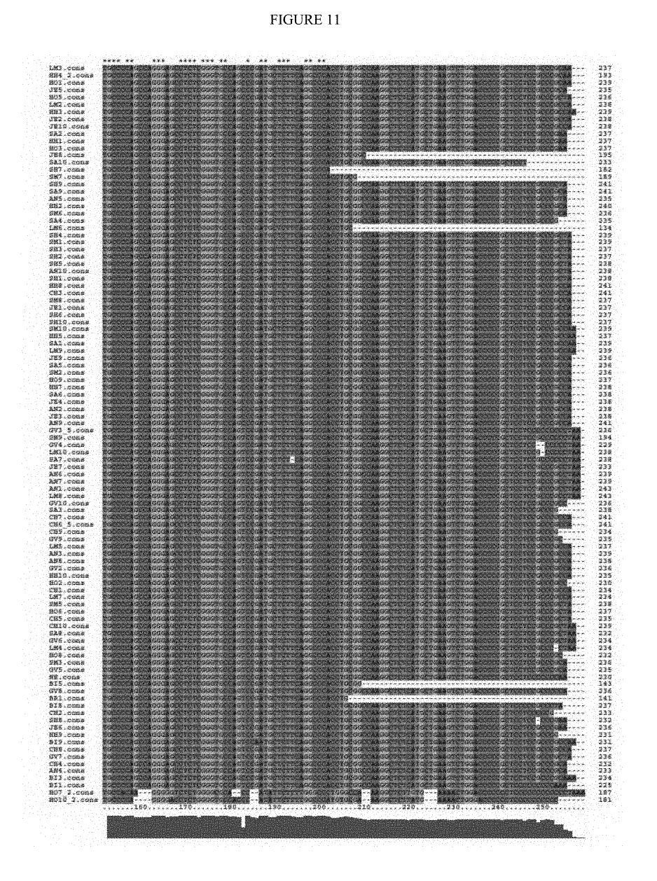

Gene locations of the reference sequence of the BSE-specific motifs were analyzed against 10 main bovine breeds and 3 rare breeds in Alberta, Canada. An example of this analysis is shown in Table 7. Multiple sequence alignments were performed to the reference sequence of the BSE-specific motifs. FIGS. 4-11 show multiple sequence alignments of the reference sequence for ATP8B3, CDK5, CSK, FSD9, LOC507825, NOTUM, POLN1, and POLN2, respectively to the 10 main bovine breeds and 3 lesser bovine breeds. Table 7 shows the results of this analysis for the 8 BSE-specific motifs of Table 4. ATP8B3, CDK5, CSK, FSD9, LOC507825, and NOTUM were conserved in most of the breeds indicating that these motifs are useful for identifying BSE infection across most commercial cattle breeds, including rare cattle breeds. In view of the timed series of sampling, the BSE-specific motifs can be mapped to the timecourse of BSE infection in the animals. See, for example, Tables 1, 2, 4, and 13. A combination of polynucleotides can therefore be selected from the pool of BSE-specific motifs in Tables 1 and 2 that provides for detection of BSE-infected animals at various stages of infection across many, if not all, commercial cattle breeds.

Another aspect of the invention is arrays comprising one or more polynucleotides of the disclosure, PCR primers and/or probes for amplifying and/or detecting polynucleotides of the inventions, and methods of detecting BSE comprising an array or PCR primers and/or probes according to the disclosure.

One or more polynucleotide sequences of the disclosure can be incorporated on a sequence array, such as a biochip, DNA chip, BiofireDX filmarray and other filmarrays, microarray, macroarray, and the like, for screening serum separated from whole blood from cattle for BSE. Alternatively, CNAs can be extracted from the sample for screening on the array. Arrays are generally solid supports upon which a collection of polynucleotides and/or primers and/or probes are placed at defined locations on the array, either by spotting, printing, or direct synthesis. The array can include probes corresponding to one or more of the polynucleotides in Tables 1, 2, or 4 and/or primers and/or probes for amplifying and/or detecting one or more polynucleotides in Tables 1, 2, or 4.

The underlying principle of arrays is base pairing or hybridization i.e., A-T and G-C for DNA, and A-U and G-C for RNA. A sample from an animal or animals is allowed to hybridize with the polynucleotides and/or primers and/or probes on the array providing an expression profile/pattern of CNA. The CNA expression pattern of BSE-specific sequences can be used to determine if an animal is infected with BSE. As the BSE-specific motifs of the disclosure correspond to various stages of BSE infection across many, if not all, commercial cattle breeds, the progression of BSE infection can also be determined by the CNA expression pattern. The array can be prepared by any method known in the art. In some embodiments, a microarray is prepared generally as disclosed in U.S. Pat. No. 7,655,397, the entirety of which is hereby incorporated by reference. The array can be customized to contain a combination of polynucleotides of the disclosure that provides for detection of BSE across many, if not all, commercial cattle breeds. See, for example, Table 1, Table 2, column 6 in Table 4 and Table 7.

In some embodiments, the array comprises at least 4 polynucleotides selected from the polynucleotides in Tables 1, 2, or 4. In another embodiment, the array comprises at least 8 polynucleotides selected from the polynucleotides in Tables 1, 2, or 4. In some embodiments, the array comprises up to 50 polynucleotides, up to 100 polynucleotides, up to 150 polynucleotides, up to 200 polynucleotides, up to 250 polynucleotides, up to 300 polynucleotides selected from Tables 1, 2, or 4. In some embodiments, the array comprises at least 4 polynucleotides selected from Table 4. The array generally includes many copies of the selected polynucleotides to facilitate detection. In some embodiments, the array comprises a million or more copies of each of the selected polynucleotides.

In some embodiments, the array comprises probes for at least 4 polynucleotides selected from the polynucleotides in Tables 1, 2, or 4. In another embodiment, the array comprises probes for at least 8 polynucleotides selected from the polynucleotides in Tables 1, 2, or 4. In some embodiments, the array comprises probes for up to 50 polynucleotides, up to 100 polynucleotides, up to 150 polynucleotides, up to 200 polynucleotides, up to 250 polynucleotides, up to 300 polynucleotides selected from Tables 1, 2, or 4. Probes for detecting polynucleotides of the disclosure can be designed and prepared using conventional methods. In some embodiments, the array comprises probes for at least 4 polynucleotides selected from Table 4. Software for modeling and designing probes, including determining hybridization and annealing conditions, for detecting a specific polynucleotide sequence are publically available, and include for example LightCycler® Probe Design Software (Roche Applied Science), Primer3 (Simgene), and FastPCR (PrimerDigital).

The array can include diagnostic sequences and/or probes for detecting BSE and negative and/or positive control sequences and/or probes. The array can be customized to contain one or more probes and/or sequences for detecting a combination of polynucleotides of the disclosure that provides for detection of BSE across many, if not all, commercial cattle breeds. See, for example, Table 1, Table 2, column 6 in Table 4, and Table 7. In some embodiments, in addition to one or more probes for BSE, the array comprises probes for detecting one or more additional diseases or disorders in cattle thereby providing a diagnostic panel for screening cattle for multiple diseases or disorders, including BSE.

Polynucleotides of the disclosure can be amplified and/or detected via PCR, including but not limited to real-time PCR, multiplex PCR, nested PCR, solid phase PCR, miniprimer PCR, and the like. Primers and probes for amplifying and/or detecting polynucleotides of the disclosure can be designed and prepared using conventional methods. Software for modeling and designing primers and probes, including determining hybridization, melting, annealing, and/or extensions conditions, for amplifying and/or detecting a specific polynucleotide sequence are publically available, and include for example LightCycler® Probe Design Software (Roche Applied Science), Primer3 (Simgene), and FastPCR (PrimerDigital). PCR conditions generally include the presence of four different nucleotide bases (adenosine, cytidine, guanosine, thymidine/uridine) and at least one polymerization-inducing agent such as a reverse transcriptase or a DNA polymerase. The primers are generally present in a suitable buffer, which may include constituents, which are co-factors or affect conditions such as pH and the like at various suitable temperatures. The primers are preferably single-strand nucleotide sequences, such that amplification efficiency of the desired polynucleotide is optimized. Double-stranded nucleotide sequences can also be utilized. The primers are typically at least about 15 nucleotides. In some embodiments, the primers can have a length of from about 15 to about 30, about 15 to about 50, about 15 to about 75, about 15 to about 100, or about 15 to about 500 nucleotides.

In some embodiments, primer sets are designed to amplify one or more of the BSE-specific polynucleotides in Tables 1, 2, or 4 and then the PCR products of the primer sets are screened for BSE-specific sequences on an array as described herein.

Diagnostic kits comprising one or more primer pairs, and optional probes, for amplifying and detecting one or more polynucleotides of the disclosure are also provided. The kit can optionally include nucleotide bases (adenosine, cytidine, guanosine, thymidine/uridine) and at least one polymerization-inducing agent such as a reverse transcriptase or a DNA polymerase. The kit can optionally include a suitable primer buffer, which may include constituents which are co-factors or affect conditions such as pH and the like at various suitable temperatures. The kit can optionally include an array as described herein.

The primers provided in the diagnostic kit are generally provided in pairs (forward primer and reverse primer) for amplifying a specific polynucleotide sequence. These primers can be used to amplify and detect CNAs in blood serum from a bovine, or any other appropriate biological sample from the bovine that may contain CNAs. Alternatively, CNAs can be extracted from the sample and then amplified by PCR using a diagnostic kit of the disclosure. The CNA expression pattern of BSE-specific sequences detected by the diagnostic kit can be used to determine if an animal is infected with BSE. As the BSE-specific motifs of the disclosure correspond to various stages of BSE infection across many, if not all, commercial cattle breeds, the progression of BSE infection can also be determined by the CNA expression pattern. The diagnostic kit can be customized to contain a combination of primers and/or probes for amplifying and/or detecting a combination polynucleotides of the disclosure that provides for detection of BSE across many, if not all, commercial cattle breeds. See, for example, Table 1, Table 2, column 6 in Table 4, and Table 7.

In some embodiments, the kit comprises primers for amplifying at least 4 polynucleotides selected from the polynucleotides in Tables 1, 2, or 4, and optionally one or more probes for detecting the amplified product. In another embodiment, the kit comprises primers for amplifying at least 8 polynucleotides selected from the polynucleotides in Tables 1, 2, or 4, and optionally one or more probes for detecting the amplified product. In some embodiments, the kit comprises primers for amplifying up to 50 polynucleotides, up to 100 polynucleotides, up to 150 polynucleotides, up to 200 polynucleotides, up to 250 polynucleotides, up to 300 polynucleotides selected from Tables 1, 2, or 4, and optionally one or more probes for detecting the amplified product. In some embodiments, the kit comprises primers for amplifying at least 4 polynucleotides selected from Table 4, and optionally one or more probes for detecting the amplified products. In some embodiments, the diagnostic kit comprises primers, and optionally probes, for amplifying and detecting one or more polynucleotides associated with additional diseases or disorders in cattle thereby providing a diagnostic panel for screening cattle for multiple diseases or disorders, including BSE.

The present invention is described in the following Examples, which are set forth to aid in an understanding of the invention, and should not be construed to limit in any way the scope of the invention as defined in the claims which follow thereafter.

### EXAMPLES

### Example 1

### Identification of Unique BSE-Associated Motifs in CNAS

One hundred seventy-seven blood serum samples from Friedrich-Loeffler-Institut in Riems, Germany were analyzed as shown in FIG. 1. The sample set contained 115 C-type BSE-infected and 62 control samples collected over 56 months from Simmental cattle breed during the German BSE pathogenesis study between 2003 and 2007. Blood samples from control and BSE inoculated animals were collected at 0, 1, 4, 8, 12, 20, 24, 28, 32, 36, 40, 44, 48, 52, and 56 months post infection (Table 8). Seventeen blood samples from a Gausepohl cattle slaughterhouse sample from Gottingen, Germany were included as an additional control set. Libraries of CNAs were isolated and amplified from these samples and then sequenced using a Roche 454 DNA sequencer at the McGill DNA Sequence Center in Montreal, Canada. Altogether 10 full runs of high-throughput titanium 454 sequencing were completed and resulted in U.S. Pat. No. 7,669,739 sequences—raw reads, with more than six million of good quality (Table 9).

For atypical BSE, 61 serum samples from Friedrich-Loeffler-Institut in Riems, Germany were analyzed. The sample set contained 21 L-Type and 21 H-Type intra-cerebrally infected and 19 control samples collected from 13 Holstein-Friesian heifer cattle during 18 months (see Tables 10-12). Blood samples from L-type and H-type inoculated animals were collected at 0, 4, 5, 8, 12, 14, 15, and 16 months post infection. (Tables 10 and 11). Bloods samples form control animals were collected at 0, 6, 12, and 18 months (Table 12). Preparation of CNA library from the samples and 454 sequencing of the libraries was performed as described for the C-Type BSE samples. Altogether 3 full runs of high-throughput titanium 454 sequencing were completed for the atypical BSE and resulted in U.S. Pat. No. 2,044,325 sequences—raw reads (Table 9).

In order to confirm the specificity of BSE-related CNA motifs, ten control animals were overdosed with amprolium to induce polioencephalomalacia (PEM) in the animals. After 28 days, all of the amprolium treated animals developed severe PEM. Blood samples were collected from the animals every 2nd or 3rd day over the period of 28 days. Approximately 150 samples were collected for each animal. All of the animals were then dissected with special emphasis on brain, cervical and lumbar spinal cord, and several different organs. The histological impression of brain from these animals showed severe necrosis. CNA samples were prepared from time point 0 (control) and day 28 of each of the 10 animals.

Libraries of CNAs were isolated and amplified from blood serum samples from normal animals and PEM animals and then sequenced as described for the C-Type BSE samples. The 454 high throughput sequencing of 20 PEM samples resulted in 1 Million sequencing reads (Table 9).

A total of approximately 10 million 454 sequencing reads were produced for animals infected with BSE, normal animals, and PEM animals. Table 9 shows the number of raw read counts from 454 sequencing, the number of samples sequenced, and the number of animals from which the samples were collected for C-Type BSE, Atypical BSE, and PEM animals.

The 454 sequencing results analyzed according to the bioinformatics processing all-against-all clustering scheme as shown in FIG. 2 and described in Gordon et al, 2009, Nucleic Acids Res., 37(2):550-556. In silico subtraction of normal reads from the BSE reads provided a pool of candidate BSE-specific motifs. Further in silico subtraction of PEM specific motifs from the pool of candidate BSE-specific motifs provided a pool of BSE-specific motifs that could be distinguished from general brain stress motifs. The identified BSE-specific motifs are shown in Table 2. The nucleotide sequence of each motif is shown in Table 2 by reference to the nucleic acid sequence numbering of the reference region identified by accession number.

Analysis of the Roche 454 sequencing results revealed that the genomic coverage per sample was quite low relative to the complexity of the CNA molecule population, based on the low redundancy of the sequencing results. Using the all-against-all clustering approach, 29 multi-animal infected-only clusters of sequences were identified (out of U.S. Pat. No. 7,711,086 total clusters). A minimal set of 8 unique motifs (cluster consensuses) was identified that covered all infected animals (symptomatic and asymptomatic). The nucleotide sequence of each of the 8 motifs is shown in Table 4 by reference to the nucleic acid sequence numbering of the reference region identified by accession number.

Although nearly 90% of the 454 sequencing reads mapped significantly to the bovine genome, issues with the quality of reference genome assembly were evident. The eight most frequent infected-only motifs were used to design PCR products amplified from genomic DNA for Sanger sequencing. A motif from the 454 sequencing data that was not found to occur in the reference genome (motif 1 in Table 2) was readily amplified from genomic DNA and conserved across breeds. Another identified 454 sequencing pattern (motif 3 in Table 2) was found to have a discontinuous match in the reference, yet was both amplifiable and highly conserved.

Fourteen of the 29 infected-only 454 sequencing read clusters corresponded to regions proximal to genes in the reference genome. Nine of these genes have some molecular characterization, which is summarized in Table 4, but none are directly linked to known prion biology. In order to determine if microbial infections were affecting the results, the 45,000 clustered sequences (0.5%) without any hits to the bovine genome were run against the NCBI public DNA sequence database (“nt”). The only significant spike in non-bovine sequences was found at month 32, where 1100 human contamination sequences were detected. Low levels of Ralstonia species, Propionibacterium acnes, and bovine parvovirus were detected in all months of the experiment, including pre-inoculation. No new microbial species were detected during the course of the experiments.

In order to obtain deeper sequencing results, Illumina sequencing was performed. Twenty-four C-Type infected samples, 16 control samples, 21 L-Type infected samples, 21 H-Type infected samples, and 19 atypical controls were selected for Illumina sequencing based on the analysis of the 454 sequencing results. CNA library preparation and amplification, as well as the Illumina sequencing of the libraries, was performed by Chronix Biomedical Inc. (San Jose, Calif.). More than 4 billion sequencing reads were obtained from the Illumina sequencing (see Table 9). Illumina sequencing of 78 samples from PEM control animals resulted in nearly 3 billion read counts (Table 9).

The Illumina sequencing results were analyzed according to the bioinformatics processing genome mapping scheme shown in FIG. 3 and described herein. In silico subtraction of normal reads from the BSE reads provided a pool of candidate BSE-specific motifs. Further in silico subtraction of PEM specific motifs from the pool of candidate BSE-specific motifs provided a pool of BSE-specific motifs that could be distinguished from general brain stress motifs. The identified BSE-specific motifs are shown in Table 1. The nucleotide sequence of each motif is shown in Table 1 by reference to the nucleic acid sequence numbering of the reference region identified by accession number.

Analysis of the Illumina sequencing results yielded 306 genomic hotspots for mapped CNAs, which were only found in animals infected with BSE, or in a few cases in the PEM animals. These regions were completely barren in all control samples. One hundred ninety-three of the CNA hotspots were inside or proximal to known genes in the genome (see Table 1). Functional annotation clustering of the genes using the DAVID system [Huang et al., 2008, Bioinformation, 2:428-430] yielded one significantly (p-value=0.0004, Benjamini FDR=0.03) enriched pathway: the phosphatidylinositol signaling system. Regions in six genes in this pathway (DGKQ, INPP5D, IMPA1, PI4KA, PRKCG, and PIK3C2B) are hotspots for CNAs from only infected animals. A phosphatidylinositol glycolipid is responsible for anchoring the prion protein to the surface of mammalian cells [Stahl et al., 1987, Cell, 51:229-240], thus some of the identified infected-only hotspots are directly linked to established prion biology.

### Example 2

### Characterization of Genetic Stability of BSE-Specific Motifs in Albertan Cattle Breeds

For the eight most prevalent CNA motifs identified in the high-throughput DNA sequencing phase (Table 4), PCR primers were designed and the candidate regions were amplified from chromosomal DNA obtained from blood or semen samples from 40 commercial cattle breeds in Alberta, Canada. A listing of the breeds and type of sample used to obtain the chromosomal DNA is shown in Table 3. The DNA fragments were subsequently sequenced in 10 main Albertan breeds and two breeds containing Bos indicus and Plains bison. A total of 1016 DNA sequences were produced using the Sanger DNA sequencing protocol (see Table 7). Six CNA patterns (ATP8B3, CDK5, CSK, FZD9, LOC507825, and NOTUM) produced nearly perfect conservation in Albertan breeds, while the other two patterns (POLN1 and POLN2) require further confirmation.

The identified CNA sequences (Tables 1 and 2) were only be found in animals infected with the C-Type of BSE, some instances of animals infected with the atypical forms of BSE and very rarely in PEM animals, but never in normal control animals. Eight of the main regions (see Table 4) were analyzed as discussed above. A total of four of the motifs, which occurred in the majority of animals from the tested breeds, is statistically sufficient to cover all bovine breeds infected with the C-Type of BSE. The case of the atypical BSE forms is more complex as no pattern set was identified, which could cover all animals infected with the atypical form of BSE universally. Given the fact that infection with C-Type of BSE always results in clinical signs, and the eventual death of the affected animal, while this is not (always) the case for animals infected with the atypical forms of BSE, the inventors' results are well-aligned with other studies [Balkema-Buschmann et al., 2011, J. Toxicol. Environ. Health, 72(2-4)L103-9].

The fact that more than one pattern is necessary to cover all of the animals infected with C-Type BSE points to the complexity of the host response to BSE infection. Several metabolic pathways seem to be involved in the mechanism. Therefore screening approaches using the identified BSE-specific motifs should be based on multiple motifs. As there is more than 300 to draw from, and only four needed to cover all the infected animals in the inventors' study, the motif set identified thus far is sufficient for serum-based screening approaches.

The use of circulating nucleic acids as markers for chronic disease and its progress is now well established in the scientific literature. Therefore, the inventors' findings for the C-Type of BSE, which as discussed above, always results in clinical symptoms and the affiliated stress on the animal's body are well-aligned with the findings related to diseases such as cancers and infections, such as aspergillosis. In essence, the apoptosis of cells caused by the effects of BSE causes the release of specific CNA molecules into the blood of the infected animals and these disease specific CNA molecules can be identified and used as described herein as markers of BSE infection.

The inventors' characterization of the sequence variation in the patterns using DNA extracted from Albertan breeds, with Bison and Zebu as the outliers included, showed that the motif sequences are virtually identical in all cattle breeds used commercially in Alberta (see Table 7). Given the variability of the breeds used in the study, the identified BSE-specific motifs can be used to identify C-Type BSE in all breeds worldwide. This is an important facet of knowledge needed for the development of a testing system based on the CNA patterns. Due to the specificity of the patterns and lack of sequence variation amongst the Albertan breeds, the BSE-specific sequences identified herein provide the basis of a monitoring system for the C-Type form of BSE.

### Example 3

### Embodiments

1. A method of detecting bovine spongiform encephalopathy in a bovine, comprising analyzing a diagnostic sample of the bovine for the presence of one or more of the polynucleotides selected from Table 1, 2, or 4.

2. The method of embodiment 1, wherein the step of analyzing the sample comprises: isolating nucleic acid from the sample; amplifying the isolated nucleic acid using primers that are specific for or capable of amplifying a sequence corresponding to the selected polynucleotides; and sequencing the amplification products.

3. The method of embodiment 1, wherein the step of analyzing the sample further includes obtaining the sample from the bovine.

4. The method of embodiment 1, wherein the nucleic acid comprises genomic DNA, mRNA, or cDNA obtained from mRNA.

5. The method of embodiment 1, wherein the step of determining the presence of the one or more polynucleotides comprises use of at least one of a PCR-based detection method and a hybridization-based method.

6. The method of embodiment 1, wherein the one or more of the polynucleotides comprise one or more nucleotide motifs.

7. The method of embodiment 6, wherein the nucleotide motif comprises ATP8B3, CDK5, CSK, FSD9, LOC507825, NOTUM, POLN1, or POLN2.

8. The method of embodiment 1, wherein the sample is obtained from a live cow.

9. The method of embodiment 1, wherein the steps are repeated on a periodic basis.

10. The method of embodiment 1, wherein the periodic basis comprises an annual basis.

11. The method of embodiment 1, wherein the nucleotide comprises a circulating nucleic acid.

12. Use of an oligonucleotide capable of identifying at least one of the polynucleotides selected from Table 1, 2, or 4 from a sample obtained from a bovine.

13. The use of embodiment 12, wherein the oligonucleotide comprises a DNA or RNA probe.

14. The use of embodiment 12, wherein the oligonucleotide is labelled with a detectable marker.

15. The use of embodiment 12, wherein the detectable marker comprises a radioactive marker or fluorescent marker.

16. The use of embodiment 12, wherein the selected polynucleotide comprises a nucleotide motif.

17. The use of embodiment 12, wherein the nucleotide motif comprises ATP8B3, CDK5, CSK, FSD9, LOC507825, NOTUM, POLN1, or POLN2.

18. The use of embodiment 12, wherein the oligonucleotide is mounted in an array.

19. The use of embodiment 12, wherein the sample is obtained from a live bovine.

20. A system for detecting bovine spongiform encephalopathy comprising an array having one or more probes for detecting polynucleotides selected from Table 1, 2, or 4.

21. The system of embodiment 20, comprising at least four different probes.

22. The system of embodiment 20, comprising at least eight different probes.

23. The system of embodiment 20, further comprising one or more probes for detecting polynucleotides associated with one or more bovine diseases or disorders other than BSE.

24. The system of embodiment 20, wherein the polynucleotides comprises one or more nucleotide motifs.

25. The system of embodiment 20, wherein the one or more nucleotide motifs comprises ATP8B3, CDK5, CSK, FSD9, LOC507825, NOTUM, POLN1, or POLN2.

26. The system of any one of embodiments 20 to 25, comprising a microarray, gene chip, DNA chip, or filmarray.

27. A kit for detecting bovine spongiform encephalopathy in a bovine comprising at least one primer pair for amplifying a polynucleotide selected from Table 1, 2, or 4.

28. The kit of embodiment 27, wherein the selected polynucleotide comprises a nucleotide motif.

29. The kit of embodiment 27, comprising primer pairs for amplifying at least four polynucleotides.

30. The kit of embodiment 27, comprising primer pairs for amplifying at least 8 polynucleotides.

31. The kit of embodiment 27, wherein the polynucleotides comprises one or more nucleotide motifs.

32. The kit of embodiment 27, wherein the one or more nucleotide motifs comprises ATP8B3, CDK5, CSK, FSD9, LOC507825, NOTUM, POLN1, or POLN2.

33. The kit of embodiment 27, wherein the bovine is a live bovine.

All publications mentioned herein are hereby incorporated by reference in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

Specific examples of methods and kits have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

The embodiments of the invention described above are intended to be exemplary only. Those skilled in this art will understand that various modifications of detail may be made to these embodiments, all of which come within the scope of the invention.

Lengthy table referenced here
US20190226025A1-20190725-T00001
Please refer to the end of the specification for access instructions.

Lengthy table referenced here
US20190226025A1-20190725-T00002
Please refer to the end of the specification for access instructions.

Lengthy table referenced here
US20190226025A1-20190725-T00003
Please refer to the end of the specification for access instructions.

Lengthy table referenced here
US20190226025A1-20190725-T00004
Please refer to the end of the specification for access instructions.

Lengthy table referenced here
US20190226025A1-20190725-T00005
Please refer to the end of the specification for access instructions.

Lengthy table referenced here
US20190226025A1-20190725-T00006
Please refer to the end of the specification for access instructions.

Lengthy table referenced here
US20190226025A1-20190725-T00007
Please refer to the end of the specification for access instructions.

Lengthy table referenced here
US20190226025A1-20190725-T00008
Please refer to the end of the specification for access instructions.

Lengthy table referenced here
US20190226025A1-20190725-T00009
Please refer to the end of the specification for access instructions.

Lengthy table referenced here
US20190226025A1-20190725-T00010
Please refer to the end of the specification for access instructions.

Lengthy table referenced here
US20190226025A1-20190725-T00011
Please refer to the end of the specification for access instructions.

Lengthy table referenced here
US20190226025A1-20190725-T00012
Please refer to the end of the specification for access instructions.

Lengthy table referenced here
US20190226025A1-20190725-T00013
Please refer to the end of the specification for access instructions.

Lengthy table referenced here
US20190226025A1-20190725-T00014
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20190226025A1). An electronic copy of the table will also be available from the USPTO upon request and payment of the fee set forth in 37 CFR 1.19(b)(3).

1. A method of detecting bovine spongiform encephalopathy (BSE) in a bovine, comprising analyzing a biological sample of the bovine for the presence of one or more of the polynucleotides selected from Table 1, 2, or 4, wherein the presence of the one or more polynucleotides in the biological sample is indicative that the bovine has BSE.

2. The method of claim 1, wherein the step of analyzing the sample comprises:
obtaining the sample from the bovine;
isolating nucleic acid from the sample;
amplifying the isolated nucleic acid using primers that are specific for or capable of amplifying a sequence corresponding to the selected polynucleotides; and
sequencing the amplification products.

3. The method of claim 1, wherein the nucleic acid comprises genomic DNA, mRNA, cDNA obtained from mRNA, or circulating nucleic acid.

4. The method of claim 1, wherein the one or more of the polynucleotides comprise one or more nucleotide motifs.

5. The method of claim 6, wherein the nucleotide motif comprises ATP8B3, CDK5, CSK, FSD9, LOC507825, NOTUM, POLN1, or POLN2.

6. The method of claim 1, wherein the sample is obtained from a live bovine.

7. Use of an oligonucleotide capable of identifying at least one of the polynucleotides selected from Table 1, 2, or 4 from a sample obtained from a bovine to detect bovine spongiform encephalopathy.

8. The use of claim 7, wherein the oligonucleotide comprises a DNA or RNA probe.

9. The use of claim 7, wherein the oligonucleotide is labelled with a detectable marker.

10. The use of claim 7, wherein the detectable marker comprises a radioactive marker or fluorescent marker.

11. The use of claim 12, wherein the nucleotide motif comprises ATP8B3, CDK5, CSK, FSD9, LOC507825, NOTUM, POLN1, or POLN2.

12. The use of claim 7, wherein the oligonucleotide is mounted in an array.

13. The use of claim 7, wherein the sample is obtained from a live bovine.

14. A system for detecting bovine spongiform encephalopathy (BSE) comprising an array having one or more probes for detecting polynucleotides selected from Table 1, 2, or 4.

15. The system of claim 14, comprising at least four different probes.

16. The system of claim 14, comprising at least eight different probes.

17. The system of claim 14, further comprising one or more probes for detecting polynucleotides associated with one or more bovine diseases or disorders other than BSE.

18. The system of claim 14, wherein the polynucleotides comprises one or more nucleotide motifs.

19. The system of claim 14, wherein the one or more nucleotide motifs comprises ATP8B3, CDK5, CSK, FSD9, LOC507825, NOTUM, POLN1, or POLN2.

20. The system of claim 14, comprising a microarray, gene chip, DNA chip, or filmarray.
